(19) United States
(12) Patent Application Publication
(10) Pub. No.: US 2022/0175834 A1
(43) Pub. Date: Jun. 9, 2022

(54) METHODS FOR CULTURING IMMUNE CELLS

(71) Applicant: LYELL IMMUNOPHARMA, INC., South San Francisco, CA (US)

(72) Inventors: Suman Kumar VODNALA, San Mateo, CA (US); Yogin PATEL, South San Francisco, CA (US); Nicholas P. RESTIFO, San Francisco, CA (US)

(73) Assignee: LYELL IMMUNOPHARMA, INC., South San Francisco, CA (US)

(21) Appl. No.: 17/456,374

(22) Filed: Nov. 23, 2021

Related U.S. Application Data
(60) Provisional application No. 63/273,138, filed on Oct. 28, 2021.
(60) Provisional application No. 63/181,218, filed on Apr. 28, 2021.
(60) Provisional application No. 63/167,592, filed on Mar. 29, 2021.
(60) Provisional application No. 63/153,922, filed on Feb. 25, 2021.
(60) Provisional application No. 63/146,477, filed on Feb. 5, 2021.
(60) Provisional application No. 63/198,933, filed on Nov. 23, 2020.
(60) Provisional application No. 63/165,023, filed on Mar. 23, 2021.

(51) Int. Cl. A61K 35/17; C12N 5/0783; A61P 35/00

(52) U.S. Cl. CPC A61K 35/17; C12N 5/0636; A61P 35/00; C12N 2501/2302; C12N 2501/515; C12N 2501/2315; C12N 2501/2321; C12N 2501/599; C12N 2501/51; C12N 2501/2307

(57) ABSTRACT

The preset disclosure provides methods of culturing TILs in a medium comprising at least about 30 mM to at least about 100 mM potassium ion. In some aspects, the methods disclosed herein enhance expansion of CD8⁺ TILs, relative to CD4⁺ TILs. In some aspects, the methods further increase the number of less-differentiated cells, e.g., less-differentiated TILs, in the population of cells. In some aspects, the methods disclosed herein enrich for tumor-reactive, e.g., tumor specific, TILs such that clonal diversity is preserved. In some aspects, the cells, e.g., the TILs, are administered to a subject in need thereof.

$$17667030.1$$

### CROSS-REFERENCE TO RELATED APPLICATIONS

This application claims priority to and benefit of U.S. Provisional Application Nos. 63/198,933 filed Nov. 23, 2020; 63/146,477 filed Feb. 5, 2021; 63/153,922 filed Feb. 25, 2021; 63/165,023 filed Mar. 23, 2021; 63/167,592 filed Mar. 29, 2021; 63/181,218 filed Apr. 28, 2021; and 63/273,138 filed Oct. 28, 2021; each of which is incorporated by reference herein in its entirety.

### REFERENCE TO SEQUENCE LISTING SUBMITTED ELECTRONICALLY

The content of the electronically submitted sequence listing in ASCII text file (Name: 4385_0430008_Seqlisting_ST25.txt; Size: 1,999 bytes; and Date of Creation: Nov. 23, 2021) filed with the application is herein incorporated by reference in its entirety.

### FIELD

The present disclosure relates to compositions comprising tumor infiltrating lymphocytes (TILs) and methods of culturing the cells. In some aspects, the methods disclosed herein preferentially promote the enrichment of oligoclonal or polyclonal tumor reactive (e.g., tumor specific) stem-like T-cells, e.g., TILs characterized by being less differentiated. Cells cultured using the methods disclosed herein can be used for various cell therapies, including, but not limited, to adoptive cell therapies such as autologous T cell therapies.

### BACKGROUND

The use of immunotherapy strategies has demonstrated considerable clinical efficacy in the treatment of certain types of advanced cancer. However, in spite of notable successes, the vast majority of patients with advanced cancers still do not benefit from immunotherapy treatments and will eventually succumb to their illness. A key limitation to cell therapy techniques such as adoptive cell therapies including chimeric antigen receptor (CAR) and engineered TCR T cells is a lack of suitable tumor targets, which may contribute to the lack of widespread clinical responses observed in patients with solid cancers that have been treated with, e.g., CAR T cells.

Another approach that has had some success in mediating clinical response in patients with advanced cancer is the isolation, expansion, and infusion of autologous tumor infiltrating lymphocytes (TILs). TILs are heterogenous, with variable compositions of tumor-reactive and irrelevant or suppressive T cells. The tumor-reactive populations are frequently highly antigen-experienced, resulting in cell products that are in a pre-dysfunctional state with limited functionality.

Traditional methods of culturing and expanding TILs have been found to lead to terminal differentiation of the TILs, resulting in poor persistence of the TILs upon transfer to patients. See, e.g., Rosenberg et al., Clinical Cancer Research 17(13):4550-557 (2011). Further, current methods of expanding heterogenous TIL populations from tumor fragments yield populations of TILs with reduced polyclonality and the loss of many tumor-dominant T cell clones (see, e.g., Poschke, et al., Clin. Cancer Res. (2020), which is incorporated by reference herein in its entirety). As a result, generating such TIL-derived infusion products often results in loss of tumor-specific T cells during expansion and an ill-defined mix of immune cells at various states of differentiation, which are ineffective at eradicating solid tumors. To be curative, TILs with enhanced self-renewing stem/effector properties are needed. Moreover, methods have not yet been described for obtaining an expanded population of less-differentiated TILs with a high level of clonal diversity that retain the ability to further divide and target and kill cancer cells.

To date, these and other critical limitations have curtailed the use of TILs as an effective therapeutic, making it difficult to obtain a sufficient number of tumor-reactive TILs for use in T cell therapy. As such, there remains a need in the art for improved methods of preparing TIL compositions and therapies using the same.

### BRIEF SUMMARY

Some aspects of the present disclosure are directed to a method of culturing tumor infiltrating lymphocytes (TILs) ex vivo or in vitro comprising placing a heterogeneous population of TILs in a metabolic reprogramming medium (“MRM”) comprising potassium ion at a concentration of about 30 mM to about 100 mM. In some aspects, the heterogeneous population of TILs is enriched in CD8⁺ TILs after being placed in the MRM.

Some aspects of the present disclosure are directed to a method of increasing a number or percentage of CD8⁺ TILs ex vivo or in vitro comprising culturing a heterogeneous population of TILs in an MRM comprising potassium ion at a concentration of about 30 mM to about 100 mM.

Some aspects of the present disclosure are directed to a method of preparing a CD8⁺-enriched population of TILs, comprising culturing a heterogeneous population of TILs ex vivo or in vitro in an MRM comprising potassium ion at a concentration of about 30 mM to about 100 mM.

In some aspects, the heterogeneous population of TILs comprises CD4⁺ TILs and CD8⁺ TILs. In some aspects, the heterogeneous population of TILs is obtained from one or more tumor sample obtained from a subject. In some aspects, the tumor sample subjected to an initial TIL culture. In some aspects, the initial TIL culture comprises culturing the tumor sample in the MRM.

In some aspects, the MRM further comprises IL-2 during the initial TIL culture. In some aspects, the MRM further comprises IL-7, IL-15, IL-21, or any combination thereof during the initial TIL culture. In some aspects, the MRM comprises IL-2 and IL-21 during the initial TIL culture. In some aspects, the initial TL culture lasts at least about 14-19 days. In some aspects, the initial TIL culture lasts at least about 11 days. In some aspects, the initial TIL culture lasts at least about 14 days. In some aspects, the proportion of CD8⁺ TILs to non-CD8⁺ TILs is increased following the initial TIL culture, as compared to the proportion of CD8⁺ TILs to non-CD8⁺ TILs prior to the initial TIL culture. In some aspects, the TILs are stimulated following the initial TIL culture. In some aspects, the TILs are stimulated by culturing the TILs with a CD3 agonist and/or a CD28 agonist.

In some aspects, the tumor sample comprises a tumor biopsy. In some aspects, the tumor sample is fragmented prior to culturing. In some aspects, the tumor sample is dissociated prior to culturing.

In some aspects, following culture of the heterogeneous population of TILs, at least about 30%, at least about 35%, at least about 40%, at least about 45%, at least about 50%, at least about 55%, at least about 60%, at least about 65%, at least about 70%, at least about 75%, or at least about 80% of the TILs in the population are CD8⁺ TILs. In some aspects, following culture of the heterogeneous population of TILs, at least about 50% of the TILs in the population are CD8⁺ TILs.

In some aspects, the MRM further comprises sodium ion, calcium ion, glucose, or any combination thereof.

In some aspects, the MRM further comprises a cell expansion agent. In some aspects, the cell expansion agent comprises a GSK3B inhibitor, an ACLY inhibitor, a PI3K inhibitor, an AKT inhibitor, or any combination thereof. In some aspects, the PI3K inhibitor comprises LY294002, pictilisib, CAL101, IC87114, or any combination thereof. In some aspects, the AKT inhibitor comprises MK2206, A443654, AKTi-VIII, or any combination thereof.

In some aspects, the concentration of potassium ion is at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, at least about 85 mM, at least about 90 mM, at least about 95 mM, or at least about 100 mM. In some aspects, the concentration of potassium ion is about 30 mM to about 100 mM, about 30 mM to about 90 mM, about 30 mM to about 80 mM, about 30 mM to about 70 mM, about 30 mM to about 60 mM, about 30 mM to about 50 mM, about 40 mM to about 100 mM, about 40 mM to about 90 mM, about 40 mM to about 80 mM, about 40 mM to about 70 mM, about 40 mM to about 60 mM, or about 40 mM to about 50 mM. In some aspects, the concentration of potassium ion is about 40 mM to about 90 mM. In some aspects, the concentration of potassium ion is about 50 mM to about 90 mM. In some aspects, the concentration of potassium ion is about 50 mM to about 80 mM.

In some aspects, the MRM further comprises sodium ion. In some aspects, the concentration of the sodium ion is from about 25 mM to about 100 mM. In some aspects, the concentration of the sodium ion is from about 30 mM to about 40 mM, about 30 mM to about 50 mM, about 30 mM to about 60 mM, about 30 mM to about 70 mM, about 30 mM to about 80 mM, about 40 mM to about 50 mM, about 40 mM to about 60 mM, about 40 mM to about 70 mM, about 40 mM to about 80 mM, about 50 mM to about 55 mM, about 50 mM to about 60 mM, about 50 mM to about 65 mM, about 50 mM to about 70 mM, about 50 mM to about 75 mM, about 50 mM to about 80 mM, about 55 mM to about 60 mM, about 55 mM to about 65 mM, about 55 mM to about 70 mM, about 55 mM to about 75 mM, about 55 mM to about 80 mM, about 60 mM to about 65 mM, about 60 mM to about 70 mM, about 60 mM to about 75 mM, about 60 mM to about 80 mM, about 70 mM to about 75 mM, about 70 mM to about 80 mM, or about 75 mM to about 80 mM. In some aspects, the concentration of the sodium ion is about 30 mM, about 35 mM, about 40 mM, about 45 mM, about 50 mM, about 55 mM, about 60 mM, about 65 mM, about 70 mM, about 75 mM, or about 80 mM. In some aspects, the concentration of the sodium ion is about 55 mM. In some aspects, the concentration of the sodium ion is about 60 mM. In some aspects, the concentration of the sodium ion is about 65 mM.

In some aspects, the MRM further comprises glucose. In some aspects, the concentration of glucose is more than about 10 mM. In some aspects, the concentration of glucose is from about 10 mM to about 25 mM, about 10 mM to about 20 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM. In some aspects, the concentration of glucose is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM.

In some aspects, the MRM further comprises calcium ion. In some aspects, the concentration of calcium ion is more than about 0.4 mM. In some aspects, the concentration of calcium ion is from about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.3 to about 1.4 mM, about 1.3 to about 1.5 mM, about 1.3 to about 1.6 mM, about 1.3 to about 1.7 mM, about 1.3 to about 1.8 mM, about 1.4 to about 1.5 mM, about 1.4 to about 1.6 mM, about 1.4 to about 1.7 mM, about 1.4 to about 1.8 mM, about 1.5 to about 1.6 mM, about 1.5 to about 1.7 mM, about 1.5 to about 1.8 mM, about 1.6 to about 1.7 mM, about 1.6 to about 1.8 mM, or about 1.7 to about 1.8 mM. In some aspects, the concentration of calcium ion is about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM.

In some aspects, the MRM comprises about 40 mM to about 90 mM potassium ion and (i) about 40 mM to about 80 mM sodium ion; (ii) about 10 mM to about 24 mM glucose; (iii) about 0.5 mM to about 2.8 mM calcium ion; or (iv) any combination of (i)-(iii).

Some aspects of the present disclosure are directed to a method of expanding TILs obtained from a human subject comprising: culturing the TILs in an initial TIL culture media; culturing the TILs in a secondary TIL culture media; culturing the TILs in a third (or final) TIL culture media, wherein the initial TIL culture media, the secondary TIL expansion media, and/or the third TIL expansion media are MRM. In some aspects, the initial TIL culture media and the secondary TIL expansion media are hyperkalemic and the third TIL expansion media are not hyperkalemic. In some aspects, the initial TIL culture media further comprise IL-2. In some aspects, the initial TIL culture media further comprise IL-21. In some aspects, the initial TIL culture media further comprise a T cell supplement, a serum replacement, glutamine, a glutamine substitute (e.g., Glutamax (L-alanine-L-glutamine)), non-essential amino acids, antibiotics (e.g., Penicillin, Streptomycin, or both), an anti-fungal agent (e.g., FUNGIN™), and/or sodium pyruvate.

In some aspects, the TILs are cultured in the initial TIL culture media for at least about 10 days, at least about 11 days, at least about 1 week, at least about 2 weeks, or at least about 3 weeks. In some aspects, the TILs are cultured in the initial TIL culture media until cell yield in the initial culture reaches at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 10×10⁶, at least about 15×10⁶, at least about 20×10⁶, at least about 25×10⁶, at least about 30×10⁶, at least about 35×10⁶, at least about 40×10⁶, at least about 45×10⁶ or at least about 50×10⁶ cells per fragment.

In some aspects, the TILs are stimulated with a CD3 agonist, a CD28 agonist, or both in or prior to the secondary TIL culture media in (b). In some aspects, the TILs are further stimulated with a CD27 agonist in or prior to the secondary TIL culture media. In some aspects, the TILs are further stimulated with a 4-1BB agonist in or prior to the secondary TIL culture media. In some aspects, the TILs are cultured for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, or at least about 26 days, after the stimulation. In some aspects, the TILs are cultured in the secondary culture media until cell yield reaches at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 10×10⁷, at least about 11×10⁷, at least about 12×10⁷, at least about 13×10⁷, at least about 14×10⁷, at least about 15×10⁷, at least about 16×10⁷, at least about 17×10⁷, at least about 18×10⁷, at least about 19×10⁷, or at least about 20×10⁷ cells.

In some aspects, the TILs are stimulated with a CD3 agonist, a CD28 agonist, a CD27 agonist, and/or a 4-1BB agonist in the third TIL culture media. In some aspects, the third TIL culture media are not hyperkalemic. In some aspects, the TILs are cultured in the third TIL culture media for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, or at least about 21 days.

Some aspects of the present disclosure are directed to a method of increasing tumor reactive, e.g., tumor specific, TILs comprising: culturing one or more tumor fragments in initial TIL culture media, which are hyperkalemic and comprise IL-2 and optionally IL-21, up to about 11 to 19 days thereby obtaining TILs from the tumor fragment; culturing the TILs in a secondary TIL culture media, which are hyperkalemic, after adding (i) a CD3 agonist and (ii) a CD28 agonist, a CD27 agonist, a 4-1BB agonist, or any combination thereof, for about 7 to at least about 14 days; culturing the TILs in a third TIL culture media, which are not hyperkalemic, after adding (i) a CD3 agonist and (ii) a CD28 agonist, a CD27 agonist, a 4-1BB agonist, or any combination thereof, for about 14 days to at least about 21 days.

In some aspects, the TILs exhibit increased expression of TCF7 following culture in the MRM, relative to TCF7 expression in a population of TILs following culture in a control medium that is not hyperkalemic. In some aspects, the population of TILs comprises an increased proportion of CD8⁺ CD62L⁺ TILs following culture in the MRM, relative to the proportion of CD8⁺ CD62L⁺ TILs following culture in a control medium that is not hyperkalemic. In some aspects, the population of TILs comprises an increased proportion of CD8⁺ PD1⁺ TILs following culture in the MRM, relative to the proportion of CD8⁺ PD1⁺ TILs following culture in a control medium that is not hyperkalemic.

In some aspects, the heterogeneous population of TILs has increased clonal diversity after being placed in the MRM, as compared to the clonal diversity of a heterogenous population of TILs placed in a control medium.

In some aspects, the heterogeneous population of TILs after being placed in the MRM has a clonal diversity that is at least about 99% to about 100%, at least about 98% to about 100%, at least about 97% to about 100%, at least about 96% to about 100%, at least about 95% to about 100%, at least about 94% to about 100%, at least about 93% to about 100%, at least about 92% to about 100%, at least about 91% to about 100%, at least about 90% to about 100%, at least about 85% to about 100%, at least about 80% to about 100%, at least about 75% to about 100%, at least about 70% to about 100%, at least about 65% to about 100%, at least about 60% to about 100%, at least about 55% to about 100%, at least about 50% to about 100%, at least about 45% to about 100%, or at least about 40% to about 100% of the clonal diversity of TILs in a tumor sample.

In some aspects, the heterogeneous population of TILs after being placed in the MRM has a clonal diversity score of less than about 0.5, less than about 0.45, less than about 0.4, less than about 0.35, less than about 0.3, less than about 0.275, less than about 0.25, less than about 0.225, less than about 0.2, less than about 0.175, less than about 0.15, less than about 0.125, less than about 0.1, less than about 0.075, less than about 0.07, less than about 0.06, or less than about 0.05 as measured by Simpsons clonality.

In some aspects, the heterogeneous population of TILs after being placed in the MRM has a clonal diversity score of less than about 0.3 as measured by Simpsons clonality.

In some aspects, the heterogeneous population of TILs after being placed in the MRM has a clonal diversity score of less than about 0.25 as measured by Simpsons clonality.

In some aspects, the heterogeneous population of TILs after being placed in the MRM has a clonal diversity score of less than about 0.2 as measured by Simpsons clonality.

In some aspects, the heterogeneous population of TILs after being placed in the MRM has a clonal diversity score of less than about 0.1 as measured by Simpsons clonality.

Some aspects of the present disclosure are directed to a composition of immune cells, comprising one or more CD8⁺ TIL cultured according to any method disclosed herein. In some aspects, at least about 30%, at least about 35%, at least about 40%, at least about 45%, at least about 50%, at least about 55%, at least about 60%, at least about 65%, at least about 70%, at least about 75%, or at least about 80% of the immune cells are CD8⁺ TILs.

Some aspects of the present disclosure are directed to a composition comprising a population of immune cells, wherein at least about 30%, at least about 35%, at least about 40%, at least about 45%, at least about 50%, at least about 55%, at least about 60%, at least about 65%, at least about 70%, at least about 75%, or at least about 80% of the immune cells are CD8⁺ TILs. In some aspects, at least about 50% of the cells are CD8⁺ TILs.

In some aspects, the cells exhibit increased expression of TCF7 following culture in the MRM, relative to TCF7 expression in a population of immune cells following culture in a control medium that is not hyperkalemic. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺/CD62L⁺ TILs. In some aspects, at least about 10%, at least about 15%, at least about 20%, at least about 25%, at least about 30%, at least about 35%, at least about 40%, or at least about 50% of the CD8⁺ TILs obtained at the end of the initial TIL culture are PD1⁺. In some aspects, at least about 10%, at least about 15%, at least about 20%, at least about 25%, at least about 30%, at least about 35%, at least about 40%, or at least about 50% of the CD8⁺ TILs obtained at the end of the initial TIL culture are CD39⁺. In some aspects, at least about 10%, at least about 15%, at least about 20%, at least about 25%, at least about 30%, at least about 35%, at least about 40%, or at least about 50% of the CD8⁺ TILs are CD27⁺. In some aspects, at least about 10%, at least about 15%, at least about 20%, at least about 25%, at least about 30%, at least about 35%, at least about 40%, or at least about 50% of the CD8⁺ TILs are CD28⁺. In some aspects, at least about 10%, at least about 15%, at least about 20%, at least about 25%, at least about 30%, at least about 35%, at least about 40%, or at least about 50% of the CD8⁺ TILs obtained at the end of the initial TIL culture are PD1⁺ CD39⁺. In some aspects, at least about 10%, at least about 15%, at least about 20%, at least about 25%, at least about 30%, at least about 35%, at least about 40%, or at least about 50% of the CD8⁺ TILs obtained at the end of the initial TIL culture are PD1⁺ CD27⁺. In some aspects, at least about 10%, at least about 15%, at least about 20%, at least about 25%, at least about 30%, at least about 35%, at least about 40%, or at least about 50% of the CD8⁺ TILs are CD27⁺ CD62L⁺. In some aspects, at least about 10%, at least about 15%, at least about 20%, at least about 25%, at least about 30%, at least about 35%, at least about 40%, or at least about 50% of the CD8⁺ TILs obtained at the end of the initial TIL culture are CD27⁺ CD28⁺ CD103⁺ PD1⁺ TCF7⁺.

In some aspects, the population of immune cells comprises at least about 2×10⁶, at least about 3×10⁶, at least about 4×10⁶, at least about 5×10⁶, at least about 6×10⁶, at least about 7×10⁶, at least about 8×10⁶, at least about 9×10⁶, or at least about 1×10⁷ cells. In some aspects, the population of immune cells comprises at least about 1×10⁶, at least about 3×10⁶, at least about 4×10⁶, at least about 5×10⁶, at least about 6×10⁶, at least about 7×10⁶, at least about 8×10⁶, at least about 9×10⁶, or at least about 1×10⁷ CD8⁺ cells.

In some aspects, the CD8⁺ TILs have a clonal diversity that is at least about 99% to about 100%, at least about 98% to about 100%, at least about 97% to about 100%, at least about 96% to about 100%, at least about 95% to about 100%, at least about 94% to about 100%, at least about 93% to about 100%, at least about 92% to about 100%, at least about 91% to about 100%, at least about 90% to about 100%, at least about 85% to about 100%, at least about 80% to about 100%, at least about 75% to about 100%, at least about 70% to about 100%, at least about 65% to about 100%, at least about 60% to about 100%, at least about 55% to about 100%, at least about 50% to about 100%, at least about 45% to about 100%, or at least about 40% to about 100% of the clonal diversity of TILs in a tumor sample.

In some aspects, the CD8⁺ TILs have a clonal diversity score of less than about 0.5, less than about 0.45, less than about 0.4, less than about 0.35, less than about 0.3, less than about 0.275, less than about 0.25, less than about 0.225, less than about 0.2, less than about 0.175, less than about 0.15, less than about 0.125, less than about 0.1, less than about 0.075, less than about 0.07, less than about 0.06, or less than about 0.05 as measured by Simpsons clonality. In some aspects, the CD8⁺ TILs have a clonal diversity score of less than about 0.3 as measured by Simpsons clonality. In some aspects, the CD8⁺ TILs have a clonal diversity score of less than about 0.25 as measured by Simpsons clonality. In some aspects, the CD8⁺ TILs have a clonal diversity score of less than about 0.2 as measured by Simpsons clonality. In some aspects, the CD8⁺ TILs have a clonal diversity score of less than about 0.1 as measured by Simpsons clonality.

Some aspects of the present disclosure are directed to a method of treating a cancer in a subject in need thereof, comprising administering a population of TILs to the subject, wherein the population of TILs are cultured according to any method disclosed here. In some aspects, the population of TILs is enriched for CD8⁺ TILs. In some aspects, at least about 30%, at least about 35%, at least about 40%, at least about 45%, at least about 50%, at least about 55%, at least about 60%, at least about 65%, at least about 70%, at least about 75%, or at least about 80% of the TILs in the population of TILs are CD8⁺ TILs. In some aspects, at least about 50% of the TILs in the population of TILs are CD8⁺ TILs.

Some aspects of the present disclosure are directed to a method treating a cancer in a subject in need thereof, comprising administering to a subject a composition disclosed herein. In some aspects, the cancer comprises a solid tumor. In some aspects, the cancer comprises a solid tumor derived from a melanoma, a colon cancer, a lung cancer, a cervical cancer, a gastrointestinal cancer, a breast cancer, a prostate cancer, a liver cancer, bone cancer, a pancreatic cancer, a small cell carcinoma of the head and neck, lung squamous cell carcinoma, lung adenocarcinoma, pancreatic adenocarcinoma, head and neck squamous cell carcinoma, testicular germ cell tumors, stomach adenocarcinoma, skin cutaneous melanoma, mesothelioma, kidney renal clear cell carcinoma, cervical squamous cell carcinoma and endocervical adenocarcinoma, esophageal carcinoma, bladder urothelial carcinoma, breast invasive carcinoma, kidney renal papillary cell carcinoma, colon adenocarcinoma, or any combination thereof.

In some aspects, the method comprises administering at least about 2×10⁹, at least about 3×10⁹, at least about 4×10⁹, at least about 5×10⁹, at least about 6×10⁹, at least about 7×10⁹, at least about 8×10⁹, at least about 9×10⁹, or at least about 1×10¹⁰, or at least about 10×10¹⁰, or at least about 15×10¹⁰, or at least about 20×10¹⁰, or at least about 25×10¹⁰, or at least about 30×10¹⁰ cells to the subject. In some aspects, the method comprises administering at least about 1×10⁹, at least about 3×10⁹, at least about 4×10⁹, at least about 5×10⁹, at least about 6×10⁹, at least about 7×10⁹, at least about 8×10⁹, at least about 9×10⁹, or at least about 1×10⁹ CD8⁺ cells to the subject. In some aspects, the method comprises administering about 1×10⁹ to about 4×10⁹, about 5×10⁹ to about 7×10⁹, about 10×10⁹ to about 30×10⁹, about 40×10⁹ to about 60×10⁹, about 70×10⁹ to about 90×10⁹ cells to the subject. In some aspects, the method comprises administering more than 90×10⁹ cell to the subject.

In some aspects, the method further comprises administering a checkpoint inhibitor. In some aspects, the checkpoint inhibitor is administered to the subject after administering the population of cells. In some aspects, the checkpoint inhibitor comprises a CTLA-4 antagonist, a PD1 antagonist, a TIM-3 antagonist, or a combination thereof. In some aspects, the checkpoint inhibitor comprises an anti-CTLA-4 antibody, an anti-PD1 antibody, an anti-PD-L1 antibody, an anti-TIM-3 antibody, or a combination thereof. In some aspects, the method further comprises administering a checkpoint activator. In some aspects, the checkpoint inhibitor is administered to the subject after administering the population of TILs. In some aspects, the checkpoint activator comprises an OX40 agonist, a LAG-3 agonist, a 4-1BB (CD137) agonist, a GITR agonist, a TIM3 agonist, or a combination thereof. In some aspects, the checkpoint activator comprises an anti-OX40 antibody, an anti-LAG-3 antibody, an anti-CD137 antibody, an anti-GITR antibody, an anti-TIM3 antibody, or a combination thereof.

In some aspects, the method further comprises administering a cytokine. In some aspects, the cytokine is administered to the subject after administering the population of TILs. In some aspects, the cytokine is IL-2.

In some aspects, the method further comprises administering a lymphodepleting therapy to the subject prior to administering the population of cells. In some aspects, the lymphodepleting therapy comprises cyclophosphamide, fludarabine, or both cyclophosphamide and fludarabine.

Some aspects of the present disclosure are directed to a population of expanded TILs having a clonal diversity that is at least about 99% to about 100%, at least about 98% to about 100%, at least about 97% to about 100%, at least about 96% to about 100%, at least about 95% to about 100%, at least about 94% to about 100%, at least about 93% to about 100%, at least about 92% to about 100%, at least about 91% to about 100%, at least about 90% to about 100%, at least about 85% to about 100%, at least about 80% to about 100%, at least about 75% to about 100%, at least about 70% to about 100%, at least about 65% to about 100%, at least about 60% to about 100%, at least about 55% to about 100%, at least about 50% to about 100%, at least about 45% to about 100%, or at least about 40% to about 100% of the clonal diversity of TILs in a tumor sample.

Some aspects of the present disclosure are directed to a population of expanded TILs having a clonal diversity score of less than about 0.5, less than about 0.45, less than about 0.4, less than about 0.35, less than about 0.3, less than about 0.275, less than about 0.25, less than about 0.225, less than about 0.2, less than about 0.175, less than about 0.15, less than about 0.125, less than about 0.1, less than about 0.075, less than about 0.07, less than about 0.06, or less than about 0.05 as measured by Simpsons clonality. In some aspects, the clonal diversity score is less than about 0.3 as measured by Simpsons clonality. In some aspects, the clonal diversity score is less than about 0.25 as measured by Simpsons clonality. In some aspects, the clonal diversity score is less than about 0.2 as measured by Simpsons clonality. In some aspects, the clonal diversity score is less than about 0.1 as measured by Simpsons clonality.

In some aspects, at least about at least about 2×10⁶, at least about 3×10⁶, at least about 4×10⁶, at least about 5×10⁶, at least about 6×10⁶, at least about 7×10⁶, at least about 8×10⁶, at least about 9×10⁶, or at least about 1×10⁷ cells. In some aspects, at least at least about 1×10⁶, at least about 3×10⁶, at least about 4×10⁶, at least about 5×10⁶, at least about 6×10⁶, at least about 7×10⁶, at least about 8×10⁶, at least about 9×10⁶, or at least about 1×10⁷ CD8⁺ cells.

In some aspects, at least about 30%, at least about 35%, at least about 40%, at least about 45%, at least about 50%, at least about 55%, at least about 60%, at least about 65%, at least about 70%, at least about 75%, or at least about 80% of the expanded TILs are CD8⁺ TILs. In some aspects, at least about 50% of the expanded TILs are CD8⁺ TILs.

In some aspects, the expanded TILs exhibit increased expression of TCF7 following culture in the MRM, relative to TCF7 expression in a population of immune cells following culture in a control medium that is not hyperkalemic. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the expanded TILs are CD8⁺/CD62L⁺ TILs. In some aspects, at least about 10%, at least about 15%, at least about 20%, at least about 25%, at least about 30%, at least about 35%, at least about 40%, or at least about 50% of the CD8⁺ TILs are PD1⁺. In some aspects, at least about 10%, at least about 15%, at least about 20%, at least about 25%, at least about 30%, at least about 35%, at least about 40%, or at least about 50% of the CD8⁺ TILs are CD39⁺. In some aspects, at least about 10%, at least about 15%, at least about 20%, at least about 25%, at least about 30%, at least about 35%, at least about 40%, or at least about 50% of the CD8⁺ TILs are CD27⁺. In some aspects, at least about 10%, at least about 15%, at least about 20%, at least about 25%, at least about 30%, at least about 35%, at least about 40%, or at least about 50% of the CD8⁺ TILs are CD28⁺. In some aspects, at least about 10%, at least about 15%, at least about 20%, at least about 25%, at least about 30%, at least about 35%, at least about 40%, or at least about 50% of the CD8⁺ TILs are PD1⁺ CD39⁺. In some aspects, at least about 10%, at least about 15%, at least about 20%, at least about 25%, at least about 30%, at least about 35%, at least about 40%, or at least about 50% of the CD8⁺ TILs are PD1⁺ CD27⁺. In some aspects, at least about 10%, at least about 15%, at least about 20%, at least about 25%, at least about 30%, at least about 35%, at least about 40%, or at least about 50% of the CD8⁺ TILs are CD27⁺ CD62L⁺. In some aspects, at least about 10%, at least about 15%, at least about 20%, at least about 25%, at least about 30%, at least about 35%, at least about 40%, or at least about 50% of the CD8⁺ TILs are CD27⁺ CD28⁺ CD103⁺ PD1⁺ TCF7⁺.

In some aspects, the composition disclosed herein or the population of expanded TILs disclosed herein comprises at least one immune cell expression one or more stem-like markers and one or more effector-like markers. In some aspects, the stem-like markers comprise CD45RA+, CD62L+, CCR7+, CD27+, CD28+, BACH2+, LEF1+, TCF7+, or any combination thereof. In some aspects the stem-like markers comprise CD45RA+, CD62L+, CCR7+, and TCF7+, or any combination thereof. In some aspects, the effector-like markers comprise pSTAT5+, STAT5+, pSTAT3+, STAT3+, or any combination thereof. In some aspects, at least about 40%, at least about 50%, at least about 60%, at least about 70%, at least about 80%, at least about 85%, at least about 90%, at least about 95%, at least about 99%, or about 100% of expanded TILs in composition or population comprise at least one immune cell expression one or more stem-like markers and one or more effector-like markers.

Some aspects of the present disclosure are directed to a TIL expressing one or more stem-like markers and one or more effector-like markers. In some aspects, the stem-like markers comprise CD45RA+, CD62L+, CCR7+, CD27+, CD28+, BACH2+, LEF1+, TCF7+, or any combination thereof. In some aspects, the stem-cell markers comprise CD45RA+, CD62L+, CCR7+, and TCF7+. In some aspects, the effector-like markers comprise pSTAT5+, STAT5+, pSTAT3+, STAT3+, or any combination thereof.

Some aspects of the present disclosure are directed to a population of expanded TILs comprising a TIL disclosed herein, e.g., a TIL comprising one or more stem-like markers and one or more effector-like markers. In some aspects, at least about 40%, at least about 50%, at least about 60%, at least about 70%, at least about 80%, at least about 85%, at least about 90%, at least about 95%, at least about 99%, or about 100% of the population of expanded TILs comprises the TILs comprising one or more stem-like markers and one or more effector-like markers.

Some aspects of the present disclosure are directed to a pharmaceutical composition comprising a TIL comprising one or more stem-like markers and one or more effector-like markers and a pharmaceutically acceptable carrier.

Certain aspects of the present disclosure are directed to a method of treating a disease or condition in a subject in need thereof comprising administering a TIL disclosed herein, a population of expanded TILs disclosed herein, or a pharmaceutical composition disclosed herein to the subject. In some aspects, the disease or condition is a cancer.

In some aspects, the population of TILs comprises an increased proportion of CD39⁻/CD69⁻ TILs following culture in the MRM, relative to the proportion of CD39⁻/CD69⁻ TILs following culture in a control medium.

In some aspects, the population of expanded TILs or any one of claims 117-136, wherein at least about 10%, at least about 15%, at least about 20%, at least about 25%, at least about 30%, at least about 35%, or at least about 40% of the total number of TILs in the population of TILs are CD39⁻/CD69⁻.

### BRIEF DESCRIPTION OF THE DRAWINGS/FIGURES

FIGS. 1A-1F are schematics showing exemplary processes of culturing and expanding TILs from tumor fragments. FIGS. 1A-1B show exemplary processes comprising an initial expansion and a secondary expansion, wherein the TILs are optionally stimulated (e.g., according to the methods disclosed herein, e.g., by contacting the cells with 4-1BBL, TRANSACT™, anti-CD3 antibody, an antigen presenting cell, or any combination thereof) at the transition from the initial TIL culture to the secondary TIL expansion (FIGS. 1A-1B) and during the initial TIL culture (FIG. 1B). FIGS. 1C-1D show exemplary processes comprising an initial expansion, a secondary expansion, and a final expansion, wherein the TILs are optionally stimulated (i) at the transition from the initial TIL culture to the secondary TIL expansion (FIGS. 1C-1D); (ii) at the transition from the secondary TIL expansion to the final TIL expansion (FIGS. 1C-1D); and (iii) during the initial TIL culture (FIG. 1D). FIGS. 1E-1F show exemplary processes for generating young TILs, wherein the initial expansion and the secondary expansion are shorter in duration, e.g., 11 days (or less) for each expansion, and wherein the TILs are optionally stimulated at the transition from the initial TIL culture to the secondary TIL expansion (FIGS. 1E-1F) and during the initial TIL culture (FIG. 1F).

FIGS. 2A-2B are graphical representations of FACS cell phenotyping of TILs after initial culture (day 14) in T cell conditioned media (e.g., CTS™ OPTIMIZER™; also referred to herein as “control media”; FIG. 2A) or metabolic reprogramming media (also referred to herein as “MRM”; FIG. 2B). FIGS. 2A and 2B show that culture in MRM produced TILs with enhanced expression of CD39 and PD1 (greater than 20%) as compared to TILs cultured in control media. FIG. 2C is a scatter plot showing the individual differences in the percentage of CD8⁺ cells obtained by culturing TILs from various tumor types in either control or MRM. Each of the linked points represent TILs obtained from the same sample such that the figure summarizes data from 13 patients. Asterisks indicate that the average percentage of CD8⁺ TILs following culture in control media is significantly different than the average percentage of CD8⁺ TILs following culture in MRM. These data show that culturing TILs in MRM results in enrichment of CD8⁺ T cells as compared to culturing TILs in control media.

FIGS. 3A-3E are graphical representations of FACS cell phenotyping based on expression of PD1 and CD27 of cultured CD4⁺ (FIGS. 3A-3B) and CD8⁺ (FIGS. 3C-3D) TILs following 14-day culture in control media (FIGS. 3A and 3C) or MRM (FIGS. 3B and 3D). FIG. 3C is a scatter plot showing the individual differences in the percentage of CD27⁺PD1⁺ cells obtained by culturing TILs from various tumor types in either control or MRM. Each of the linked points represent TILs obtained from the same sample such that FIG. 3C summarizes data from 9 patients. Asterisks indicate that the average percentage of CD27⁺PD1⁺ TILs following culture in control media is significantly different than the average percentage of CD27⁺1PD1⁺ TILs following culture in MRM. These data show that culturing TILs in MRM results in enrichment of CD27⁺1PD1⁺ T cells as compared to culturing TILs in control media.

FIG. 4 is a graphical representation illustrating the statistically significant difference in the percentages of CD27⁺CD28⁺ cells obtained by culturing TILs from various tumor types in either control media or MRM after the initial culture (day 14). Each of the linked points represent TILs obtained from the same sample such that FIG. 4 summarizes data from 9 patients. These data show that culturing TILs in MRM results in enrichment of CD27⁺CD28⁺ T cells as compared to TILs cultured in control media.

FIGS. 5A-5B are graphical representations of FACS cell phenotyping of TILs cultured (day 14) in control media (FIG. 5A) or MRM (FIG. 5B), gated first by CD8 or CD4 expression, followed by CD28 and CD27 expression, followed by CD103 and CD27 expression, followed by PD1 and CD103 expression, and finally by TCF7 and CD27 expression. FIG. 5C is a graphical representation illustrating the mean fluorescence intensity (MFI) of TCF7⁺ TILs following initial culture in control media (1) or MRM (2) (about day 14).

FIGS. 6A-6H are graphical representations of FACS cell phenotyping of TILs expanded in control media (FIGS. 6A-6D) or MRM (FIGS. 6E-6H) after the secondary expansion (about day 21-26), gated first by CD8 or CD4 expression (FIGS. 6A and 6E), CD28 and CD27 expression gated on CD8⁺ cells (FIGS. 6B and 6F), PD1 and CD27 expression gated on CD8⁺ cells (FIGS. 6C and 6G), and finally by TCF7 and CD39 expression gated on CD8⁺ cells (FIGS. 6D and 6H). FIGS. 6B-6D and 6F-6H are CD8⁺ cells.

FIGS. 7A-7H are graphical representations of FACS cell phenotyping of CD8⁺ TILs expanded by co-culture with mutant KRAS-pulsed dendritic cells in control media (FIGS. 7A-7D) or MRM (FIGS. 7E-7H) after the secondary expansion (about day 21), gated first by CD8 or CD4 expression (FIGS. 7A and 7E), followed by CD28 and CD27 expression gated on CD8⁺ cells (FIGS. 7B and 7F), followed by PD1 and CD27 expression gated on CD8⁺ cells (FIGS. 7C and 7G), and finally by TCF7 and CD8 expression gated on PD1⁺ only and CD27⁺, PD1⁺ cells (FIGS. 7D and 7H). FIGS. 7B-7D and 7F-7H are CD8⁺ cells.

FIGS. 8A-8H are graphical representations of FACS cell phenotyping of TILs expanded by co-culture with wild-type KRAS-pulsed dendritic cells in control media (FIGS. 8A-8D) or MRM (FIGS. 8E-8H) after the secondary expansion (about day 21), gated first by CD8 or CD4 expression (FIGS. 8A and 8E), followed by CD28 and CD27 expression gated on CD8⁺ cells (FIGS. 8B and 8F), followed by PD1 and CD27 expression gated on CD8⁺ cells (FIGS. 8C and 8G), and finally by TCF7 and CD8 expression gated on PD1⁺ only and CD27⁺, PD1⁺ cells (FIGS. 8D and 8H). FIGS. 8B-8D and 8F-8H are CD8⁺ cells.

FIGS. 9A-9B are graphical representations of FACS cell phenotyping of cultured TILs following secondary expansion (about day 21-26) in control media (FIG. 9A) or MRM (FIG. 9B).

FIG. 10 is a bar graph showing the fold-change (FC) in gene expression of IL-2, B2M, GZMB, IFNγ, and TCF7 in TILs cultured in control media or MRM after the secondary expansion (about day 21). Expression of each gene is normalized to the expression in TILs cultured in control media.

FIGS. 11A-11L are graphical representations of FACS cell sorting of CD4⁺ or CD8⁺ TILs cultured in control media (FIGS. 11A, 11B, 11E, and 11F) or MRM (FIGS. 11C, 11D, and 11G-11L) after secondary expansion (about day 21-26), gated by PD1 expression (FIGS. 11A-11D) or CD103 expression (FIGS. 11E-11H) and CD39 expression (FIGS. 11A-11H). FIGS. 11I-11L show gating of CD4⁺ TILs (FIGS. 11I and 11K) and CD8⁺ TILs (FIGS. 11J and 11L) gated on PD1 and CD39 expression (FIGS. 11I-11J) followed by CD45RO and CD103 expression (FIGS. 11K-11L).

FIG. 12 is a bar graph illustrating the Simpsons clonality values for immune cells in tumor fragments (“tumor”), TILs expanded using control media (“control”), and TILs expanded using metabolic reprogramming media (“MRM”).

FIGS. 13A-13B are differential abundance (DA) plots generated using the data presented in FIG. 12 for TILs expanded in control media (FIG. 13A) and TILs expanded in MRM (FIG. 13B). FIGS. 13C-13D are graphical representations of tumor antigen recognition of the top 50 dominant tumor TCRs in a TIL population cultured in control media (FIG. 13C) or in MRM (FIG. 13D).

FIG. 14 is a diagram showing KRAS mutant activity of TIL cultured in MRM. ¹SEQ ID NO: 6; ²SEQ ID NO: 1; ³SEQ ID NO: 7; ⁴SEQ ID NO: 8; ⁵SEQ ID NO: 9; ⁶SEQ ID NO: 10; ⁷SEQ ID NO: 11.

FIGS. 15A-15D are bar graphs illustrating the tumor recognition and tumor killing activity of TILs generated using control media or MRM, as evidenced by secreted IFN-gamma (FIGS. 15A and 15D), secreted IL-2 (FIG. 15A), secreted TNF-alpha (FIG. 15B), percent tumor cell killing (FIG. 15C). A=TILs generated using control media, and B=TILs generated using MRM (FIGS. 15A, 15B, and 15D); “Control TIL”=TILs generated using control media and “MRM TIL”=TILs generated using MRM (FIG. 15C); and “TC line”=tumor cell line (FIG. 15D).

FIG. 16 is a graphical representation of the percent of cell lysis of autologous melanoma tumor cells cultured ex vivo over time, following contact and co-culture with TILs (at the time indicated by the arrow). TILs were cultured in either control media or MRM and added to the cultured tumor cells at a ratio of 1:1 effector T cell (E) to tumor cell (T), 2:1 E:T, and 4:1 E:T, as indicated.

FIGS. 17A-17H are graphical representations, illustrating the expression of marker genes in NSCLC TILs expanded using a control process (FIGS. 17A-17D) or MRM (17E-17H). TILs expanded in MRM exhibited superior phenotypic characteristics as measured by CD8+ T cell fraction, low CD39/CD69 expression (FIGS. 17B and 17D), central memory (CD45RO+CD6L+; FIGS. 17C and 17G) and high CD27 expression (FIGS. 17D and 17H). Dashed line highlighted box indicates unfavorable phenotype and solid line highlighted box indicates favorable phenotype.

FIGS. 18A-18C are graphical representations, illustrating negative expression by CD8+ T cells of both CD39 and CD69 within the T cell compartment in TILs obtained from a melanoma (FIG. 8A), a NSCLC (FIG. 18B), or a colorectal cancer (FIG. 18C). Cultures were initiated from freshly supplied human tumor samples and cells were expanded under control or MRM conditions. After final rapid expansion process (REP), TILs were analyzed for negative expression by CD8+ T cells of both CD39 and CD69 within the T cell compartment. For each analysis, TILs expanded from melanoma (n=6 independent tumors), NSCLC (n=5 independent tumors) and colorectal cancer (n=11 independent tumors) were assessed. Statistical significance was measured by paired t test. *** p<0.001, *p<0.05.

FIGS. 19A and 19B are bar graphs illustrating the Simpsons clonality values for immune cells in tumor fragments (“tumor”), TILs expanded using control media (“control”), and TILs expanded using metabolic reprogramming media (“MRM”) for non-small cell lung cancer (NSCLC) (FIG. 19A) and melanoma (FIG. 19B).

### DETAILED DESCRIPTION

The present disclosure is directed to methods of culturing immune cells (e.g., TILs), cells prepared by the methods (e.g., compositions comprising enrichment of oligoclonal or polyclonal tumor reactive, e.g., tumor specific, stem-like T-cells and/or CD8⁺ TILs), and/or methods of treating a subject using the immune cells described herein. The cell culturing methods of the present disclosure are capable of enhancing the expansion of CD8⁺ TILs and/or increasing multipotency and/or pluripotency of the cultured TILs. In some aspects, the culturing methods are capable of reducing and/or preventing immune cell exhaustion, e.g., TIL exhaustion, when the immune cells are cultured and/or the immune cells are used in therapy in vivo. In some aspects the culturing methods of the present disclosure are capable of preserving clonal diversity of the TILs derived from cancer patients.

In some aspects, the disclosure is directed to methods of culturing TILs ex vivo or in vitro comprising culturing a heterogeneous population of TILs in a metabolic reprogramming medium, e.g., a hyperkalemic medium comprising potassium ion at a concentration higher than 40 mM, wherein the hyperkalemic medium is not hypertonic. In some aspects, the disclosure is directed to methods of increasing the number or percentage of CD8+ TILs ex vivo or in vitro comprising culturing a heterogeneous population of TILs in a metabolic reprogramming medium, e.g., a hyperkalemic medium comprising potassium ion at a concentration of at least 5 mM. In other aspects, the disclosure is directed to methods of preparing a CD8⁺-enriched population of tumor infiltrating lymphocytes (TILs), comprising culturing a heterogeneous population of TILs ex vivo or in vitro in a metabolic reprogramming medium, e.g., a hyperkalemic medium comprising potassium ion at a concentration of at least 5 mM. In some aspects, the disclosure is directed to methods of preparing a CD8⁺-enriched population of tumor infiltrating lymphocytes (TILs), comprising culturing a heterogeneous population of TILs ex vivo or in vitro in a metabolic reprogramming medium, e.g., a medium comprising potassium ion at a concentration between 40 mM and 80 mM and NaCl at a concentration between 100 mM and 30 mM, wherein the total concentration of potassium ion and NaCl is between 110 and 140 mM.

In some aspects, the hyperkalemic medium is not hypertonic. In some aspects, the hyperkalemic medium is hypotonic. In some aspects, the hyperkalemic medium is isotonic. In some aspects, the hyperkalemic medium further comprises interleukin (IL)-2, IL-21, IL-7, IL-15, or any combination thereof. In some aspects, the hyperkalemic medium further comprises sodium ion, calcium ion, glucose, or any combination thereof.

Before the present disclosure is described in greater detail, it is to be understood that this disclosure is not limited to the particular compositions or process steps described, which, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “a chimeric polypeptide,” is understood to represent one or more chimeric polypeptides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). In addition, “or” is used mean an open list of the components in the list. For example, “wherein X comprises A or B” means X comprises A, X comprises B, X comprises A and B, or X comprises A or B and any other components.

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range.

Abbreviations used herein are defined throughout the present disclosure. Various aspects of the disclosure are described in further detail in the following subsections.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “approximately,” as applied to one or more values of interest, refers to a value that is similar to a stated reference value. In some aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

The term “control media” as used herein refers to any media in comparison to the metabolic reprogramming media (“MRM”) disclosed herein. Control media can comprise the same components as the metabolic reprogramming media except certain ion concentrations, e.g, potassium ion. In some aspects, metabolic reprogramming media described herein are prepared from control media by adjusting one or more ion concentrations, e.g., potassium ion concentration, as described herein. In some aspects, control media comprise basal media, e.g., CTS™ OPTIMIZER™. In some aspects, control media comprise AIM V, RPMI, or a mixture comprising AIM V and RPMI. In some aspects, control media comprise (i) 50% AIM V, (ii) 50% RPMI1640, (iii) 5% or 10% human serum, and (iv) IL-2. In some aspects, control media thus comprises one or more additional components, including, but not limited to, amino acids, glucose, glutamine, T cell stimulators, antibodies, substituents, etc. that are also being added in the metabolic reprogramming media, but control media have certain ion concentrations different from the metabolic reprogramming media. Unless indicated otherwise, the terms “media” and “medium” can be used interchangeably.

As used herein, the term “immune cell” refers to a cell of the immune system. In some aspects, the immune cell is selected from a T lymphocyte (“T cell”), B lymphocyte (“B cell”), natural killer (NK) cell, macrophage, eosinophil, mast cell, dendritic cell or neutrophil). In some aspects, the immune cell is a tumor-infiltrating cell (TIL). As used herein, a “TIL” refers to T cell that has at least once entered into a tumor or is capable of entering a tumor, e.g., within the parenchyma of a tumor. In some aspects, the tumor is a solid tumor. In some aspects, the tumor is a liquid tumor, e.g., a hematopoietic cancer. TILs prepared by the present methods can have one or more properties that are the same as the naturally occurring TILs. In some aspects, TILs prepared by the present methods have one or more properties that are not present in the naturally occurring TILs. TILs can be obtained using any methods. In some aspects, the TILs are obtained from a tumor sample from a subject. In some aspects, the tumor sample, or a portion thereof, is cultured under conditions that promote evasion of the TILs from the tumor tissue, proliferation of the TILs, and/or expansion of the TILs. In some aspects, the medium used to promote evasion, proliferation, and/or expansion of the TILs is any metabolic reprogramming medium, e.g., hyperkalemic medium, disclosed herein.

As used herein, a “population” of cells refers to a collection of more than one cell, e.g., a plurality of cells. In some aspects, the population of cells comprises more than one TILs, e.g., a plurality of TILs. In some aspects, the population of cells is comprises a heterogeneous mixture of cells, comprising multiple types of cells, e.g., a heterogeneous mixture of TILs and cells other than TILs.

TILs include, but are not limited to, CD8+ T cells (i.e. cytotoxic T cells), CD4+ T cells, B cells, and natural killer cells. TILs include both primary (e.g., obtained from a patient tissue sample) and secondary TILs (e.g., TIL cell populations that have been cultured, expanded or proliferated from primary TILs. In some aspects the TILs are genetically modified. In some aspects, the TIL is a CD8⁺ T cell. CD8⁺ TILs are generally considered to be the subpopulation of TILs responsible for destroying cancer cells. Conversely, CD4⁺ TILs are generally considered to act as suppressors of the immune response, which can limit the immune response against the tumor.

In some aspects, TILs can be defined biochemically using cell surface markers. TILs can be generally categorized by expressing one or more of the following biomarkers: CD4, CD8, TCR αβ, CD27, CD28, CD56, CCR7, CD45RA, CD95, PD-1, and CD25. In some aspects, TILs can be defined functionally by their ability to infiltrate tumors and selectively kill the cancer cells.

As used herein, the terms “T cell” and “T lymphocyte” are interchangeable and refer to any lymphocytes produced or processed by the thymus gland. Non-limiting classes of T cells include effector T cells (such as CD8⁺ T cell) and Th cells (such as CD4⁺ T cells). In some aspects, the immune cell is a Th1 cell. In some aspects, the immune cell is a Th2 cell. In some aspects, the immune cell is a Tc17 cell. In some aspects, the immune cell is a Th17 cell. In some aspects, the immune cell is a T_{reg} cell.

As used herein, the term “memory” T cells refers to T cells that have previously encountered and responded to their cognate antigen (e.g., in vivo, in vitro, or ex vivo) or which have been stimulated with, e.g., an anti-CD3 antibody (e.g., in vitro or ex vivo). Immune cells, e.g., TILs, having a “memory-like” phenotype, upon secondary exposure to antigen or stimulation, reproduce or proliferate to mount a faster and strong immune response than during the primary exposure. In some aspects, memory T cells comprise central memory T cells (T_{CM} cells), effector memory T cells (TEM cells), tissue resident memory T cells (T_{RM} cells), stem cell-like memory T cells (T_{SCM} cells), or any combination thereof.

As used herein, the term “stem-like” or “stem cell-like” refers to a property or an ability of a cell to self-renew and has the multipotent capacity to generate and reconstitute the entire spectrum of memory and effector T cell subsets. In some aspects, a stem-like cell can be measured by specific markers expressed by the cell. In some aspects, those stem-like markers can be one or more of CD45RA+, CD62L+, CCR7+, CD27+, CD28+, BACH2+, LEF1+, and TCF7+. In some aspects, the stem-like cells can be identified by a transcriptome analysis, e.g., using stemness gene signatures disclosed herein. In some aspects, the effector-like marker comprises a marker disclosed in Krishna et al., Science 370:1328-34 (Dec. 11, 2020); and/or Galletti et al., Nature Immunology (October 2018), each of which is incorporated by reference herein in its entirety.

As used herein, the term “stem cell-like memory T cells,” “T memory stem cells,” or “T_{SCM} cells” refer to memory T cells that express CD95, CD45RA, CCR7, and CD62L and are endowed with the stem cell-like ability to self-renew and the multipotent capacity to reconstitute the entire spectrum of memory and effector T cell subsets.

As used herein, the term “central memory T cells” or “T_{CM} cells” refer to memory T cells that express CD45RO, CCR7, and CD62L. Central memory T cells are generally found within the lymph nodes and in peripheral circulation.

As used herein, the term “effector-like” or “effector cell-like” refers to tumor cell killing capacity and cytokine polyfunctionality, e.g., ability of a cell to produce inflammatory cytokines and/or cytotoxic molecules. In some aspects, an effector-like cell can be measured by specific markers expressed by the cell. In some aspects, those effector-like markers can be one or more of pSTAT5+, STAT5+, pSTAT3+, and STAT3+. In some aspects, the effector-like marker comprises a STAT target selected from the group consisting of AKT1, AKT2, AKT3, BCL2L1, CBL, CBLB, CBLC, CCND1, CCND2, CCND3, CISH, CLCF1, CNTF, CNTFR, CREBBP, CRLF2, CSF2, CSF2RA, CSF2RB, CSF3, CSF3R, CSH1, CTF1, EP300, EPO, EPOR, GH1, GH2, GHR, GRB2, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNLR1, IFNW1, IL 10, IL 10RA, IL10RB, IL11, IL11RA, IL12A, IL12B, IL12RB1, IL12RB2, IL13, IL13RA1, IL13RA2, IL15, IL15RA, IL19, IL2, IL20, IL20RA, IL20RB, IL21, IL21R, IL22, IL22RA1, IL22RA2, IL23A, IL23R, IL24, IL26, IL2RA, IL2RB, IL2RG, IL3, IL3RA, IL4, IL4R, IL5, IL5RA, IL6, IL6R, IL6ST, IL7, IL7R, IL9, IL9R, IRF9, JAK1, JAK2, JAK3, LEP, LEPR, LIF, LIFR, MPL, MYC, OSM, OSMR, PIAS1, PIAS2, PIAS3, PIAS4, PIK3CA, PIK3CB, PIK3CD, PIK3CG, PIK3R1, PIK3R2, PIK3R3, PIK3R5, PIM1, PRL, PRLR, PTPN11, PTPN6, SOCS1, SOCS2, SOCS3, SOCS4, SOCS5, SOCS7, SOS1, SOS2, SPRED1, SPRED2, SPRY1, SPRY2, SPRY3, SPRY4, STAM, STAM2, STAT1, STAT2, STAT3, STAT4, STAT5A, STAT5B, STATE, TPO, TSLP, TYK2, and any combination thereof. In some aspects, the effector-like cells can be identified by a transcriptome analysis. In some aspects, the effector-like marker comprises a marker disclosed in Kaech et al., Cell 111:837-51 (2002); Tripathi et al., J. Immunology 185:2116-24 (2010); and/or Johnnidis et al., Science Immunology 6:eabe3702 (Jan. 15, 2021), each of which is incorporated by reference herein in its entirety.

In some aspects, the effector-like cells are characterized using an effector-associated gene set described in Gattinoni, L., et al., Nat Med 17(10):1290-97 (2011). In some aspects, the gene signature for effector-like cells comprises one or more genes selected from MTCH2, RAB6C, KIAA0195, SETD2, C2orf24, NRD1, GNA13, COPA, SELT, TNIP1, CBFA2 T2, LRP10, PRKCI, BRE, ANKS1A, PNPLA6, ARL6IP1, WDFY1, MAPK1, GPR153, SHKBP1, MAP1LC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CHP, RUNX3, TMEM43, REEP4, MEF2D, ABL1, TMEM39A, PCBP4, PLCD1, CHST12, RASGRP1, C1orf58, C11orf63, C6orf129, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYO5A, KIR2DL3, DLG3, MXD4, RALGDS, S1PR5, WSB2, CCR3, TIPARP, SP140, CD151, SOX13, KRTAP5-2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFBI, DNAJC1, GPR114, LRP8, CD69, CMI, NAT13, TGFB1, F1100049, ANTXR2, NR4A3, IL12RB1, NTNG2, RDX, MLLT4, GPRIN3, ADCY9, CD300A, SCD5, ABI3, PTPN22, LGALS1, SYTL3, BMPR1A, TBK1, PMAIP1, RASGEF1A, GCNT1, GABARAPL1, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSF1B, ACTN4, TBKBP1, ATXN1, ARAP2, ARHGEF12, FAM53B, MAN1A1, FAM38A, PLXNC1, GRLF1, SRGN, HLA-DRB5, B4GALT5, WIPI1, PTPRJ, SLFN11, DUSP2, ANXA5, AHNAK, NEO1, CLIC1, EIF2C4, MAP3K5, IL2RB, PLEKHG1, MYO6, GTDC1, EDARADD, GALM, TARP, ADAMS, MSC, HNRPLL, SYT11, ATP2B4, NHSL2, MATK, ARHGAP18, SLFN12L, SPATS2L, RAB27B, PIK3R3, TP53INP1, MBOAT1, GYG1, KATNAL1, FAM46C, ZC3HAV1L, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHA5, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEE1, PYHIN1, F2R, PLD1, THBS1, CD58, FAS, NETO2, CXCR6, ST6GALNAC2, DUSP4, AUTS2, C1orf21, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, KLRB1, and any combination thereof (see, e.g., Gattinoni, L., et al., Nat Med 17(10):1290-97 (2011).

As used herein, the term “effector memory T cells” or “TEM cells” refer to memory T cells that express CD45RO but lack expression of CCR7 and CD62L. Because effector memory T cells lack lymph node-homing receptors (e.g., CCR7 and CD62L), these cells are typically found in peripheral circulation and in non-lymphoid tissues.

As used herein, the term “tissue resident memory T cells” or “T_{RM} cells” refer to memory T cells that do not circulate and remain resident in peripheral tissues, such as the skin, lung, and the gastrointestinal tract. In some aspects, tissue resident memory T cells are also effector memory T cells.

As used herein, the term “naïve T cells,” “T_{N} cells,” or “naïve” TILs” refers to T cells and/or TILs that express CD45RA, CCR7, and CD62L, but which do not express CD95. These cells represent the most undifferentiated cell in the T cell lineage. The interaction between a naïve T cell and an antigen presenting cell (APC) induces differentiation of the naïve T cell towards an activated TEFF cell and an immune response.

As used herein, the term “fragmenting,” “fragment,” and “fragmented” describe processes for disrupting a tumor, including mechanical fragmentation methods such as crushing, slicing, dividing, and morcellating tumor tissue as well as any other methods for disrupting the physical structure of tumor tissue.

The term “culturing” as used herein refers to the controlled growth of cells ex vivo and/or in vitro. As used herein, “culturing” includes the growth of cells, e.g., TILs, during cell expansion. In some aspects, the cultured cells are obtained from a subject, e.g., a human subject. In some aspects, the cultured cells comprise TILs obtained from a human subject. In some aspects, the culturing comprises placing a tumor sample or tumor fragment into a medium disclosed herein, wherein the medium promotes TIL evasion from the tumor sample and TIL expansion. In some aspects, the TILs are isolated or purified prior to the culture. In some aspects, the cell culturing is intended to expand the number of cultured cells, e.g., to increase proliferation of the cells.

“Expand” or “expansion,” as used herein in reference to TILs refers to the process of stimulating or activating the cells and culturing the cells. The expansion process can lead to an increase in the proportion or the total number of desired cells, e.g., an increase in the proportion or total number of TILs, in a population of cultured cells, after the cells are stimulated or activated and cultured. Expansion does not require that all cell types in a population of cultured cells are increased in number. Rather, in some aspects, only a subset of cells in a population of cultured cells are increased in number during expansion, while the number of other cell types may not change or may decrease.

As used herein, the term “yield” refers to the total number of cells following a culture method or a portion thereof. In some aspects, the term “yield” refers to a particular population of cells, e.g., stem-like TILs in a population of TILs. The yield can be determined using any methods, including, but not limited to, estimating the yield based on a representative sample.

As used herein, the term “stem cell-like,” “stem-like,” or “less-differentiated” refers to a cell, e.g., an immune cell (e.g., a TIL), that expresses markers consistent with a more naïve phenotype. For example, a less differentiated TIL can express one or more markers characteristic of a T_{N} or a T_{SCM} cell. In some aspects, a “less-differentiated” or “stem-like” TIL expresses CD45RA, CCR7, and CD62L. In some aspects, a “less-differentiated” or “stem-like” TIL expresses CD45RA, CCR7, and CD62L, and is CD45RO^{low}. In some aspects, a “less-differentiated” or “stem-like” immune cell (e.g., TIL) expresses CD45RA, CCR7, and CD62L, and does not express CD45RO. In some aspects, a “less-differentiated” or “stem-like” T cell expresses CD45RA, CCR7, CD62L, and TCF7. In some aspects, the methods disclosed herein promote the growth and/or proliferation of cells, e.g., TILs, having a less-differentiated phenotype. Without being bound by any particular mechanism, in some aspects, the methods disclosed herein block, inhibit, or limit differentiation of less-differentiated cells, e.g., TILs, resulting in an increased number of stem-like cells in culture. For example, it is generally thought that to effectively control tumors, adoptive transfer of less-differentiated TILs with a stem cell-like memory or central memory phenotype are preferred. See, e.g., Gattinoni, L., et al., J. Clin. Invest. 115:1616-1626 (2005); Gattinoni, L., et al. Nat Med 15(7):808-814 (2009); Lynn, R. C., et al., Nature 576(7786): 293-300 (2019); Gattinoni, L., et al., J. Clin. Invest. 115:1616-1626 (2005); Gattinoni, L., et al. Nat Med 15(7):808-814 (2009); and Gattinoni, L., et al., Nat Med 17(10): 1290-1297 (2011).

Sternness is characterized by the capacity to self-renew, the multipotency, and the persistence of proliferative potential. In some aspects, sternness is characterized by a particular gene signature, e.g., a combined pattern of expression across a multitude of genes. In some aspects, the gene signature comprises one or more genes selected from ACTN1, DSC1, TSHZ2, MYB, LEF1, TIMD4, MAL, KRT73, SESN3, CDCA7L, LOC283174, TCF7, SLC16A10, LASS6, UBE2E2, IL7R, GCNT4, TAF4B, SULT1B1, SELP, KRT72, STXBP1, TCEA3, FCGBP, CXCR5, GPA33, NELL2, APBA2, SELL, VIPR1, FAM153B, PPFIBP2, FCER1G, GJB6, OCM2, GCET2, LRRN1, IL6ST, LRRC16A, IGSF9B, EFHA2, LOC129293, APP, PKIA, ZC3H12D, CHMP7, KIAA0748, SLC22A17, FLJ13197, NRCAM, C5orf13, GIPC3, WNT7A, FAM117B, BENDS, LGMN, FAM63A, FAM153B, ARHGEF11, RBM11, RIC3, LDLRAP1, PELI1, PTK2, KCTD12, LMO7, CEP68, SDK2, MCOLN3, ZNF238, EDAR, FAM153C, FAAH2, BCL9, C17orf48, MAP1D, ZSWIM1, SORBS3, IL4R, SERPINFL C16orf45, SPTBN1, KCNQ1, LDHB, BZW2, NBEA, GAL3ST4, CRTC3, MAP3K1, HLA-DOA, RAB43, SGTB, CNN3, CWH43, KLHL3, PIM2, RGMB, C16orf74, AEBP1, SNORD115-11, SNORD115-11, GRAP, and any combination thereof (see, e.g., Gattinoni (2011)). In some aspects, the gene signature comprises one or more gene selected from NOG, TIMD4, MYB, UBE2E2, FCER1G, HAVCR1, FCGBP, PPFIBP2, TPST1, ACTN1, IGF1R, KRT72, SLC16A10, GJB6, LRRN1, PRAGMIN, GIPC3, FLNB, ARRB1, SLC7A8, NUCB2, LRRC7, MYO15B, MAL, AEBP1, SDK2, BZW2, GAL3ST4, PITPNM2, ZNF496, FAM117B, C16orf74, TDRD6, TSPAN32, C18orf22, C3orf44, LOC129293, ZC3H12D, MLXIP, C7orf10, STXBP1, KCNQ1, FLJ13197, LDLRAP1, RAB43, RIN3, SLC22A17, AGBL3, TCEA3, NCRNA00185, FAM153B, FAM153C, VIPR1, MMP19, HBS1L, EEF2K, SNORA5C, UBASH3A, FLJ43390, RP6-213H19.1, INPP5A, PIM2, TNFRSF10D, SNRK, LOC100128288, PIGV, LOC100129858, SPTBN1, PROS1, MMP28, HES1, CACHD1, NSUN5C, LEF1, TTTY14, SNORA54, HSF2, C16orf67, NSUN5B, KIAA1257, NRG2, CAD, TARBP1, STRADB, MT1F, TMEM41B, PDHX, KDM6B, LOC100288322, UXS1, LGMN, NANOS2, PYGB, RASGRP2, C14orf80, XPO6, SLC24A6, FAM113A, MRM1, FBXW8, NDUFS2, KCTD12, and any combination thereof (see, e.g., Gattinoni, L., et al., Nat Med 17(10): 1290-1297 (2011) or Galletti et al. Nat Immunol 21, 1552-1562 (2020)).

In the presence of prolonged antigen exposure, such as in many cancers, more differentiated immune cells, e.g., effector and effector memory T cells, often become exhausted and lose their anti-tumor function. Biomarkers, e.g., T cell markers, can be measured using any methods. In some aspects, T cells are identified using antibody-staining following by gated flow cytometry.

The term “clonotype,” as used herein, refers to a population of T cells with unique DNA sequences that result from TCRα or TCRß rearrangements. A unique variable a chain (VA) sequence may pair up with more than one variable ß chain (VB) sequence. Conversely, a unique VB sequence may pair up with more than one VA sequence.

As used herein, the term “tonicity” refers to the measure of the effective osmotic pressure gradient across a cell membrane. Tonicity can be measured or calculated based on the level of potassium ion and sodium chloride (NaCl) in a solution. Herein, tonicity is calculated as the sum of the concentration of potassium ion (K+) and the concentration of sodium chloride (NaCl), multiplied by two. Tonicity can be expressed in terms of the osmolality of the solution, e.g., the media. As used herein, a solution, e.g., medium, is considered “isotonic” when the concentration of solutes in the media is equivalent to the concentration of solutes inside the cell. As used herein, an isotonic medium has an osmolality of about 280 mOsm/L (e.g., ([K+]+[NaCl])×2=280).

As used herein, a solution, e.g., a medium, is considered “hypotonic” if the concentration of solutes in the solution is lower than the concentration of solutes in the cell. As used herein, a hypotonic solution has a tonicity of less than 280 mOsm/L (e.g., ([K+]+[NaCl])×2<280). In some aspects, a hypotonic medium described herein has an osmolality of about 240 mOsm/L or about 250 mOsm/L. In some aspects, a hypotonic medium has a tonicity from at least about 220 mOsm/L to less than about 280 mOsm/L. In some aspects, a hypotonic medium has a tonicity from at least about 230 mOsm/L to less than about 280 mOsm/L. In some aspects, a hypotonic medium has a tonicity from at least about 240 mOsm/L to less than about 280 mOsm/L. In some aspects, a hypotonic medium described herein has a tonicity of about 250 mOsm/L (e.g., ([K+]+[NaCl])×2=250).

As used herein, a solution, e.g., a medium, is considered “hypertonic” if the concentration of solutes in the solution is higher than the concentration of solutes in the cell. As used herein, a hypertonic solution has an osmolality of greater than 300 mOsm/L (e.g., ([K+]+[NaCl])×2>280). In some aspects, a hypertonic medium described herein has an osmolality of about 320 mOsm/L. In certain aspects, the tonicity of the solution, e.g., medium is adjusted by increasing or decreasing the concentration of one or more solute selected from potassium ions, sodium ions, glucose, and any combination thereof. In some aspects, the tonicity of the solution, e.g., medium is adjusted by increasing or decreasing the concentration of potassium ions and NaCl. In some aspects, the tonicity of a medium can be maintained by offsetting the increase of one solute with a decrease in a second solute. For example, increasing the concentration of potassium ion in a medium without changing the concentration of sodium ions can increase the tonicity of the medium. However, if the concentration of potassium ions is increased and the concentration of sodium ions is decreased, the tonicity of the original medium can be maintained. As used herein, the tonicity of a medium is defined by the sum of the potassium concentration and the NaCl concentration, multiplied by two. See, e.g., Table 2.

As used herein, the terms “potassium,” “potassium ion,” “potassium cation,” and “K+” are used interchangeably to refer to elemental potassium. Elemental potassium exists in solution as a positive ion. However, it would be readily apparent to a person of ordinary skill in the art that standard means of preparing a solution comprising potassium ion include diluting a potassium containing salt (e.g., KCl) into a solution. As such, a solution, e.g., a medium, comprising a molar (M) concentration of potassium ion, can be described as comprising an equal molar (M) concentration of a salt comprising potassium.

As used herein, the terms “sodium ion” and “sodium cation” are used interchangeably to refer to elemental sodium. Elemental sodium exists in solution as a monovalent cation. However, it would be readily apparent to a person of ordinary skill in the art that standard means of preparing a solution comprising sodium ion include diluting a sodium-containing salt (e.g., NaCl) into a solution. As such, a solution, e.g., a medium, comprising a molar (M) concentration of sodium ion, can be described as comprising an equal molar (M) concentration of a salt comprising sodium.

As used herein, the terms “calcium ion” and “calcium cation” are used interchangeably to refer to elemental calcium. Elemental calcium exists in solution as a divalent cation. However, it would be readily apparent to a person of ordinary skill in the art that standard means of preparing a solution comprising calcium ion include diluting a calcium-containing salt (e.g., CaCl₂)) into a solution. As such, a solution, e.g., a medium, comprising a molar (M) concentration of calcium ion, can be described as comprising an equal molar (M) concentration of a salt comprising calcium.

As used herein, the term “hyperkalemic,” e.g., “hyperkalemic medium,” refers to a medium that has an increased potassium concentration. In some aspects, the hyperkalemic medium comprises potassium ion at a concentration of greater than 5 mM. In some aspects, the hyperkalemic medium comprises potassium ion at a concentration higher than 40 mM. In some aspects, the hyperkalemic medium a concentration of potassium ion of at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, about 75 mM, about 80 mM, about 85 mM, about 90 mM, about 95 mM, or about 100 mM. The term “metabolic reprogramming media,” “metabolic reprogramming medium,” or “MRM,” as used herein, refers to a hyperkalemic medium of the present disclosure. In certain aspects, the metabolic reprogramming media comprises about 40 mM to about 80 mM NaCl, about 40 mM to about 90 mM KCl, about 0.5 mM to about 2.8 mM calcium, and about 10 mM to about 24 mM glucose. In some aspects, the metabolic reprograming media further comprises an osmolality of about 250 to about 340 mOsmol.

As used herein, the term “basal” media refers to any starting media that is supplemented with one or more of the additional elements disclosed herein, e.g., potassium, sodium, calcium, glucose, IL-2, IL-7, IL-15, IL-21, or any combination thereof. The basal media can be any media for culturing immune cells, e.g., TILs. In some aspects, the basal media is selected from a balanced salt solution (e.g., PBS, DPBS, HBSS, EBSS), Dulbecco's Modified Eagle's Medium (DMEM), Click's medium, Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OPTMIZER™ CTS™ T-Cell Expansion Basal Medium (ThermoFisher), OPTMIZER™ Complete, IMMUNOCULT™ XF (STEMCELL™ Technologies), IMMUNOCULT™ XF, AIM V, TEXMACS™ medium, TRANSACT™ TIL expansion medium, TIL rapid expansion protocol medium, and any combination thereof. In some aspects, the basal medium is serum free. In some aspects, the basal media comprises PRIME-XV T cell CDM. In some aspects, the basal media comprises OPTMIZER™. In some aspects, the basal media comprises OPTMIZER™ Pro. In some aspects, the basal media comprises X-VIVO™ 15 (LONZA). In some aspects, the basal media comprises IMMUNOCULT™. In some aspects, the basal media comprises Click's medium. In some aspects, the basal media comprises TRANSACT™ TIL expansion medium. In some aspects, the basal media comprises TIL rapid expansion medium. In some aspects, the basal medium further comprises immune cell serum replacement (ICSR). For example, in some aspects, the basal medium comprises OPTMIZER™ Complete supplemented with ICSR, AIM V supplemented with ICSR, IMMUNOCULT™ XF supplemented with ICSR, RPMI supplemented with ICSR, TEXMACS™ supplemented with ICSR, or any combination thereof. In some aspects, suitable basal media include Click's medium, OpTimizer® (CTS®) medium, Stemline® T cell expansion medium (Sigma-Aldrich), AIM V® medium (CTS®), TexMACS® medium (Miltenyi Biotech), ImmunoCult® medium (Stem Cell Technologies), PRIME-XV® T-Cell Expansion XSFM (Irvine Scientific), Iscoves medium, and/or RPMI-1640 medium. In some aspects, the basal media comprises NaCl free CTS™ OPTIMIZER™. In some aspects, suitable basal media include Click's medium, OpTimizer® (CTS®) medium, Stemline® T cell expansion medium (Sigma-Aldrich), AIM V® medium (CTS®), TexMACS® medium (Miltenyi Biotech), ImmunoCult® medium (Stem Cell Technologies), PRIME-XV® T-Cell Expansion XSFM (Irvine Scientific), Iscoves medium, and/or RPMI-1640 medium. In some aspects, the basal media comprises NaCl free CTS™ OpTimizer™. In some aspects, the basal media comprises one or more sodium salt in addition to the NaCl that is added to control the tonicity, e.g., NaCl added in combination with potassium ion.

As used herein, the term “cytokine” refers to small, secreted proteins released by cells that have a specific effect on the interactions and communications between cells. Non-limiting examples of cytokines include interleukins (e.g., interleukin (IL)-1, IL-2, IL-4, IL-7, IL-9, IL-13, IL-15, IL-3, IL-5, IL-6, IL-11, IL-10, IL-20, IL-14, IL-16, IL-17, IL-21, IL-23, and IL-29), interferons (IFN; e.g., IFN-α, IFN-β, and IFN-γ), tumor necrosis factor (TNF) family members, and transforming growth factor (TGF) family members. Some aspects of the present disclosure are directed to methods of culturing cells, e.g., T cells and/or NK cells, in a medium comprising a cytokine. Some aspects of the present disclosure are directed to methods of culturing TILs in a medium comprising a cytokine. Some aspects of the present disclosure are directed to methods of expanding TILs in a medium comprising a cytokine. In some aspects, the cytokine is an interleukin. In some aspects, the cytokine is selected from IL-2, IL-7, IL-15, IL-21, and a combination thereof. IL-2 (UniProtKB—P60568) is produced by T cells in response to antigenic or mitogenic stimulation. IL-2 is known to stimulate T cell proliferation and other activities crucial to regulation of the immune response. IL-7 (UniProtKB—P13232) is a hematopoietic growth factor capable of stimulating the proliferation of lymphoid progenitors. IL-7 is believed to play a role in proliferation during certain stages of B-cell maturation. IL-15 (UniProtKB—P40933), like IL-2, is a cytokine that stimulates the proliferation of T-lymphocytes. IL-21 (UniProtKB—Q9HBE4) is a cytokine with immunoregulatory activity. IL-21 is thought to promote the transition between innate and adaptive immunity and to induce the production of IgG1 and IgG3 in B-cells. IL-21 may also play a role in proliferation and maturation of natural killer (NK) cells in synergy with IL-15, and IL-21 may regulate proliferation of mature B- and T-cells in response to activating stimuli. In synergy with IL-15 and IL-18, IL-15 also stimulates interferon gamma production in T-cells and NK cells, and IL-21 may also inhibit dendritic cell activation and maturation during a T-cell-mediated immune response.

As used herein, the term “higher than” means greater than but not equal to. For example, “higher than 5 mM” means any amount that is more than 5 mM, but which does not include 5 mM.

The term “preferentially,” as used herein, refers to the predominant outcome. For example, if the methods disclosed herein preferentially promote expansion of CD8⁺ TILs, it is to be understood that the predominant product of the expansion is CD8⁺ TILs. The term “preferentially” does not necessarily mean that 100% of, e.g., the resulting TILs are CD8⁺, rather the term suggests that CD8⁺ TILs are expanded to a greater extent than CD8⁻ TILs.

As used herein, “administering” refers to the physical introduction of a therapeutic agent or a composition comprising a therapeutic agent to a subject, using any of the various methods and delivery systems. The different routes of administration for a therapeutic agent described herein (e.g., a TIL cultured as described herein) include intravenous, intraperitoneal, intramuscular, subcutaneous, spinal or other parenteral routes of administration, for example by injection or infusion.

The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricular, intravitreal, epidural, and intrasternal injection and infusion, as well as in vivo electroporation.

Alternatively, a therapeutic agent described herein (e.g., a TIL cultured as described herein) can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten. As used herein, the term “cognate antigen” refers to an antigen which an immune cell (e.g., a TIL) recognizes and thereby, induces the activation of the immune cell (e.g., triggering intracellular signals that induce effector functions, such as cytokine production, and/or for proliferation of the cell).

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

The term “hematological malignancy” or “hematological cancer” refers to mammalian cancers and tumors of the hematopoietic and lymphoid tissues. Non-limiting examples of hematological malignancies include those affecting tissues of the blood, bone marrow, lymph nodes, and lymphatic system, including acute lymphoblastic leukemia (ALL), chronic lymphocytic lymphoma (CLL), small lymphocytic lymphoma (SLL), acute myelogenous leukemia (AML), chronic myelogenous leukemia (CIVIL), acute monocytic leukemia (AMoL), Hodgkin's lymphoma, and non-Hodgkin's lymphomas. Hematological malignancies are also referred to as “liquid tumors.” Liquid tumor cancers include, but are not limited to, leukemias, myelomas, and lymphomas, as well as other hematological malignancies. TILs obtained from liquid tumors may also be referred to herein as marrow infiltrating lymphocytes (MILs).

A “solid tumor,” as used herein, refers to an abnormal mass of tissue. Solid tumors may be benign or malignant. Nonlimiting examples of solid tumors include sarcomas, carcinomas, and lymphomas, such as cancers of the lung, breast, prostate, colon, rectum, and bladder. The tissue structure of a solid tumor includes interdependent tissue compartments including the parenchyma (cancer cells) and the supporting stromal cells in which the cancer cells are dispersed, and which may provide a supporting microenvironment.

As used herein, the term “immune response” refers to a biological response within a vertebrate against foreign agents, which response protects the organism against these agents and diseases caused by them. An immune response is mediated by the action of a cell of the immune system (e.g., a T lymphocyte (e.g., a TIL), B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ TIL, or the inhibition of a Treg cell. As used herein, the terms “T cell” and “T lymphocytes” are interchangeable and refer to any lymphocytes produced or processed by the thymus gland. In some aspects, a TIL is a CD8⁺ TIL. In some aspects, a TIL is a CD4⁺ TIL.

As used herein, the term “anti-tumor immune response” refers to an immune response against a tumor antigen.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein. As used herein, the phrase “subject in need thereof” includes subjects, such as mammalian subjects, that would benefit, e.g., from administration of immune cells, e.g., TILs, cultured as described herein to control tumor growth.

The term “therapeutically effective amount” or “therapeutically effective dosage” refers to an amount of an agent (e.g., a TIL cultured as described herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations.

The effective amount of the composition (e.g., cells cultured as described herein) can, for example, (i) reduce the number of cancer cells; (ii) reduce tumor size; (iii) inhibit, delay, slow to some extent and can stop cancer cell infiltration into peripheral organs; (iv) inhibit (i.e., slow to some extent and can stop tumor metastasis); (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of a composition disclosed herein (e.g., T cells cultured as described herein), which is clinically proven to effect a significant decrease in cancer or slowing of progression (regression) of cancer, such as an advanced solid tumor. The ability of a therapeutic agent of the present disclosure (e.g., T cells cultured as described herein) to promote disease regression can be evaluated using a variety of methods known to the skilled practitioner, such as in human subjects during clinical trials, in animal model systems predictive of efficacy in humans, or by assaying the activity of the agent in in vitro assays.

The terms “effective” and “effectiveness” with regard to a treatment include both pharmacological effectiveness and physiological safety. Pharmacological effectiveness refers to the ability of a composition disclosed herein (e.g., cells cultured as described herein) to promote cancer regression in the patient. Physiological safety refers to the level of toxicity, or other adverse physiological effects at the cellular, organ, and/or organism level (adverse effects) resulting from administration of a composition disclosed herein (e.g., cells cultured as described herein).

As used herein, the term “tumor reactive” refers to the ability of an immune cell, e.g., a TIL, to target and kill a tumor cell. As used herein, the term “tumer specific” refers to a tumor reactive immune cell, e.g., TIL, that specifically targets a tumor cell.

As used herein, the term “T cell receptor” or “TCR” refers to a heterodimer composed of 2 different transmembrane polypeptide chains: an α chain and a β chain, each consisting of a constant region, which anchors the chain inside the T-cell surface membrane, and a variable region, which recognizes and binds to the antigen presented by MHCs. The TCR complex is associated with 6 polypeptides forming 2 heterodimers, CD3γε and CD3δε, and 1 homodimer CD3 ζ, which together forms the CD3 complex. T-cell receptor-engineered T-cell therapy utilizes the modification of T cells that retain these complexes to specifically target the antigens expressed by particular tumor cells. As used herein, the term “TCR” includes naturally occurring TCRs and engineered TCRs.

As used herein, the terms “ug” and “uM” are used interchangeably with “μg” and “μM,” respectively.

Various aspects described herein are described in further detail in the following subsections.

### II. Methods of the Disclosure

The present disclosure is directed to methods of culturing immune cells, e.g., TILs, ex vivo or in vitro. In some aspects, the methods of the present disclosure comprise culturing or placing immune cells, e.g., TILs, in a culture condition, wherein the culture (e.g., certain ion concentrations, tonicity of the medium, cytokines, and or any combination thereof) is capable of enhancing the expansion of CD8⁺ TILs. In some aspects, the culture (e.g., certain ion concentrations, tonicity of the medium, cytokines, and or any combination thereof) is capable of reducing, limiting, or preventing the differentiation of the immune cells, e.g., the TILs (e.g., CD8⁺ TILs and/or CD4⁺ TILs), thereby affecting or improving their use in a cell therapy. In some aspects, the present disclosure comprises culturing of TILs in a metabolic reprogramming media that is high in potassium concentration. Increased potassium was surprisingly found to correlate with increased expansion of CD8⁺ TILs that have increased expression of stem-like markers and increased clonal diversity, while maintaining tumor-reactivity (e.g., tumor specificity), as compared to conventional methods using lower potassium levels, e.g., less than about 40 mM potassium ion, e.g., 5 mM potassium ion. Further, though exceedingly high concentrations of potassium (e.g., >80 mM, >90 mM, or >100 mM) reduced TIL expansion, the methods described herein yielded therapeutically effective numbers of TILs following culture conditions, e.g., durations, consistent with conventional methods.

The use of immunotherapy strategies has demonstrated considerable clinical efficacy in the treatment of certain types of advanced cancer. Immune checkpoint blockade (ICB) can result in objective and sometimes durable responses in patients with metastatic melanoma. Certain cohorts of colon cancer, lung cancer patients and small proportions of patients with additional malignancies can also benefit from ICB. Chimeric antigen receptor (CAR) T cell therapy has mediated dramatic clinical responses in patients with blood cell malignancies, most notably B cell-lineage tumors that can be targeted with CD19 or B cell maturation antigen (BCMA) CARs. Treatment with T cells transduced with T cell receptors (TCRs) that recognize shared, non-mutated tumor antigens such as NY-ESO-1 can also mediate clinical responses in patients who express TCR matched human leukocyte antigens (HLAs). However, in spite of these notable successes, the vast majority of patients with advanced cancers still do not benefit from immunotherapy treatments and will eventually succumb to their illness.

TIL therapy has also shown a potential in mediating clinical responses in patients with advanced cancer. Emerging evidence has demonstrated that TILs are a heterogenous population composed of both tumor-reactive and non-specific bystander cells. This heterogenous population of TILs causes difficulty and unwanted effects in the TIL therapy and/or dilution of the efficacy of the TIL therapy as the non-specific bystander cells in the heterogenous population are not preferred. Bystander cells are nonspecific T cells, which can dilute the diversity of reactive TILs. Bystander cells include TILs that recognize epitopes that are not tumor related. In addition, the efficacy of TIL therapy has demonstrated diverse responses in patients with melanoma, advanced cervical, lung, breast, and/or gastrointestinal cancers.

In some aspects, the present disclosure provides methods of reducing the heterogeneity of TIL population ex vivo or in vitro for an in vivo therapy. In some aspects, the methods disclosed herein enrich for a particular type of a TIL population, e.g., CD8+ TILs and/or tumor-reactive CD8+ TILs. In some aspects the methods disclosed herein enrich for stem-like T cell populations, e.g., stem-like tumor-reactive TILs and/or stem-like tumor-reactive CD8+ TILs.

Not being bound by any theory, the present disclosure sets forth a method of enriching a TIL population with a particular cell type, i.e., tumor-reactive TIL, CD8+ TIL, tumor-reactive CD8+ TIL, stem-like tumor-reactive TIL, stem-like CD8+ TIL, and/or stem-like tumor-reactive CD8+ TIL, using a hyperkalemic medium. Therefore, some aspects of the present disclosure are directed to methods of culturing TILs ex vivo or in vitro comprising placing a heterogeneous population of TILs in a hyperkalemic medium comprising potassium ion at a concentration higher than 40 mM. In some aspects, the heterogeneous population of TILs is enriched in CD8+ TILs after being placed in the hyperkalemic medium.

Some aspects of the present disclosure are directed to methods of increasing a number or percentage of CD8+ TILs (e.g., tumor reactive, e.g., tumor specific, CD8+ TILs) ex vivo or in vitro comprising culturing a heterogeneous population of TILs in a hyperkalemic medium comprising potassium ion at a concentration of at least 5 mM. Other aspects of the present disclosure are directed to methods of preparing a CD8⁺-enriched (e.g., tumor reactive CD8⁺-enriched) population of TILs, comprising culturing a heterogeneous population of TILs ex vivo or in vitro in a hyperkalemic medium comprising potassium ion at a concentration of at least 5 mM.

Some aspects of the present disclosure are directed to methods of increasing a number or percentage of tumor reactive TILs ex vivo or in vitro comprising culturing a heterogeneous population of TILs in a hyperkalemic medium comprising potassium ion at a concentration of at least 5 mM. Other aspects of the present disclosure are directed to methods of preparing a tumor reactive-enriched population of TILs, comprising culturing a heterogeneous population of TILs ex vivo or in vitro in a hyperkalemic medium comprising potassium ion at a concentration of at least 5 mM.

Some aspects of the present disclosure are directed to methods of increasing a number or percentage of stem-like TILs (e.g., stem-like tumor reactive TILs, stem-like CD8+ TILs, or stem-like tumor reactive CD8+ TILs) ex vivo or in vitro comprising culturing a heterogeneous population of TILs in a hyperkalemic medium comprising potassium ion at a concentration of at least 5 mM. Other aspects of the present disclosure are directed to methods of preparing a population of TILs enriched for stem-like TILs (e.g., stem-like tumor reactive TILs, stem-like CD8+ TILs, or stem-like tumor reactive CD8+ TILs), comprising culturing a heterogeneous population of TILs ex vivo or in vitro in a hyperkalemic medium comprising potassium ion at a concentration of at least 5 mM.

In some aspects, the methods and/or compositions disclosed herein increase the clonal diversity of TILs in culture, as compared to TILs cultured under control conditions (e.g., in a media comprising potassium ion at a concentration of less than about 5 mM).

Clonal diversity can be assessed using any methods. In some aspects, clonal diversity is assessed using a subset of TILs cultured according to the methods disclosed herein. Non-limiting examples of methods of assessing clonal diversity of a population of TILs can be found, for example, in Venturi et al., J. Immunolog. Mtd. 321:182-95 (2007), which is incorporated by reference herein in its entirety. In some aspects, clonal diversity is assessed using IMMUNOSEQ® (ADAPTIVE BIOTECHNOLOGIES®). In some aspects, clonal diversity is assessed using TCR deep sequencing. In certain aspects, the clonal diversity is assessed by sequencing TCRß CDR3 sequences in total RNA isolated from the population of TILs (e.g., cDNA prepare from the total RNA). In some aspects, clonal diversity is assessed using Simpsons clonality.

In some aspects, TILs cultured according to the methods disclosed herein have a clonal diversity that is the same as the clonal diversity of TILs in a tumor sample. In some aspects, the TILs cultured according to the methods disclosed herein have a clonal diversity that is at least about 99% to about 100%, at least about 98% to about 100%, at least about 97% to about 100%, at least about 96% to about 100%, at least about 95% to about 100%, at least about 94% to about 100%, at least about 93% to about 100%, at least about 92% to about 100%, at least about 91% to about 100%, at least about 90% to about 100%, at least about 85% to about 100%, at least about 80% to about 100%, at least about 75% to about 100%, at least about 70% to about 100%, at least about 65% to about 100%, at least about 60% to about 100%, at least about 55% to about 100%, at least about 50% to about 100%, at least about 45% to about 100%, or at least about 40% to about 100% of the clonal diversity of TILs in a tumor sample. In certain aspects, the TILs cultured according to the methods disclosed herein have a clonal diversity that is at least about 95% to about 100% of the clonal diversity of TILs in a tumor sample. In certain aspects, the TILs cultured according to the methods disclosed herein have a clonal diversity that is at least about 90% to about 100% of the clonal diversity of TILs in a tumor sample. In certain aspects, the TILs cultured according to the methods disclosed herein have a clonal diversity that is at least about 85% to about 100% of the clonal diversity of TILs in a tumor sample. In certain aspects, the TILs cultured according to the methods disclosed herein have a clonal diversity that is at least about 80% to about 100% of the clonal diversity of TILs in a tumor sample. In certain aspects, the TILs cultured according to the methods disclosed herein have a clonal diversity that is at least about 75% to about 100% of the clonal diversity of TILs in a tumor sample. In certain aspects, the TILs cultured according to the methods disclosed herein have a clonal diversity that is at least about 70% to about 100% of the clonal diversity of TILs in a tumor sample. In certain aspects, the TILs cultured according to the methods disclosed herein have a clonal diversity that is at least about 60% to about 100% of the clonal diversity of TILs in a tumor sample. In certain aspects, the TILs cultured according to the methods disclosed herein have a clonal diversity that is at least about 50% to about 100% of the clonal diversity of TILs in a tumor sample. In certain aspects, the TILs cultured according to the methods disclosed herein have a clonal diversity that is at least about 40% to about 100% of the clonal diversity of TILs in a tumor sample.

In some aspects, clonal diversity is assessed using Simpsons clonality (√Σpi² where, pi is the proportional abundance of clone i in a given sample). Simpsons clonality is commonly used to assess for productive rearrangements within a sample thus measuring the magnitude of the clone frequency distribution (see, e.g., Venturi et al., J. Immunol. Meth. 321:182-95 (2007), which is incorporated by reference herein in its entirety). The values of the Simpsons clonality range from 0 to 1, where values approaching 1 represent a less clonally diverse and thus a more monoclonal TIL population.

In some aspects, the clonal diversity of TILs cultured according to the methods disclosed herein have a clonal diversity score of less than about 0.5, less than about 0.45, less than about 0.4, less than about 0.35, less than about 0.3, less than about 0.275, less than about 0.25, less than about 0.225, less than about 0.2, less than about 0.175, less than about 0.15, less than about 0.125, less than about 0.1, less than about 0.075, less than about 0.07, less than about 0.06, or less than about 0.05 as measured by Simpsons clonality. In some aspects, the clonal diversity of TILs cultured according to the methods disclosed herein have a clonal diversity score of less than about 0.5, as measured by Simpsons clonality. In some aspects, the clonal diversity of TILs cultured according to the methods disclosed herein have a clonal diversity score of less than about 0.4, as measured by Simpsons clonality. In some aspects, the clonal diversity of TILs cultured according to the methods disclosed herein have a clonal diversity score of less than about 0.3, as measured by Simpsons clonality. In some aspects, the clonal diversity of TILs cultured according to the methods disclosed herein have a clonal diversity score of less than about 0.275, as measured by Simpsons clonality. In some aspects, the clonal diversity of TILs cultured according to the methods disclosed herein have a clonal diversity score of less than about 0.25, as measured by Simpsons clonality. In some aspects, the clonal diversity of TILs cultured according to the methods disclosed herein have a clonal diversity score of less than about 0.24, as measured by Simpsons clonality. In some aspects, the clonal diversity of TILs cultured according to the methods disclosed herein have a clonal diversity score of less than about 0.23, as measured by Simpsons clonality. In some aspects, the clonal diversity of TILs cultured according to the methods disclosed herein have a clonal diversity score of less than about 0.22, as measured by Simpsons clonality. In some aspects, the clonal diversity of TILs cultured according to the methods disclosed herein have a clonal diversity score of less than about 0.21, as measured by Simpsons clonality. In some aspects, the clonal diversity of TILs cultured according to the methods disclosed herein have a clonal diversity score of less than about 0.2, as measured by Simpsons clonality. In some aspects, the clonal diversity of TILs cultured according to the methods disclosed herein have a clonal diversity score of less than about 0.19, as measured by Simpsons clonality. In some aspects, the clonal diversity of TILs cultured according to the methods disclosed herein have a clonal diversity score of less than about 0.18, as measured by Simpsons clonality. In some aspects, the clonal diversity of TILs cultured according to the methods disclosed herein have a clonal diversity score of less than about 0.17, as measured by Simpsons clonality. In some aspects, the clonal diversity of TILs cultured according to the methods disclosed herein have a clonal diversity score of less than about 0.16, as measured by Simpsons clonality. In some aspects, the clonal diversity of TILs cultured according to the methods disclosed herein have a clonal diversity score of less than about 0.15, as measured by Simpsons clonality. In some aspects, the clonal diversity of TILs cultured according to the methods disclosed herein have a clonal diversity score of less than about 0.14, as measured by Simpsons clonality. In some aspects, the clonal diversity of TILs cultured according to the methods disclosed herein have a clonal diversity score of less than about 0.13, as measured by Simpsons clonality. In some aspects, the clonal diversity of TILs cultured according to the methods disclosed herein have a clonal diversity score of less than about 0.12, as measured by Simpsons clonality. In some aspects, the clonal diversity of TILs cultured according to the methods disclosed herein have a clonal diversity score of less than about 0.11, as measured by Simpsons clonality. In some aspects, the clonal diversity of TILs cultured according to the methods disclosed herein have a clonal diversity score of less than about 0.1, as measured by Simpsons clonality. In some aspects, the clonal diversity of TILs cultured according to the methods disclosed herein have a clonal diversity score of less than about 0.09, as measured by Simpsons clonality. In some aspects, the clonal diversity of TILs cultured according to the methods disclosed herein have a clonal diversity score of less than about 0.08, as measured by Simpsons clonality. In some aspects, the clonal diversity of TILs cultured according to the methods disclosed herein have a clonal diversity score of less than about 0.07, as measured by Simpsons clonality. In some aspects, the clonal diversity of TILs cultured according to the methods disclosed herein have a clonal diversity score of less than about 0.06, as measured by Simpsons clonality. In some aspects, the clonal diversity of TILs cultured according to the methods disclosed herein have a clonal diversity score of less than about 0.05, as measured by Simpsons clonality.

In some aspects, the present disclosure includes a method of expanding TILs obtained from a human subject comprising:
a. culturing the TILs in initial TIL culture media (“Initial TIL Culturing”);
b. culturing the TILs in secondary TIL culture media (“Second TIL Culturing”); and
c. culturing the TILs in third (or final) TIL culture media (“Final TIL Culturing”), wherein the initial TIL culture media, the secondary TIL culture media, and/or the third TIL culture media are hyperkalemic. In some aspects, the Final TIL Culturing further comprises T cell stimulation or activation. In some aspects, the Second TIL Culturing further comprises T cell stimulation or activation.

In some aspects, the present disclosure includes a method of expanding TILs obtained from a human subject comprising:
a. culturing the TILs in initial TIL culture media (“Initial TIL Culturing”); and
b. expanding the TILs in secondary TIL culture media (“Second TIL Expansion”);
   wherein the initial TIL culture media and/or the secondary TIL culture media are hyperkalemic.

In some aspects, the present disclosure includes a method of expanding TILs obtained from a human subject comprising:
a. culturing the TILs in initial TIL culture media (“Initial TIL Culturing”);
b. expanding the TILs in secondary TIL culture media (“Second TIL Expansion”); and
c. expanding the TILs in third (or final) TIL culture media (“Final TIL Expansion”),
   wherein the initial TIL culture media, the secondary TIL culture media, and/or the third TIL culture media are hyperkalemic.

In some aspects, only the initial TIL culture media are hyperkalemic. In some aspects, only the secondary TIL culture media are hyperkalemic. In some aspects, both the initial TIL culture media and the secondary TIL culture media are hyperkalemic. In some aspects, the initial TIL culture media and the secondary TIL culture media are hyperkalemic and the third TIL culture media are not hyperkalemic. In some aspects, the initial TIL culture media further comprises IL-2, IL-21, or both. In some aspects the initial TIL culture, the secondary TIL culture and the third or final TIL culture comprises IL-2 with or without IL-21.

In some aspects, the initial TIL culture media, the secondary TIL culture and/or the third or final TIL culture further comprises a T cell supplement, a serum replacement, glutamine, a glutamine substitute (e.g., Glutamax (L-alanine-L-glutamine)), non-essential amino acids, an antibiotics (e.g., Penicillin, Streptomycin, or both), an anti-fungal agent (e.g., FUNGIN™), and/or sodium pyruvate.

In some aspects, the TILs are cultured in the initial TIL culture media up to about six days, about seven days, about eight days, about nine days, about 10 days, about 11 days, about 12 days, about 13 days, about 14 days, about 15 days, about 16 days, about 17 days, about 18 days or about 19 days. In some aspects the TILs are cultured in the initial TIL culture media for about 14 days to about 19 days.

In some aspects, the TILs in the second TIL Culturing are stimulated with a CD3 agonist, a CD28 agonist, or both in the secondary TIL culture media in (b). In some aspects, the TILs in the second TIL Culturing are further stimulated with a CD27 ligand in the secondary TIL culture media. In some aspects, the TILs in the second TIL Culturing are further stimulated with a 4-1BB ligand in the secondary TIL culture media.

In some aspects, the TILs in the second TIL Expansion are cultured for at least about 6 days, at least about 7 days, at least about 8 days, at least about 9 days, at least about 10 days, at least about 11 days after the stimulation or activation.

In some aspects, the TILs in the second TIL Expansion are cultured for about 6 days to about 12 days, about 7 days to about 11 days, about 7 days to about 10 days, about 8 days to about 12 days, after stimulation or activation.

In some aspects, the TILs in the third or final TIL Expansion are cultured for at least about 7 days, at least about 8 days, at least about 9 days, at least about 10 days, at least about 11 days, at least about 12 days, at least about 13 days, at least about 14 days, at least about 15 days after the second stimulation or activation.

In some aspects, the TILs in the third or final TIL Expansion are cultured for about 7 days to about 14 days, about 7 days to about 12 days, about 7 days to about 11 days, about 8 days to about 14 days, about 8 days to about 13 days, about 8 days to about 12 days, after the second stimulation or activation.

The present disclosure also provides culturing the TILs in the metabolic reprogramming media disclosed herein, the cell culture disclosed herein, or the cell bag or bioreactor disclosed herein as an initial TIL culture. In some aspects, the initial TIL culture culturing is maintained for at least about six days, at least about seven days, at least about eight days, at least about 9 days, at least about 10 days, at least about 11 days, at least about 12 days, at least about 13 days, at least about 14 days, at least about 15 days, at least about 16 days, at least about 17 days, at least about 18 days, at least about 19 days. In some aspects, the initial TIL culture culturing is maintained for 14 days to about 19 days.

The present methods can further be developed into a secondary TIL expansion. In order to start a secondary TIL expansion, the TILs are stimulated or activated with a CD3 agonist and/or a CD28 agonist, e.g., TRANSACT™. In some aspects, the TILs in the media are further stimulated with a CD27 ligand. In some aspects, the TILs in the media are further stimulated with a 4-1BB ligand. In some aspects, the second TIL expansion is maintained for at least about 6 days, at least about 7 days, at least about 8 days, at least about 9 days, at least about 10 days, at least about 11 days. In some aspects, the secondary TIL expansion culturing is maintained for about 7 days (about one week).

In some aspects, the TILs are cultured in secondary TIL culture media until cell yield in the secondary expansion reaches at least about 1×10⁷ to at least about 50×10⁷, at least about 2×10⁷ to at least about 40×10⁷, at least about 3×10⁷ to at least about 30×10⁷, at least about 4×10⁷ to at least about 25×10⁷, at least about 5×10⁷ to at least about 20×10⁷, at least about 1×10⁷ to at least about 20×10⁷, at least about 2×10⁷ to at least about 20×10⁷, at least about 3×10⁷ to at least about 20×10⁷, or at least about 4×10⁷ to at least about 20×10⁷ cells. In some aspects, the TILs are cultured in secondary TIL culture media until cell yield in the secondary expansion reaches at least about 5×10⁷ to at least about 20×10⁷ cells. In some aspects, the TILs are cultured in secondary TIL culture media until cell yield in the secondary expansion reaches at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 10×10⁷, at least about 11×10⁷, at least about 12×10⁷, at least about 13×10⁷, at least about 14×10⁷, at least about 15×10⁷, at least about 16×10⁷, at least about 17×10⁷, at least about 18×10⁷, at least about 19×10⁷, or at least about 20×10⁷ cells. In some aspects, the TILs are cultured in secondary TIL culture media until cell yield in the secondary expansion reaches at least about 5×10⁷ cells. In some aspects, the TILs are cultured in secondary TIL culture media until cell yield in the secondary expansion reaches at least about 6×10⁷ cells. In some aspects, the TILs are cultured in secondary TIL culture media until cell yield in the secondary expansion reaches at least about 7×10⁷ cells. In some aspects, the TILs are cultured in secondary TIL culture media until cell yield in the secondary expansion reaches at least about 8×10⁷ cells. In some aspects, the TILs are cultured in secondary TIL culture media until cell yield in the secondary expansion reaches at least about 9×10⁷ cells. In some aspects, the TILs are cultured in secondary TIL media until cell yield in the secondary expansion reaches at least about 10×10⁷ cells. In some aspects, the TILs are cultured in secondary TIL culture media until cell yield in the secondary expansion reaches at least about 15×10⁷ cells. In some aspects, the TILs are cultured in secondary TIL culture media until cell yield in the secondary expansion reaches at least about 20×10⁷ cells.

After the secondary TIL expansion, the TILs can be expanded further in the final expansion stage. In order to start the final TIL expansion, the TILs from the second TIL expansion culture are transferred to control media (i.e., non-hyperkalemic media). At the start of the final TIL expansion culture, the TILs are further stimulated with a CD3 agonist and/or a CD28 agonist e.g., TRANSACT™. In some aspects, the TILs in the media are further stimulated with a CD27 ligand. In some aspects, the TILs in the media are further stimulated with a 4-1BB ligand.

In some aspects, the TILs are cultured in secondary TIL culture media until cell yield in the secondary expansion reaches at least about 1×10⁷ to at least about 50×10⁷, at least about 2×10⁷ to at least about 40×10⁷, at least about 3×10⁷ to at least about 30×10⁷, at least about 4×10⁷ to at least about 25×10⁷, at least about 5×10⁷ to at least about 20×10⁷, at least about 1×10⁷ to at least about 20×10⁷, at least about 2×10⁷ to at least about 20×10⁷, at least about 3×10⁷ to at least about 20×10⁷, or at least about 4×10⁷ to at least about 20×10⁷ cells. In some aspects, the TILs are cultured in secondary TIL culture media until cell yield in the secondary expansion reaches at least about 5×10⁷ to at least about 20×10⁷ cells. In some aspects, the TILs are cultured in secondary TIL culture media until cell yield in the secondary expansion reaches at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 10×10⁷, at least about 11×10⁷, at least about 12×10⁷, at least about 13×10⁷, at least about 14×10⁷, at least about 15×10⁷, at least about 16×10⁷, at least about 17×10⁷, at least about 18×10⁷, at least about 19×10⁷, or at least about 20×10⁷ cells. In some aspects, the TILs are cultured in secondary TIL culture media until cell yield in the secondary expansion reaches at least about 5×10⁷ cells. In some aspects, the TILs are cultured in secondary TIL culture media until cell yield in the secondary expansion reaches at least about 6×10⁷ cells. In some aspects, the TILs are cultured in secondary TIL culture media until cell yield in the secondary expansion reaches at least about 7×10⁷ cells. In some aspects, the TILs are cultured in secondary TIL culture media until cell yield in the secondary expansion reaches at least about 8×10⁷ cells. In some aspects, the TILs are cultured in secondary TIL culture media until cell yield in the secondary expansion reaches at least about 9×10⁷ cells. In some aspects, the TILs are cultured in secondary TIL culture media until cell yield in the secondary expansion reaches at least about 10×10⁷ cells. In some aspects, the TILs are cultured in secondary TIL culture media until cell yield in the secondary expansion reaches at least about 15×10⁷ cells. In some aspects, the TILs are cultured in secondary TIL culture media until cell yield in the secondary expansion reaches at least about 20×10⁷ cells.

In some aspects, TILs are subjected to a final expansion. In some aspects, the final expansion comprises a stimulation. In some aspects the stimulation is the same as the stimulation used during the secondary expansion. In some aspects, the TILs are stimulated during the final expansion by culturing the cells in a medium comprising TRANSACT™ with or without 4-1BBL and/or CD27L. In some aspects, the TILs are stimulated during the final expansion by culturing the cells in a medium comprising TRANSACT™ and 4-1BBL and/or CD27L. In some aspects, the TILs are stimulated during the final expansion by culturing the cells in a medium comprising at least about 1:100 TRANSACT™, at least about 1 μg/ml 4-1BBL, and at least about 5 μg/ml CD27L.

In some aspects, the final expansion step is carried out in static GREX. In some aspects, the final expansion is carried out in a stirred tank. In some aspects, the final expansion is continued until the cell yield in the final TIL culture media reaches at least about 40×10⁹ to at least about 100×10⁹, at least about 40×10⁹ to at least about 90×10⁹, at least about 40×10⁹ to at least about 80×10⁹, at least about 40×10⁹ to at least about 70×10⁹, at least about 40×10⁹ to at least about 60×10⁹, at least about 40×10⁹ to at least about 50×10⁹, at least about 10×10⁹ to at least about 100×10⁹, at least about 20×10⁹ to at least about 100×10⁹, at least about 30×10⁹ to at least about 100×10⁹, at least about 30×10⁹ to at least about 50×10⁹, or at least about 35×10⁹ to at least about 45×10⁹ cells. In some aspects, the final expansion is continued until the cell yield in the final TIL culture media reaches at least about 40×10⁹ to at least about 100×10⁹ cells. In some aspects, the final expansion is continued until the cell yield in the final TIL culture media reaches at least about 40×10⁹, at least about 45×10⁹, at least about 50×10⁹, at least about 55×10⁹, at least about 60×10⁹, at least about 65×10⁹, at least about 70×10⁹, at least about 75×10⁹, at least about 80×10⁹, at least about 85×10⁹, at least about 90×10⁹, at least about 95×10⁹, or at least about 100×10⁹ cells. In some aspects, the final expansion is continued until the cell yield in the final TIL culture media reaches at least about 40×10⁹ cells. In some aspects, the final expansion is continued until the cell yield in the final TIL culture media reaches at least about 50×10⁹ cells. In some aspects, the final expansion is continued until the cell yield in the final TIL culture media reaches at least about 60×10⁹ cells. In some aspects, the final expansion is continued until the cell yield in the final TIL culture media reaches at least about 70×10⁹ cells. In some aspects, the final expansion is continued until the cell yield in the final TIL culture media reaches at least about 80×10⁹ cells. In some aspects, the final expansion is continued until the cell yield in the final TIL culture media reaches at least about 90×10⁹ cells. In some aspects, the final expansion is continued until the cell yield in the final TIL culture media reaches at least about 100×10⁹ cells.

In some aspects, the final expansion is continued until the cell yield in the final TIL culture media for at least about 7 to at least about 21 days. In some aspects, the final expansion is continued until the cell yield in the final TIL culture media for at least about 7 days. In some aspects, the final expansion is continued until the cell yield in the final TIL culture media for at least about 8 days. In some aspects, the final expansion is continued until the cell yield in the final TIL culture media for at least about 9 days. In some aspects, the final expansion is continued until the cell yield in the final TIL culture media for at least about 10 days. In some aspects, the final expansion is continued until the cell yield in the final TIL culture media for at least about 11 days. In some aspects, the final expansion is continued until the cell yield in the final TIL culture media for at least about 12 days. In some aspects, the final expansion is continued until the cell yield in the final TIL culture media for at least about 13 days. In some aspects, the final expansion is continued until the cell yield in the final TIL culture media for at least about 14 days. In some aspects, the final expansion is continued until the cell yield in the final TIL culture media for at least about 15 days. In some aspects, the final expansion is continued until the cell yield in the final TIL culture media for at least about 16 days. In some aspects, the final expansion is continued until the cell yield in the final TIL culture media for at least about 17 days. In some aspects, the final expansion is continued until the cell yield in the final TIL culture media for at least about 18 days. In some aspects, the final expansion is continued until the cell yield in the final TIL culture media for at least about 19 days. In some aspects, the final expansion is continued until the cell yield in the final TIL culture media for at least about 20 days. In some aspects, the final expansion is continued until the cell yield in the final TIL culture media for at least about 21 days.

In some aspects, the hyperkalemic medium is not hypotonic. In some aspects, the hyperkalemic medium is not isotonic. In some aspects, the hyperkalemic medium is not hypertonic.

In some aspects, the heterogeneous population of TILs comprises CD4⁺ TILs and CD8⁺ TILs. In some aspects, the heterogeneous population of TILs is obtained from one or more tumor sample obtained from a subject. Any tumor sample obtained from a subject can be used in the methods disclosed herein. In some aspects, the tumor sample comprises a tumor biopsy. In some aspects, the tumor biopsy comprises a punch biopsy. In some aspects, the tumor sample comprises tumor tissue obtained during a tumor resection surgery. In some aspects, the tumor sample comprises a core needle biopsy. In some aspects, the tumor sample is collected taken from an inflamed tumor, e.g., a tumor comprising a high number of TILs.

In some aspects, the tumor sample is plated and subjected to an initial TIL culture. In some aspects, the initial TIL culture comprises culturing the tumor sample in the metabolic reprogramming medium, e.g., hyperkalemic medium. Any methods for TIL expansion from a tumor sample can be used in the methods disclosed herein. In some aspects, the tumor sample is fractionated prior to plating and initial TIL culture. In some aspects, the initial TIL culture lasts for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days. In some aspects, the initial TIL culture lasts at least about 14 days to about 19 days. In some aspects the initial TIL culture lasts at least about 14 days.

In some aspects, the proportion of CD8⁺ TILs (e.g., tumor reactive CD8+ TILs and/or stem-like CD8+ TILs) to non-CD8⁺ TILs is increased following the initial TIL culture, as compared to the proportion of CD8⁺ TILs to non-CD8⁺ TILs prior to the initial TIL culture. In some aspects, the proportion of CD8⁺ TILs (e.g., tumor reactive CD8+ TILs and/or stem-like CD8+ TILs) to non-CD8⁺ TILs is increased by at least about 1.5-fold, at least about 2-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 60-fold, at least about 70-fold, at least about 80-fold, at least about 90-fold, or at least about 100-fold. In some aspects, the proportion of CD8⁺ TILs (e.g., tumor reactive CD8+ TILs and/or stem-like CD8+ TILs) to non-CD8⁺ TILs is increased by at least about 50-fold.

In some aspects, following culture of the heterogeneous population of TILs, at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the TILs in the population are CD8⁺ TILs (e.g., tumor reactive CD8+ TILs and/or stem-like CD8+ TILs). In some aspects, following culture of the heterogeneous population of TILs, at least about 50% of the TILs in the population are CD8⁺ TILs (e.g., tumor reactive CD8+ TILs and/or stem-like CD8+ TILs). In some aspects, following culture of the heterogeneous population of TILs, at least about 25% of the TILs in the population are CD8⁺ TILs (e.g., tumor reactive CD8+ TILs and/or stem-like CD8+ TILs). In some aspects, following culture of the heterogeneous population of TILs, at least about 75% of the TILs in the population are CD8⁺ TILs (e.g., tumor reactive CD8+ TILs and/or stem-like CD8+ TILs).

In some aspects, the TILs are stimulated or activated following the initial TIL culture. Any methods for expansion and/or stimulation of TILs can be used during the stimulation of the TILs. In some aspects, the TILs are stimulated following the initial TIL culture. In some aspects, the TILs are stimulated by subjecting the TILs to TRANSACT™ TIL expansion, TIL rapid expansion protocol, or a combination thereof. In some aspects, the TILs are stimulated in a hyperkalemic medium disclosed herein.

In some aspects, a population of immune cells, e.g., TILs (e.g., CD8⁺ TILs (e.g., tumor reactive CD8+ TILs)), cultured using the methods disclosed herein exhibits an increased number of stem-like TILs relative to a population of cells cultured using conventional methods, e.g., in a medium having less than about 40 mM potassium ion. In some aspects, the immune cells, e.g., TILs (e.g., CD8⁺ TILs (e.g., tumor reactive CD8+ TILs)), exhibit increased expression of markers characteristic of stem-like cells relative to the starting population of cells. In some aspects, the starting population of cells comprises cells obtained from a human subject. In some aspects, the starting population of cells comprises TILs obtained from a human subject.

Increased cell multipotency can be measured using any methods. In some aspects, cell stemness is measured by antibody staining followed by gated flow cytometry. In some aspects, the cell stemness is measured by autophagy flux. In some aspects, the cell stemness is measured by glucose uptake. In some aspects, the cell stemness is measured by fatty acid uptake. In some aspects, the cell stemness is measured by mitochondrial biomass. In some aspects, the cell stemness is measured by RNA quantification/expression analysis (e.g., microarray, qPCR (TaqMan), RNA-Seq., single-cell RNA-Seq., or any combinations thereof). In some aspects, the cell stemness is measured by (e.g., transcripts that are linked to) a metabolism assay (e.g., a Seahorse metabolism assay, analysis of extracellular acidification rate (ECAR); analysis of oxygen consumption rate (OCR); analysis of spare respiratory capacity; and/or analysis of mitochondrial membrane potential). In some aspects, stemness is measured using one or more in vivo functional assays (e.g., assaying cell persistence, antitumor capacity, antitumor clearance, viral clearance, multipotency, cytokine release, cell killing, or any combination thereof).

In some aspects, the differentiation status of the immune cells, e.g., TILs (e.g., CD8⁺ TILs (e.g., tumor reactive CD8+ TILs)), is characterized by increased numbers of cells expressing markers typical of less differentiated cells. In some aspects, an increase in the number of stem-like immune cells, e.g., TILs (e.g., CD8⁺ TILs (e.g., tumor reactive CD8+ TILs)), is characterized by increased numbers of immune cells, e.g., TILs (e.g., CD8⁺ TILs (e.g., tumor reactive CD8+ TILs)), expressing markers typical of T_{N} and/or T_{SCM} cells. In some aspects, an increase in the number of stem-like immune cells, e.g., TILs (e.g., CD8⁺ TILs (e.g., tumor reactive CD8+ TILs)), is characterized by increased numbers of immune cells, e.g., TILs (e.g., CD8⁺ TILs (e.g., tumor reactive CD8+ TILs)), expressing markers typical of T_{SCM} cells. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that express CD45RA. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that express CCR7. In some aspects, the population of TILs exhibits an increased number of immune cells, e.g., TILs (e.g., CD8⁺ TILs (e.g., tumor reactive CD8+ TILs)), that express CD62L. In some aspects, the population of TILs exhibits an increased number of immune cells, e.g., TILs (e.g., CD8⁺ TILs (e.g., tumor reactive CD8+ TILs)), that express CD28. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that express CD95. In some aspects, the immune cells, e.g., TILs (e.g., CD8⁺ TILs (e.g., tumor reactive CD8+ TILs)), are CD45RO^{low}. In some aspects, the immune cells, e.g., TILs (e.g., CD8⁺ TILs (e.g., tumor reactive CD8+ TILs)), do not express CD45RO. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that are CD8⁺, CD45RA⁺, CCR7⁺, and CD62L⁺. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that are CD8⁺, CD95⁺, CD45RA⁺, CCR7⁺, and CD62L⁺. In some aspects, the population of immune cells, e.g., TILs (e.g., CD8⁺ TILs (e.g., tumor reactive CD8+ TILs)), exhibits an increased number of cells that express TCF7. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that are CD8⁺, CD45RA⁺, CCR7⁺, CD62L⁺, and TCF7⁺. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that are CD8⁺, CD95⁺, CD45RA⁺, CCR7⁺, CD62L⁺, and TCF7⁺. In some aspects, the immune cells, e.g., TILs (e.g., CD8⁺ TILs (e.g., tumor reactive CD8+ TILs)), express CD3. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that are CD8⁺, CD3⁺, CD45RA⁺, CCR7⁺, CD62L⁺, TCF7⁺. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that are CD8⁺, CD3⁺, CD95⁺, CD45RA⁺, CCR7⁺, CD62L⁺, TCF7⁺. In some aspects, the immune cells, e.g., TILs (e.g., CD8⁺ TILs (e.g., tumor reactive CD8+ TILs)), express CD27. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that are CD8⁺, CD27⁺, CD3⁺, CD95⁺, CD45RA⁺, CCR7⁺, CD62L⁺, TCF7⁺. In some aspects, the population of immune cells, e.g., TILs (e.g., CD8⁺ TILs (e.g., tumor reactive CD8+ TILs)), exhibits an increased number of immune cells, e.g., TILs (e.g., CD8⁺ TILs (e.g., tumor reactive CD8+ TILs)), that are CD8⁺, CD27⁺, CD3⁺, CD95⁺, CD45RA⁺, CCR7⁺, CD62L⁺, TCF7⁺. In some aspects, the population of immune cells, e.g., TILs (e.g., CD8⁺ TILs (e.g., tumor reactive CD8+ TILs)), exhibits an increased number of T_{SCM} cells. In some aspects, the population of immune cells, e.g., TILs (e.g., CD8⁺ TILs (e.g., tumor reactive CD8+ TILs)), exhibits an increased number of T_{N} cells. In some aspects, the population of immune cells, e.g., TILs (e.g., CD8⁺ TILs (e.g., tumor reactive CD8+ TILs)), exhibits an increased number of T_{SCM} and T_{N} cells. In some aspects, the population of cell exhibits an increased number of stem-like TILs.

In some aspects, the number of stem-like immune cells, e.g., TILs (e.g., CD8⁺ TILs (e.g., tumor reactive CD8+ TILs)), in the culture is increased by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%, relative to the number of stem-like immune cells, e.g., TILs (e.g., CD8⁺ TILs (e.g., tumor reactive CD8+ TILs)), prior to culture. In some aspects, the number of stem-like immune cells, e.g., TILs (e.g., CD8⁺ TILs (e.g., tumor reactive CD8+ TILs)), in the culture is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, or at least about 20-fold, relative to the number of stem-like immune cells, e.g., TILs (e.g., CD8⁺ TILs (e.g., tumor reactive CD8+ TILs)), prior to culture.

In some aspects, following culture of immune cells, e.g., TILs (e.g., CD8⁺ TILs (e.g., tumor reactive CD8+ TILs)), according to the methods disclosed herein, stem-like CD8⁺ TILs (e.g., stem-like tumor reactive CD8+ TILs) constitute at least about 1%, at least about 2%, at least about 3%, at least about 4%, at least about 5%, at least about 10%, at least about 15%, of the total number of CD8⁺ TILs in the culture.

In some aspects, following culture of TILs according to the methods disclosed herein, stem-like TILs constitute at least about 10% to at least about 70% of the total number of TILs in the culture. In some aspects, following culture of TILs according to the methods disclosed herein, stem-like TILs constitute at least about 10%, at least about 20%, at least about 30%, at least about 40%, at least about 50%, at least about 60%, or at least about 70% of the total number of CD8⁺ TILs in the culture. In some aspects, following culture of TILs according to the methods disclosed herein, stem-like TILs constitute at least about 10%, at least about 20%, at least about 30%, at least about 40%, at least about 50%, at least about 60%, or at least about 70% of the total number of CD4⁺ TILs in the culture.

In some aspects, following culture of TILs according to the methods disclosed herein, at least about 10% to at least about 40% of the total number of TILs in the culture are CD39⁻/CD69⁻ TILs. In some aspects, following culture of TILs according to the methods disclosed herein, at least about 10%, at least about 15%, at least about 20%, at least about 25%, at least about 30%, at least about 35%, or at least about 40% of the total number of TILs in the culture are CD39⁻/CD69⁻ TILs.

In some aspects, following culture of TILs according to the methods disclosed herein, at least about 10% to at least about 70% of the total number of TILs in the culture are CD39⁻/TCF7⁺ TILs. In some aspects, following culture of TILs according to the methods disclosed herein, at least about 10%, at least about 15%, at least about 20%, at least about 25%, at least about 30%, at least about 35%, or at least about 40% of the total number of TILs in the culture are CD39⁻/TCF7⁺ TILs. In some aspects the TILs are CD4⁺ T cells. In some aspects the TILs are CD8⁺ TILs.

In some aspects, upon adoptive transfer of the immune cells, e.g., TILs (e.g., CD8⁺ TILs (e.g., tumor reactive CD8+ TILs)), cultured according to the methods disclosed herein, the transferred cells exhibit decreased cell exhaustion, as compared to cells cultured using conventional culture conditions. In some aspects, upon adoptive transfer of the cultured TILs, the transferred CD8⁺-enriched TILs persist for a longer period of time in vivo, as compared to TILs cultured using conventional culture conditions. Such increased persistence refers to the ability of the TIL to infilitrate and function in the tumor microenvironment, ability to resist exhaustion, and the persistence of stemness to ensure continued expansion and durability of response. In some aspects, immune cells, e.g. T cells, cultured according to the methods disclosed herein, are stem-like cells. Such cells are capable of self-renewal, proliferation and differentiation. In some aspects, immune cells, e.g. T cells, cultured according to the methods disclosed herein, are stem-like cells which also express effector-like markers. In some aspects, immune cells, e.g. T cells, cultured according to the methods disclosed herein, are stem-like cells which also maintain the ability to target and kill tumor cells.

In some aspects, the transferred CD8⁺-enriched TILs, have a greater in vivo efficacy, e.g., tumor-killing activity, as compared to TILs cultured using conventional culture conditions. In some aspects, a lower dose of the CD8⁺-enriched TILs cultured according to the methods disclosed herein is needed to elicit a response, e.g., decreased tumor volume, in a subject as compared to cells cultured using conventional culture conditions.

In some aspects, the TILs are cultured in the metabolic reprogramming media, e.g., hyperkalemic medium disclosed herein for the entirety of ex vivo culture, e.g., from the time the tumor sample is first plated through the entire expansion process, and until administration. In some aspects, the TILs are cultured in the medium disclosed herein for the duration of expansion.

In some aspects, the metabolic reprogramming media, e.g., hyperkalemic culture medium comprises a mitochondrial fuel. In some aspects, the metabolic reprogramming media, e.g., hyperkalemic culture medium, comprises O-Acetyl-L-carnitine hydrochloride. In some aspects, the metabolic reprogramming media, e.g., hyperkalemic culture medium, comprises at least about 0.1 mM, at least about 0.5 mM, at least about 1.0 mM, at least about 5 mM, or at least about 10 mM O-Acetyl-L-carnitine hydrochloride. In some aspects, the metabolic reprogramming media, e.g., hyperkalemic culture medium, comprises at least about 1.0 mM O-Acetyl-L-carnitine hydrochloride.

In some aspects, the metabolic reprogramming media, e.g., hyperkalemic culture medium, comprises inhibitor of glycolysis-mediated metabolism, e.g., a kinase inhibitor, e.g., a phosphoinositide 3-kinase inhibitor. In some aspects, the metabolic reprogramming media, e.g., hyperkalemic culture medium, comprises a phosphatidylinositol-3-kinase (PI3K) inhibitor, e.g., idelalisib (e.g., CAL-101; Selleckchem). In some aspects, the metabolic reprogramming media, e.g., hyperkalemic culture medium, comprises at least about 0.1 mM, at least about 0.5 mM, at least about 1.0 mM, at least about 5 mM, or at least about 10 mM idelalisib. In some aspects, the metabolic reprogramming media, e.g., hyperkalemic culture medium, comprises at least about 1.0 mM idelalisib.

In some aspects, the metabolic reprogramming media, e.g., hyperkalemic culture medium, further comprises one or more of (i) one or more cell expansion agents, (ii) sodium ion, (iii) one or more saccharides, (iv) calcium ion, and (v) one or more cytokines.

II.A. Potassium

Some aspects of the present disclosure are directed to methods of culturing TILs ex vivo or in vitro comprising placing a heterogeneous population of TILs in a metabolic reprogramming media, e.g., hyperkalemic medium. Some aspects of the present disclosure are directed to methods of increasing a number or percentage of CD8⁺ TILs ex vivo or in vitro comprising culturing a heterogeneous population of TILs in a metabolic reprogramming media, e.g., hyperkalemic medium. Other aspects of the present disclosure are directed to methods of preparing a CD8⁺-enriched population of tumor infiltrating lymphocytes (TILs), comprising culturing a heterogeneous population of TILs ex vivo or in vitro in a metabolic reprogramming media, e.g., hyperkalemic medium. In some aspects, the concentration of potassium ion is at least about 30 mM to at least about 100 mM. In some aspects, the concentration of potassium ion is at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, at least about 85 mM, at least about 90 mM, at least about 95 mM, or at least about 100 Mm. In some aspects, the concentration of potassium ion is at least about 50 mM. In some aspects, the concentration of potassium ion is about 40 mM. In some aspects, the concentration of potassium ion is about 45 mM. In some aspects, the concentration of potassium ion is about 50 mM.

In some aspects, the concentration of potassium ion is at least about 55 mM, at least about 60 mM, at least about 65 mM, at least about 70 mM, at least about 75 mM, at least about 80 mM, at least about 85 mM, at least about 90 mM, at least about 95 mM, or at least about 100 mM, at least about 105 mM, at least about 110 mM, at least about 115 mM, at least about 120 mM. In some aspects, the concentration of potassium ion is about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM. In some aspects, the concentration of potassium ion is about 55 mM. In some aspects, the concentration of potassium ion is about 60 mM. In some aspects, the concentration of potassium ion is about 65 mM. In some aspects, the concentration of potassium ion is about 70 mM. In some aspects the concentration of potassium ion is about 40 mM to about 90 mM.

In some aspects, the concentration of potassium ion is about 40 mM to about 90 mM. In some aspects, the concentration of potassium ion is about 40 mM to about 85 mM, about 40 mM to about 80 mM, about 40 mM to about 75 mM, about 40 mM to about 70 mM, about 40 mM to about 65 mM, about 40 mM to about 60 mM, about 40 mM to about 55 mM, or about 40 mM to about 50 mM. In some aspects, the concentration of potassium ion is about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM.

In some aspects, the concentration of potassium ion is about 50 mM to about 100 mM. In some aspects, the concentration of potassium ion is about 50 mM to about 100 mM, about 50 mM to about 95 mM, about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM.

In some aspects, the concentration of potassium ion is about 55 mM to about 100 mM. In some aspects, the concentration of potassium ion is about 55 mM to about 100 mM, about 55 mM to about 95 mM, about 55 mM to about 90 mM, about 55 mM to about 85 mM, about 55 mM to about 80 mM, about 55 mM to about 75 mM, about 55 mM to about 70 mM, about 55 mM to about 65 mM, or about 55 mM to about 60 mM.

In some aspects, the concentration of potassium ion is about 60 mM to about 100 mM. In some aspects, the concentration of potassium ion is about 60 mM to about 100 mM, about 60 mM to about 95 mM, about 60 mM to about 90 mM, about 60 mM to about 85 mM, about 60 mM to about 80 mM, about 60 mM to about 75 mM, about 60 mM to about 70 mM, or about 60 mM to about 65 mM.

In some aspects, the concentration of potassium ion is about 65 mM to about 100 mM. In some aspects, the concentration of potassium ion is about 65 mM to about 100 mM, about 65 mM to about 95 mM, about 65 mM to about 90 mM, about 65 mM to about 85 mM, about 65 mM to about 80 mM, about 65 mM to about 75 mM, or about 65 mM to about 70 mM.

In some aspects, the concentration of potassium ion is about 70 mM to about 100 mM. In some aspects, the concentration of potassium ion is about 70 mM to about 100 mM, about 70 mM to about 95 mM, about 70 mM to about 90 mM, about 70 mM to about 85 mM, about 70 mM to about 80 mM, or about 70 mM to about 75 mM.

In some aspects, the concentration of potassium ion is about 75 mM to about 100 mM. In some aspects, the concentration of potassium ion is about 75 mM to about 100 mM, about 75 mM to about 95 mM, about 75 mM to about 90 mM, about 75 mM to about 85 mM, or about 75 mM to about 80 mM.

In some aspects, the concentration of potassium ion is about 80 mM to about 100 mM. In some aspects, the concentration of potassium ion is about 80 mM to about 100 mM, about 80 mM to about 95 mM, about 80 mM to about 90 mM, or about 80 mM to about 85 mM.

In some aspects, the concentration of potassium ion is about 85 mM to about 100 mM. In some aspects, the concentration of potassium ion is about 85 mM to about 100 mM, about 85 mM to about 95 mM, or about 85 mM to about 90 mM.

In some aspects, the concentration of potassium ion is about 90 mM to about 100 mM. In some aspects, the concentration of potassium ion is about 90 mM to about 95 mM.

In some aspects, the concentration of potassium ion is about 95 mM to about 100 mM.

In some aspects, the concentration of potassium ion is about 50 mM to about 90 mM. In some aspects, the concentration of potassium ion is about 50 mM to about 80 mM. In some aspects, the concentration of potassium ion is about 60 mM to about 90 mM. In some aspects, the concentration of potassium ion is about 60 mM to about 80 mM. In some aspects, the concentration of potassium ion is about 70 mM to about 90 mM. In some aspects, the concentration of potassium ion is about 70 mM to about 80 mM. In some aspects, the concentration of potassium ion is about 80 mM to about 90 mM. In some aspects, the medium is hypertonic. In some aspects, the medium is isotonic. In some aspects, the medium comprises at least about 50 mM potassium ion and less than about 90 mM NaCl. In some aspects, the total concentration of potassium ion and NaCl is between 110 mM and 140 mM.

In some aspects, the concentration of potassium ion is about 50 mM to about 55 mM. In some aspects, the concentration of potassium ion is about 55 mM to about 60 mM. In some aspects, the concentration of potassium ion is about 60 mM to about 65 mM. In some aspects, the concentration of potassium ion is about 65 mM to about 70 mM. In some aspects, the concentration of potassium ion is about 70 mM to about 75 mM. In some aspects, the concentration of potassium ion is about 75 mM to about 80 mM. In some aspects, the concentration of potassium ion is about 80 mM to about 85 mM. In some aspects, the concentration of potassium ion is about 85 mM to about 90 mM. In some aspects, the concentration of potassium ion is about 90 mM to about 95 mM. In some aspects, the concentration of potassium ion is about 95 mM to about 100 mM. In some aspects, the concentration of potassium ion is about 100 mM to about 105 mM. In some aspects, the concentration of potassium ion is about 105 mM to about 110 mM. In some aspects, the concentration of potassium ion is about 110 mM to about 115 mM. In some aspects, the concentration of potassium ion is about 115 mM to about 120 mM.

In some aspects, the concentration of potassium ion is about 40 mM to about 90 mM. In some aspects, the concentration of potassium ion is about 40 mM to about 80 mM. In some aspects, the concentration of potassium ion is about 40 mM to about 70 mM. In some aspects, the concentration of potassium ion is about 50 mM to about 90 mM. In some aspects, the concentration of potassium ion is about 50 mM to about 80 mM. In some aspects, the concentration of potassium ion is about 50 mM to about 70 mM. In some aspects, the concentration of potassium ion is about 55 mM to about 90 mM. In some aspects, the concentration of potassium ion is about 55 mM to about 80 mM. In some aspects, the concentration of potassium ion is about 55 mM to about 70 mM. In some aspects, the concentration of potassium ion is about 60 mM to about 90 mM. In some aspects, the concentration of potassium ion is about 60 mM to about 80 mM. In some aspects, the concentration of potassium ion is about 60 mM to about 70 mM. In some aspects, the concentration of potassium ion is about 65 mM to about 90 mM. In some aspects, the concentration of potassium ion is about 65 mM to about 80 mM. In some aspects, the concentration of potassium ion is about 65 mM to about 70 mM.

In some aspects, the concentration of potassium ion is higher than about 40 mM. In some aspects, the concentration of potassium ion is about 40 mM. In some aspects, the concentration of potassium ion is higher than about 41 mM. In some aspects, the concentration of potassium ion is about 41 mM. In some aspects, the concentration of potassium ion is higher than about 42 mM. In some aspects, the concentration of potassium ion is about 42 mM. In some aspects, the concentration of potassium ion is higher than about 43 mM. In some aspects, the concentration of potassium ion is about 43 mM. In some aspects, the concentration of potassium ion is higher than about 44 mM. In some aspects, the concentration of potassium ion is about 44 mM. In some aspects, the concentration of potassium ion is higher than about 45 mM. In some aspects, the concentration of potassium ion is about 45 mM. In some aspects, the concentration of potassium ion is higher than about 46 mM. In some aspects, the concentration of potassium ion is about 46 mM. In some aspects, the concentration of potassium ion is higher than about 47 mM. In some aspects, the concentration of potassium ion is about 47 mM. In some aspects, the concentration of potassium ion is higher than about 48 mM. In some aspects, the concentration of potassium ion is about 48 mM. In some aspects, the concentration of potassium ion is higher than about 49 mM. In some aspects, the concentration of potassium ion is about 49 mM.

In some aspects, the concentration of potassium ion is higher than about 50 mM. In some aspects, the concentration of potassium ion is about 50 mM. In some aspects, the concentration of potassium ion is higher than about 51 mM. In some aspects, the concentration of potassium ion is about 51 mM. In some aspects, the concentration of potassium ion is higher than about 52 mM. In some aspects, the concentration of potassium ion is about 52 mM. In some aspects, the concentration of potassium ion is higher than about 53 mM. In some aspects, the concentration of potassium ion is about 53 mM. In some aspects, the concentration of potassium ion is higher than about 54 mM. In some aspects, the concentration of potassium ion is about 54 mM. In some aspects, the concentration of potassium ion is higher than about 55 mM. In some aspects, the concentration of potassium ion is about 55 mM. In some aspects, the concentration of potassium ion is higher than about 56 mM. In some aspects, the concentration of potassium ion is about 56 mM. In some aspects, the concentration of potassium ion is higher than about 57 mM. In some aspects, the concentration of potassium ion is about 57 mM. In some aspects, the concentration of potassium ion is higher than about 58 mM. In some aspects, the concentration of potassium ion is about 58 mM. In some aspects, the concentration of potassium ion is higher than about 59 mM. In some aspects, the concentration of potassium ion is about 59 mM.

In some aspects, the concentration of potassium ion is higher than about 60 mM. In some aspects, the concentration of potassium ion is about 60 mM. In some aspects, the concentration of potassium ion is higher than about 61 mM. In some aspects, the concentration of potassium ion is about 61 mM. In some aspects, the concentration of potassium ion is higher than about 62 mM. In some aspects, the concentration of potassium ion is about 62 mM. In some aspects, the concentration of potassium ion is higher than about 63 mM. In some aspects, the concentration of potassium ion is about 63 mM. In some aspects, the concentration of potassium ion is higher than about 64 mM. In some aspects, the concentration of potassium ion is about 64 mM. In some aspects, the concentration of potassium ion is higher than about 65 mM. In some aspects, the concentration of potassium ion is about 65 mM. In some aspects, the concentration of potassium ion is higher than about 66 mM. In some aspects, the concentration of potassium ion is about 66 mM. In some aspects, the concentration of potassium ion is higher than about 67 mM. In some aspects, the concentration of potassium ion is about 67 mM. In some aspects, the concentration of potassium ion is higher than about 68 mM. In some aspects, the concentration of potassium ion is about 68 mM. In some aspects, the concentration of potassium ion is higher than about 69 mM. In some aspects, the concentration of potassium ion is about 69 mM.

In some aspects, the concentration of potassium ion is higher than about 70 mM. In some aspects, the concentration of potassium ion is about 70 mM. In some aspects, the concentration of potassium ion is higher than about 71 mM. In some aspects, the concentration of potassium ion is about 71 mM. In some aspects, the concentration of potassium ion is higher than about 72 mM. In some aspects, the concentration of potassium ion is about 72 mM. In some aspects, the concentration of potassium ion is higher than about 73 mM. In some aspects, the concentration of potassium ion is about 73 mM. In some aspects, the concentration of potassium ion is higher than about 74 mM. In some aspects, the concentration of potassium ion is about 74 mM. In some aspects, the concentration of potassium ion is higher than about 75 mM. In some aspects, the concentration of potassium ion is about 75 mM. In some aspects, the concentration of potassium ion is higher than about 76 mM. In some aspects, the concentration of potassium ion is about 76 mM. In some aspects, the concentration of potassium ion is higher than about 77 mM. In some aspects, the concentration of potassium ion is about 77 mM. In some aspects, the concentration of potassium ion is higher than about 78 mM. In some aspects, the concentration of potassium ion is about 78 mM. In some aspects, the concentration of potassium ion is higher than about 79 mM. In some aspects, the concentration of potassium ion is about 79 mM.

In some aspects, the concentration of potassium ion is higher than about 80 mM. In some aspects, the concentration of potassium ion is about 80 mM. In some aspects, the concentration of potassium ion is higher than about 81 mM. In some aspects, the concentration of potassium ion is about 81 mM. In some aspects, the concentration of potassium ion is higher than about 82 mM. In some aspects, the concentration of potassium ion is about 82 mM. In some aspects, the concentration of potassium ion is higher than about 83 mM. In some aspects, the concentration of potassium ion is about 83 mM. In some aspects, the concentration of potassium ion is higher than about 84 mM. In some aspects, the concentration of potassium ion is about 84 mM. In some aspects, the concentration of potassium ion is higher than about 85 mM. In some aspects, the concentration of potassium ion is about 85 mM. In some aspects, the concentration of potassium ion is higher than about 86 mM. In some aspects, the concentration of potassium ion is about 86 mM. In some aspects, the concentration of potassium ion is higher than about 87 mM. In some aspects, the concentration of potassium ion is about 87 mM. In some aspects, the concentration of potassium ion is higher than about 88 mM. In some aspects, the concentration of potassium ion is about 88 mM. In some aspects, the concentration of potassium ion is higher than about 89 mM. In some aspects, the concentration of potassium ion is about 89 mM.

In some aspects, the concentration of potassium ion is higher than about 90 mM. In some aspects, the concentration of potassium ion is about 90 mM. In some aspects, the concentration of potassium ion is higher than about 91 mM. In some aspects, the concentration of potassium ion is about 91 mM. In some aspects, the concentration of potassium ion is higher than about 92 mM. In some aspects, the concentration of potassium ion is about 92 mM. In some aspects, the concentration of potassium ion is higher than about 93 mM. In some aspects, the concentration of potassium ion is about 93 mM. In some aspects, the concentration of potassium ion is higher than about 94 mM. In some aspects, the concentration of potassium ion is about 94 mM. In some aspects, the concentration of potassium ion is higher than about 95 mM. In some aspects, the concentration of potassium ion is about 95 mM. In some aspects, the concentration of potassium ion is higher than about 96 mM. In some aspects, the concentration of potassium ion is about 96 mM. In some aspects, the concentration of potassium ion is higher than about 97 mM. In some aspects, the concentration of potassium ion is about 97 mM. In some aspects, the concentration of potassium ion is higher than about 98 mM. In some aspects, the concentration of potassium ion is about 98 mM. In some aspects, the concentration of potassium ion is higher than about 99 mM. In some aspects, the concentration of potassium ion is about 99 mM.

In some aspects, the concentration of potassium ion is higher than about 100 mM. In some aspects, the concentration of potassium ion is about 100 mM.

In some aspects, the concentration of potassium ion is about 50 mM to about 90 mM, and the concentration of NaCl is less than about 90 mM to about 50 mM. In some aspects, the concentration of potassium ion is about 50 mM to about 80 mM, and the concentration of NaCl is less than about 90 mM to about 60 mM. In some aspects, the concentration of potassium ion is about 60 mM to about 90 mM, and the concentration of NaCl is less than about 90 mM to about 60 mM. In some aspects, the concentration of potassium ion is about 60 mM to about 80 mM, and the concentration of NaCl is less than about 80 mM to about 60 mM. In some aspects, the concentration of potassium ion is about 70 mM to about 90 mM, and the concentration of NaCl is less than about 70 mM to about 50 mM. In some aspects, the concentration of potassium ion is about 70 mM to about 80 mM, and the concentration of NaCl is less than about 70 mM to about 60 mM. In some aspects, the concentration of potassium ion is about 80 mM to about 90 mM, and the concentration of NaCl is less than about 60 mM to about 50 mM. In some aspects, the total concentration of potassium ion and NaCl is between 110 mM and 140 mM.

In some aspects, the concentration of potassium ion is about 50 mM to about 55 mM. In some aspects, the concentration of potassium ion is about 50 mM to about 55 mM, and the concentration of NaCl is less than about 90 mM to about 85 mM. In some aspects, the concentration of potassium ion is about 55 mM to about 60 mM. In some aspects, the concentration of potassium ion is about 55 mM to about 60 mM, and the concentration of NaCl is less than about 85 mM to about 80 mM. In some aspects, the concentration of potassium ion is about 60 mM to about 65 mM. In some aspects, the concentration of potassium ion is about 60 mM to about 65 mM, and the concentration of NaCl is less than about 80 mM to about 75 mM. In some aspects, the concentration of potassium ion is about 65 mM to about 70 mM. In some aspects, the concentration of potassium ion is about 65 mM to about 70 mM, and the concentration of NaCl is less than about 75 mM to about 70 mM. In some aspects, the concentration of potassium ion is about 70 mM to about 75 mM. In some aspects, the concentration of potassium ion is about 70 mM to about 75 mM, and the concentration of NaCl is less than about 70 mM to about 65 mM. In some aspects, the concentration of potassium ion is about 75 mM to about 80 mM. In some aspects, the concentration of potassium ion is about 75 mM to about 80 mM, and the concentration of NaCl is less than about 65 mM to about 60 mM. In some aspects, the concentration of potassium ion is about 80 mM to about 85 mM. In some aspects, the concentration of potassium ion is about 80 mM to about 85 mM, and the concentration of NaCl is less than about 60 mM to about 55 mM. In some aspects, the concentration of potassium ion is about 85 mM to about 90 mM. In some aspects, the concentration of potassium ion is about 85 mM to about 90 mM, and the concentration of NaCl is less than about 55 mM to about 50 mM. In some aspects, the concentration of potassium ion is about 90 mM to about 95 mM. In some aspects, the concentration of potassium ion is about 90 mM to about 95 mM, and the concentration of NaCl is less than about 50 mM to about 45 mM. In some aspects, the concentration of potassium ion is about 95 mM to about 100 mM. In some aspects, the concentration of potassium ion is about 95 mM to about 100 mM, and the concentration of NaCl is less than about 45 mM to about 40 mM. In some aspects, the concentration of potassium ion is about 100 mM to about 105 mM. In some aspects, the concentration of potassium ion is about 100 mM to about 105 mM, and the concentration of NaCl is less than about 40 mM to about 35 mM. In some aspects, the concentration of potassium ion is about 105 mM to about 110 mM. In some aspects, the concentration of potassium ion is about 105 mM to about 110 mM, and the concentration of NaCl is less than about 35 to about 30. In some aspects, the concentration of potassium ion is about 110 mM to about 115 mM. In some aspects, the concentration of potassium ion is about 110 mM to about 115 mM, and the concentration of NaCl is less than about 30 mM to about 25 mM. In some aspects, the concentration of potassium ion is about 115 mM to about 120 mM. In some aspects, the concentration of potassium ion is about 115 mM to about 120 mM, and the concentration of NaCl is less than about 25 mM to about 20 mM. In some aspects, the total concentration of potassium ion and NaCl is between 110 mM and 140 mM.

In some aspects, the concentration of potassium ion is higher than about 40 mM, wherein the total concentration of potassium ion and NaCl in the medium is between 110 mM and 140 mM. In some aspects, the concentration of potassium ion is about 40 mM, wherein the total concentration of potassium ion and NaCl in the medium is between 110 mM and 140 mM. In some aspects, the concentration of potassium ion is higher than about 41 mM, wherein the total concentration of potassium ion and NaCl in the medium is between 110 mM and 140 mM. In some aspects, the concentration of potassium ion is about 41 mM, wherein the total concentration of potassium ion and NaCl in the medium is between 110 mM and 140 mM. In some aspects, the concentration of potassium ion is higher than about 42 mM, wherein the total concentration of potassium ion and NaCl in the medium is between 110 mM and 140 mM. In some aspects, the concentration of potassium ion is about 42 mM, wherein the total concentration of potassium ion and NaCl in the medium is between 110 mM and 140 mM. In some aspects, the concentration of potassium ion is higher than about 43 mM, wherein the total concentration of potassium ion and NaCl in the medium is between 110 mM and 140 mM. In some aspects, the concentration of potassium ion is about 43 mM, wherein the total concentration of potassium ion and NaCl in the medium is between 110 mM and 140 mM. In some aspects, the concentration of potassium ion is higher than about 44 mM, wherein the total concentration of potassium ion and NaCl in the medium is between 110 mM and 140 mM. In some aspects, the concentration of potassium ion is about 44 mM, wherein the total concentration of potassium ion and NaCl in the medium is between 110 mM and 140 mM. In some aspects, the concentration of potassium ion is higher than about 45 mM, wherein the total concentration of potassium ion and NaCl in the medium is between 110 mM and 140 mM. In some aspects, the concentration of potassium ion is about 45 mM, wherein the total concentration of potassium ion and NaCl in the medium is between 110 mM and 140 mM. In some aspects, the concentration of potassium ion is higher than about 46 mM, wherein the total concentration of potassium ion and NaCl in the medium is between 110 mM and 140 mM. In some aspects, the concentration of potassium ion is about 46 mM, wherein the total concentration of potassium ion and NaCl in the medium is between 110 mM and 140 mM. In some aspects, the concentration of potassium ion is higher than about 47 mM, wherein the total concentration of potassium ion and NaCl in the medium is between 110 mM and 140 mM. In some aspects, the concentration of potassium ion is about 47 mM, wherein the total concentration of potassium ion and NaCl in the medium is between 110 mM and 140 mM. In some aspects, the concentration of potassium ion is higher than about 48 mM, wherein the total concentration of potassium ion and NaCl in the medium is between 110 mM and 140 mM. In some aspects, the concentration of potassium ion is about 48 mM, wherein the total concentration of potassium ion and NaCl in the medium is between 110 mM and 140 mM. In some aspects, the concentration of potassium ion is higher than about 49 mM, wherein the total concentration of potassium ion and NaCl in the medium is between 110 mM and 140 mM. In some aspects, the concentration of potassium ion is about 49 mM, wherein the total concentration of potassium ion and NaCl in the medium is between 110 mM and 140 mM.

In some aspects, the concentration of potassium ion is higher than about 50 mM, wherein the total concentration of potassium ion and NaCl in the medium is between 110 mM and 140 mM. In some aspects, the concentration of potassium ion is about 50 mM, wherein the total concentration of potassium ion and NaCl in the medium is between 110 mM and 140 mM. In some aspects, the concentration of potassium ion is higher than about 51 mM, wherein the total concentration of potassium ion and NaCl in the medium is between 110 mM and 140 mM. In some aspects, the concentration of potassium ion is about 51 mM, wherein the total concentration of potassium ion and NaCl in the medium is between 110 mM and 140 mM. In some aspects, the concentration of potassium ion is higher than about 52 mM, wherein the total concentration of potassium ion and NaCl in the medium is between 110 mM and 140 mM. In some aspects, the concentration of potassium ion is about 52 mM, wherein the total concentration of potassium ion and NaCl in the medium is between 110 mM and 140 mM. In some aspects, the concentration of potassium ion is higher than about 53 mM, wherein the total concentration of potassium ion and NaCl in the medium is between 110 mM and 140 mM. In some aspects, the concentration of potassium ion is about 53 mM, wherein the total concentration of potassium ion and NaCl in the medium is between 110 mM and 140 mM. In some aspects, the concentration of potassium ion is higher than about 54 mM, wherein the total concentration of potassium ion and NaCl in the medium is between 110 mM and 140 mM. In some aspects, the concentration of potassium ion is about 54 mM, wherein the total concentration of potassium ion and NaCl in the medium is between 110 mM and 140 mM. In some aspects, the concentration of potassium ion is higher than about 55 mM, wherein the total concentration of potassium ion and NaCl in the medium is between 110 mM and 140 mM. In some aspects, the concentration of potassium ion is about 55 mM, wherein the total concentration of potassium ion and NaCl in the medium is between 110 mM and 140 mM. In some aspects, the concentration of potassium ion is higher than about 56 mM, wherein the total concentration of potassium ion and NaCl in the medium is between 110 mM and 140 mM. In some aspects, the concentration of potassium ion is about 56 mM, wherein the total concentration of potassium ion and NaCl in the medium is between 110 mM and 140 mM. In some aspects, the concentration of potassium ion is higher than about 57 mM, wherein the total concentration of potassium ion and NaCl in the medium is between 110 mM and 140 mM. In some aspects, the concentration of potassium ion is about 57 mM, wherein the total concentration of potassium ion and NaCl in the medium is between 110 mM and 140 mM. In some aspects, the concentration of potassium ion is higher than about 58 mM, wherein the total concentration of potassium ion and NaCl in the medium is between 110 mM and 140 mM. In some aspects, the concentration of potassium ion is about 58 mM, wherein the total concentration of potassium ion and NaCl in the medium is between 110 mM and 140 mM. In some aspects, the concentration of potassium ion is higher than about 59 mM, wherein the total concentration of potassium ion and NaCl in the medium is between 110 mM and 140 mM. In some aspects, the concentration of potassium ion is about 59 mM, wherein the total concentration of potassium ion and NaCl in the medium is between 110 mM and 140 mM.

In some aspects, the concentration of potassium ion is higher than about 60 mM, wherein the total concentration of potassium ion and NaCl in the medium is between 110 mM and 140 mM. In some aspects, the concentration of potassium ion is about 60 mM, wherein the total concentration of potassium ion and NaCl in the medium is between 110 mM and 140 mM. In some aspects, the concentration of potassium ion is higher than about 61 mM, wherein the total concentration of potassium ion and NaCl in the medium is between 110 mM and 140 mM. In some aspects, the concentration of potassium ion is about 61 mM, wherein the total concentration of potassium ion and NaCl in the medium is between 110 mM and 140 mM. In some aspects, the concentration of potassium ion is higher than about 62 mM, wherein the total concentration of potassium ion and NaCl in the medium is between 110 mM and 140 mM. In some aspects, the concentration of potassium ion is about 62 mM, wherein the total concentration of potassium ion and NaCl in the medium is between 110 mM and 140 mM. In some aspects, the concentration of potassium ion is higher than about 63 mM, wherein the total concentration of potassium ion and NaCl in the medium is between 110 mM and 140 mM. In some aspects, the concentration of potassium ion is about 63 mM, wherein the total concentration of potassium ion and NaCl in the medium is between 110 mM and 140 mM. In some aspects, the concentration of potassium ion is higher than about 64 mM, wherein the total concentration of potassium ion and NaCl in the medium is between 110 mM and 140 mM. In some aspects, the concentration of potassium ion is about 64 mM, wherein the total concentration of potassium ion and NaCl in the medium is between 110 mM and 140 mM. In some aspects, the concentration of potassium ion is higher than about 65 mM, wherein the total concentration of potassium ion and NaCl in the medium is between 110 mM and 140 mM. In some aspects, the concentration of potassium ion is about 65 mM, wherein the total concentration of potassium ion and NaCl in the medium is between 110 mM and 140 mM. In some aspects, the concentration of potassium ion is higher than about 66 mM, wherein the total concentration of potassium ion and NaCl in the medium is between 110 mM and 140 mM. In some aspects, the concentration of potassium ion is about 66 mM, wherein the total concentration of potassium ion and NaCl in the medium is between 110 mM and 140 mM. In some aspects, the concentration of potassium ion is higher than about 67 mM, wherein the total concentration of potassium ion and NaCl in the medium is between 110 mM and 140 mM. In some aspects, the concentration of potassium ion is about 67 mM, wherein the total concentration of potassium ion and NaCl in the medium is between 110 mM and 140 mM. In some aspects, the concentration of potassium ion is higher than about 68 mM, wherein the total concentration of potassium ion and NaCl in the medium is between 110 mM and 140 mM. In some aspects, the concentration of potassium ion is about 68 mM, wherein the total concentration of potassium ion and NaCl in the medium is between 110 mM and 140 mM. In some aspects, the concentration of potassium ion is higher than about 69 mM, wherein the total concentration of potassium ion and NaCl in the medium is between 110 mM and 140 mM. In some aspects, the concentration of potassium ion is about 69 mM, wherein the total concentration of potassium ion and NaCl in the medium is between 110 mM and 140 mM.

In some aspects, the concentration of potassium ion is higher than about 70 mM, wherein the total concentration of potassium ion and NaCl in the medium is between 110 mM and 140 mM. In some aspects, the concentration of potassium ion is about 70 mM, wherein the total concentration of potassium ion and NaCl in the medium is between 110 mM and 140 mM. In some aspects, the concentration of potassium ion is higher than about 71 mM, wherein the total concentration of potassium ion and NaCl in the medium is between 110 mM and 140 mM. In some aspects, the concentration of potassium ion is about 71 mM, wherein the total concentration of potassium ion and NaCl in the medium is between 110 mM and 140 mM. In some aspects, the concentration of potassium ion is higher than about 72 mM, wherein the total concentration of potassium ion and NaCl in the medium is between 110 mM and 140 mM. In some aspects, the concentration of potassium ion is about 72 mM, wherein the total concentration of potassium ion and NaCl in the medium is between 110 mM and 140 mM. In some aspects, the concentration of potassium ion is higher than about 73 mM, wherein the total concentration of potassium ion and NaCl in the medium is between 110 mM and 140 mM. In some aspects, the concentration of potassium ion is about 73 mM, wherein the total concentration of potassium ion and NaCl in the medium is between 110 mM and 140 mM. In some aspects, the concentration of potassium ion is higher than about 74 mM, wherein the total concentration of potassium ion and NaCl in the medium is between 110 mM and 140 mM. In some aspects, the concentration of potassium ion is about 74 mM, wherein the total concentration of potassium ion and NaCl in the medium is between 110 mM and 140 mM. In some aspects, the concentration of potassium ion is higher than about 75 mM, wherein the total concentration of potassium ion and NaCl in the medium is between 110 mM and 140 mM. In some aspects, the concentration of potassium ion is about 75 mM, wherein the total concentration of potassium ion and NaCl in the medium is between 110 mM and 140 mM. In some aspects, the concentration of potassium ion is higher than about 76 mM, wherein the total concentration of potassium ion and NaCl in the medium is between 110 mM and 140 mM. In some aspects, the concentration of potassium ion is about 76 mM, wherein the total concentration of potassium ion and NaCl in the medium is between 110 mM and 140 mM. In some aspects, the concentration of potassium ion is higher than about 77 mM, wherein the total concentration of potassium ion and NaCl in the medium is between 110 mM and 140 mM. In some aspects, the concentration of potassium ion is about 77 mM, wherein the total concentration of potassium ion and NaCl in the medium is between 110 mM and 140 mM. In some aspects, the concentration of potassium ion is higher than about 78 mM, wherein the total concentration of potassium ion and NaCl in the medium is between 110 mM and 140 mM. In some aspects, the concentration of potassium ion is about 78 mM, wherein the total concentration of potassium ion and NaCl in the medium is between 110 mM and 140 mM. In some aspects, the concentration of potassium ion is higher than about 79 mM, wherein the total concentration of potassium ion and NaCl in the medium is between 110 mM and 140 mM. In some aspects, the concentration of potassium ion is about 79 mM, wherein the total concentration of potassium ion and NaCl in the medium is between 110 mM and 140 mM.

In some aspects, the concentration of potassium ion is higher than about 80 mM, wherein the total concentration of potassium ion and NaCl in the medium is between 110 mM and 140 mM. In some aspects, the concentration of potassium ion is about 80 mM, wherein the total concentration of potassium ion and NaCl in the medium is between 110 mM and 140 mM. In some aspects, the concentration of potassium ion is higher than about 81 mM, wherein the total concentration of potassium ion and NaCl in the medium is between 110 mM and 140 mM. In some aspects, the concentration of potassium ion is about 81 mM, wherein the total concentration of potassium ion and NaCl in the medium is between 110 mM and 140 mM. In some aspects, the concentration of potassium ion is higher than about 82 mM, wherein the total concentration of potassium ion and NaCl in the medium is between 110 mM and 140 mM. In some aspects, the concentration of potassium ion is about 82 mM, wherein the total concentration of potassium ion and NaCl in the medium is between 110 mM and 140 mM. In some aspects, the concentration of potassium ion is higher than about 83 mM, wherein the total concentration of potassium ion and NaCl in the medium is between 110 mM and 140 mM. In some aspects, the concentration of potassium ion is about 83 mM, wherein the total concentration of potassium ion and NaCl in the medium is between 110 mM and 140 mM. In some aspects, the concentration of potassium ion is higher than about 84 mM, wherein the total concentration of potassium ion and NaCl in the medium is between 110 mM and 140 mM. In some aspects, the concentration of potassium ion is about 84 mM, wherein the total concentration of potassium ion and NaCl in the medium is between 110 mM and 140 mM. In some aspects, the concentration of potassium ion is higher than about 85 mM, wherein the total concentration of potassium ion and NaCl in the medium is between 110 mM and 140 mM. In some aspects, the concentration of potassium ion is about 85 mM, wherein the total concentration of potassium ion and NaCl in the medium is between 110 mM and 140 mM. In some aspects, the concentration of potassium ion is higher than about 86 mM, wherein the total concentration of potassium ion and NaCl in the medium is between 110 mM and 140 mM. In some aspects, the concentration of potassium ion is about 86 mM, wherein the total concentration of potassium ion and NaCl in the medium is between 110 mM and 140 mM. In some aspects, the concentration of potassium ion is higher than about 87 mM, wherein the total concentration of potassium ion and NaCl in the medium is between 110 mM and 140 mM. In some aspects, the concentration of potassium ion is about 87 mM, wherein the total concentration of potassium ion and NaCl in the medium is between 110 mM and 140 mM. In some aspects, the concentration of potassium ion is higher than about 88 mM, wherein the total concentration of potassium ion and NaCl in the medium is between 110 mM and 140 mM. In some aspects, the concentration of potassium ion is about 88 mM, wherein the total concentration of potassium ion and NaCl in the medium is between 110 mM and 140 mM. In some aspects, the concentration of potassium ion is higher than about 89 mM, wherein the total concentration of potassium ion and NaCl in the medium is between 110 mM and 140 mM. In some aspects, the concentration of potassium ion is about 89 mM, wherein the total concentration of potassium ion and NaCl in the medium is between 110 mM and 140 mM.

In some aspects, the concentration of potassium ion is higher than about 90 mM, wherein the total concentration of potassium ion and NaCl in the medium is between 110 mM and 140 mM. In some aspects, the concentration of potassium ion is about 90 mM, wherein the total concentration of potassium ion and NaCl in the medium is between 110 mM and 140 mM. In some aspects, the concentration of potassium ion is higher than about 91 mM, wherein the total concentration of potassium ion and NaCl in the medium is between 110 mM and 140 mM. In some aspects, the concentration of potassium ion is about 91 mM, wherein the total concentration of potassium ion and NaCl in the medium is between 110 mM and 140 mM. In some aspects, the concentration of potassium ion is higher than about 92 mM, wherein the total concentration of potassium ion and NaCl in the medium is between 110 mM and 140 mM. In some aspects, the concentration of potassium ion is about 92 mM, wherein the total concentration of potassium ion and NaCl in the medium is between 110 mM and 140 mM. In some aspects, the concentration of potassium ion is higher than about 93 mM, wherein the total concentration of potassium ion and NaCl in the medium is between 110 mM and 140 mM. In some aspects, the concentration of potassium ion is about 93 mM, wherein the total concentration of potassium ion and NaCl in the medium is between 110 mM and 140 mM. In some aspects, the concentration of potassium ion is higher than about 94 mM, wherein the total concentration of potassium ion and NaCl in the medium is between 110 mM and 140 mM. In some aspects, the concentration of potassium ion is about 94 mM, wherein the total concentration of potassium ion and NaCl in the medium is between 110 mM and 140 mM. In some aspects, the concentration of potassium ion is higher than about 95 mM, wherein the total concentration of potassium ion and NaCl in the medium is between 110 mM and 140 mM. In some aspects, the concentration of potassium ion is about 95 mM, wherein the total concentration of potassium ion and NaCl in the medium is between 110 mM and 140 mM. In some aspects, the concentration of potassium ion is higher than about 96 mM, wherein the total concentration of potassium ion and NaCl in the medium is between 110 mM and 140 mM. In some aspects, the concentration of potassium ion is about 96 mM, wherein the total concentration of potassium ion and NaCl in the medium is between 110 mM and 140 mM. In some aspects, the concentration of potassium ion is higher than about 97 mM, wherein the total concentration of potassium ion and NaCl in the medium is between 110 mM and 140 mM. In some aspects, the concentration of potassium ion is about 97 mM, wherein the total concentration of potassium ion and NaCl in the medium is between 110 mM and 140 mM. In some aspects, the concentration of potassium ion is higher than about 98 mM, wherein the total concentration of potassium ion and NaCl in the medium is between 110 mM and 140 mM. In some aspects, the concentration of potassium ion is about 98 mM, wherein the total concentration of potassium ion and NaCl in the medium is between 110 mM and 140 mM. In some aspects, the concentration of potassium ion is higher than about 99 mM, wherein the total concentration of potassium ion and NaCl in the medium is between 110 mM and 140 mM. In some aspects, the concentration of potassium ion is about 99 mM, wherein the total concentration of potassium ion and NaCl in the medium is between 110 mM and 140 mM.

In some aspects, the concentration of potassium ion is higher than about 100 mM, wherein the total concentration of potassium ion and NaCl in the medium is between 110 mM and 140 mM. In some aspects, the concentration of potassium ion is about 100 mM, wherein the total concentration of potassium ion and NaCl in the medium is between 110 mM and 140 mM. In some aspects, the concentration of potassium ion is higher than about 101 mM, wherein the total concentration of potassium ion and NaCl in the medium is between 110 mM and 140 mM. In some aspects, the concentration of potassium ion is about 101 mM, wherein the total concentration of potassium ion and NaCl in the medium is between 110 mM and 140 mM. In some aspects, the concentration of potassium ion is higher than about 102 mM, wherein the total concentration of potassium ion and NaCl in the medium is between 110 mM and 140 mM. In some aspects, the concentration of potassium ion is about 102 mM, wherein the total concentration of potassium ion and NaCl in the medium is between 110 mM and 140 mM. In some aspects, the concentration of potassium ion is higher than about 103 mM, wherein the total concentration of potassium ion and NaCl in the medium is between 110 mM and 140 mM. In some aspects, the concentration of potassium ion is about 103 mM, wherein the total concentration of potassium ion and NaCl in the medium is between 110 mM and 140 mM. In some aspects, the concentration of potassium ion is higher than about 104 mM, wherein the total concentration of potassium ion and NaCl in the medium is between 110 mM and 140 mM. In some aspects, the concentration of potassium ion is about 104 mM, wherein the total concentration of potassium ion and NaCl in the medium is between 110 mM and 140 mM. In some aspects, the concentration of potassium ion is higher than about 105 mM, wherein the total concentration of potassium ion and NaCl in the medium is between 110 mM and 140 mM. In some aspects, the concentration of potassium ion is about 105 mM, wherein the total concentration of potassium ion and NaCl in the medium is between 110 mM and 140 mM. In some aspects, the concentration of potassium ion is higher than about 106 mM, wherein the total concentration of potassium ion and NaCl in the medium is between 110 mM and 140 mM. In some aspects, the concentration of potassium ion is about 106 mM, wherein the total concentration of potassium ion and NaCl in the medium is between 110 mM and 140 mM. In some aspects, the concentration of potassium ion is higher than about 107 mM, wherein the total concentration of potassium ion and NaCl in the medium is between 110 mM and 140 mM. In some aspects, the concentration of potassium ion is about 107 mM, wherein the total concentration of potassium ion and NaCl in the medium is between 110 mM and 140 mM. In some aspects, the concentration of potassium ion is higher than about 108 mM, wherein the total concentration of potassium ion and NaCl in the medium is between 110 mM and 140 mM. In some aspects, the concentration of potassium ion is about 108 mM, wherein the total concentration of potassium ion and NaCl in the medium is between 110 mM and 140 mM. In some aspects, the concentration of potassium ion is higher than about 109 mM, wherein the total concentration of potassium ion and NaCl in the medium is between 110 mM and 140 mM. In some aspects, the concentration of potassium ion is about 109 mM, wherein the total concentration of potassium ion and NaCl in the medium is between 110 mM and 140 mM.

In some aspects, the concentration of potassium ion is higher than about 110 mM, wherein the total concentration of potassium ion and NaCl in the medium is between 110 mM and 140 mM. In some aspects, the concentration of potassium ion is about 110 mM, wherein the total concentration of potassium ion and NaCl in the medium is between 110 mM and 140 mM. In some aspects, the concentration of potassium ion is higher than about 111 mM, wherein the total concentration of potassium ion and NaCl in the medium is between 110 mM and 140 mM. In some aspects, the concentration of potassium ion is about 111 mM, wherein the total concentration of potassium ion and NaCl in the medium is between 110 mM and 140 mM. In some aspects, the concentration of potassium ion is higher than about 112 mM, wherein the total concentration of potassium ion and NaCl in the medium is between 110 mM and 140 mM. In some aspects, the concentration of potassium ion is about 112 mM, wherein the total concentration of potassium ion and NaCl in the medium is between 110 mM and 140 mM. In some aspects, the concentration of potassium ion is higher than about 113 mM, wherein the total concentration of potassium ion and NaCl in the medium is between 110 mM and 140 mM. In some aspects, the concentration of potassium ion is about 113 mM, wherein the total concentration of potassium ion and NaCl in the medium is between 110 mM and 140 mM. In some aspects, the concentration of potassium ion is higher than about 114 mM, wherein the total concentration of potassium ion and NaCl in the medium is between 110 mM and 140 mM. In some aspects, the concentration of potassium ion is about 114 mM, wherein the total concentration of potassium ion and NaCl in the medium is between 110 mM and 140 mM. In some aspects, the concentration of potassium ion is higher than about 115 mM, wherein the total concentration of potassium ion and NaCl in the medium is between 110 mM and 140 mM. In some aspects, the concentration of potassium ion is about 115 mM, wherein the total concentration of potassium ion and NaCl in the medium is between 110 mM and 140 mM. In some aspects, the concentration of potassium ion is higher than about 116 mM, wherein the total concentration of potassium ion and NaCl in the medium is between 110 mM and 140 mM. In some aspects, the concentration of potassium ion is about 116 mM, wherein the total concentration of potassium ion and NaCl in the medium is between 110 mM and 140 mM. In some aspects, the concentration of potassium ion is higher than about 117 mM, wherein the total concentration of potassium ion and NaCl in the medium is between 110 mM and 140 mM. In some aspects, the concentration of potassium ion is about 117 mM, wherein the total concentration of potassium ion and NaCl in the medium is between 110 mM and 140 mM. In some aspects, the concentration of potassium ion is higher than about 118 mM, wherein the total concentration of potassium ion and NaCl in the medium is between 110 mM and 140 mM. In some aspects, the concentration of potassium ion is about 118 mM, wherein the total concentration of potassium ion and NaCl in the medium is between 110 mM and 140 mM. In some aspects, the concentration of potassium ion is higher than about 119 mM, wherein the total concentration of potassium ion and NaCl in the medium is between 110 mM and 140 mM. In some aspects, the concentration of potassium ion is about 119 mM, wherein the total concentration of potassium ion and NaCl in the medium is between 110 mM and 140 mM.

In some aspects, the concentration of potassium ion is higher than about 120 mM, wherein the total concentration of potassium ion and NaCl in the medium is between 110 mM and 140 mM. In some aspects, the concentration of potassium ion is about 120 mM, wherein the total concentration of potassium ion and NaCl in the medium is between 110 mM and 140 mM. In some aspects, the concentration of potassium ion is higher than about 121 mM, wherein the total concentration of potassium ion and NaCl in the medium is between 110 mM and 140 mM. In some aspects, the concentration of potassium ion is about 121 mM, wherein the total concentration of potassium ion and NaCl in the medium is between 110 mM and 140 mM. In some aspects, the concentration of potassium ion is higher than about 122 mM, wherein the total concentration of potassium ion and NaCl in the medium is between 110 mM and 140 mM. In some aspects, the concentration of potassium ion is about 122 mM, wherein the total concentration of potassium ion and NaCl in the medium is between 110 mM and 140 mM. In some aspects, the concentration of potassium ion is higher than about 123 mM, wherein the total concentration of potassium ion and NaCl in the medium is between 110 mM and 140 mM. In some aspects, the concentration of potassium ion is about 123 mM, wherein the total concentration of potassium ion and NaCl in the medium is between 110 mM and 140 mM. In some aspects, the concentration of potassium ion is higher than about 124 mM, wherein the total concentration of potassium ion and NaCl in the medium is between 110 mM and 140 mM. In some aspects, the concentration of potassium ion is about 124 mM, wherein the total concentration of potassium ion and NaCl in the medium is between 110 mM and 140 mM. In some aspects, the concentration of potassium ion is higher than about 125 mM, wherein the total concentration of potassium ion and NaCl in the medium is between 110 mM and 140 mM. In some aspects, the concentration of potassium ion is about 125 mM, wherein the total concentration of potassium ion and NaCl in the medium is between 110 mM and 140 mM. In some aspects, the concentration of potassium ion is higher than about 126 mM, wherein the total concentration of potassium ion and NaCl in the medium is between 110 mM and 140 mM. In some aspects, the concentration of potassium ion is about 126 mM, wherein the total concentration of potassium ion and NaCl in the medium is between 110 mM and 140 mM. In some aspects, the concentration of potassium ion is higher than about 127 mM, wherein the total concentration of potassium ion and NaCl in the medium is between 110 mM and 140 mM. In some aspects, the concentration of potassium ion is about 127 mM, wherein the total concentration of potassium ion and NaCl in the medium is between 110 mM and 140 mM. In some aspects, the concentration of potassium ion is higher than about 128 mM, wherein the total concentration of potassium ion and NaCl in the medium is between 110 mM and 140 mM. In some aspects, the concentration of potassium ion is about 128 mM, wherein the total concentration of potassium ion and NaCl in the medium is between 110 mM and 140 mM. In some aspects, the concentration of potassium ion is higher than about 129 mM, wherein the total concentration of potassium ion and NaCl in the medium is between 110 mM and 140 mM. In some aspects, the concentration of potassium ion is about 129 mM, wherein the total concentration of potassium ion and NaCl in the medium is between 110 mM and 140 mM. In some aspects, the concentration of potassium ion is higher than about 130 mM, wherein the total concentration of potassium ion and NaCl in the medium is between 110 mM and 140 mM. In some aspects, the concentration of potassium ion is about 130 mM, wherein the total concentration of potassium ion and NaCl in the medium is between 110 mM and 140 mM.

In some aspects, the hyperkalemic medium comprising a high concentration of potassium ion can be prepared by adding a sufficient amount of a potassium salt in a medium. In some aspects, non-limiting examples of potassium salt include potassium aminetrichloroplatinate, potassium aquapentachlororuthenate, potassium bis(oxalato)platinate(II) dihydrate, potassium bisulfate, potassium borohydride, potassium bromide, potassium carbonate, potassium chloride, potassium chromate, potassium dichromate, potassium dicyanoargentate, potassium dicyanoaurate, potassium fluoride, potassium fluorosulfate, potassium hexachloroiridate, potassium hexachloroosmate, potassium hexachloropalladate, potassium hexachloroplatinate, potassium hexachlororhenate, potassium hexacyanochromate, potassium hexacyanoferrate, potassium hexacyanoruthenate(II) hydrate, potassium hexafluoroantimonate, potassium hexafluoronickelate, potassium hexafluorophosphate, potassium hexafluorotitanate, potassium hexafluorozirconate, potassium hexahydroxoantimonate, potassium hexaiodoplatinate, potassium hexaiodorhenate, potassium hydroxide, potassium iodate, potassium iodide, potassium manganate, potassium metavanadate, potassium molybdate, potassium nitrate, potassium nitrosodisulfonate, potassium osmate(VI) dihydrate, potassium pentachloronitrosylruthenate, potassium perchlorate, potassium perrhenate, potassium perruthenate, potassium persulfate, potassium phosphate dibasic, potassium phosphate monobasic, potassium pyrophosphate, potassium selenocyanate, potassium selenocyanate, potassium stannate trihydrate, potassium sulfate, potassium tellurate hydrate, potassium tellurite, potassium tetraborate tetrahydrate, potassium tetrabromoaurate, potassium tetrabromopalladate, potassium tetrachloropalladate, potassium tetrachloroplatinate, potassium tetracyanopalladate, potassium tetracyanoplatinate, potassium tetrafluoroborate, potassium tetranitroplatinate, potassium tetrathionate, potassium p-toluenethiosulfonate, and potassium hydroxycitrate tribasic monohydrate. In some aspects, the potassium salt comprises potassium chloride (KCl). In some aspects, the potassium salt comprises potassium gluconate. In certain aspects, the potassium salt comprises potassium citrate. In certain aspects, the potassium salt comprises potassium hydroxycitrate. In some aspects, the potassium salt comprises a combination of any of the potassium salts disclosed herein.

II.B. Cell Expansion Agents

In some aspects, the metabolic reprogramming media, e.g., hyperkalemic medium, further comprises a cell expansion agent. As used herein, a “cell expansion agent” refers to an agent, e.g., small molecule, polypeptide, or any combination thereof, that promotes the in vitro and/or ex vivo growth and proliferation of cultured immune cells, e.g., TILs. In some aspects, the cell expansion agent comprises a PI3K inhibitor. In some aspects, the medium further comprises an AKT inhibitor. In some aspects, the medium further comprises a PI3K inhibitor and an AKT inhibitor. In some aspects, the PI3K inhibitor comprises LY294002. In some aspects, the PI3K inhibitor comprises IC87114. In some aspects, the PI3K inhibitor comprises idelalisib (see, e.g., Peterson et al., Blood Adv. 2(3):210-23 (2018)). In some aspects, the medium further comprises a GSK3B inhibitor. In some aspects, the GSK3B inhibitor comprises TWS119. In some aspects, the medium further comprises an ACLY inhibitor. In some aspects, the ACLY inhibitor comprises potassium hydroxycitrate tribasic monohydrate. In some aspects, the PI3K inhibitor comprises hydroxyl citrate. In some aspects, the PI3K inhibitor comprises pictilisib. In some aspects, the PI3K inhibitor comprises CAL-101. In some aspects, the AKT inhibitor comprises MK2206, A443654, or AKTi-VIII (CAS 612847-09-3).

II.C. Sodium

In some aspects, the metabolic reprogramming media, e.g., hyperkalemic medium, further comprises sodium ion (e.g., NaCl). In some aspects, the metabolic reprogramming media comprises sodium ion (e.g., NaCl) at a concentration of less than about 115 mM. In some aspects the metabolic reprogramming media, comprises sodium ion (e.g., NaCl) at a concentration of 40 mM to about 80 mM.

In some aspects, the target concentration of sodium is reached by starting with a basal medium comprising a higher concentration of sodium ion (e.g., NaCl) and diluting the solution to reach the target concentration of sodium ion (e.g., NaCl). In some aspects, the target concentration of sodium is reached by raising the concentration of sodium ion (e.g., NaCl) by adding one or more sodium salts (e.g., more NaCl). Non-limiting examples of sodium salts include sodium (meta)periodate, sodium arsenyl tartrate hydrate, sodium azide, sodium benzyloxide, sodium bromide, sodium carbonate, sodium chloride, sodium chromate, sodium cyclohexanebutyrate, sodium ethanethiolate, sodium fluoride, sodium fluorophosphate, sodium formate, sodium hexachloroiridate(III) hydrate, sodium hexachloroiridate(IV) hexahydrate, sodium hexachloroplatinate(IV) hexahydrate, sodium hexachlororhodate(III), sodium hexafluoroaluminate, sodium hexafluoroantimonate(V), sodium hexafluoroarsenate(V), sodium hexafluoroferrate(III), sodium hexafluorophosphate, sodium hexafluorosilicate, sodium hexahydroxyplatinate(IV), sodium hexametaphosphate, sodium hydrogen difluoride, sodium hydrogen sulfate, sodium hydrogencyanamide, sodium hydroxide, sodium iodide, sodium metaborate tetrahydrate, sodium metasilicate nonahydrate, sodium metavanadate, sodium molybdate, sodium nitrate, sodium nitrite, sodium oxalate, sodium perborate monohydrate, sodium percarbonate, sodium perchlorate, sodium periodate, sodium permanganate, sodium perrhenate, sodium phosphate, sodium pyrophosphate, sodium selenate, sodium selenite, sodium stannate, sodium sulfate, sodium tellurite, sodium tetraborate, sodium tetrachloroaluminate, sodium tetrachloroaurate(III), sodium tetrachloropalladate(II), sodium tetrachloroplatinate(II), sodium thiophosphate tribasic, sodium thiosulfate, sodium thiosulfate pentahydrate, sodium yttrium oxyfluoride, Trisodium trimetaphosphate, and any combination thereof. In some aspects, the sodium salt comprises sodium chloride (NaCl). In some aspects, the sodium salt comprises sodium gluconate. In certain aspects, the sodium salt comprises sodium bicarbonate. In certain aspects, the sodium salt comprises sodium hydroxycitrate. In certain aspects, the sodium salt comprises sodium phosphate.

In some aspects, the concentration of the sodium ion (e.g., NaCl) is less than that of the basal medium. In some aspects, the concentration of the sodium ion (e.g., NaCl) is reduced as the concentration of potassium ion is increased. In some aspects, the concentration of the sodium ion (e.g., NaCl) is from about 25 mM to about 115 mM. In some aspects, the concentration of the sodium ion (e.g., NaCl) is from about 25 mM to about 100 mM, about 30 mM to about 40 mM, about 30 mM to about 50 mM, about 30 mM to about 60 mM, about 30 mM to about 70 mM, about 30 mM to about 80 mM, about 40 mM to about 50 mM, about 40 mM to about 60 mM, about 40 mM to about 70 mM, about 40 mM to about 80 mM, about 50 mM to about 55 mM, about 50 mM to about 60 mM, about 50 mM to about 65 mM, about 50 mM to about 70 mM, about 50 mM to about 75 mM, about 50 mM to about 80 mM, about 55 mM to about 60 mM, about 55 mM to about 65 mM, about 55 mM to about 70 mM, about 55 mM to about 75 mM, about 55 mM to about 80 mM, about 60 mM to about 65 mM, about 60 mM to about 70 mM, about 60 mM to about 75 mM, about 60 mM to about 80 mM, about 70 mM to about 75 mM, about 70 mM to about 80 mM, or about 75 mM to about 80 mM. In certain aspects, the concentration of the sodium ion (e.g., NaCl) is from about 40 mM to about 80 mM. In some aspects, the concentration of the sodium ion (e.g., NaCl) is from about 50 mM to about 85 mM. In some aspects, the concentration of the sodium ion (e.g., NaCl) is from about 55 mM to about 80 mM. In some aspects, the concentration of the sodium ion (e.g., NaCl) is from about 30 mM to about 35 mM. In some aspects, the concentration of the sodium ion (e.g., NaCl) is from about 35 mM to about 40 mM. In some aspects, the concentration of the sodium ion (e.g., NaCl) is from about 40 mM to about 45 mM. In some aspects, the concentration of the sodium ion (e.g., NaCl) is from about 45 mM to about 50 mM. In some aspects, the concentration of the sodium ion (e.g., NaCl) is from about 50 mM to about 55 mM. In some aspects, the concentration of the sodium ion (e.g., NaCl) is from about 55 mM to about 60 mM. In some aspects, the concentration of the sodium ion (e.g., NaCl) is from about 60 mM to about 65 mM. In some aspects, the concentration of the sodium ion (e.g., NaCl) is from about 65 mM to about 70 mM. In some aspects, the concentration of the sodium ion (e.g., NaCl) is from about 70 mM to about 75 mM. In some aspects, the concentration of the sodium ion (e.g., NaCl) is from about 75 mM to about 80 mM. In some aspects, the concentration of the sodium ion (e.g., NaCl) is from about 80 mM to about 85 mM.

In some aspects, the concentration of the sodium ion (e.g., NaCl) is about 30 mM, about 35 mM, about 40 mM, about 45 mM, about 50 mM, about 55 mM, about 60 mM, about 65 mM, about 70 mM, about 75 mM, about 80 mM, about 85 mM, or about 90 mM. In some aspects, the concentration of sodium ion (e.g., NaCl) is about 40 mM. In some aspects, the concentration of sodium ion (e.g., NaCl) is about 45 mM. In some aspects, the concentration of sodium ion (e.g., NaCl) is about 50 mM. In some aspects, the concentration of sodium ion (e.g., NaCl) is about 55 mM. In some aspects, the concentration of sodium ion (e.g., NaCl) is about 60 mM. In some aspects, the concentration of sodium ion (e.g., NaCl) is about 65 mM. In some aspects, the concentration of sodium ion (e.g., NaCl) is about 70 mM. In some aspects, the concentration of sodium ion (e.g., NaCl) is about 75 mM. In some aspects, the concentration of sodium ion (e.g., NaCl) is about 80 mM.

In some aspects, the medium comprises about 40 mM to about 90 mM potassium ion and about 40 mM to about 80 mM sodium ion (e.g., NaCl).

In some aspects, the medium comprises about 50 mM to about 75 mM potassium ion and about 80 mM to about 90 mM sodium ion (e.g., NaCl). In some aspects, the medium comprises about 55 mM to about 75 mM potassium ion and about 80 mM to about 90 mM sodium ion (e.g., NaCl). In some aspects, the medium comprises about 60 mM to about 75 mM potassium ion and about 80 mM to about 90 mM sodium ion (e.g., NaCl). In some aspects, the medium comprises about 65 mM to about 75 mM potassium ion and about 80 mM to about 85 mM sodium ion (e.g., NaCl). In some aspects, the medium comprises about 65 mM potassium ion and about 80 mM to about 85 mM sodium ion (e.g., NaCl). In some aspects, the medium comprises about 66 mM potassium ion and about 80 mM to about 85 mM sodium ion (e.g., NaCl). In some aspects, the medium comprises about 67 mM potassium ion and about 80 mM to about 85 mM sodium ion (e.g., NaCl). In some aspects, the medium comprises about 68 mM potassium ion and about 80 mM to about 85 mM sodium ion (e.g., NaCl). In some aspects, the medium comprises about 69 mM potassium ion and about 80 mM to about 85 mM sodium ion (e.g., NaCl). In some aspects, the medium comprises about 70 mM potassium ion and about 80 mM to about 85 mM sodium ion (e.g., NaCl). In some aspects, the medium comprises about 71 mM potassium ion and about 80 mM to about 85 mM sodium ion (e.g., NaCl). In some aspects, the medium comprises about 72 mM potassium ion and about 80 mM to about 85 mM sodium ion (e.g., NaCl). In some aspects, the medium comprises about 73 mM potassium ion and about 80 mM to about 85 mM sodium ion (e.g., NaCl). In some aspects, the medium comprises about 74 mM potassium ion and about 80 mM to about 85 mM sodium ion (e.g., NaCl). In some aspects, the medium comprises about 75 mM potassium ion and about 80 mM to about 85 mM sodium ion (e.g., NaCl). In some aspects, the medium comprises about 65 mM potassium ion and about 80 mM sodium ion (e.g., NaCl). In some aspects, the medium comprises about 65 mM potassium ion and about 85 mM sodium ion (e.g., NaCl). In some aspects, the medium comprises about 65 mM potassium ion and about 90 mM sodium ion (e.g., NaCl). In some aspects, the medium comprises about 70 mM potassium ion and about 80 mM sodium ion (e.g., NaCl). In some aspects, the medium comprises about 70 mM potassium ion and about 85 mM sodium ion (e.g., NaCl). In some aspects, the medium comprises about 70 mM potassium ion and about 90 mM sodium ion (e.g., NaCl). In some aspects, the medium comprises about 75 mM potassium ion and about 80 mM sodium ion (e.g., NaCl). In some aspects, the medium comprises about 75 mM potassium ion and about 85 mM sodium ion (e.g., NaCl). In some aspects, the medium comprises about 75 mM potassium ion and about 90 mM sodium ion (e.g., NaCl).

In some aspects, the medium comprises about 40 mM to about 90 mM potassium ion and about 30 mM to about 109 mM NaCl, wherein the concentration of NaCl (mM) is equal to or lower than (135—potassium ion concentration). In some aspects, the medium comprises about 40 mM potassium ion and less than or equal to about 95 mM NaCl (e.g., about 95 mM, about 94 mM, about 93 mM, about 92 mM, about 91 mM, about 90 mM, about 85 mM, about 80 mM, about 75 mM, about 70 mM, about 65 mM, about 60 mM, about 55 mM, or about 50 mM NaCl). In some aspects, the medium comprises about 45 mM potassium ion and less than or equal to about 90 mM NaCl (e.g., about 90 mM, about 89 mM, about 88 mM, about 87 mM, about 86 mM, about 85 mM, about 80 mM, about 75 mM, about 70 mM, about 65 mM, about 60 mM, about 55 mM, or about 50 mM NaCl). In some aspects, the medium comprises about 50 mM potassium ion and less than or equal to about 85 mM NaCl (e.g., about 85 mM, about 84 mM, about 83 mM, about 82 mM, about 81 mM, about 80 mM, about 75 mM, about 70 mM, about 65 mM, about 60 mM, about 55 mM, or about 50 mM NaCl). In some aspects, the medium comprises about 55 mM potassium ion and less than or equal to about 80 mM NaCl (e.g., about 80 mM, about 79 mM, about 78 mM, about 77 mM, about 76 mM, about 75 mM, about 70 mM, about 65 mM, about 60 mM, about 55 mM, or about 50 mM NaCl). In some aspects, the medium comprises about 60 mM potassium ion and less than or equal to about 75 mM NaCl (e.g., about 75 mM, about 74 mM, about 73 mM, about 72 mM, about 71 mM, about 70 mM, about 65 mM, about 60 mM, about 55 mM, or about 50 mM NaCl). In some aspects, the medium comprises about 65 mM potassium ion and less than or equal to about 70 mM NaCl (e.g., about 70 mM, about 69 mM, about 68 mM, about 67 mM, about 66 mM, about 65 mM, about 60 mM, about 55 mM, or about 50 mM NaCl). In some aspects, the medium comprises about 70 mM potassium ion and less than or equal to about 70 mM NaCl (e.g., about 65 mM, about 64 mM, about 63 mM, about 62 mM, about 61 mM, about 60 mM, about 55 mM, or about 50 mM NaCl). In some aspects, the medium comprises about 75 mM potassium ion and less than or equal to about 60 mM NaCl (e.g., about 60 mM, about 59 mM, about 58 mM, about 57 mM, about 56 mM, about 55 mM, about 50 mM, about 45 mM, or about 40 mM NaCl). In some aspects, the medium comprises about 80 mM potassium ion and less than or equal to about 55 mM NaCl (e.g., about 55 mM, about 54 mM, about 53 mM, about 52 mM, about 51 mM, about 50 mM, about 45 mM, about 40 mM, or about 35 mM NaCl). In some aspects, the medium comprises about 85 mM potassium ion and less than or equal to about 50 mM NaCl (e.g., about 50 mM, about 49 mM, about 48 mM, about 47 mM, about 46 mM, about 45 mM, about 40 mM, about 35 mM, or about 30 mM NaCl). In some aspects, the medium comprises about 90 mM potassium ion and less than or equal to about 45 mM NaCl (e.g., about 45 mM, about 44 mM, about 43 mM, about 42 mM, about 41 mM, about 40 mM, about 35 mM, about 30 mM, or about 25 mM NaCl). In some aspects, the medium comprises about 70 mM potassium ion and about 60 mM NaCl. In some aspects, the medium comprises about 70 mM potassium ion and about 61 mM NaCl. In some aspects, the medium comprises about 70 mM potassium ion and about 62 mM NaCl.

In some aspects, the medium comprises about 50 mM potassium ion and about 75 mM NaCl. In some aspects, the medium is hypotonic. In some aspects, the medium is isotonic.

Some aspects of the present disclosure are directed to methods of culturing cells, e.g., pluripotent, multipotent, and/or immune cells (e.g., T cells, NK cells, and/or TILs), comprising placing the cells in a medium comprising (i) potassium ion at a concentration higher than 40 mM and (ii) NaCl at a concentration of less than about 100 mM. Certain aspects of the present disclosure are directed to methods of culturing T cells, comprising placing the T cells in a medium comprising (i) potassium ion at a concentration of at least about 50 mM and (ii) NaCl at a concentration of less than about 90 mM.

II.D. Saccharides

In some aspects, the metabolic reprograming media (MRM), e.g., hyperkalemic media, comprises a saccharide. In some aspects, the MRM is hypotonic. In some aspects, the MRM is isotonic. In some aspects, the target concentration of the saccharide is reached by starting with a basal medium comprising a higher concentration of the saccharide and diluting the solution to reach the target concentration of the saccharide. In some aspects, the target concentration of the saccharide is reached by raising the concentration of the saccharide by adding the saccharide until the desired concentration is reached.

In some aspects, the saccharide is a monosaccharide, a disaccharide, or a polysaccharide. In some aspects, the saccharide is selected from glucose, fructose, galactose, mannose, maltose, sucrose, lactose, trehalose, or any combination thereof. In some aspects, the saccharide is glucose. In some aspects, the MRM comprises (i) potassium ion at a concentration of at least about 30 mM to at least about 100 mM and (ii) glucose. In some aspects, the MRM comprises (i) potassium ion at a concentration higher than 40 mM and (ii) glucose. In some aspects, the MRM comprises (i) potassium ion at a concentration of at least about 30 mM to at least about 100 mM and (ii) mannose. In some aspects, the MRM comprises (i) potassium ion at a concentration of higher than 40 mM and (ii) mannose. In some aspects, the MRM comprises (i) potassium ion at a concentration of at least about 50 mM and (ii) mannose. In some aspects, the MRM is hypotonic. In some aspects, the MRM is isotonic. In some aspects, the MRM comprises (i) potassium ion at a concentration higher than 40 mM and (ii) glucose; wherein the total concentration of potassium ion and NaCl is between 110 mM and 140 mM. In some aspects, the MRM comprises (i) potassium ion at a concentration higher than 50 mM and (ii) glucose; wherein the total concentration of potassium ion and NaCl is between 110 mM and 140 mM. In some aspects, the MRM comprises (i) potassium ion at a concentration of at least about 40 mM and (ii) mannose; wherein the total concentration of potassium ion and NaCl is between 110 mM and 140 mM. In some aspects, the MRM comprises (i) potassium ion at a concentration of at least about 50 mM and (ii) mannose; wherein the total concentration of potassium ion and NaCl is between 110 mM and 140 mM. In some aspects, the MRM comprises (i) potassium ion at a concentration higher than 40 mM and (ii) glucose; wherein the total concentration of potassium ion and NaCl is between 110 mM and 140 mM. In some aspects, the MRM comprises (i) potassium ion at a concentration higher than 50 mM and (ii) glucose; wherein the total concentration of potassium ion and NaCl is between 110 mM and 140 mM. In some aspects, the MRM comprises (i) potassium ion at a concentration of at least about 40 mM and (ii) mannose; wherein the total concentration of potassium ion and NaCl is between 110 mM and 140 mM. In some aspects, the MRM comprises (i) potassium ion at a concentration of at least about 50 mM and (ii) mannose; wherein the total concentration of potassium ion and NaCl is between 110 mM and 140 mM.

In some aspects, the concentration of the saccharide, e.g., glucose, is about 10 mM to about 24 mM. In some aspects, the concentration of the saccharide, e.g., glucose, is less than about 24 mM. In some aspects, the concentration of the saccharide, e.g., glucose, is more than about 10 mM. In some aspects, the concentration of the saccharide, e.g., glucose, is about 5 mM. In some aspects, the concentration of the saccharide, e.g., glucose, is from about 5 mM to about 20 mM. In some aspects, the concentration of the saccharide, e.g., glucose, is from about 10 mM to about 20 mM. In some aspects, the concentration of the saccharide, e.g., glucose, is from about 10 mM to about 25 mM, about 10 mM to about 20 mM, about 10 mM to about 5 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM. In some aspects, the concentration of the saccharide, e.g., glucose, is from about 5 mM to about 20 mM. In some aspects, the concentration of the saccharide, e.g., glucose, is from about 10 mM to about 20 mM. In some aspects, the concentration of the saccharide, e.g., glucose, is from about 10 mM to about 15 mM. In some aspects, the concentration of the saccharide, e.g., glucose, is from about 14 mM to about 14.5 mM. In some aspects, the concentration of the saccharide, e.g., glucose, is from about 14.5 mM to about 15 mM. In some aspects, the concentration of the saccharide, e.g., glucose, is from about 15 mM to about 15.5 mM. In some aspects, the concentration of the saccharide, e.g., glucose, is from about 15.5 mM to about 16 mM. In some aspects, the concentration of the saccharide, e.g., glucose, is from about 16 mM to about 16.5 mM. In some aspects, the concentration of the saccharide, e.g., glucose, is from about 16.5 mM to about 17 mM. In some aspects, the concentration of the saccharide, e.g., glucose, is from about 17 mM to about 17.5 mM. In some aspects, the concentration of the saccharide, e.g., glucose, is from about 17.5 mM to about 18 mM.

In some aspects, the concentration of the saccharide, e.g., glucose, is about 5 mM, about 6 mM, about 7 mM, about 8 mM, about 9 mM, about 10 mM, is about 10.5 mM, about 11 mM, about 11.5 mM, about 12 mM, about 12.5 mM, about 13 mM, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, about 20 mM, about 20.5 mM, about 21 mM, about 22 mM, about 23 mM, about 24 mM, or about 25 mM.

In some aspects, the concentration of the saccharide, e.g., glucose, is about 5 mM. In some aspects, the concentration of the saccharide, e.g., glucose, is about 6 mM. In some aspects, the concentration of the saccharide, e.g., glucose, is about 7 mM. In some aspects, the concentration of the saccharide, e.g., glucose, is about 8 mM. In some aspects, the concentration of the saccharide, e.g., glucose, is about 9 mM. In some aspects, the concentration of the saccharide, e.g., glucose, is about 10 mM. In some aspects, the concentration of the saccharide, e.g., glucose, is about 10.5 mM. In some aspects, the concentration of the saccharide, e.g., glucose, is about 11 mM. In some aspects, the concentration of the saccharide, e.g., glucose, is about 11.5 mM. In some aspects, the concentration of the saccharide, e.g., glucose, is about 12 mM. In some aspects, the concentration of the saccharide, e.g., glucose, is about 12.5 mM. In some aspects, the concentration of the saccharide, e.g., glucose, is about 13 mM. In some aspects, the concentration of the saccharide, e.g., glucose, is about 13.5 mM. In some aspects, the concentration of the saccharide, e.g., glucose, is about 14 mM. In some aspects, the concentration of the saccharide, e.g., glucose, is about 14.5 mM. In some aspects, the concentration of the saccharide, e.g., glucose, is about 15 mM. In some aspects, the concentration of the saccharide, e.g., glucose, is about 15.4 mM. In some aspects, the concentration of the saccharide, e.g., glucose, is about 15.9 mM. In some aspects, the concentration of the saccharide, e.g., glucose, is about 16.3 mM. In some aspects, the concentration of the saccharide, e.g., glucose, is about 16.8 mM. In some aspects, the concentration of the saccharide, e.g., glucose, is about 17.2 mM. In some aspects, the concentration of the saccharide, e.g., glucose, is about 17.7 mM.

II.E. Calcium

In some aspects, the MRM, e.g, hyperkalemic media, comprises calcium ion. In some aspects, the target concentration of calcium is reached by starting with a basal medium comprising a higher concentration of calcium ion and diluting the solution to reach the target concentration of calcium ion. In some aspects, the target concentration of calcium is reached by raising the concentration of calcium ion by adding one or more calcium salts. Non-limiting examples of calcium salts include calcium bromide, calcium carbonate, calcium chloride, calcium cyanamide, calcium fluoride, calcium hydride, calcium hydroxide, calcium iodate, calcium iodide, calcium nitrate, calcium nitrite, calcium oxalate, calcium perchlorate tetrahydrate, calcium phosphate monobasic, calcium phosphate tribasic, calcium sulfate, calcium thiocyanate tetrahydrate, hydroxyapatite, and any combination thereof. In some aspects, the calcium salt comprises calcium chloride (CaCl₂)). In some aspects, the calcium salt comprises calcium gluconate.

In some aspects, the concentration of the calcium ion is less than that of the basal medium. In some aspects, the concentration of the calcium ion is greater than that of the basal medium. In some aspects, the concentration of calcium ion is more than about 0.4 mM. In some aspects, the concentration of calcium ion is less than about 2.8 mM. In some aspects, the concentration of calcium ion is less than about 2.5 mM. In some aspects, the concentration of calcium ion is less than about 2.0 mM. In some aspects, the concentration of calcium ion is less than about 1.9 mM. In some aspects, the concentration of calcium ion is less than about 1.8 mM. In some aspects, the concentration of calcium ion is less than about 1.7 mM. In some aspects, the concentration of calcium ion is less than about 1.6 mM. In some aspects, the concentration of calcium ion is less than about 1.5 mM. In some aspects, the concentration of calcium ion is less than about 1.4 mM. In some aspects, the concentration of calcium ion is less than about 1.3 mM. In some aspects, the concentration of calcium ion is less than about 1.2 mM. In some aspects, the concentration of calcium ion is less than about 1.1 mM. In some aspects, the concentration of calcium ion is less than about 1.0 mM.

In some aspects, the concentration of calcium ion is from about 0.4 mM to about 2.8 mM, about 0.4 mM to about 2.7 mM, about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 0.8 to about 0.9 mM, about 0.8 to about 1.0 mM, about 0.8 to about 1.1 mM, about 0.8 to about 1.2 mM, about 0.8 to about 1.3 mM, about 0.8 to about 1.4 mM, about 0.8 to about 1.5 mM, about 0.8 to about 1.6 mM, about 0.8 to about 1.7 mM, about 0.8 to about 1.8 mM, about 0.9 to about 1.0 mM, about 0.9 to about 1.1 mM, about 0.9 to about 1.2 mM, about 0.9 to about 1.3 mM, about 0.9 to about 1.4 mM, about 0.9 to about 1.5 mM, about 0.9 to about 1.6 mM, about 0.9 to about 1.7 mM, about 0.9 to about 1.8 mM, about 1.0 to about 1.1 mM, about 1.0 to about 1.2 mM, about 1.0 to about 1.3 mM, about 1.0 to about 1.4 mM, about 1.0 to about 1.5 mM, about 1.0 to about 1.6 mM, about 1.0 to about 1.7 mM, about 1.0 to about 1.8 mM, about 1.1 to about 1.2 mM, about 1.1 to about 1.3 mM, about 1.1 to about 1.4 mM, about 1.1 to about 1.5 mM, about 1.1 to about 1.6 mM, about 1.1 to about 1.7 mM, about 1.1 to about 1.8 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.3 to about 1.4 mM, about 1.3 to about 1.5 mM, about 1.3 to about 1.6 mM, about 1.3 to about 1.7 mM, about 1.3 to about 1.8 mM, about 1.4 to about 1.5 mM, about 1.4 to about 1.6 mM, about 1.4 to about 1.7 mM, about 1.4 to about 1.8 mM, about 1.5 to about 1.6 mM, about 1.5 to about 1.7 mM, about 1.5 to about 1.8 mM, about 1.6 to about 1.7 mM, about 1.6 to about 1.8 mM, or about 1.7 to about 1.8 mM.

In some aspects, the concentration of calcium ion is from about 0.8 mM to about 1.8 mM. In some aspects, the concentration of calcium ion is from about 0.9 mM to about 1.8 mM. In some aspects, the concentration of calcium ion is from about 1.0 mM to about 1.8 mM. In some aspects, the concentration of calcium ion is from about 1.1 mM to about 1.8 mM. In some aspects, the concentration of calcium ion is from about 1.2 mM to about 1.8 mM. In some aspects, the concentration of calcium ion is from about 0.8 mM to about 1.8 mM. In some aspects, the concentration of calcium ion is from about 0.8 mM to about 0.9 mM. In some aspects, the concentration of calcium ion is from about 0.9 mM to about 1.0 mM. In some aspects, the concentration of calcium ion is from about 1.0 mM to about 1.1 mM. In some aspects, the concentration of calcium ion is from about 1.1 mM to about 1.2 mM. In some aspects, the concentration of calcium ion is from about 1.2 mM to about 1.3 mM. In some aspects, the concentration of calcium ion is from about 1.3 mM to about 1.4 mM. In some aspects, the concentration of calcium ion is from about 1.4 mM to about 1.5 mM. In some aspects, the concentration of calcium ion is from about 1.5 mM to about 1.6 mM. In some aspects, the concentration of calcium ion is from about 1.7 mM to about 1.8 mM.

In some aspects, the concentration of calcium ion is about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM. In some aspects, the concentration of calcium ion is about 0.6 mM. In some aspects, the concentration of calcium ion is about 0.7 mM. In some aspects, the concentration of calcium ion is about 0.8 mM. In some aspects, the concentration of calcium ion is about 0.9 mM. In some aspects, the concentration of calcium ion is about 1.0 mM. In some aspects, the concentration of calcium ion is about 1.1 mM. In some aspects, the concentration of calcium ion is about 1.2 mM. In some aspects, the concentration of calcium ion is about 1.3 mM. In some aspects, the concentration of calcium ion is about 1.4 mM. In some aspects, the concentration of calcium ion is about 1.5 mM. In some aspects, the concentration of calcium ion is about 1.6 mM. In some aspects, the concentration of calcium ion is about 1.7 mM. In some aspects, the concentration of calcium ion is about 1.8 mM.

In some aspects, the MRM comprises about 40 mM to about 90 mM potassium ion and about 0.5 mM to about 2.8 mM calcium ion. In some aspects, the MRM comprises about 40 mM to about 90 mM potassium ion, NaCl, and about 0.5 mM to about 2.8 mM calcium ion; wherein the total concentration of potassium ion and NaCl is between 110 mM and 140 mM.

II.F. Cytokines

In some aspects, the MRM comprises a cytokine. In some aspects, the MRM is hypotonic. In some aspects, the MRM is isotonic. In some aspects, the cytokine is selected from IL-2, IL-7, IL-15, IL-21, and any combination thereof. In some aspects, the MRM does not comprise IL-2. In some aspects, the MRM comprises IL2 and IL21. In some aspects, the MRM comprises IL2, IL21, and IL15.

The cytokine can be added to the MRM at any point. In some aspects, the cytokine is added to the MRM before the TILs (e.g., the tumor sample), are added to the medium. In some aspects, the TILs (e.g., the tumor sample), are cultured in the MRM comprising (i) potassium at a concentration disclosed herein, and (ii) a cytokine throughout TIL culture including expansion. In some aspects, the TILs (e.g., the tumor sample), are cultured in the MRM comprising (i) potassium at a concentration disclosed herein, and (ii) a cytokine throughout TIL expansion.

In some aspects, the MRM comprises (i) at least about 30 mM to at least about 100 mM potassium ion and (ii) IL-2. In some aspects, the MRM comprises (i) more than 40 mM potassium ion and (ii) IL-2. In some aspects, the MRM comprises (i) at least about 50 mM potassium ion and (ii) IL-2. In some aspects, the MRM comprises (i) at least about 30 mM to at least about 100 mM potassium ion and (ii) IL-7. In some aspects, the MRM comprises (i) more than 40 mM potassium ion and (ii) IL-7. In some aspects, the MRM comprises (i) at least about 50 mM potassium ion and (ii) IL-7. In some aspects, the MRM comprises (i) at least about 30 mM to at least about 100 mM potassium ion and (ii) IL-15. In some aspects, the MRM comprises (i) more than 40 mM potassium ion and (ii) IL-15. In some aspects, the MRM comprises (i) at least about 50 mM potassium ion and (ii) IL-15. In some aspects, the MRM comprises (i) at least about 30 mM to at least about 100 mM potassium ion and (ii) IL-21. In some aspects, the MRM comprises (i) more than 40 mM potassium ion and (ii) IL-21. In some aspects, the MRM comprises (i) at least about 50 mM potassium ion and (ii) IL-21. In some aspects, the MRM does not comprise IL-7 and/or IL-15.

In some aspects, the MRM comprises (i) at least about 30 mM to at least about 100 mM potassium ion, (ii) NaCl, and (iii) IL-2; wherein the total concentration of potassium ion and NaCl is from 110 mM to 140 mM. In some aspects, the MRM comprises (i) more than 40 mM potassium ion, (ii) NaCl, and (iii) IL-2; wherein the total concentration of potassium ion and NaCl is from 110 mM to 140 mM. In some aspects, the MRM comprises (i) at least about 50 mM potassium ion, (ii) NaCl, and (iii) IL-2; wherein the total concentration of potassium ion and NaCl is from 110 mM to 140 mM. In some aspects, the MRM comprises (i) at least about 30 mM to at least about 100 mM potassium ion, (ii) NaCl, and (iii) IL-7; wherein the total concentration of potassium ion and NaCl is from 110 mM to 140 mM. In some aspects, the MRM comprises (i) more than 40 mM potassium ion, (ii) NaCl, and (iii) IL-7; wherein the total concentration of potassium ion and NaCl is from 110 mM to 140 mM. In some aspects, the MRM comprises (i) at least about 50 mM potassium ion, (ii) NaCl, and (iii) IL-7; wherein the total concentration of potassium ion and NaCl is from 110 mM to 140 mM. In some aspects, the MRM comprises (i) at least about 30 mM to at least about 100 mM potassium ion, (ii) NaCl, and (iii) IL-15; wherein the total concentration of potassium ion and NaCl is from 110 mM to 140 mM. In some aspects, the MRM comprises (i) more than 40 mM potassium ion, (ii) NaCl, and (iii) IL-15; wherein the total concentration of potassium ion and NaCl is from 110 mM to 140 mM. In some aspects, the MRM comprises (i) at least about 50 mM potassium ion, (ii) NaCl, and (iii) IL-15; wherein the total concentration of potassium ion and NaCl is from 110 mM to 140 mM. In some aspects, the MRM comprises (i) at least about 30 mM to at least about 100 mM potassium ion, (ii) NaCl, and (iii) IL-21; wherein the total concentration of potassium ion and NaCl is from 110 mM to 140 mM. In some aspects, the MRM comprises (i) more than 40 mM potassium ion, (ii) NaCl, and (iii) IL-21; wherein the total concentration of potassium ion and NaCl is from 110 mM to 140 mM. In some aspects, the MRM comprises (i) at least about 50 mM potassium ion, (ii) NaCl, and (iii) IL-21; wherein the total concentration of potassium ion and NaCl is from 110 mM to 140 mM. In some aspects, the MRM does not comprise IL-7 and/or IL-15; wherein the total concentration of potassium ion and NaCl is from 110 mM to 140 mM.

In some aspects, the MRM comprises at least about 0.1 ng/mL IL-2. In some aspects, the MRM comprises from about 50 ng/mL to about 600 ng/mL, about 50 ng/mL to about 500 ng/mL, about 50 ng/mL to about 450 ng/mL, about 50 ng/mL to about 400 ng/mL, about 50 ng/mL to about 350 ng/mL, about 50 ng/mL to about 300 ng/mL, about 100 ng/mL to about 600 ng/mL, about 100 ng/mL to about 500 ng/mL, about 100 ng/mL to about 450 ng/mL, about 100 ng/mL to about 400 ng/mL, about 100 ng/mL to about 350 ng/mL, about 100 ng/mL to about 300 ng/mL, about 200 ng/mL to about 500 ng/mL, about 200 ng/mL to about 450 ng/mL, about 200 ng/mL to about 400 ng/mL, about 200 ng/mL to about 350 ng/mL, about 200 ng/mL to about 300 ng/mL, about 250 ng/mL to about 350 ng/mL, about 300 ng/mL to about 600 ng/mL, about 300 ng/mL to about 500 ng/mL, about 300 ng/mL to about 450 ng/mL, about 300 ng/mL to about 400 ng/mL, about 300 ng/mL to about 350 ng/mL, about 250 ng/mL to about 300 ng/mL, or about 275 ng/mL to about 325 ng/mL IL-2.

In some aspects, the MRM comprises at least about 50 ng/mL, at least about 60 ng/mL, at least about 70 ng/mL, at least about 80 ng/mL, at least about 90 ng/mL, at least about 100 ng/mL, at least about 110 ng/mL, at least about 120 ng/mL, at least about 130 ng/mL, at least about 140 ng/mL, at least about 150 ng/mL, at least about 160 ng/mL, at least about 170 ng/mL, at least about 180 ng/mL, at least about 190 ng/mL, at least about 200 ng/mL, at least about 210 ng/mL, at least about 220 ng/mL, at least about 230 ng/mL, at least about 240 ng/mL, at least about 250 ng/mL, at least about 260 ng/mL, at least about 270 ng/mL, at least about 280 ng/mL, at least about 290 ng/mL, at least about 300 ng/mL, at least about 310 ng/mL, at least about 320 ng/mL, at least about 330 ng/mL, at least about 340 ng/mL, at least about 350 ng/mL, at least about 360 ng/mL, at least about 370 ng/mL, at least about 380 ng/mL, at least about 390 ng/mL, at least about 400 ng/mL, at least about 410 ng/mL, at least about 420 ng/mL, at least about 430 ng/mL, at least about 440 ng/mL, at least about 450 ng/mL, at least about 460 ng/mL, at least about 470 ng/mL, at least about 480 ng/mL, at least about 490 ng/mL, at least about 500 ng/mL, at least about 510 ng/mL, at least about 520 ng/mL, at least about 530 ng/mL, at least about 540 ng/mL, at least about 550 ng/mL, at least about 560 ng/mL, at least about 570 ng/mL, at least about 580 ng/mL, at least about 590 ng/mL, or at least about 600 ng/mL IL-2. In some aspects, the MRM comprises at least about 50 ng/mL IL-2. In some aspects, the MRM comprises at least about 60 ng/mL IL-2. In some aspects, the MRM comprises at least about 70 ng/mL IL-2. In some aspects, the MRM comprises at least about 73.6 ng/mL IL-2. In some aspects, the MRM comprises at least about 75 ng/mL IL-2. In some aspects, the MRM comprises at least about 80 ng/mL IL-2. In some aspects, the MRM comprises at least about 90 ng/mL IL-2. In some aspects, the MRM comprises at least about 100 ng/mL IL-2. In some aspects, the MRM comprises at least about 200 ng/mL IL-2. In some aspects, the MRM comprises at least about 300 ng/mL IL-2. In some aspects, the MRM comprises at least about 400 ng/mL IL-2. In some aspects, the MRM comprises at least about 500 ng/mL IL-2. In some aspects, the MRM comprises at least about 600 ng/mL IL-2.

In some aspects, the MRM comprises at least about 1500 IU/mL IL-2. In some aspects, the MRM comprises from about 1500 IU/mL to about 12,000 IU/mL IL-2. In some aspects, the MRM comprises at least about 1500 IU/mL, at least about 1600 IU/mL, at least about 1700 IU/mL, at least about 1800 IU/mL, at least about 1900 IU/mL, at least about 2000 IU/mL, at least about 2100 IU/mL, at least about 2200 IU/mL, at least about 2300 IU/mL, at least about 2400 IU/mL, at least about 2500 IU/mL, at least about 2600 IU/mL, at least about 2700 IU/mL, at least about 2800 IU/mL, at least about 2900 IU/mL, at least about 3000 IU/mL, at least about 3100 IU/mL, at least about 3200 IU/mL, at least about 3300 IU/mL, at least about 3400 IU/mL, at least about 3500 IU/mL, at least about 3600 IU/mL, at least about 3700 IU/mL, at least about 3800 IU/mL, at least about 3900 IU/mL, at least about 4000 IU/mL, at least about 4100 IU/mL, at least about 4200 IU/mL, at least about 4300 IU/mL, at least about 4400 IU/mL, at least about 4500 IU/mL, at least about 4600 IU/mL, at least about 4700 IU/mL, at least about 4800 IU/mL, at least about 4900 IU/mL, at least about 5000 IU/mL, at least about 5100 IU/mL, at least about 5200 IU/mL, at least about 5300 IU/mL, at least about 5400 IU/mL, at least about 5500 IU/mL, at least about 5600 IU/mL, at least about 5700 IU/mL, at least about 5800 IU/mL, at least about 5900 IU/mL, at least about 6000 IU/mL, at least about 6100 IU/mL, at least about 6200 IU/mL, at least about 6300 IU/mL, at least about 6400 IU/mL, at least about 6500 IU/mL, at least about 6600 IU/mL, at least about 6700 IU/mL, at least about 6800 IU/mL, at least about 6900 IU/mL, at least about 7000 IU/mL IL-2, at least about 7100 IU/mL, at least about 7200 IU/mL, at least about 7300 IU/mL, at least about 7400 IU/mL, at least about 7500 IU/mL, at least about 7600 IU/mL, at least about 7700 IU/mL, at least about 7800 IU/mL, at least about 7900 IU/mL, or at least about 8000 IU/mL IL-2. In some aspects, the MRM comprises at least about 3000 IU/mL IL-2. In some aspects, TILs are cultured in MRM during a second culture (e.g., REP culture), as described herein, wherein the MRM comprises about 3000 IU/mL. In some aspects, the MRM comprises at least about 6000 IU/mL IL-2. In some aspects, TILs are cultured in MRM during an initial culture, as described herein, wherein the MRM comprises about 6000 IU/mL.

In some aspects, the MRM comprises at least about 0.1 ng/mL IL-21. In some aspects, the MRM comprises from about 0.1 ng/mL to about 30 ng/mL, about 1 ng/mL to about 30 ng/mL, about 1 ng/mL to about 25 ng/mL, about 1 ng/mL to about 20 ng/mL, about 1 ng/mL to about 15 ng/mL, about 1 ng/mL to about 10 ng/mL, about 5 ng/mL to about 30 ng/mL, about 5 ng/mL to about 20 ng/mL, about 10 ng/mL to about 30 ng/mL, about 10 ng/mL to about 20 ng/mL, or about 15 ng/mL to about 30 ng/mL IL-21.

In some aspects, the MRM comprises at least about 0.1 ng/mL, at least about 0.5 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, at least about 20 ng/mL, at least about 25 ng/mL, at least about 30 ng/mL, at least about 35 ng/mL, or at least about 40 ng/mL IL-21. In some aspects, the MRM comprises at least about 1.0 ng/mL IL-21. In some aspects, the MRM comprises at least about 2.0 ng/mL IL-21. In some aspects, the MRM comprises at least about 3.0 ng/mL IL-21. In some aspects, the MRM comprises at least about 4.0 ng/mL IL-21. In some aspects, the MRM comprises at least about 5.0 ng/mL IL-21. In some aspects, the MRM comprises at least about 6.0 ng/mL IL-21. In some aspects, the MRM comprises at least about 7.0 ng/mL IL-21. In some aspects, the MRM comprises at least about 8.0 ng/mL IL-21. In some aspects, the MRM comprises at least about 9.0 ng/mL IL-21. In some aspects, the MRM comprises at least about 10 ng/mL IL-21. In some aspects, the MRM comprises at least about 15 ng/mL IL-21. In some aspects, the MRM comprises at least about 20 ng/mL IL-21. In some aspects, the MRM comprises at least about 25 ng/mL IL-21. In some aspects, the MRM comprises at least about 30 ng/mL IL-21. In some aspects, the MRM comprises at least about 35 ng/mL IL-21.

In some aspects, the MRM comprises between about 50 IU/mL to about 500 IU/mL of IL-21. In some aspects, the MRM comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-21.

In some aspects, the MRM comprises at least about 0.1 ng/mL IL-7. In some aspects, the MRM comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-7.

In some aspects, the MRM comprises at least about 0.1 ng/mL, at least about 0.5 ng/mL, at least about 1 ng/mL, at least about 1.3 ng/mL, at least about 1.5 ng/mL, at least about 1.7 ng/mL, at least about 2 ng/mL, at least about 2.3 ng/mL, at least about 2.5 ng/mL, at least about 2.7 ng/mL, at least about 3 ng/mL, at least about 3.3 ng/mL, at least about 3.5 ng/mL, at least about 3.7 ng/mL, at least about 4 ng/mL, at least about 4.3 ng/mL, at least about 4.5 ng/mL, at least about 4.7 ng/mL, at least about 5 ng/mL, at least about 5.3 ng/mL, at least about 5.5 ng/mL, at least about 5.7 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-7. In some aspects, the medium comprises at least about 1.0 ng/mL IL-7. In some aspects, the MRM comprises at least about 2.0 ng/mL IL-7. In some aspects, the MRM comprises at least about 2.3 ng/mL IL-7. In some aspects, the MRM comprises at least about 2.5 ng/mL IL-7. In some aspects, the MRM comprises at least about 2.7 ng/mL IL-7. In some aspects, the MRM comprises at least about 3.0 ng/mL IL-7. In some aspects, the MRM comprises at least about 3.3 ng/mL IL-7. In some aspects, the MRM comprises at least about 3.5 ng/mL IL-7. In some aspects, the MRM comprises at least about 3.7 ng/mL IL-7.

In some aspects, the MRM comprises between about 500 IU/mL to about 1,500 IU/mL of IL-7. In some aspects, the MRM comprises about 500 IU/mL, about 550 IU/mL, about 600 IU/mL, about 650 IU/mL, about 700 IU/mL, about 750 IU/mL, about 800 IU/mL, about 850 IU/mL, about 900 IU/mL, about 950 IU/mL, about 1,000 IU/mL, about 1,050 IU/mL, about 1,100 IU/mL, about 1,150 IU/mL, about 1,200 IU/mL, about 1,250 IU/mL, about 1,300 IU/mL, about 1,350 IU/mL, about 1,400 IU/mL, about 1,450 IU/mL, or about 1,500 IU/mL of IL-7.

In some aspects, the MRM comprises at least about 0.1 ng/mL IL-15. In some aspects, the MRM comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-15.

In some aspects, the MRM comprises at least about 0.1 ng/mL, at least about 0.2 ng/mL, at least about 0.3 ng/mL, at least about 0.4 ng/mL, at least about 0.5 ng/mL, at least about 0.6 ng/mL, at least about 0.7 ng/mL, at least about 0.8 ng/mL, at least about 0.9 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-15. In some aspects, the MRM comprises at least about 0.1 ng/mL IL-15. In some aspects, the MRM comprises at least about 0.2 ng/mL IL-15. In some aspects, the MRM comprises at least about 0.3 ng/mL IL-15. In some aspects, the MRM comprises at least about 0.4 ng/mL IL-15. In some aspects, the MRM comprises at least about 0.5 ng/mL IL-15. In some aspects, the MRM comprises at least about 0.6 ng/mL IL-15. In some aspects, the MRM comprises at least about 0.7 ng/mL IL-15. In some aspects, the MRM comprises at least about 0.8 ng/mL IL-15. In some aspects, the MRM comprises at least about 0.9 ng/mL IL-15. In some aspects, the MRM comprises at least about 1.0 ng/mL IL-15. In some aspects, the MRM comprises at least about 2.0 ng/mL IL-15. In some aspects, the MRM comprises at least about 3.0 ng/mL IL-15. In some aspects, the MRM comprises at least about 4.0 ng/mL IL-15. In some aspects, the MRM comprises at least about 5.0 ng/mL IL-15. In some aspects, the MRM comprises at least about 6.0 ng/mL IL-15. In some aspects, the MRM comprises at least about 7.0 ng/mL IL-15. In some aspects, the MRM comprises at least about 8.0 ng/mL IL-15. In some aspects, the MRM comprises at least about 9.0 ng/mL IL-15. In some aspects, the MRM comprises at least about 10 ng/mL IL-15.

In some aspects, the MRM comprises between about 50 IU/mL to about 500 IU/mL of IL-15. In some aspects, the MRM comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-15.

In some aspects, the MRM comprises at least about 30 mM to at least about 100 mM potassium ion and about 300 ng/mL IL-2. In some aspects, the MRM comprises more than 40 mM potassium ion and about 300 ng/mL IL-2. In some aspects, the MRM comprises at least about 45 mM potassium ion and about 300 ng/mL IL-2. In some aspects, the MRM comprises at least about 50 mM potassium ion and about 300 ng/mL IL-2. In some aspects, the MRM comprises at least about 55 mM potassium ion and about 300 ng/mL IL-2. In some aspects, the MRM comprises at least about 60 mM potassium ion and about 300 ng/mL IL-2. In some aspects, the MRM comprises at least about 65 mM potassium ion and about 300 ng/mL IL-2. In some aspects, the MRM comprises at least about 70 mM potassium ion and about 300 ng/mL IL-2. In some aspects, the MRM comprises at least about 75 mM potassium ion and about 300 ng/mL IL-2. In some aspects, the MRM comprises at least about 80 mM potassium ion and about 300 ng/mL IL-2. In some aspects, the MRM comprises at least about 85 mM potassium ion and about 300 ng/mL IL-2. In some aspects, the MRM comprises at least about 90 mM potassium ion and about 300 ng/mL IL-2. In some aspects, the MRM comprises (i) at least about 70 mM potassium ion, (ii) about 60 mM sodium, (iii) about 1.4 mM calcium, (iv) about 16 mM glucose, and (v) about 10 ng/mL IL-2.

In some aspects, the MRM comprises at least about 30 mM to at least about 100 mM potassium ion, about 300 ng/mL IL-2, and about 290 ng/mL IL-7. In some aspects, the MRM comprises more than 40 mM potassium ion and about 300 ng/mL IL-2 and about 290 ng/mL IL-7. In some aspects, the MRM comprises at least about 45 mM potassium ion, about 300 ng/mL IL-2, and about 290 ng/mL IL-7. In some aspects, the MRM comprises at least about 40 mM potassium ion, about 300 ng/mL IL-2, and about 290 ng/mL IL-7. In some aspects, the MRM comprises at least about 55 mM potassium ion, about 300 ng/mL IL-2, and about 290 ng/mL IL-7. In some aspects, the MRM comprises at least about 60 mM potassium ion, about 300 ng/mL IL-2, and about 290 ng/mL IL-7. In some aspects, the MRM comprises at least about 65 mM potassium ion, about 300 ng/mL IL-2, and about 290 ng/mL IL-7. In some aspects, the MRM comprises at least about 70 mM potassium ion, about 300 ng/mL IL-2, and about 290 ng/mL IL-7. In some aspects, the MRM comprises at least about 75 mM potassium ion, about 300 ng/mL IL-2, and about 290 ng/mL IL-7. In some aspects, the MRM comprises at least about 80 mM potassium ion, about 300 ng/mL IL-2, and about 290 ng/mL IL-7. In some aspects, the MRM comprises at least about 85 mM potassium ion, about 300 ng/mL IL-2, and about 290 ng/mL IL-7. In some aspects, the MRM comprises at least about 90 mM potassium ion, about 300 ng/mL IL-2, and about 290 ng/mL IL-7. In some aspects, the MRM comprises (i) at least about 70 mM potassium ion, (ii) about 60 mM sodium, (iii) about 1.4 mM calcium, (iv) about 16 mM glucose, (v) about 300 ng/mL IL-2, and (vi) about 290 ng/mL IL-7.

In some aspects, the MRM comprises at least about 30 mM to at least about 100 mM potassium ion, about 300 ng/mL IL-2, and about 0.4 ng/mL IL-15. In some aspects, the MRM comprises more than 40 mM potassium ion, about 300 ng/mL IL-2, and about 0.4 ng/mL IL-15. In some aspects, the MRM comprises at least about 45 mM potassium ion, about 300 ng/mL IL-2, and about 0.4 ng/mL IL-15. In some aspects, the MRM comprises at least about 50 mM potassium ion, about 300 ng/mL IL-2, and about 0.4 ng/mL IL-15. In some aspects, the MRM comprises at least about 55 mM potassium ion, about 300 ng/mL IL-2, and about 0.4 ng/mL IL-15. In some aspects, the MRM comprises at least about 60 mM potassium ion, about 300 ng/mL IL-2, and about 0.4 ng/mL IL-15. In some aspects, the MRM comprises at least about 65 mM potassium ion, about 300 ng/mL IL-2, and about 0.4 ng/mL IL-15. In some aspects, the MRM comprises at least about 70 mM potassium ion, about 300 ng/mL IL-2, and about 0.4 ng/mL IL-15. In some aspects, the MRM comprises at least about 75 mM potassium ion, about 300 ng/mL IL-2, and about 0.4 ng/mL IL-15. In some aspects, the MRM comprises at least about 80 mM potassium ion, about 300 ng/mL IL-2, and about 0.4 ng/mL IL-15. In some aspects, the MRM comprises at least about 85 mM potassium ion, about 300 ng/mL IL-2, and about 0.4 ng/mL IL-15. In some aspects, the MRM comprises at least about 90 mM potassium ion, about 300 ng/mL IL-2, and about 0.4 ng/mL IL-15. In some aspects, the MRM comprises (i) at least about 70 mM potassium ion, (ii) about 60 mM sodium, (iii) about 1.4 mM calcium, (iv) about 16 mM glucose, (v) about 300 ng/mL IL-2, and (vi) about 0.4 ng/mL IL-15.

In some aspects, the MRM comprises at least about 30 mM to at least about 100 mM potassium ion, about 300 ng/mL IL-2, about 290 ng/mL IL-7, and about 0.4 ng/mL IL-15. In some aspects, the MRM comprises more than 40 mM potassium ion, about 300 ng/mL IL-2, about 290 ng/mL IL-7, and about 0.4 ng/mL IL-15. In some aspects, the MRM comprises at least about 45 mM potassium ion, about 300 ng/mL IL-2, about 290 ng/mL IL-7, and about 0.4 ng/mL IL-15. In some aspects, the MRM comprises at least about 50 mM potassium ion, about 300 ng/mL IL-2, about 290 ng/mL IL-7, and about 0.4 ng/mL IL-15. In some aspects, the MRM comprises at least about 55 mM potassium ion, about 300 ng/mL IL-2, about 290 ng/mL IL-7, and about 0.4 ng/mL IL-15. In some aspects, the MRM comprises at least about 60 mM potassium ion, about 300 ng/mL IL-2, about 290 ng/mL IL-7, and about 0.4 ng/mL IL-15. In some aspects, the MRM comprises at least about 65 mM potassium ion, about 300 ng/mL IL-2, about 290 ng/mL IL-7, and about 0.4 ng/mL IL-15. In some aspects, the MRM comprises at least about 70 mM potassium ion, about 300 ng/mL IL-2, about 290 ng/mL IL-7, and about 0.4 ng/mL IL-15. In some aspects, the MRM comprises at least about 75 mM potassium ion and about 10 ng/mL IL-2, about 1 ng/mL IL-7, and about 1 ng/mL IL-15. In some aspects, the MRM comprises at least about 80 mM potassium ion, about 300 ng/mL IL-2, about 290 ng/mL IL-7, and about 0.4 ng/mL IL-15. In some aspects, the MRM comprises at least about 85 mM potassium ion, about 300 ng/mL IL-2, about 290 ng/mL IL-7, and about 0.4 ng/mL IL-15. In some aspects, the MRM comprises at least about 90 mM potassium ion, about 300 ng/mL IL-2, about 290 ng/mL IL-7, and about 0.4 ng/mL IL-15. In some aspects, the MRM comprises (i) at least about 70 mM potassium ion, (ii) about 60 mM sodium, (iii) about 1.4 mM calcium, (iv) about 16 mM glucose, (v) about 300 ng/mL IL-2, (vi) about 290 ng/mL IL-7, and (vii) about 0.4 ng/mL IL-15.

In some aspects, the MRM comprises at least about 30 mM to at least about 100 5 mM potassium ion, about 300 ng/mL IL-2, and about 30 ng/mL IL-21. In some aspects, the MRM comprises more than 40 mM potassium ion, about 300 ng/mL IL-2, and about 30 ng/mL IL-21. In some aspects, the MRM comprises at least about 45 mM potassium ion, about 300 ng/mL IL-2, and about 30 ng/mL IL-21. In some aspects, the MRM comprises at least about 50 mM potassium ion, about 300 ng/mL IL-2, and about 30 ng/mL IL-21. In some aspects, the MRM comprises at least about 55 mM potassium ion, about 300 ng/mL IL-2, and about 30 ng/mL IL-21. In some aspects, the MRM comprises at least about 60 mM potassium ion, about 300 ng/mL IL-2, and about 30 ng/mL IL-21. In some aspects, the MRM comprises at least about 65 mM potassium ion, about 300 ng/mL IL-2, and about 30 ng/mL IL-21. In some aspects, the MRM comprises at least about 70 mM potassium ion, about 300 ng/mL IL-2, and about 30 ng/mL IL-21. In some aspects, the MRM comprises at least about 75 mM potassium ion, about 300 ng/mL IL-2, and about 30 ng/mL IL-21. In some aspects, the MRM comprises at least about 80 mM potassium ion, about 300 ng/mL IL-2, and about 30 ng/mL IL-21. In some aspects, the MRM comprises at least about 85 mM potassium ion, about 300 ng/mL IL-2, and about 30 ng/mL IL-21. In some aspects, the MRM comprises at least about 90 mM potassium ion, about 300 ng/mL IL-2, and about 30 ng/mL IL-21. In some aspects, the MRM comprises (i) at least about 70 mM potassium ion, (ii) about 60 mM sodium, (iii) about 1.4 mM calcium, (iv) about 16 mM glucose, (v) about 300 ng/mL IL-2, and (vi) about 30 ng/mL IL-21.

In some aspects, the MRM comprises at least about 30 mM to at least about 100 mM potassium ion, about 290 ng/mL IL-7, and about 20 ng/mL IL-21. In some aspects, the MRM comprises more than 40 mM potassium ion, about 290 ng/mL IL-7, and about 20 ng/mL IL-21. In some aspects, the MRM comprises at least about 45 mM potassium ion, about 290 ng/mL IL-7, and about 20 ng/mL IL-21. In some aspects, the MRM comprises at least about 50 mM potassium ion, about 290 ng/mL IL-7, and about 20 ng/mL IL-21. In some aspects, the MRM comprises at least about 55 mM potassium ion, about 290 ng/mL IL-7, and about 20 ng/mL IL-21. In some aspects, the MRM comprises at least about 60 mM potassium ion, about 290 ng/mL IL-7, and about 20 ng/mL IL-21. In some aspects, the MRM comprises at least about 65 mM potassium ion, about 290 ng/mL IL-7, and about 20 ng/mL IL-21. In some aspects, the MRM comprises at least about 70 mM potassium ion, about 290 ng/mL IL-7, and about 20 ng/mL IL-21. In some aspects, the MRM comprises at least about 75 mM potassium ion, about 290 ng/mL IL-7, and about 20 ng/mL IL-21. In some aspects, the MRM comprises at least about 80 mM potassium ion, about 290 ng/mL IL-7, and about 20 ng/mL IL-21. In some aspects, the MRM comprises at least about 85 mM potassium ion, about 290 ng/mL IL-7, and about 20 ng/mL IL-21. In some aspects, the MRM comprises at least about 90 mM potassium ion, about 290 ng/mL IL-7, and about 20 ng/mL IL-21. In some aspects, the MRM comprises (i) at least about 70 mM potassium ion, (ii) about 60 mM sodium, (iii) about 1.4 mM calcium, (iv) about 16 mM glucose, (v) about 290 ng/mL IL-7, and (vi) about 20 ng/mL IL-21.

In some aspects, the MRM comprises at least about 30 mM to at least about 100 mM potassium ion, about 0.4 ng/mL IL-15, and about 20 ng/mL IL-21. In some aspects, the MRM comprises more than 40 mM potassium ion, about 0.4 ng/mL IL-15, and about 20 ng/mL IL-21. In some aspects, the MRM comprises at least about 45 mM potassium ion, about 0.4 ng/mL IL-15, and about 20 ng/mL IL-21. In some aspects, the MRM comprises at least about 50 mM potassium ion, about 0.4 ng/mL IL-15, and about 20 ng/mL IL-21. In some aspects, the MRM comprises at least about 55 mM potassium ion, about 0.4 ng/mL IL-15, and about 20 ng/mL IL-21. In some aspects, the MRM comprises at least about 60 mM potassium ion, about 0.4 ng/mL IL-15, and about 20 ng/mL IL-21. In some aspects, the MRM comprises at least about 65 mM potassium ion, about 0.4 ng/mL IL-15, and about 20 ng/mL IL-21. In some aspects, the MRM comprises at least about 70 mM potassium ion, about 0.4 ng/mL IL-15, and about 20 ng/mL IL-21. In some aspects, the MRM comprises at least about 75 mM potassium ion, about 0.4 ng/mL IL-15, and about 20 ng/mL IL-21. In some aspects, the MRM comprises at least about 80 mM potassium ion, about 0.4 ng/mL IL-15, and about 20 ng/mL IL-21. In some aspects, the MRM comprises at least about 85 mM potassium ion, about 0.4 ng/mL IL-15, and about 20 ng/mL IL-21. In some aspects, the MRM comprises at least about 90 mM potassium ion, about 0.4 ng/mL IL-15, and about 20 ng/mL IL-21. In some aspects, the MRM comprises (i) at least about 70 mM potassium ion, (ii) about 60 mM sodium, (iii) about 1.4 mM calcium, (iv) about 16 mM glucose, (v) about 0.4 ng/mL IL-15, and (vi) about 20 ng/mL IL-21.

In some aspects, the MRM comprises at least about 30 mM to at least about 100 mM potassium ion, NaCl, and about 300 ng/mL IL-2; wherein the total concentration of potassium ion and NaCl is from 110 mM to 140 mM. In some aspects, the MRM comprises more than 40 mM potassium ion, NaCl, and about 300 ng/mL IL-2; wherein the total concentration of potassium ion and NaCl is from 110 mM to 140 mM. In some aspects, the MRM comprises at least about 45 mM potassium ion, NaCl, and about 300 ng/mL IL-2; wherein the total concentration of potassium ion and NaCl is from 110 mM to 140 mM. In some aspects, the MRM comprises at least about 50 mM potassium ion, NaCl, and about 300 ng/mL IL-2; wherein the total concentration of potassium ion and NaCl is from 110 mM to 140 mM. In some aspects, the MRM comprises at least about 55 mM potassium ion, NaCl, and about 300 ng/mL IL-2; wherein the total concentration of potassium ion and NaCl is from 110 mM to 140 mM. In some aspects, the MRM comprises at least about 60 mM potassium ion, NaCl, and about 300 ng/mL IL-2; wherein the total concentration of potassium ion and NaCl is from 110 mM to 140 mM. In some aspects, the MRM comprises at least about 65 mM potassium ion, NaCl, and about 300 ng/mL IL-2; wherein the total concentration of potassium ion and NaCl is from 110 mM to 140 mM. In some aspects, the MRM comprises at least about 70 mM potassium ion, NaCl, and about 300 ng/mL IL-2; wherein the total concentration of potassium ion and NaCl is from 110 mM to 140 mM. In some aspects, the MRM comprises at least about 75 mM potassium ion, NaCl, and about 300 ng/mL IL-2; wherein the total concentration of potassium ion and NaCl is from 110 mM to 140 mM. In some aspects, the MRM comprises at least about 80 mM potassium ion, NaCl, and about 300 ng/mL IL-2; wherein the total concentration of potassium ion and NaCl is from 110 mM to 140 mM. In some aspects, the MRM comprises at least about 85 mM potassium ion, NaCl, and about 300 ng/mL IL-2; wherein the total concentration of potassium ion and NaCl is from 110 mM to 140 mM. In some aspects, the MRM comprises at least about 90 mM potassium ion, NaCl, and about 300 ng/mL IL-2; wherein the total concentration of potassium ion and NaCl is from 110 mM to 140 mM. In some aspects, the MRM comprises (i) at least about 70 mM potassium ion, (ii) about 60 mM sodium (e. g., NaCl), (iii) about 1.4 mM calcium, (iv) about 16 mM glucose, and (v) about 10 ng/mL IL-2.

In some aspects, the MRM comprises at least about 30 mM to at least about 100 mM potassium ion, NaCl, about 300 ng/mL IL-2, and about 290 ng/mL IL-7; wherein the total concentration of potassium ion and NaCl is from 110 mM to 140 mM. In some aspects, the MRM comprises more than 40 mM potassium ion, NaCl, and about 300 ng/mL IL-2 and about 290 ng/mL IL-7; wherein the total concentration of potassium ion and NaCl is from 110 mM to 140 mM. In some aspects, the MRM comprises at least about 45 mM potassium ion, NaCl, about 300 ng/mL IL-2, and about 290 ng/mL IL-7; wherein the total concentration of potassium ion and NaCl is from 110 mM to 140 mM. In some aspects, the MRM comprises at least about 40 mM potassium ion, NaCl, about 300 ng/mL IL-2, and about 290 ng/mL IL-7; wherein the total concentration of potassium ion and NaCl is from 110 mM to 140 mM. In some aspects, the MRM comprises at least about 55 mM potassium ion, NaCl, about 300 ng/mL IL-2, and about 290 ng/mL IL-7; wherein the total concentration of potassium ion and NaCl is from 110 mM to 140 mM. In some aspects, the MRM comprises at least about 60 mM potassium ion, NaCl, about 300 ng/mL IL-2, and about 290 ng/mL IL-7; wherein the total concentration of potassium ion and NaCl is from 110 mM to 140 mM. In some aspects, the MRM comprises at least about 65 mM potassium ion, NaCl, about 300 ng/mL IL-2, and about 290 ng/mL IL-7; wherein the total concentration of potassium ion and NaCl is from 110 mM to 140 mM. In some aspects, the MRM comprises at least about 70 mM potassium ion, NaCl, about 300 ng/mL IL-2, and about 290 ng/mL IL-7; wherein the total concentration of potassium ion and NaCl is from 110 mM to 140 mM. In some aspects, the MRM comprises at least about 75 mM potassium ion, NaCl, about 300 ng/mL IL-2, and about 290 ng/mL IL-7; wherein the total concentration of potassium ion and NaCl is from 110 mM to 140 mM. In some aspects, the MRM comprises at least about 80 mM potassium ion, NaCl, about 300 ng/mL IL-2, and about 290 ng/mL IL-7; wherein the total concentration of potassium ion and NaCl is from 110 mM to 140 mM. In some aspects, the MRM comprises at least about 85 mM potassium ion, NaCl, about 300 ng/mL IL-2, and about 290 ng/mL IL-7; wherein the total concentration of potassium ion and NaCl is from 110 mM to 140 mM. In some aspects, the MRM comprises at least about 90 mM potassium ion, NaCl, about 300 ng/mL IL-2, and about 290 ng/mL IL-7; wherein the total concentration of potassium ion and NaCl is from 110 mM to 140 mM. In some aspects, the MRM comprises (i) at least about 70 mM potassium ion, (ii) about 60 mM sodium (e. g; NaCl), (iii) about 1.4 mM calcium, (iv) about 16 mM glucose, (v) about 300 ng/mL IL-2, and (vi) about 290 ng/mL IL-7.

In some aspects, the MRM comprises at least about 30 mM to at least about 100 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises more than 40 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises at least about 45 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises at least about 50 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises at least about 55 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises at least about 60 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises at least about 65 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises at least about 70 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises at least about 75 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises at least about 80 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises at least about 85 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises at least about 90 mM potassium ion, NaCl, about 300 ng/mL IL-2, and about 0.4 ng/mL IL-15; wherein the total concentration of potassium ion and NaCl is from 110 mM to 140 mM. In some aspects, the MRM comprises (i) at least about 70 mM potassium ion, (ii) about 60 mM sodium (e. g; NaCl), (iii) about 1.4 mM calcium, (iv) about 16 mM glucose, (v) about 300 ng/mL IL-2, and (vi) about 0.4 ng/mL IL-15.

In some aspects, the MRM comprises at least about 30 mM to at least about 100 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises more than 40 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises at least about 45 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises at least about 50 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises at least about 55 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises at least about 60 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises at least about 65 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises at least about 70 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises at least about 75 mM potassium ion, NaCl, and about 10 ng/mL IL-2, about 1 ng/mL IL-7, and about 1 ng/mL IL-15; wherein the total concentration of potassium ion and NaCl is from 110 mM to 140 mM. In some aspects, the MRM comprises at least about 80 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises at least about 85 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises at least about 90 mM potassium ion, NaCl, about 300 ng/mL IL-2, about 290 ng/mL IL-7, and about 0.4 ng/mL IL-15; wherein the total concentration of potassium ion and NaCl is from 110 mM to 140 mM. In some aspects, the MRM comprises (i) at least about 70 mM potassium ion, (ii) about 60 mM sodium (e. g., NaCl), (iii) about 1.4 mM calcium, (iv) about 16 mM glucose, (v) about 300 ng/mL IL-2, (vi) about 290 ng/mL IL-7, and (vii) about 0.4 ng/mL IL-15.

In some aspects, the MRM comprises at least about 30 mM to at least about 100 mM potassium ion, NaCl, about 300 ng/mL IL-2, and about 30 ng/mL IL-21; wherein the total concentration of potassium ion and NaCl is from 110 mM to 140 mM. In some aspects, the MRM comprises more than 40 mM potassium ion, NaCl, about 300 ng/mL IL-2, and about 30 ng/mL IL-21; wherein the total concentration of potassium ion and NaCl is from 110 mM to 140 mM. In some aspects, the MRM comprises at least about 45 mM potassium ion, NaCl, about 300 ng/mL IL-2, and about 30 ng/mL IL-21; wherein the total concentration of potassium ion and NaCl is from 110 mM to 140 mM. In some aspects, the MRM comprises at least about 50 mM potassium ion, NaCl, about 300 ng/mL IL-2, and about 30 ng/mL IL-21; wherein the total concentration of potassium ion and NaCl is from 110 mM to 140 mM. In some aspects, the MRM comprises at least about 55 mM potassium ion, NaCl, about 300 ng/mL IL-2, and about 30 ng/mL IL-21; wherein the total concentration of potassium ion and NaCl is from 110 mM to 140 mM. In some aspects, the MRM comprises at least about 60 mM potassium ion, NaCl, about 300 ng/mL IL-2, and about 30 ng/mL IL-21; wherein the total concentration of potassium ion and NaCl is from 110 mM to 140 mM. In some aspects, the MRM comprises at least about 65 mM potassium ion, NaCl, about 300 ng/mL IL-2, and about 30 ng/mL IL-21; wherein the total concentration of potassium ion and NaCl is from 110 mM to 140 mM. In some aspects, the MRM comprises at least about 70 mM potassium ion, NaCl, about 300 ng/mL IL-2, and about 30 ng/mL IL-21; wherein the total concentration of potassium ion and NaCl is from 110 mM to 140 mM. In some aspects, the MRM comprises at least about 75 mM potassium ion, NaCl, about 300 ng/mL IL-2, and about 30 ng/mL IL-21; wherein the total concentration of potassium ion and NaCl is from 110 mM to 140 mM. In some aspects, the MRM comprises at least about 80 mM potassium ion, NaCl, about 300 ng/mL IL-2, and about 30 ng/mL IL-21; wherein the total concentration of potassium ion and NaCl is from 110 mM to 140 mM. In some aspects, the MRM comprises at least about 85 mM potassium ion, NaCl, about 300 ng/mL IL-2, and about 30 ng/mL IL-21; wherein the total concentration of potassium ion and NaCl is from 110 mM to 140 mM. In some aspects, the MRM comprises at least about 90 mM potassium ion, NaCl, about 300 ng/mL IL-2, and about 30 ng/mL IL-21; wherein the total concentration of potassium ion and NaCl is from 110 mM to 140 mM. In some aspects, the MRM comprises (i) at least about 70 mM potassium ion, (ii) about 60 mM sodium (e. g; NaCl), (iii) about 1.4 mM calcium, (iv) about 16 mM glucose, (v) about 300 ng/mL IL-2, and (vi) about 30 ng/mL IL-21.

In some aspects, the MRM comprises at least about 30 mM to at least about 100 mM potassium ion, NaCl, about 290 ng/mL IL-7, and about 20 ng/mL IL-21; wherein the total concentration of potassium ion and NaCl is from 110 mM to 140 mM. In some aspects, the MRM comprises more than 40 mM potassium ion, NaCl, about 290 ng/mL IL-7, and about 20 ng/mL IL-21; wherein the total concentration of potassium ion and NaCl is from 110 mM to 140 mM. In some aspects, the MRM comprises at least about 45 mM potassium ion, NaCl, about 290 ng/mL IL-7, and about 20 ng/mL IL-21; wherein the total concentration of potassium ion and NaCl is from 110 mM to 140 mM. In some aspects, the MRM comprises at least about 50 mM potassium ion, NaCl, about 290 ng/mL IL-7, and about 20 ng/mL IL-21; wherein the total concentration of potassium ion and NaCl is from 110 mM to 140 mM. In some aspects, the MRM comprises at least about 55 mM potassium ion, NaCl, about 290 ng/mL IL-7, and about 20 ng/mL IL-21; wherein the total concentration of potassium ion and NaCl is from 110 mM to 140 mM. In some aspects, the MRM comprises at least about 60 mM potassium ion, NaCl, about 290 ng/mL IL-7, and about 20 ng/mL IL-21; wherein the total concentration of potassium ion and NaCl is from 110 mM to 140 mM. In some aspects, the MRM comprises at least about 65 mM potassium ion, NaCl, about 290 ng/mL IL-7, and about 20 ng/mL IL-21; wherein the total concentration of potassium ion and NaCl is from 110 mM to 140 mM. In some aspects, the MRM comprises at least about 70 mM potassium ion, NaCl, about 290 ng/mL IL-7, and about 20 ng/mL IL-21; wherein the total concentration of potassium ion and NaCl is from 110 mM to 140 mM. In some aspects, the MRM comprises at least about 75 mM potassium ion, NaCl, about 290 ng/mL IL-7, and about 20 ng/mL IL-21; wherein the total concentration of potassium ion and NaCl is from 110 mM to 140 mM. In some aspects, the MRM comprises at least about 80 mM potassium ion, NaCl, about 290 ng/mL IL-7, and about 20 ng/mL IL-21; wherein the total concentration of potassium ion and NaCl is from 110 mM to 140 mM. In some aspects, the MRM comprises at least about 85 mM potassium ion, NaCl, about 290 ng/mL IL-7, and about 20 ng/mL IL-21; wherein the total concentration of potassium ion and NaCl is from 110 mM to 140 mM. In some aspects, the MRM comprises at least about 90 mM potassium ion, NaCl, about 290 ng/mL IL-7, and about 20 ng/mL IL-21; wherein the total concentration of potassium ion and NaCl is from 110 mM to 140 mM. In some aspects, the MRM comprises (i) at least about 70 mM potassium ion, (ii) about 60 mM sodium (e. g; NaCl), (iii) about 1.4 mM calcium, (iv) about 16 mM glucose, (v) about 290 ng/mL IL-7, and (vi) about 20 ng/mL IL-21.

In some aspects, the MRM comprises at least about 30 mM to at least about 100 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises more than 40 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises at least about 45 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises at least about 50 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises at least about 55 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises at least about 60 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises at least about 65 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises at least about 70 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises at least about 75 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises at least about 80 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises at least about 85 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises at least about 90 mM potassium ion, NaCl, about 0.4 ng/mL IL-15, and about 20 ng/mL IL-21; wherein the total concentration of potassium ion and NaCl is from 110 mM to 140 mM. In some aspects, the MRM comprises (i) at least about 70 mM potassium ion, (ii) about 60 mM sodium (e. g; NaCl), (iii) about 1.4 mM calcium, (iv) about 16 mM glucose, (v) about 0.4 ng/mL IL-15, and (vi) about 20 ng/mL IL-21.

II.G. T Cell Culture Media (e.g., Metabolic Reprograming Media)

In some aspects, the MRM is prepared by adding potassium ion to a basal medium. Any basal medium that is used to culture immune cells, (e.g., T cells, NK cells, and/or TILs), can be used.

In some aspects, the MRM further comprises one or more essential amino acids. In some aspects, the basal media comprises one or more essential amino acids selected form L-arginine, L-cystine, L-isoleucine, L-leucine, L-lysine, L-methionine, L-phenylalanine, L-threonine, L-tryptophan, L-histidine, L-tyrosine, L-valine, and L-glutamine, or any combination thereof. In some aspects, the basal media comprises L-glutamine.

In some aspects, the MRM comprises at least about 0.01 mM of one or more essential amino acids. In some aspects, the MRM comprises about 0.01 mM to about 10 mM of one or more essential amino acids. In some aspects, the MRM comprises about 0.01 mM to about 10 mM, about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, or about 1 mM to about 2 mM of one or more essential amino acids.

In some aspects, the MRM comprises at least about 0.01 mM, at least about 0.1 mM, at least about 0.5 mM, at least about 1.0 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM or at least about 50 mM of one or more essential amino acids.

In some aspects, the MRM comprises about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.1 mM, about 2.2 mM, about 2.3 mM, about 2.4 mM, about 2.5 mM, about 2.6 mM, about 2.7 mM, about 2.8 mM, about 2.9 mM, about 3.0 mM, about 3.1 mM, about 3.2 mM, about 3.3 mM, about 3.4 mM, about 3.5 mM, about 3.6 mM, about 3.7 mM, about 3.8 mM, about 3.9 mM, about 4.0 mM, about 4.1 mM, about 4.2 mM, about 4.3 mM, about 4.4 mM, about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, about 5.5 mM, about 5.6 mM, about 5.7 mM, about 5.8 mM, about 5.9 mM, about 6.0 mM, about 6.1 mM, about 6.2 mM, about 6.3 mM, about 6.4 mM, about 6.5 mM, about 6.6 mM, about 6.7 mM, about 6.8 mM, about 6.9 mM, or about 7.0 mM of one or more essential amino acids.

In some aspects, the MRM comprises L-glutamine. In some aspects, MRM comprises at least about 0.01 mM L-glutamine. In some aspects, the MRM comprises about 0.01 mM to about 10 mM L-glutamine. In some aspects, the MRM comprises about 0.01 mM to about 10 mM, about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, or about 1 mM to about 2 mM L-glutamine.

In some aspects, the MRM comprises at least about 0.01 mM, at least about 0.1 mM, at least about 0.5 mM, at least about 1.0 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM or at least about 50 mM L-glutamine.

In some aspects, the MRM comprises about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.1 mM, about 2.2 mM, about 2.3 mM, about 2.4 mM, about 2.5 mM, about 2.6 mM, about 2.7 mM, about 2.8 mM, about 2.9 mM, about 3.0 mM, about 3.1 mM, about 3.2 mM, about 3.3 mM, about 3.4 mM, about 3.5 mM, about 3.6 mM, about 3.7 mM, about 3.8 mM, about 3.9 mM, about 4.0 mM, about 4.1 mM, about 4.2 mM, about 4.3 mM, about 4.4 mM, about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, about 5.5 mM, about 5.6 mM, about 5.7 mM, about 5.8 mM, about 5.9 mM, about 6.0 mM, about 6.1 mM, about 6.2 mM, about 6.3 mM, about 6.4 mM, about 6.5 mM, about 6.6 mM, about 6.7 mM, about 6.8 mM, about 6.9 mM, or about 7.0 mM L-glutamine. In some aspects, the MRM comprises about 1.7 mM L-glutamine. In some aspects, the MRM comprises about 1.68 mM L-glutamine.

In some aspects, the MRM comprises about 0.14 mM L-glutamine. In some aspects, the MRM comprises about 0.15 mM L-glutamine. In some aspects, the MRM comprises about 1.76 mM L-glutamine. In some aspects, the MRM comprises about 1.83 mM L-glutamine. In some aspects, the MRM comprises about 1.84 mM L-glutamine. In some aspects, the MRM comprises about 1.97 mM L-glutamine. In some aspects, the MRM comprises about 2.05 mM L-glutamine. In some aspects, the MRM comprises about 2.11 mM L-glutamine. In some aspects, the MRM comprises about 2.18 mM L-glutamine. In some aspects, the MRM comprises about 5.41 mM L-glutamine. In some aspects, the MRM comprises about 5.47 mM L-glutamine. In some aspects, the MRM comprises about <0.10 mM L-glutamine.

In some aspects, the MRM comprises L-glutamic acid. In some aspects, the MRM comprises at least about 0.01 mM L-glutamic acid. In some aspects, the MRM comprises about 0.01 mM to about 10 mM L-glutamic acid. In some aspects, the MRM comprises about 0.01 mM to about 10 mM, about 0.01 mM to about 9 mM, about 0.01 mM to about 8 mM, about 0.01 mM to about 7 mM, about 0.01 mM to about 6 mM, about 0.01 mM to about 5 mM, about 0.01 mM to about 4 mM, about 0.01 mM to about 3 mM, about 0.01 mM to about 2 mM, about 0.01 mM to about 1 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, about 0.1 mM to about 1 mM, about 1 mM to about 10 mM, about 1 mM to about 9 mM, about 1 mM to about 8 mM, about 1 mM to about 7 mM, about 1 mM to about 6 mM, about 1 mM to about 5 mM, about 1 mM to about 4 mM, about 1 mM to about 3 mM, or about 1 mM to about 2 mM L-glutamic acid.

In some aspects, the MRM comprises at least about 0.01 mM, at least about 0.1 mM, at least about 0.5 mM, at least about 1.0 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, or at least about 15 mM or at least about 50 mM L-glutamic acid.

In some aspects, the MRM comprises about 0.01 mM, about 0.05 mM, about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.1 mM, about 2.2 mM, about 2.3 mM, about 2.4 mM, about 2.5 mM, about 2.6 mM, about 2.7 mM, about 2.8 mM, about 2.9 mM, about 3.0 mM, about 3.1 mM, about 3.2 mM, about 3.3 mM, about 3.4 mM, about 3.5 mM, about 3.6 mM, about 3.7 mM, about 3.8 mM, about 3.9 mM, about 4.0 mM, about 4.1 mM, about 4.2 mM, about 4.3 mM, about 4.4 mM, about 4.5 mM, about 4.6 mM, about 4.7 mM, about 4.8 mM, about 4.9 mM, about 5.0 mM, about 5.1 mM, about 5.2 mM, about 5.3 mM, about 5.4 mM, about 5.5 mM, about 5.6 mM, about 5.7 mM, about 5.8 mM, about 5.9 mM, about 6.0 mM, about 6.1 mM, about 6.2 mM, about 6.3 mM, about 6.4 mM, about 6.5 mM, about 6.6 mM, about 6.7 mM, about 6.8 mM, about 6.9 mM, or about 7.0 mM L-glutamic acid.

In some aspects, the MRM comprises about 0.15 mM L-glutamic acid. In some aspects, the MRM comprises about 0.17 mM L-glutamic acid. In some aspects, the MRM comprises about 0.18 mM L-glutamic acid. In some aspects, the MRM comprises about 0.19 mM L-glutamic acid. In some aspects, the MRM comprises about 0.85 mM L-glutamic acid. In some aspects, the MRM comprises about 0.86 mM L-glutamic acid. In some aspects, the MRM comprises about 0.9 mM L-glutamic acid. In some aspects, the MRM comprises about 0.95 mM L-glutamic acid. In some aspects, the MRM comprises about 1.06 mM L-glutamic acid. In some aspects, the MRM comprises about 1.09 mM L-glutamic acid. In some aspects, the MRM comprises about <0.10 mM L-glutamic acid.

In some aspects, the MRM comprises a dipeptide. In some aspects, the MRM comprises glutamine-glutamine (Gin-Gin). In some aspects, the MRM comprises alanyl-glutamine (Ala-Gin).

In some aspects, the MRM comprises at least about 0.1 mM dipeptide (e.g., Ala-Gln). In some aspects, the MRM comprises about 0.1 mM to about 50 mM dipeptide (e.g., Ala-Gln). In some aspects, the MRM comprises about 0.1 mM to about 40 mM, about 0.1 mM to about 35 mM, about 0.1 mM to about 30 mM, about 0.1 mM to about 25 mM, about 0.1 mM to about 20 mM, about 1 mM to about 20 mM, about 2 mM to about 20 mM, about 3 mM to about 20 mM, about 4 mM to about 20 mM, about 5 mM to about 20 mM, about 6 mM to about 20 mM, about 7 mM to about 20 mM, about 8 mM to about 20 mM, about 9 mM to about 20 mM, about 10 mM to about 20 mM, about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, or about 9 mM to about 10 mM dipeptide (e.g., Ala-Gin).

In some aspects, the MRM comprises at least about 0.1 mM, at least about 1.0 mM, at least about 2 mM, at least about 3 mM, at least about 4 mM, at least about 5 mM, at least about 6 mM, at least about 7 mM, at least about 8 mM, at least about 9 mM, at least about 10 mM, at least about 11 mM, at least about 12 mM, at least about 13 mM, at least about 14 mM, at least about 15 mM, at least about 16 mM, at least about 17 mM, at least about 18 mM, at least about 19 mM, at least about 20 mM, at least about 25 mM, at least about 30 mM, or at least about 50 mM dipeptide (e.g., Ala-Gin).

In some aspects, the MRM comprises about 1 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM dipeptide (e.g., Ala-Gin). In some aspects, the basal medium comprises about 1.7 mM dipeptide (e.g., Ala-Gin). In some aspects, the MRM comprises about 1.68 mM dipeptide (e.g., Ala-Gin).

In some aspects, the MRM comprises about 6 mM, about 6.1 mM, about 6.2 mM, about 6.3 mM, about 6.4 mM, about 6.5 mM, about 6.6 mM, about 6.7 mM, about 6.8 mM, about 6.9 mM, about 7.0 mM, about 7.1 mM, or about 7.2 mM dipeptide (e.g., Ala-Gin). In some aspects, the MRM comprises about 6.8 mM dipeptide (e.g., Ala-Gin). In some aspects, the MRM comprises about 6.81 mM dipeptide (e.g., Ala-Gin). In some aspects, the MRM comprises about 6.9 mM dipeptide (e.g., Ala-Gin). In some aspects, the MRM comprises about 6.96 mM dipeptide (e.g., Ala-Gin). In some aspects, the MRM comprises about 7.0 mM dipeptide (e.g., Ala-Gin).

In some aspects, the MRM comprises less than about 5 mM ammonia (NH₃). In some aspects, the MRM comprises less than about 4 mM, less than about 3.5 mM, less than about 3 mM, less than about 2.5 mM, less than about 2 mM, less than about 1.5 mM, less than about 1 mM, less than about 0.5 mM, less than about 0.4 mM, less than about 0.3 mM, less than about 0.2 mM, or less than about 0.1 mM ammonia. In some aspects, the MRM comprises about 0.01 mM ammonia to less than about 2 mM ammonia, about 0.01 mM ammonia to less than about 1.9 mM ammonia, about 0.01 mM ammonia to less than about 1.8 mM ammonia, about 0.01 mM ammonia to less than about 1.7 mM ammonia, about 0.01 mM ammonia to less than about 1.6 mM ammonia, about 0.01 mM ammonia to less than about 1.5 mM ammonia, about 0.01 mM ammonia to less than about 1.4 mM ammonia, about 0.01 mM ammonia to less than about 1.3 mM ammonia, about 0.01 mM ammonia to less than about 1.2 mM ammonia, about 0.01 mM ammonia to less than about 1.1 mM ammonia, about 0.01 mM ammonia to less than about 1 mM ammonia, about 0.01 mM ammonia to less than about 0.9 mM ammonia, about 0.01 mM ammonia to less than about 0.8 mM ammonia, about 0.01 mM ammonia to less than about 0.7 mM ammonia, about 0.01 mM ammonia to less than about 0.6 mM ammonia, about 0.01 mM ammonia to less than about 0.5 mM ammonia, about 0.01 mM ammonia to less than about 0.4 mM ammonia, about 0.01 mM ammonia to less than about 0.3 mM ammonia, about 0.01 mM ammonia to less than about 0.2 mM ammonia, or about 0.01 mM ammonia to less than about 0.1 mM ammonia. In some aspects, the MRM comprises about 1.2 mM ammonia. In some aspects, the MRM comprises about 1.25 mM ammonia. In some aspects, the MRM comprises about 1.259 mM ammonia. In some aspects, the MRM comprises about 1.28 mM ammonia. In some aspects, the MRM comprises about 1.3 mM ammonia. In some aspects, the MRM comprises about 0.3 mM ammonia. In some aspects, the MRM comprises about 0.34 mM ammonia. In some aspects, the MRM comprises about 0.35 mM ammonia. In some aspects, the MRM comprises about 0.36 mM ammonia. In some aspects, the MRM comprises about 0.37 mM ammonia. In some aspects, the MRM comprises less than about 0.3 mM ammonia. In some aspects, the MRM comprises less than about 0.29 mM ammonia. In some aspects, the MRM comprises less than about 0.28 mM ammonia. In some aspects, the MRM comprise less than about 0.278 mM ammonia. In some aspects, the MRM do not comprise ammonia.

In some aspects, the MRM comprises lactate. In some aspects, the MRM does not comprise lactate.

In some aspects, the MRM, e.g., secondary TIL expansion medium and/or third (or final) TIL expansion medium, further comprises a CD3 agonist and/or a CD28 agonist. The CD3 agonist and/or the CD28 agonist can stimulate TILs that are being cultured in the media. In some aspects, a CD3 agonist can be any molecule that is capable of binding to CD3 complex and activating CD3. In some aspects, a CD3 agonist is a small molecule. In some aspects, a CD3 agonist is a protein. In some aspects, a CD3 agonist is an anti-CD3 antibody. The term “anti-CD3 antibody” as used herein refers to an antibody or variant thereof, e.g., a monoclonal antibody and including human, humanized, chimeric or murine antibodies which are directed against the CD3 complex in T cells. In some aspects, an anti-CD3 antibody comprises OKT-3, also known as muromonab, and UCHT-1. Other anti-CD3 antibodies include, for example, visilizumab otelixizumab, and teplizumab.

The term “OKT-3” or “OKT3” refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotech, Inc., San Diego, Calif., USA) and muromonab or variants, conservative amino acid substitutions, glycoforms, or biosimilars thereof. A hybridoma capable of producing OKT-3 is deposited with European Collection of Authenticated Cell Cultures (ECACC) and assigned Catalogue No. 86022706. A hybridoma capable of producing OKT-3 is also deposited with the American Type Culture Collection and assigned the ATCC accession number CRL 8001.

In some aspects, a CD28 agonist can be any molecule that is capable of activating CD28 or its downstream pathway. In some aspects, a CD28 agonist is a small molecule. In some aspects, a CD28 agonist is a protein. In some aspects, a CD28 agonist is an anti-CD28 antibody. The term “anti-CD28 antibody” as used herein refers to an antibody or variant thereof, e.g., a monoclonal antibody and including human, humanized, chimeric or murine antibodies which are directed against CD28 and activate T cells. In some aspects, an anti-CD28 antibody comprises Catalog #100182-1 (BPS Bioscicence), Catalog #100186-1 (BPS Bioscience).

In some aspects, the CD3 agonist and the CD28 agonist are added in the MRM together. In some aspects, the CD3 agonist and the CD28 agonist are added in the MRM concurrently in one composition. In some aspects, the CD3 agonist and the CD28 agonist are added in sequence. In some aspects, the MRM, e.g., secondary TIL expansion media and/or third (or final) TIL expansion media, comprises and/or is supplemented with a substituent comprising both a CD3 agonist and a CD28 agonist, e.g., TRANSACT™. In some aspects, the MRM comprises at least about 1:100 TRANSACT™. In some aspects, the MRM comprises at least about 1:150 TRANSACT™. In some aspects, the MRM comprises at least about 1:200 TRANSACT™. In some aspects, the MRM comprises at least about 1:250 TRANSACT™. In some aspects, the MRM comprises at least about 1:300 TRANSACT™. In some aspects, the MRM comprises at least about 1:350 TRANSACT™. In some aspects, the MRM comprises at least about 1:400 TRANSACT™. In some aspects, the MRM comprises at least about 1:450 TRANSACT™. In some aspects, the MRM comprises at least about 1:500 TRANSACT™.

In some aspects, the MRM, e.g., secondary TIL expansion media and/or third (or final) TIL expansion media, comprises and/or is supplemented with a TRANSACT™ alternative. Artificial antigen presenting cells (aAPCs) such as genetically engineered human K562 aAPCs can be used for rapid expansion of TILs. In some aspects, the aAPC is generated by transducing K562 cells with a polycistronic lentiviral vector comprising genes encoding CD70, CD80, CD86, 41BB ligand, and OX40 ligand. K562 cells do not express HLA-A, HLA-B, or HLA-DR molecules, which makes them a powerful tool for T cell expansion when transduced with the above mentioned co-stimulatory ligands (See, e.g., Suhoski et al., Molecular therapy, 2007). In some aspects, secondary TIL expansion and/or third TIL expansion comprises co-culturing the TILs with aAPCs+OKT3. In some aspects, secondary TIL expansion and/or third TIL expansion comprises co-culturing the TILs with irradiated APCs (e.g., PBMC) in the presense of OKT3 (e.g., at least about 30 ng/mL OKT3) instead of TRANSACT™. In some aspects, the ratio of immune cells (e.g., TILs) to feeder cells (e.g., aAPCs) is at least about 1:50, at least about 1:100, at least about 1:150, at least about 1:200, at least about 1:250, at least about 1:300, at least about 1:350, at least about 1:400, at least about 1:450, or at least about 1:500. In some aspects, the ratio of immune cells (e.g., TILs) to feeder cells (e.g., aAPCs) is at least about 1:100. In some aspects, the ratio of immune cells (e.g., TILs) to feeder cells (e.g., aAPCs) is at least about 1:200.

In some aspects, the MRM, e.g., secondary TIL expansion media and/or third (or final) TIL expansion media, comprise and/or are supplemented with a CD27 ligand (CD27L). CD27 ligand (CD70) is capable of binding to its receptor, and then upon binding, the receptor is capable of generating and long-term maintenance of T cell immunity. CD27 is a member of the TNF-receptor superfamily. CD27, a transmembrane homodimeric phosphoglycoprotein of 120 kDa, also appears to have a costimulatory role. CD27L, CD70, is a transmembrane glycoprotein expressed on T and B cells in response to antigen stimulation; it is thus considered a marker of the early stages of activation. In vitro, the interaction of CD27 on a T cell and CD70 on a B cell enhances T cell activation in terms of proliferation but only relatively low amounts of IL-2 are secreted. Studies of knockout mice have shown that CD27 plays a minor part in naive T cell activation but is crucial for the generation of T cell memory.

In some aspects, the MRM, e.g., secondary TIL expansion media and/or third (or final) TIL expansion media, comprises about 0.1 μg/ml to about 50 μg/ml CD27L. In some aspects, the MRM comprises and/or is supplemented with about 0.1 μg/ml to about 40 μg/ml, about 0.1 μg/ml to about 30 μg/ml, about 0.1 μg/ml to about 20 μg/ml, about 0.1 μg/ml to about 10 μg/ml, about 0.1 μg/ml to about 5 μg/ml, about 1 μg/ml to about 10 μg/ml, about 2 μg/ml to about 10 μg/ml, about 3 μg/ml to about 10 μg/ml, about 4 μg/ml to about 10 μg/ml, about 5 μg/ml to about 10 μg/ml, about 1 μg/ml to about 9 μg/ml, about 1 μg/ml to about 8 μg/ml, about 1 μg/ml to about 7 μg/ml, about 1 μg/ml to about 6 μg/ml, about 1 μg/ml to about 5 μg/ml, about 3 μg/ml to about 7 μg/ml, about 4 μg/ml to about 6 μg/ml, about 3 μg/ml to about 6 μg/ml, or about 4 μg/ml to about 7 μg/ml CD27L.

In some aspects, the MRM, e.g., secondary TIL expansion media and/or third (or final) TIL expansion media, comprises and/or is supplemented with at least about 0.1 μg/ml, at least about 1 μg/ml, at least about 2 μg/ml, at least about 3 μg/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 11 μg/ml, at least about 12 μg/ml, at least about 13 μg/ml, at least about 14 μg/ml, at least about 15 μg/ml, at least about 16 μg/ml, at least about 17 μg/ml, at least about 18 μg/ml, at least about 19 μg/ml, at least about 20 μg/ml, at least about 25 μg/ml, at least about 30 μg/ml, or at least about 50 μg/ml CD27L. In some aspects, the MRM comprises and/or is supplemented with at least about 5 μg/ml CD27L.

In some aspects, the MRM, e.g., secondary TIL expansion medium and/or third (or final) TIL expansion medium, comprises and/or is supplemented with 4-1BB ligand (4-1BBL). 4-1BBL (4-1BB ligand, CD137L) is found on APCs (antigen presenting cells) and binds to 4-1BB (also known as CD137), a type 2 transmembrane glycoprotein receptor belonging to the TNF superfamily, which is expressed on activated T Lymphocytes. 4-1BB ligand can be used to activate T cells in vitro. In some aspects, the MRM, e.g., secondary TIL expansion media and/or third (or final) TIL expansion media, comprise about 0.1 μg/ml to about 50 μg/ml CD27L. In some aspects, the MRM comprises and/or is supplemented with about 0.1 μg/ml to about 10 μg/ml, about 0.1 μg/ml to about 9 μg/ml, about 0.1 μg/ml to about 8 μg/ml, about 0.1 μg/ml to about 7 μg/ml, about 0.1 μg/ml to about 6 μg/ml, about 0.1 μg/ml to about 5 μg/ml, about 0.1 μg/ml to about 4 μg/ml, about 0.1 μg/ml to about 3 μg/ml, about 0.1 μg/ml to about 2 μg/ml, about 0.1 μg/ml to about 1 μg/ml, 1 μg/ml to about 10 μg/ml, about 1 μg/ml to about 5 μg/ml, about 1 μg/ml to about 4 μg/ml, about 1 μg/ml to about 3 μg/ml, or about 1 μg/ml to about 2 μg/ml 4-1BBL.

In some aspects, the MRM, e.g., secondary TIL expansion media and/or third (or final) TIL expansion media, comprise and/or are supplemented with at least about 0.1 μg/ml, at least about 0.2 μg/ml, at least about 0.3 μg/ml, at least about 0.4 μg/ml, at least about 0.5 μg/ml, at least about 0.6 μg/ml, at least about 0.7 μg/ml, at least about 0.8 μg/ml, at least about 0.9 μg/ml, at least about 1 μg/ml, at least about 1.1 μg/ml, at least about 1.2 μg/ml, at least about 1.3 μg/ml, at least about 1.4 μg/ml, at least about 1.5 μg/ml, at least about 1.6 μg/ml, at least about 1.7 μg/ml, at least about 1.8 μg/ml, at least about 1.9 μg/ml, at least about μg/ml, at least about 2 μg/ml, at least about 3 μg/ml, at least about 4 μg/ml, at least about 5 μg/ml, or at least about 10 μg/ml 4-1BBL. In some aspects, the MRM comprises and/or is supplemented with at least about 1 μg/ml 4-1BBL.

In some aspects, a 4-1BBL is added in the MRM together with a CD27L. In some aspects, a 4-1BBL is added in the MRM concurrently with a CD27L. In some aspects, a 4-1BBL is added in the MRM with a CD27L in sequence. In some aspects, the MRM used during an expansion process (e.g., a secondary expansion and/or a final expansion) comprises TRANSACT™, 4-1BBL, and CD27L. In some aspects, the MRM comprises at least about 1:100 TRANSACT™, at least about 1 μg/ml 4-1BBL, and at least about 5 μg/ml CD27L. In some aspects, the MRM used during an expansion process (e.g., a secondary expansion and/or a final expansion) comprises at least about 1:100 TRANSACT™, at least about 1 μg/ml 4-1BBL, and at least about 5 μg/ml CD27L.

In some aspects, the MRM, e.g., initial TIL culture medium, secondary TIL expansion medium and/or third (or final) TIL expansion medium, is modified from a basal medium selected from a balanced salt solution (e.g., PBS, DPBS, HBSS, EBSS), Dulbecco's Modified Eagle's Medium (DMEM), Click's medium, Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OpTmizer™ CTS™ T-Cell Expansion Basal Medium (ThermoFisher), OPTMIZER™ Complete, IMMUNOCULT™ XF (STEMCELL™ Technologies), IMMUNOCULT™ XF, AIM V, TEXMACS™ medium, and any combination thereof. In some aspects, the basal medium is serum free. In some aspects, the basal medium further comprises immune cell serum replacement (ICSR). For example, in some aspects, the basal medium comprises OPTMIZER™ Complete supplemented with ICSR, AIM V supplemented with ICSR, IMMUNOCULT™ XF supplemented with ICSR, RPMI supplemented with ICSR, TEXMACS™ supplemented with ICSR, or any combination thereof. In particular aspects, the basal media comprises OPTMIZER™ complete. In some aspects, suitable basal medium includes Click's medium, OpTimizer® (CTS®) medium, Stemline® T cell expansion medium (Sigma-Aldrich), AIM V® medium (CTS®), TexMACS® medium (Miltenyi Biotech), ImmunoCult® medium (Stem Cell Technologies), PRIME-XV® T-Cell Expansion XSFM (Irvine Scientific), Iscoves medium, and/or RPMI-1640 medium.

The present disclosure comprises a MRM comprising basal media, NaCl, KCl, calcium, and glucose, wherein the concentration of NaCl is between about 40 mM and about 80 mM, the concentration of KCl is between 40 and 90 mM, the concentration of calcium is between about 0.5 mM and about 2.8 mM, and the concentration of glucose between about 10 mM and about 24 mM.

In some aspects, the MRM further comprises immune cells. In some aspects, the immune cells comprises TILs.

In some aspects, the MRM further comprises IL-2, IL-7, IL-15, IL-21, or any combination thereof. In some aspects, the MRM further comprises IL-2 and IL-21. In some aspects, the concentration of IL-2 is about 200 ng/ml to about 400 ng/ml (e.g., about 200 ng/ml, about 300 ng/ml, or about 400 ng/ml). In some aspects, the concentration of IL-21 is about 20 ng/ml to about 40 ng/ml, (e.g., about 20 ng/ml, about 30 ng/ml, or about 40 ng/ml).

In some aspects, the MRM further comprises about 2.5% serum supplement (CTS™ Immune Cell SR, Thermo Fisher), 2 mM L-glutamine, 2 mM L-glutamax, MEM Non-Essential Amino Acids Solution, Pen-strep, 20 μg/ml FUNGIN™, Sodium pyruvate, or any combination thereof. In some aspects, the MRM further comprises O-Acetyl-L-carnitine hydrochloride. In some aspects, the MRM further comprises a kinase inhibitor.

In some aspects, the MRM further comprises a CD3 agonist. In some aspects, the CD3 agonist is an anti-CD3 antibody. In some aspects, the anti-CD3 antibody comprises OKT-3.

In some aspects, the MRM further comprises a CD28 agonist. In some aspects, the CD28 agonist is an anti-CD28 antibody. In some aspects, the MRM further comprises a CD27 ligand (CD27L). In some aspects, the MRM further comprises a 4-1BB ligand (4-1BBL).

In some aspects, the present disclosure includes a cell culture comprising the MRM disclosed herein, a cell bag comprising the MRM disclosed herein, or a bioreactor comprising the MRM disclosed herein.

II.H. Cells

Some aspects of the present disclosure are directed to methods of culturing TILs, comprising placing the TILs in a medium comprising potassium ion at a concentration of greater than 5 mM, as disclosed herein. Some aspects of the present disclosure are directed to methods of culturing TILs, comprising placing the TILs in a medium comprising potassium ion at a concentration higher than 40 mM, as disclosed herein. Some aspects of the present disclosure are directed to methods of culturing TILs, comprising placing the TILs in a medium comprising potassium ion at a concentration of at least about 50 mM, as disclosed herein. Some aspects of the present disclosure are directed to methods of culturing TILs, comprising placing the TILs in a medium comprising potassium ion at a concentration of at least about 40 mM to at least about 90 mM, as disclosed herein.

Some aspects of the present disclosure are directed to methods of culturing TILs, comprising placing the TILs in a medium comprising potassium ion at a concentration higher than 40 mM and NaCl at a concentration of less than 100 mM, as disclosed herein. Some aspects of the present disclosure are directed to methods of culturing TILs, comprising placing the TILs in a medium comprising potassium ion at a concentration of at least about 50 mM and NaCl at a concentration of less than 90 mM, as disclosed herein. Some aspects of the present disclosure are directed to methods of culturing TILs, comprising placing the TILs in a medium comprising potassium ion at a concentration of at least about 40 mM to at least about 90 mM and NaCl at a concentration of less than 100 mM to 50 mM, as disclosed herein.

The TILs that are placed in the MRM can be TILs that are collected and/or isolated from a subject in need of a therapy. In some aspects, the TILs that are placed in the medium have been expanded prior to being placed in a MRM disclosed herein. The TILs that are placed in the medium can be referred to as starting (initial, i.e., patient sample, apheresis sample, buffy coat) TILs. The TILs that result from culturing them in the media disclosed herein can be referred to as resulting (cultured) TILs.

In some aspects, the TILs are present in a tumor sample obtained from a subject. Accordingly, in some aspects, the method comprises placing a tumor sample into an MRM disclosed herein. During standard TIL culture, tumor samples, e.g., a tumor biopsy or a fragment thereof, is plated in an initial TIL culture medium, and cultured for at least about 14-19 days. In some aspects, the tumor sample, e.g., the tumor biopsy, is cultured in an MRM in an initial TIL culture for at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 day. In some aspects, the initial TIL culture lasts about 14 days. In some aspects the initial TIL culture lasts sufficient number of days until a cell yield of about 2×10⁶ to about 10×10⁶ cells are produced.

In some aspects, the proportion of CD8⁺ TILs to non-CD8⁺ TILs (e.g., the proportion of CD8⁺ TILs to CD4⁺ TILs) is increased following the initial TIL culture, as compared to the proportion of CD8⁺ TILs to non-CD8⁺ TILs prior to the initial TIL culture. In some aspects, the proportion of CD8⁺ TILs to non-CD8⁺ TILs (e.g., the proportion of CD8⁺ TILs to CD4⁺ TILs) is increased following the initial TIL culture, as compared to the proportion of CD8⁺ TILs to non-CD8⁺ TILs following an initial TIL culture in a basal medium or a medium that does not comprise an increased concentration of potassium ion (control medium). In some aspects, the proportion of CD8⁺ TILs is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 45-fold, or at least about 50-fold. In some aspects, the proportion of CD8⁺ TILs is increased by at least about 40-fold. In some aspects, the proportion of CD8⁺ TILs is increased by at least about 50-fold.

In some aspects, the proportion of CD8⁺ TILs is increased by at least about 20%, at least about 25%, at least about 30%, at least about 35%, at least about 40%, at least about 45%, at least about 50%, at least about 60%, at least about 70%, at least about 75%, at least about 80%, at least about 90%, at least about 100%, at least about 125%, at least about 150%, at least about 175%, at least about 200%, at least about 250%, at least about 300%, at least about 350%, at least about 400%, at least about 450%, or at least about 500%. In some aspects, the proportion of CD8⁺ TILs is increased by at least about 20%. In some aspects, the proportion of CD8⁺ TILs is increased by at least about 40%. In some aspects, the proportion of CD8⁺ TILs is increased by at least about 60%. In some aspects, the proportion of CD8⁺ TILs is increased by at least about 80%. In some aspects, the proportion of CD8⁺ TILs is increased by at least about 100%.

In some aspects, the proportion of CD8⁺ TILs is increased to at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% of the total number of TILs in the culture. In some aspects, the proportion of CD8⁺ TILs is increased to at least about 20% of the total number of TILs in the culture. In some aspects, the proportion of CD8⁺ TILs is increased to at least about 30% of the total number of TILs in the culture. In some aspects, the proportion of CD8⁺ TILs is increased to at least about 40% of the total number of TILs in the culture. In some aspects, the proportion of CD8⁺ TILs is increased to at least about 50% of the total number of TILs in the culture. In some aspects, the proportion of CD8⁺ TILs is increased to at least about 60% of the total number of TILs in the culture. In some aspects, the proportion of CD8⁺ TILs is increased to at least about 70% of the total number of TILs in the culture. In some aspects, the proportion of CD8⁺ TILs is increased to at least about 75% of the total number of TILs in the culture. In some aspects, the proportion of CD8⁺ TILs is increased to at least about 80% of the total number of TILs in the culture. In some aspects, the proportion of CD8⁺ TILs is increased to at least about 90% of the total number of TILs in the culture.

In some aspects, the number of tumor-reactive cells in the culture is increased by about 2-fold to about 500-fold, about 2-fold to about 250-fold, about 2-fold to about 200-fold, about 2-fold to about 150-fold, about 2-fold to about 100-fold, about 2-fold to about 90-fold, about 2-fold to about 80-fold, about 2-fold to about 70-fold, about 2-fold to about 60-fold, about 2-fold to about 50-fold, about 2-fold to about 40-fold, about 2-fold to about 30-fold, about 2-fold to about 20-fold, about 2-fold to about 10-fold, about 5-fold to about 200-fold, about 5-fold to about 150-fold, about 5-fold to about 100-fold, about 5-fold to about 90-fold, about 5-fold to about 80-fold, about 5-fold to about 70-fold, about 5-fold to about 60-fold, about 5-fold to about 50-fold, about 5-fold to about 40-fold, about 5-fold to about 30-fold, about 5-fold to about 20-fold, about 5-fold to about 10-fold, about 10-fold to about 150-fold, about 10-fold to about 100-fold, about 10-fold to about 90-fold, about 10-fold to about 80-fold, about 10-fold to about 70-fold, about 10-fold to about 60-fold, about 10-fold to about 50-fold, about 10-fold to about 40-fold, about 10-fold to about 30-fold, or about 10-fold to about 20-fold following the culture methods disclosed herein, as compared to the number of tumor-reactive cells prior to the initial TIL culture. In some aspects, the number of tumor-reactive cells in the culture is increased by at least about 2-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 60-fold, at least about 70-fold, at least about 80-fold, at least about 90-fold, at least about 100-fold, at least about 125-fold, at least about 150-fold, at least about 175-fold, at least about 200-fold, at least about 250-fold, at least about 300-fold, at least about 350-fold, at least about 400-fold, at least about 450-fold, or at least about 500-fold following the culture methods disclosed herein, as compared to the number of tumor-reactive cells prior to the initial TIL culture. In some aspects, the number of tumor-reactive cells in the culture is increased by at least about 2-fold following the culture methods disclosed herein, as compared to the number of tumor-reactive cells prior to the initial TIL culture. In some aspects, the number of tumor-reactive cells in the culture is increased by at least about 3-fold following the culture methods disclosed herein, as compared to the number of tumor-reactive cells prior to the initial TIL culture. In some aspects, the number of tumor-reactive cells in the culture is increased by at least about 4-fold following the culture methods disclosed herein, as compared to the number of tumor-reactive cells prior to the initial TIL culture. In some aspects, the number of tumor-reactive cells in the culture is increased by at least about 5-fold following the culture methods disclosed herein, as compared to the number of tumor-reactive cells prior to the initial TIL culture. In some aspects, the number of tumor-reactive cells in the culture is increased by at least about 10-fold following the culture methods disclosed herein, as compared to the number of tumor-reactive cells prior to the initial TIL culture.

In some aspects, the number of tumor-reactive cells in the culture is increased by about 2-fold to about 500-fold, about 2-fold to about 250-fold, about 2-fold to about 200-fold, about 2-fold to about 150-fold, about 2-fold to about 100-fold, about 2-fold to about 90-fold, about 2-fold to about 80-fold, about 2-fold to about 70-fold, about 2-fold to about 60-fold, about 2-fold to about 50-fold, about 2-fold to about 40-fold, about 2-fold to about 30-fold, about 2-fold to about 20-fold, about 2-fold to about 10-fold, about 5-fold to about 200-fold, about 5-fold to about 150-fold, about 5-fold to about 100-fold, about 5-fold to about 90-fold, about 5-fold to about 80-fold, about 5-fold to about 70-fold, about 5-fold to about 60-fold, about 5-fold to about 50-fold, about 5-fold to about 40-fold, about 5-fold to about 30-fold, about 5-fold to about 20-fold, about 5-fold to about 10-fold, about 10-fold to about 150-fold, about 10-fold to about 100-fold, about 10-fold to about 90-fold, about 10-fold to about 80-fold, about 10-fold to about 70-fold, about 10-fold to about 60-fold, about 10-fold to about 50-fold, about 10-fold to about 40-fold, about 10-fold to about 30-fold, or about 10-fold to about 20-fold following the culture methods disclosed herein, as compared to the number of tumor-reactive cells following expansion using control methods (e.g., in medium comprising less than 40 mM potassium ion, e.g., 4 mM potassium ion). In some aspects, the number of tumor-reactive cells in the culture is increased by at least about 2-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 60-fold, at least about 70-fold, at least about 80-fold, at least about 90-fold, at least about 100-fold, at least about 125-fold, at least about 150-fold, at least about 175-fold, at least about 200-fold, at least about 250-fold, at least about 300-fold, at least about 350-fold, at least about 400-fold, at least about 450-fold, or at least about 500-fold following the culture methods disclosed herein, as compared to the number of tumor-reactive cells following expansion using control methods (e.g., in medium comprising less than 40 mM potassium ion, e.g., 4 mM potassium ion). In some aspects, the number of tumor-reactive cells in the culture is increased by at least about 2-fold following the culture methods disclosed herein, as compared to the number of tumor-reactive cells following expansion using control methods (e.g., in medium comprising less than 40 mM potassium ion, e.g., 4 mM potassium ion). In some aspects, the number of tumor-reactive cells in the culture is increased by at least about 3-fold following the culture methods disclosed herein, as compared to the number of tumor-reactive cells following expansion using control methods (e.g., in medium comprising less than 40 mM potassium ion, e.g., 4 mM potassium ion). In some aspects, the number of tumor-reactive cells in the culture is increased by at least about 4-fold following the culture methods disclosed herein, as compared to the number of tumor-reactive cells following expansion using control methods (e.g., in medium comprising less than 40 mM potassium ion, e.g., 4 mM potassium ion). In some aspects, the number of tumor-reactive cells in the culture is increased by at least about 5-fold following the culture methods disclosed herein, as compared to the number of tumor-reactive cells following expansion using control methods (e.g., in medium comprising less than 40 mM potassium ion, e.g., 4 mM potassium ion). In some aspects, the number of tumor-reactive cells in the culture is increased by at least about 10-fold following the culture methods disclosed herein, as compared to the number of tumor-reactive cells following expansion using control methods (e.g., in medium comprising less than 40 mM potassium ion, e.g., 4 mM potassium ion).

In some aspects, the tumor sample is isolated from a human subject. In some aspects, the starting tumor sample isolated from a human subject, and the TILs therein are expanded for an allogeneic cell therapy. In some aspects, the tumor sample is isolated from a human subject, and the TILs therein are expanded for an autologous cell therapy.

II.I TIL Isolation, Expansion, and Harvest

Any method of TIL isolation, culture, and/or expansion can be modified according to the methods disclosed herein, e.g., by culturing and/or expanding the TILs in a culture medium described herein.

II.I.1. Initial Expansion

In general, TILs are obtained from a tumor sample obtained from a human subject. Any methods for obtaining a tumor biopsy from a subject can be used in the methods disclosed herein, so long as the tumor sample contains a mixture of tumor and TILs. In some aspects, the tumor sample is isolated through a tumor resection. In some aspects, the tumor sample is isolated by a needle biopsy (see, e.g., US Publication No. US 2020/0277573, which is incorporated by reference herein in its entirety). In some aspects, the tumor sample comprises a solid tumor, including a primary tumor, invasive tumor or metastatic tumor. In other aspects, the tumor sample comprises a liquid tumor, such as a tumor obtained from a hematological malignancy. The tumor may be of any cancer type, including, but not limited to, breast, pancreatic, prostate, colorectal, cervical, lung, brain, renal, stomach, liver (including but not limited to hepatocellular carcinoma) and skin (including but not limited to squamous cell carcinoma, basal cell carcinoma, and melanoma). In some aspects, the tumor comprises a melanoma. In some aspects, the tumor comprises a colorectal cancer. In some aspects, the tumor comprises a pancreatic cancer. In some aspects, the tumor comprises a head and neck cancer. In some aspects, the tumor comprises a cervical cancer. In some aspects, the tumor comprises an ovarian cancer. In some aspects, the tumor sample is cryopreserved prior to TIL isolation/expansion. In some aspects the tumor sample is fresh, e.g., not cryopreserved. In some aspects, the tumor sample is placed directly into MRM media.

In some aspects, the donor patient (e.g., the subject from which the tumor is obtained) is treatment naïve (i.e., the patient has not received a prior therapy for the treatment of the tumor). In some aspects, the donor patient has received one or more prior therapy for the treatment of the tumor. In some aspects, the subject has received at least one prior therapy, at least two prior therapies, at least three prior therapies, or at least four prior therapies. In some aspects, the subject is relapsed or refractory to one or more prior therapy.

In some aspects, the subject has received one or more prior anticancer therapy. In some aspects, the prior anticancer therapy comprises a standard of care therapy. In some aspects, the prior anticancer therapy comprises an immunotherapy. In some aspects, the prior therapy comprises an immunotherapy comprising a checkpoint inhibitor. In some aspects, the prior therapy comprises an immunotherapy comprising an anti-PD-1 antibody, an anti-CTLA-4 antibody, an anti-LAG-3 antibody, or any combination thereof.

In some aspects, the subject is administered one or more therapy that enhances the isolation and/or expansion of TILs prior to resection of the tumor sample. In some aspects, the subject is administered a kinase inhibitor or an ITK inhibitor. Examples of kinase inhibitors and/or ITK inhibitors can be found, for example, in Int'l Publication No. WO2019217753, which is incorporated by reference herein in its entirety. In some aspects, the kinase inhibitor and/or the ITK inhibitor is added to the culture medium during the initial expansion and/or the second expansion. In some aspects, the ITK inhibitor is selected from the group consisting of aminothiazole-based ITK inhibitors, benzimidazole-based ITK inhibitors, aminopyrimidine-based ITK inhibitors, 3-aminopyride-2-ones-based ITK inhibitors, indolylndazole-based ITK inhibitors, pyrazolyl-indole-based inhibitors, thienopyrazole inhibitors, and ITK inhibitors targeting cysteine-442 in the ATP pocket. In some aspects, the ITK inhibitor is selected from the group consisting of ibrutinib, dasatinib, bosutinib, nilotinib, erlotinib, BMS509744, CTA056, GSK2250665A, PF06465469, and any combination thereof.

In some aspects, the tumor sample is cut into smaller fragments. In some aspects, the one or more of the smaller fragments is at least about 1 mm², at least about 1.5 mm², at least about 2 mm², at least about 2.5 mm², at least about 3 mm², at least about 3.5 mm², at least about 4 mm², at least about 4.5 mm², at least about 5 mm², at least about 5.5 mm², at least about 6 mm², or at least about 6.5 mm². In some aspects, the one or more of the smaller fragments is at least about 1 mm³, at least about 1.5 mm³, at least about 2 mm³, at least about 2.5 mm³, at least about 3 mm³, at least about 3.5 mm³, at least about 4 mm³, at least about 4.5 mm³, at least about 5 mm³, at least about 5.5 mm³, at least about 6 mm³, at least about 6.5 mm³, at least about 7 mm³, at least about 7.5 mm³, at least about 8 mm³, at least about 8.5 mm³, at least about 9 mm³, at least about 9.5 mm³, or at least about 10 mm³. In some aspects, the tumor samples are subjected to an enzymatic digest, by culturing the tumor samples in an enzymatic media (e.g., RPMI 1640 buffer or MRM supplemented with glutamate (e.g., about 2 mM), gentamicine (e.g., about 10 mcg/mL), DNase (e.g., about 30 units/mL), and collagenase (e.g., about 1.0 mg/mL)). In some aspects, the tumor digests are produced by placing the tumor in the enzymatic media and/or mechanically dissociating (i.e., disaggregating) the tumor (e.g., for about 1 minute), followed by incubation at 37° C. in 5% CO₂ (e.g., for 30 minutes), followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide can be performed to remove these cells. The mechanical and/or enzymatic dissociation can be performed in any medium. In some aspects, the mechanical and/or enzymatic dissociation is performed in an MRM medium disclosed herein.

In some aspects, the mechanical dissociation comprises applying a physical pressure to the resected tumor. In some aspects, the mechanical dissociation comprises repeated physical pressure. In some aspects, the repeated physical pressure is applied at least about 50 times, at least about 60 times, at least about 70 times, at least about 80 times, at least about 90 times, at least about 100 times, at least about 110 times, at least about 120 times, at least about 130 times, at least about 140 times, at least about 150 times, at least about 160 times, at least about 170 times, at least about 180 times, at least about 190 times, at least about 200 times, at least about 210 times, at least about 220 times, at least about 230 times, at least about 240 times, at least about 250 times, at least about 260 times, at least about 270 times, at least about 280 times, at least about 290 times, at least about 300 times, at least about 310 times, at least about 320 times, at least about 330 times, at least about 340 times, at least about 350 times, or at least about 360 times per minute. In some aspects, the repeated physical pressure is applied at least about 120 to 260 times per minute. In some aspects, the repeated physical pressure is applied up to about 6 N/cm², up to about 5.5 N/cm², up to about 5.0 N/cm², up to about 4.5 N/cm², up to about 4.0 N/cm², up to about 3.5 N/cm², up to about 3.0 N/cm². In some aspects, the mechanical dissociation proceeds for about 90 minutes or less, about 85 minutes or less, about 80 minutes or less, about 75 minutes or less, about 70 minutes or less, about 65 minutes or less, about 60 minutes or less, about 55 minutes or less, or about 50 minutes or less. In some aspects, the mechanical dissociation is applied at room temperature. In some aspects, the mechanical dissociation is applied at less than room temperature. In some aspects, the mechanical dissociation is applied according to the methods disclosed in and/or using a device disclosed in Int'l Publication No. WO 2021/123832, which is incorporated by reference herein in its entirety.

In some aspects, the tumor sample (i.e., the resected tumor tissue sample or the dissociated tumor sample) or the fragments thereof is placed into a culture medium, e.g., a culture medium disclosed herein, wherein the culture medium further comprises IL-2. In some aspects, the culture medium comprises at least about 4000 IU/ml IL-2, at least about 4500 IU/ml IL-2, at least about 5500 IU/ml IL-2, at least about 6000 IU/ml IL-2, or at least about 6500 IU/ml IL-2. In some aspects, the culture medium comprises at least about 600 IU/ml IL-2. In some aspects, the culture medium comprises at least about 100 ng/mL IL-2. In some aspects, the culture medium comprises at least about 200 ng/mL IL-2. In some aspects, the culture medium comprises at least about 300 ng/mL IL-2. In some aspects, the culture medium comprises at least about 400 ng/mL IL-2. In some aspects, the culture medium comprises at least about 500 ng/mL IL-2. In some aspects, the culture medium comprises at least about 600 ng/mL IL-2.

In other aspects, the tumor sample or the fragments thereof is placed into a culture medium, e.g., a culture medium disclosed herein, wherein the culture medium further comprises IL-21. In some aspects, the culture medium comprises at least about 1.0 ng/mL IL-21. In some aspects, the culture medium comprises at least about 2.0 ng/mL IL-21. In some aspects, the culture medium comprises at least about 3.0 ng/mL IL-21. In some aspects, the culture medium comprises at least about 4.0 ng/mL IL-21. In some aspects, the culture medium comprises at least about 5.0 ng/mL IL-21. In some aspects, the culture medium comprises at least about 6.0 ng/mL IL-21. In some aspects, the culture medium comprises at least about 7.0 ng/mL IL-21. In some aspects, the culture medium comprises at least about 8.0 ng/mL IL-21. In some aspects, the culture medium comprises at least about 9.0 ng/mL IL-21. In some aspects, the culture medium comprises at least about 10 ng/mL IL-21. In some aspects, the culture medium comprises at least about 15 ng/mL IL-21. In some aspects, the culture medium comprises at least about 20 ng/mL IL-21. In some aspects, the culture medium comprises at least about 30 ng/mL IL-21.

Individual tumor fragments can be cultured together in a single culture chamber, e.g., well, or individual tumor fragments can be cultured in separate culture chambers, e.g., wells. A standard culture medium for promoting TIL evasion from cultured tumor samples comprises RPMI 1640 supplemented with Glutamax (Gibco/Invitrogen; Carlsbad, Calif.), 1×Pen-Strep (Gibco/Invitrogen; Carlsbad, Calif.), 50 μm 2-mercaptoethanol (Gibco/Invitrogen; Carlsbad, Calif.), 20 μg/ml Gentamicin (Gibco/Invitrogen; Carlsbad, Calif.), and 1 mM pyruvate (Gibco/Invitrogen; Carlsbad, Calif.). In some aspects, a standard culture medium is modified according to the present disclosure. In some aspects, a standard culture medium comprises CTS™ OpTimizer™ supplemented with serum supplement (CTS™ Immune Cell SR, Thermo Fisher), L-glutamine (Gibco), L-glutamax (Gibco), MEM Non-Essential Amino Acids Solution (Gibco), Pen-strep (Gibco), Fungin™ (InvivoGen), Sodium pyruvate (Gibco), IL-2, IL-21, O-Acetyl-L-carnitine hydrochloride (Sigma), or any combination thereof. In some aspects, a standard culture medium comprises CTS™ OpTimizer™ supplemented with about 2.5% serum supplement (CTS™ Immune Cell SR, Thermo Fisher), about 2 mM L-glutamine (Gibco), about 2 mM L-glutamax (Gibco), MEM Non-Essential Amino Acids Solution (Gibco), Pen-strep (Gibco), about 20 μg/ml Fungin™ (InvivoGen), Sodium pyruvate (Gibco), about IL-2 (300 ng/mL), about IL-21 (30 ng/ml), and about 1 mM of O-Acetyl-L-carnitine hydrochloride (Sigma).

In some aspects, tumor samples or fragments thereof are cultured in an initial culture for at least about 1 week, at least about 2 weeks, or at least about 3 weeks. In some aspects, tumor samples or fragments thereof are cultured for at least about 2 weeks. As used herein, “tumor samples” refers to tumor tissue and/or disaggregated tumor tissue (i.e., a cell suspension resulting from mechanical and/or chemical disaggregation of tumor tissue). In some aspects, the tumor samples or fragments are cultured in an initial culture for about 7 days, about 8 days, about 9 days, about 10 days, about 11 days, about 12 days, about 13 days, or about 14 days.

In some aspects, the initial culture further comprises contacting the tumor samples or fragments with a tumor necrosis factor receptor superfamily (TNFRSF) agonist. In some aspects, the TNFRSF agonist comprises a 4-1BB agonist, an OX40 agonist, a CD27 agonist, a GITR agonist, a HVEM agonist, a CD95 agonist, or any combination thereof. In some aspects, the TNFRSF agonist is any TNFRSF agonist disclosed in U.S. Publication No. US 2020/0121719 A1, which is incorporated by reference herein in its entirety. In some aspects, the initial culture further comprises contacting the tumor samples or fragments thereof with about 10-500 ng/ml 4-1BB ligand. In some aspects, initial culture further comprises contacting the tumor samples or fragments thereof with about 50 ng/ml, about 60 ng/ml, about 70 ng/ml, about 75 ng/ml, about 80 ng/ml, about 90 ng/ml, about 100 ng/ml, about 125 ng/ml, about 150 ng/ml, about 175 ng/ml, about 200 ng/ml, about 250 ng/ml, about 300 ng/ml, about 350 ng/ml, about 400 ng/ml, about 450 ng/ml, about 500 ng/ml, about 550 ng/ml, about 600 ng/ml, about 650 ng/ml, about 700 ng/ml, about 750 ng/ml, about 800 ng/ml, about 850 ng/ml, about 900 ng/ml, about 950 ng/ml, about 1000 ng/ml, or about 1100 ng/ml 4-1BB ligand. In some aspects, initial culture further comprises contacting the tumor samples or fragments thereof with about 100 ng/ml 4-1BB ligand. In some aspects, the tumor samples or fragments thereof are contacted with the 4-1BB ligand on about day 3 of the initial culture, on about day 4 of the initial culture, on about day 5 of the initial culture, on about day 6 of the initial culture, or on about day 7 of the initial culture. In some aspects, the tumor samples or fragments thereof are contacted with the 4-1BB ligand on about day 5 of the initial culture.

In some aspects, the initial culture further comprises contacting the tumor samples or fragments thereof with TRANSACT™. In some aspects, initial culture further comprises contacting the tumor samples or fragments thereof with TRANSACT™ (e.g., about 1:50, about 1:100, about 1:150, about 1:200, about 1:250, about 1:300, about 1:350, or about 1:400). In some aspects, the tumor samples or fragments thereof are contacted with the TRANSACT™ on about day 4 of the initial culture, on about day 5 of the initial culture, on about day 6 of the initial culture, or on about day 7 of the initial culture. In some aspects, the tumor samples or fragments thereof are contacted with the TRANSACT™ on about day 5 of the initial culture. In some aspects, the initial culture further comprises contacting the tumor samples or fragments thereof with both 4-1BB ligand and TRANSACT™. In some aspects, the tumor samples or fragments thereof are contacted with both 4-1BB ligand and TRANSACT™ on about day 3 of the initial culture. In some aspects, the tumor samples or fragments thereof are contacted with both 4-1BB ligand and TRANSACT™ on about day 4 of the initial culture. In some aspects, the tumor samples or fragments thereof are contacted with both 4-1BB ligand and TRANSACT™ on about day 5 of the initial culture. In some aspects, the tumor samples or fragments thereof are contacted with both 4-1BB ligand and TRANSACT™ on about day 6 of the initial culture. In some aspects, the tumor samples or fragments thereof are contacted with both 4-1BB ligand and TRANSACT™ on about day 7 of the initial culture. In some aspects, the tumor samples or fragments thereof are contacted with both 4-1BB ligand and TRANSACT™ on about day 8 of the initial culture.

In some aspects, tumor samples or fragments thereof are cultured in an initial culture until cell yield in the initial culture reaches at least about 1×10⁵ to at least about 1×10⁸, at least about 5×10⁵ to at least about 1×10⁸, at least about 1×10⁶ to at least about 1×10⁸, at least about 2×10⁶ to at least about 1×10⁸, at least about 3×10⁶ to at least about 1×10⁸, at least about 4×10⁶ to at least about 1×10⁸, at least about 5×10⁶ to at least about 1×10⁸, at least about 1×10⁵ to at least about 5×10⁷, at least about 5×10⁵ to at least about 10×10⁶, at least about 1×10⁶ to at least about 10×10⁶, at least about 2×10⁶ to at least about 10×10⁶, at least about 3×10⁶ to at least about 10×10⁶, at least about 4×10⁶ to at least about 10×10⁶, or at least about 5×10⁶ to at least about 10×10⁶ cells per cultured fragment. In some aspects, tumor samples or fragments thereof are cultured in an initial culture until cell yield in the initial culture reaches at least about 2×10⁶-10×10⁶ cells per fragment.

In some aspects, tumor samples or fragments thereof are cultured in an initial culture until cell yield in the initial culture reaches at least about 1×10⁵, at least about 2×10⁵, at least about 3×10⁵, at least about 4×10⁵, at least about 5×10⁵, at least about 6×10⁵, at least about 7×10⁵, at least about 8×10⁵, at least about 9×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, or at least about 10×10⁶ cells per fragment. In some aspects, tumor samples or fragments thereof are cultured in an initial culture until cell yield in the initial culture reaches at least about 2×10⁶ cells per fragment. In some aspects, tumor samples or fragments thereof are cultured in an initial culture until cell yield in the initial culture reaches at least about 3×10⁶ cells per fragment. In some aspects, tumor samples or fragments thereof are cultured in an initial culture until cell yield in the initial culture reaches at least about 4×10⁶ cells per fragment. In some aspects, tumor samples or fragments thereof are cultured in an initial culture until cell yield in the initial culture reaches at least about 5×10⁶ cells per fragment. In some aspects, tumor samples or fragments thereof are cultured in an initial culture until cell yield in the initial culture reaches at least about 6×10⁶ cells per fragment. In some aspects, tumor samples or fragments thereof are cultured in an initial culture until cell yield in the initial culture reaches at least about 7×10⁶ cells per fragment. In some aspects, tumor samples or fragments thereof are cultured in an initial culture until cell yield in the initial culture reaches at least about 8×10⁶ cells per fragment. In some aspects, tumor samples or fragments thereof are cultured in an initial culture until cell yield in the initial culture reaches at least about 9×10⁶ cells per fragment. In some aspects, tumor samples or fragments thereof are cultured in an initial culture until cell yield in the initial culture reaches at least about 10×10⁶ cells per fragment. In some aspects, the cells (e.g., TILs) are passed through a strainer following the initial culture. In some aspects, the cells (e.g., TILs) are passed through an at least about 10 μm, an at least about 15 μm, an at least about 20 μm, an at least about 25 μm, an at least about 30 μm, an at least about 35 μm, an at least about 40 μm, an at least about 45 μm, an at least about 50 μm strainer following the initial culture. In some aspects, the cells (e.g., TILs) are passed through an about 40 μm strainer following the initial culture.

In some aspects, the initial expansion step is carried out in one or more gas permeable flasks (e.g., GREX flasks). In some aspects, the initial expansion step is carried out in static GREX. In some aspects, the initial expansion is carried out in a stirred tank. In some aspects the initial expansion step is carried out in a bioreactor. In some aspects, the initial expansion is carried out in a closed system (e.g., using a GREX closed system).

II.I.2. Secondary Expansion

In some aspects, the TILs are subjected to a secondary expansion. In some aspects, the secondary expansion step is carried out in one or more gas permeable flasks (e.g., GREX flasks). In some aspects, the TILs are transitioned to the secondary expansion without opening the closed system. In some aspects, the TILs from the first expansion are screened for tumor-specific cytolytic activity prior to advancing the TILs to the secondary expansion. In some aspects, the TILs are screened for expression of one or more biomarkers prior to advancing to secondary expansion. In some aspects, the biomarker comprises expression of one or more gene typically expressed by more naïve TILs, e.g., CD8⁺, CD27⁺, CD3⁺, CD95⁺, CD45RA⁺, CCR7⁺, CD62L⁺, TCF7⁺, or any combination thereof. In some aspects, the TILs are screened for expression of PD-1 prior to advancing to secondary expansion. In some aspects, the TILs from the first expansion are not screened prior to advancing the TILs to the secondary expansion. In some aspects, all TILs obtained in the initial expansion are subjected to the secondary expansion. In some aspects, the TILs from the first expansion are pooled prior to advancement to secondary expansion.

In some aspects, the TILs are subjected to a secondary expansion using a Rapid Expansion Protocol (REP). See, e.g., Dudley, et al., Science 298:850-54 (2002); Dudley, et al., J. Clin. Oncol. 23:2346-57 (2005); Dudley, et al., J. Clin. Oncol. 26:5233-39 (2008); Riddell, et al., Science 257:238-41 (1992); and Dudley, et al., J. Immunother. 26:332-42 (2003), each of which is incorporated by reference herein in its entirety. In some aspects, TILs are rapidly expanded using non-specific T-cell receptor stimulation in the presence of feeder lymphocytes and interleukin-2 (IL-2), IL-7, IL-15, IL-21, or combinations thereof. In certain aspects, the TILs are rapidly expanded in the presence of IL-2, IL-15, and IL-21. In some aspects, the concentration of IL-2 in the media during rapid expansion is lower than the concentration of IL-2 in the media during the initial culture. In some aspects, the concentration of IL-2 during rapid expansion is less than 300 ng/ml. In some aspects, the concentration of IL-2 during rapid expansion is about 50 ng/ml, about 55 ng/ml, about 60 ng/ml, about 65 ng/ml, about 70 ng/ml, about 73.6 ng/ml, about 75 ng/ml, about 80 ng/ml, about 85 ng/ml, about 90 ng/ml, about 95 ng/ml, about 100 ng/ml, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 175 ng/ml, about 200 ng/ml, about 225 ng/ml, about 250 ng/ml, or about 275 ng/ml. In some aspects, the concentration of IL-2 during rapid expansion is about 50 ng/ml. In some aspects, the concentration of IL-2 during rapid expansion is about 55 ng/ml. In some aspects, the concentration of IL-2 during rapid expansion is about 60 ng/ml. In some aspects, the concentration of IL-2 during rapid expansion is about 65 ng/ml. In some aspects, the concentration of IL-2 during rapid expansion is about 70 ng/ml. In some aspects, the concentration of IL-2 during rapid expansion is about 73.6 ng/ml. In some aspects, the concentration of IL-2 during rapid expansion is about 75 ng/ml. In some aspects, the concentration of IL-2 during rapid expansion is about 80 ng/ml. In some aspects, the concentration of IL-2 during rapid expansion is about 85 ng/ml. In some aspects, the concentration of IL-2 during rapid expansion is about 90 ng/ml. In some aspects, the concentration of IL-2 during rapid expansion is about 95 ng/ml. In some aspects, the concentration of IL-2 during rapid expansion is about 100 ng/ml.

In some aspects, the concentration of IL-21 in the media during rapid expansion is lower than the concentration of IL-21 in the media during the initial culture. In some aspects, the concentration of IL-21 during rapid expansion is less than 30 ng/ml. In some aspects, the concentration of IL-21 during rapid expansion is about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, or about 29 ng/ml. In some aspects, the concentration of IL-21 during rapid expansion is about 5 ng/ml. In some aspects, the concentration of IL-21 during rapid expansion is about 6 ng/ml. In some aspects, the concentration of IL-21 during rapid expansion is about 7 ng/ml. In some aspects, the concentration of IL-21 during rapid expansion is about 8 ng/ml. In some aspects, the concentration of IL-21 during rapid expansion is about 9 ng/ml. In some aspects, the concentration of IL-21 during rapid expansion is about 10 ng/ml. In some aspects, the concentration of IL-21 during rapid expansion is about 11 ng/ml. In some aspects, the concentration of IL-21 during rapid expansion is about 12 ng/ml. In some aspects, the concentration of IL-21 during rapid expansion is about 13 ng/ml. In some aspects, the concentration of IL-21 during rapid expansion is about 14 ng/ml. In some aspects, the concentration of IL-21 during rapid expansion is about 15 ng/ml.

In some aspects, the concentration of IL-15 in the media during rapid expansion is about 0.1 ng/ml, about 0.2 ng/ml, about 0.3 ng/ml, about 0.4 ng/ml, about 0.5 ng/ml, about 0.6 ng/ml, about 0.7 ng/ml, about 0.8 ng/ml, about 0.9 ng/ml, about 1.0 ng/ml, about 1.1 ng/ml, about 1.2 ng/ml, about 1.3 ng/ml, about 1.4 ng/ml, about 1.5 ng/ml, about 1.6 ng/ml, about 1.7 ng/ml, about 1.8 ng/ml, about 1.9 ng/ml, about 2.0 ng/ml, about 2.25 ng/ml, about 2.5 ng/ml, about 2.75 ng/ml, about 3.0 ng/ml, about 3.5 ng/ml, about 4.0 ng/ml, about 4.5 ng/ml, or about 5.0 ng/ml. In some aspects, the concentration of IL-15 during rapid expansion is about 0.1 ng/ml. In some aspects, the concentration of IL-15 during rapid expansion is about 0.2 ng/ml. In some aspects, the concentration of IL-15 during rapid expansion is about 0.3 ng/ml. In some aspects, the concentration of IL-15 during rapid expansion is about 0.4 ng/ml. In some aspects, the concentration of IL-15 during rapid expansion is about 0.5 ng/ml. In some aspects, the concentration of IL-15 during rapid expansion is about 0.6 ng/ml. In some aspects, the concentration of IL-15 during rapid expansion is about 0.7 ng/ml. In some aspects, the concentration of IL-15 during rapid expansion is about 0.8 ng/ml. In some aspects, the concentration of IL-15 during rapid expansion is about 0.9 ng/ml. In some aspects, the concentration of IL-15 during rapid expansion is about 1.0 ng/ml.

The non-specific T-cell receptor stimulus can include, e.g., OKT3 (e.g., about 30 ng/ml), a mouse monoclonal anti-CD3 antibody (available from Ortho-McNeil®, Raritan, N.J. or Miltenyi Biotec, Bergisch Gladbach, Germany). In some aspects, TILs are rapidly expanded by stimulation of peripheral blood mononuclear cells (PBMC) in vitro with one or more antigens (including antigenic portions thereof, such as epitope(s), or a cell of the cancer, which can be optionally expressed from a vector, such as an human leukocyte antigen A2 (HLA-A2) binding peptide, e.g., approximately 0.3 μM MART-1:26-35 (27 L) or gp100:209-217 (210M)), in the presence of a T-cell growth factor, such as around 200-400 IU/ml of a T-cell growth factor, such as 300 IU/ml IL-2 or IL-15. In some aspects, TILs are expanded by stimulation using TRANSACT™. In some aspects, the in vitro-induced TILs are rapidly expanded by stimulation with the same antigen(s) of the cancer pulsed onto HLA-A2-expressing antigen-presenting cells. In some aspects, the TILs can be stimulated with irradiated, autologous lymphocytes or with irradiated HLA-A2+ allogeneic lymphocytes and IL-2.

In some aspects, the TILs are stimulated during the second expansion by culturing the cells in a medium comprising TRANSACT™ and optionally 4-1BBL and/or CD27L. In some aspects, the TILs are stimulated during the second expansion by culturing the cells in a medium comprising TRANSACT™, 4-1BBL, and CD27L. In some aspects, the TILs are stimulated during the second expansion by culturing the cells in a medium comprising at least about 1:100 TRANSACT™, at least about 1 μg/ml 4-1BBL, and at least about 5 μg/ml CD27L.

In some aspects, one or more TILs are genetically modified before, during, or after TIL expansion. Genetic modification of the TILs can be achieved using any methods known in the art. In some aspects, one or more TILs are modified using a Cas9 endonuclease (CRISPR; see, e.g., US2017067021A1, which is incorporated by reference herein in its entirety), TALEN, a zing-finger endonuclease, site directed mutagenesis, or any combination thereof. In some aspects, one or more TILs are genetically modified to disrupt or ablate expression of human cytokine inducible SH2-containing protein (CISH; see, e.g., U.S. Ser. No. 10/406,177B2, which is incorporated by reference herein in its entirety). In some aspects, one or more TILs is modified using an AAV, e.g., one or more of the TILs comprise an AAV. In some aspects one or more TILs is modified using a lentivirus or a retrovirus. In some aspects, one or more TILs are genetically modified to express an exogenous modified or engineered T cell receptor (TCR). In some aspects, one or more TILs are genetically modified to express chimeric antigen receptor (CAR). In some aspects, one or more TILs are genetically modified to express CD86. In some aspects, one or more TILs are genetically modified to express OX40L. In some aspects, one or more TILs are genetically modified to express 4-1BBL. In some aspects, one or more TILs are genetically modified to express an anti-PD1 antibody.

In some aspects, the TILs are expanded in a culture medium that further comprises a tumor necrosis factor receptor superfamily (TNFRSF) agonist. Any TNFRSF agonist can be used in the methods disclosed herein. Non-limiting examples of TNFRSF agonists can be found, for example, in US20200121719A1, which is incorporated by reference herein in its entirety. In some aspects, the TNFRSF agonist is added after the initial culture. In some aspects, the TNFRSF agonist is added during the second and/or or final expansion.

In some aspects, the TILs are expanded in a culture medium that further comprises a 4-1BB agonist. Any 4-1BB agonist can be used in the methods disclosed herein. In some aspects, the 4-1BB agonist comprises a 4-1BB antibody. Non-limiting examples of 4-1BB agonists can be found, for example, in US20200032209A1, which is incorporated by reference herein in its entirety. In some aspects, the 4-1BB agonist is added after the initial culture. In some aspects, the 4-1BB agonist is added during the second or final expansion.

In some aspects, the TILs are stimulated during the second expansion by culturing the cells in a medium comprising TRANSACT™ and optionally 4-1BBL and/or CD27L. In some aspects, the TILs are stimulated during the second expansion by culturing the cells in a medium comprising TRANSACT™, 4-1BBL, and CD27L. In some aspects, the TILs are stimulated during the second expansion by culturing the cells in a medium comprising at least about 1:100 TRANSACT™, at least about 1 μg/ml 4-1BBL, and at least about 5 μg/ml CD27L.

In some aspects, the TILs are expanded in a culture medium that further comprises an adenosine a2a receptor antagonist. Any adenosine a2a receptor antagonist can be used in the methods disclosed herein. Non-limiting examples of adenosine a2a receptor antagonist can be found, for example, in US20210137930A1, which is incorporated by reference herein in its entirety. In some aspects, the adenosine a2a receptor antagonist is selected from the group consisting of vipadenant, CPI-444 (ciforadenant), SCH58261, ZM241385, SCH420814, SYN115, 8-CSC, KW-6002, A2A receptor antagonist 1, ADZ4635, ST4206, KF21213, SCH412348, and 7MMG-49, or pharmaceutically acceptable salts, solvates, hydrates, cocrystals, or prodrugs thereof, and combinations thereof. In some aspects, the adenosine a2a receptor antagonist is added during the initial culture. In some aspects, the adenosine a2a receptor antagonist is added during the second and/or or final expansion.

In some aspects, the TILs are expanded in a culture medium that further comprises an AKT pathway inhibitor (AKTi). Any AKTi can be used in the methods disclosed herein. Non-limiting examples of AKTi that can be used in the present disclosure can be found, for example, in WO2020096927, which is incorporated by reference herein in its entirety. In some aspects, the AKTi is selected from the group consisting of afuresertib, uprosertib, ipatasertib, AT7867, AT13148, and pharmaceutically acceptable salts, solvates, hydrates, cocrystals, or prodrugs thereof. In some aspects, the AKTi is an mTOR inhibitor, e.g., AZD8055 or pharmaceutically acceptable salts, solvates, hydrates, cocrystals, or prodrugs thereof. In some aspects, the AKTi is an PI3K inhibitor, e.g., LY294002 or pharmaceutically acceptable salts, solvates, hydrates, cocrystals, or prodrugs thereof. In some aspects, the AKTi is added during the initial culture. In some aspects, the AKTi is added during the second and/or or final expansion.

In some aspects, the expanded cells are reactivated or stimulated by contacting the expanded TILs with one or more antigen presenting cell. Any antigen presenting cell can be used in the methods disclosed herein. In some aspects, the antigen presenting cell is a genetically modified cell. In some aspects, the antigen presenting cell comprises a tumor antigen or a fragment thereof on the cell surface. In some aspects, the expanded TILs are contacted with antigen presenting cells which comprises more than one tumor antigen or a fragment thereof on the cell surface.

In some aspects, the antigen presenting cell (APC) is genetically engineered. In some aspects, the APC is genetically engineered for tunable expression of one or more transgene, e.g., an antigen or a stimulatory signal. In some aspects, the APC is genetically engineered according to a method disclosed in WO2020/086742, which is incorporated by reference herein in its entirety. In some aspects, the APC is genetically engineered to express one or more stimulatory molecule. In some aspects, the APC is genetically engineered to express CD86, OC40L, 4-1BBL, or any combination thereof. In some aspects, the APC is an APC disclosed in U.S. Pat. No. 10,415,015, which is incorporated by reference herein in its entirety.

In some aspects, the TILs are cultured in a secondary TIL media until cell yield in the secondary expansion reaches at least about 1×10⁷ to at least about 50×10⁷, at least about 2×10⁷ to at least about 40×10⁷, at least about 3×10⁷ to at least about 30×10⁷, at least about 4×10⁷ to at least about 25×10⁷, at least about 5×10⁷ to at least about 20×10⁷, at least about 1×10⁷ to at least about 20×10⁷, at least about 2×10⁷ to at least about 20×10⁷, at least about 3×10⁷ to at least about 20×10⁷, or at least about 4×10⁷ to at least about 20×10⁷ cells. In some aspects, the TILs are cultured in a secondary TIL media until cell yield in the secondary expansion reaches at least about 5×10⁷ to at least about 20×10⁷ cells. In some aspects, the TILs are cultured in a secondary TIL media until cell yield in the secondary expansion reaches at least about 1×10⁷, at least about 2×10⁷, at least about 3×10⁷, at least about 4×10⁷, at least about 5×10⁷, at least about 6×10⁷, at least about 7×10⁷, at least about 8×10⁷, at least about 9×10⁷, at least about 10×10⁷, at least about 11×10⁷, at least about 12×10⁷, at least about 13×10⁷, at least about 14×10⁷, at least about 15×10⁷, at least about 16×10⁷, at least about 17×10⁷, at least about 18×10⁷, at least about 19×10⁷, or at least about 20×10⁷ cells. In some aspects, the TILs are cultured in a secondary TIL media until cell yield in the secondary expansion reaches at least about 5×10⁷ cells. In some aspects, the TILs are cultured in a secondary TIL media until cell yield in the secondary expansion reaches at least about 6×10⁷ cells. In some aspects, the TILs are cultured in a secondary TIL media until cell yield in the secondary expansion reaches at least about 7×10⁷ cells. In some aspects, the TILs are cultured in a secondary expansion until cell yield in the secondary TIL media reaches at least about 8×10⁷ cells. In some aspects, the TILs are cultured in a secondary expansion until cell yield in the secondary TIL media reaches at least about 9×10⁷ cells. In some aspects, the TILs are cultured in a secondary TIL media until cell yield in the secondary expansion reaches at least about 10×10⁷ cells. In some aspects, the TILs are cultured in a secondary TIL media until cell yield in the secondary expansion reaches at least about 15×10⁷ cells. In some aspects, the TILs are cultured in a secondary TIL media until cell yield in the secondary expansion reaches at least about 20×10⁷ cells.

In some aspects, TILs are subjected to a final expansion. In some aspects, the TILs are transitioned from the secondary expansion to the final expansion without opening the closed system (e.g., the GREX closed system). In some aspects, the final expansion step is carried out in one or more gas permeable flasks (e.g., GREX flasks). In some aspects, the secondary expansion corresponds with a first phase of the REP protocol (i.e., the REP protocol up until the cells are split), and the final expansion corresponds with the second phase of the REP protocol (i.e., the REP protocol after the cells are split). As such, in some aspects, the secondary expansion has a duration of about 3 to 7 days (e.g., about 5 days, about 6 days, or about 7 days), and the final expansion has a duration of about 3 to 7 days (e.g., about 5 days, about 6 days, or about 7 days).

In some aspects, the media during final expansion comprises IL-2, IL-7, IL-15, IL-21, or combinations thereof. In certain aspects, the media during final expansion comprises IL-2, IL-15, and IL-21. In some aspects, the concentration of IL-2 in the media during final expansion is lower than the concentration of IL-2 in the media during the initial culture. In some aspects, the concentration of IL-2 during final expansion is less than 300 ng/ml. In some aspects, the concentration of IL-2 during final expansion is about 50 ng/ml, about 55 ng/ml, about 60 ng/ml, about 65 ng/ml, about 70 ng/ml, about 73.6 ng/ml, about 75 ng/ml, about 80 ng/ml, about 85 ng/ml, about 90 ng/ml, about 95 ng/ml, about 100 ng/ml, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 175 ng/ml, about 200 ng/ml, about 225 ng/ml, about 250 ng/ml, or about 275 ng/ml. In some aspects, the concentration of IL-2 during final expansion is about 50 ng/ml. In some aspects, the concentration of IL-2 during final expansion is about 55 ng/ml. In some aspects, the concentration of IL-2 during final expansion is about 60 ng/ml. In some aspects, the concentration of IL-2 during final expansion is about 65 ng/ml. In some aspects, the concentration of IL-2 during final expansion is about 70 ng/ml. In some aspects, the concentration of IL-2 during final expansion is about 73.6 ng/ml. In some aspects, the concentration of IL-2 during final expansion is about 75 ng/ml. In some aspects, the concentration of IL-2 during final expansion is about 80 ng/ml. In some aspects, the concentration of IL-2 during final expansion is about 85 ng/ml. In some aspects, the concentration of IL-2 during final expansion is about 90 ng/ml. In some aspects, the concentration of IL-2 during final expansion is about 95 ng/ml. In some aspects, the concentration of IL-2 during final expansion is about 100 ng/ml.

In some aspects, the concentration of IL-21 in the media during final expansion is lower than the concentration of IL-21 in the media during the initial culture. In some aspects, the concentration of IL-21 during final expansion is less than 30 ng/ml. In some aspects, the concentration of IL-21 during final expansion is about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, or about 29 ng/ml. In some aspects, the concentration of IL-21 during final expansion is about 5 ng/ml. In some aspects, the concentration of IL-21 during final expansion is about 6 ng/ml. In some aspects, the concentration of IL-21 during final expansion is about 7 ng/ml. In some aspects, the concentration of IL-21 during final expansion is about 8 ng/ml. In some aspects, the concentration of IL-21 during final expansion is about 9 ng/ml. In some aspects, the concentration of IL-21 during final expansion is about 10 ng/ml. In some aspects, the concentration of IL-21 during final expansion is about 11 ng/ml. In some aspects, the concentration of IL-21 during final expansion is about 12 ng/ml. In some aspects, the concentration of IL-21 during final expansion is about 13 ng/ml. In some aspects, the concentration of IL-21 during final expansion is about 14 ng/ml. In some aspects, the concentration of IL-21 during final expansion is about 15 ng/ml.

In some aspects, the concentration of IL-15 in the media during final expansion is about 0.1 ng/ml, about 0.2 ng/ml, about 0.3 ng/ml, about 0.4 ng/ml, about 0.5 ng/ml, about 0.6 ng/ml, about 0.7 ng/ml, about 0.8 ng/ml, about 0.9 ng/ml, about 1.0 ng/ml, about 1.1 ng/ml, about 1.2 ng/ml, about 1.3 ng/ml, about 1.4 ng/ml, about 1.5 ng/ml, about 1.6 ng/ml, about 1.7 ng/ml, about 1.8 ng/ml, about 1.9 ng/ml, about 2.0 ng/ml, about 2.25 ng/ml, about 2.5 ng/ml, about 2.75 ng/ml, about 3.0 ng/ml, about 3.5 ng/ml, about 4.0 ng/ml, about 4.5 ng/ml, or about 5.0 ng/ml. In some aspects, the concentration of IL-15 during final expansion is about 0.1 ng/ml. In some aspects, the concentration of IL-15 during final expansion is about 0.2 ng/ml. In some aspects, the concentration of IL-15 during final expansion is about 0.3 ng/ml. In some aspects, the concentration of IL-15 during final expansion is about 0.4 ng/ml. In some aspects, the concentration of IL-15 during final expansion is about 0.5 ng/ml. In some aspects, the concentration of IL-15 during final expansion is about 0.6 ng/ml. In some aspects, the concentration of IL-15 during final expansion is about 0.7 ng/ml. In some aspects, the concentration of IL-15 during final expansion is about 0.8 ng/ml. In some aspects, the concentration of IL-15 during final expansion is about 0.9 ng/ml. In some aspects, the concentration of IL-15 during final expansion is about 1.0 ng/ml.

In some aspects, the final expansion comprises a stimulation. In some aspects the stimulation is the same as the stimulation used during the secondary expansion. In some aspects, the TILs are stimulated during the final expansion by culturing the cells in an MRM comprising TRANSACT™, 4-1BBL, CD27L, or any combination thereof. In some aspects, the TILs are stimulated during the final expansion by culturing the cells in an MRM comprising TRANSACT™ and optionally 4-1BBL and/or CD27L. In some aspects, the TILs are stimulated during the final expansion by culturing the cells in an MRM comprising at least about 1:100 TRANSACT™, at least about 1 μg/ml 4-1BBL, and at least about 5 μg/ml CD27L.

In some aspects, the final expansion step is carried out in static GREX. In some aspects, the final expansion is carried out in a stirred tank. In some aspects the final expansion step is carried out in a bioreactor. In some aspects, the final expansion is continued until the cell yield in the final TIL media reaches at least about 40×10⁹ to at least about 100×10⁹, at least about 40×10⁹ to at least about 90×10⁹, at least about 40×10⁹ to at least about 80×10⁹, at least about 40×10⁹ to at least about 70×10⁹, at least about 40×10⁹ to at least about 60×10⁹, at least about 40×10⁹ to at least about 50×10⁹, at least about 10×10⁹ to at least about 100×10⁹, at least about 20×10⁹ to at least about 100×10⁹, at least about 30×10⁹ to at least about 100×10⁹, at least about 30×10⁹ to at least about 50×10⁹, or at least about 35×10⁹ to at least about 45×10⁹ cells. In some aspects, the final expansion is continued until the cell yield in the final TIL media reaches at least about 40×10⁹ to at least about 100×10⁹ cells. In some aspects, the final expansion is continued until the cell yield in the final TIL media reaches at least about 40×10⁹, at least about 45×10⁹, at least about 50×10⁹, at least about 55×10⁹, at least about 60×10⁹, at least about 65×10⁹, at least about 70×10⁹, at least about 75×10⁹, at least about 80×10⁹, at least about 85×10⁹, at least about 90×10⁹, at least about 95×10⁹, or at least about 100×10⁹ cells. In some aspects, the final expansion is continued until the cell yield in the final TIL media reaches at least about 40×10⁹ cells. In some aspects, the final expansion is continued until the cell yield in the final TIL media reaches at least about 50×10⁹ cells. In some aspects, the final expansion is continued until the cell yield in the final TIL media reaches at least about 60×10⁹ cells. In some aspects, the final expansion is continued until the cell yield in the final TIL media reaches at least about 70×10⁹ cells. In some aspects, the final expansion is continued until the cell yield in the final TIL media reaches at least about 80×10⁹ cells. In some aspects, the final expansion is continued until the cell yield in the final TIL media reaches at least about 90×10⁹ cells. In some aspects, the final expansion is continued until the cell yield in the final TIL media reaches at least about 100×10⁹ cells.

In some aspects, the final expansion is continued until the cell yield in the final TIL media for at least about 7 to at least about 21 days. In some aspects, the final expansion is continued until the cell yield in the final TIL media for at least about 7 days. In some aspects, the final expansion is continued until the cell yield in the final TIL media for at least about 8 days. In some aspects, the final expansion is continued until the cell yield in the final TIL media for at least about 9 days. In some aspects, the final expansion is continued until the cell yield in the final TIL media for at least about 10 days. In some aspects, the final expansion is continued until the cell yield in the final TIL media for at least about 11 days. In some aspects, the final expansion is continued until the cell yield in the final TIL media for at least about 12 days. In some aspects, the final expansion is continued until the cell yield in the final TIL media for at least about 13 days. In some aspects, the final expansion is continued until the cell yield in the final TIL media for at least about 14 days. In some aspects, the final expansion is continued until the cell yield in the final TIL media for at least about 15 days. In some aspects, the final expansion is continued until the cell yield in the final TIL media for at least about 16 days. In some aspects, the final expansion is continued until the cell yield in the final TIL media for at least about 17 days. In some aspects, the final expansion is continued until the cell yield in the final TIL media for at least about 18 days. In some aspects, the final expansion is continued until the cell yield in the final TIL media for at least about 19 days. In some aspects, the final expansion is continued until the cell yield in the final TIL media for at least about 20 days. In some aspects, the final expansion is continued until the cell yield in the final TIL media for at least about 21 days.

In some aspects, the secondary expansion and the final expansion are merged into a single secondary expansion. In some aspects, the single secondary expansion comprises all aspects of the secondary expansion and the final expansion. In some aspects, the single secondary expansion takes place in a closed system (e.g., a GREX closed system), wherein the closed system is not opened for the duration of the single secondary expansion. In some aspects, the cells are split during the single secondary expansion once the cells reach high confluence.

In some aspects, the full duration of the expansion process (e.g., (i) the initial expansion process, the secondary expansion process, and the final expansion process; or (ii) the initial expansion process and the single secondary expansion process) is 22 days or less. Generation of young TILs using shorter expansion processes confers various benefits on the resulting TIL composition. As such, the culture conditions and methods disclosed herein confer additional benefits, e.g., increased stem-like characteristics, expanded clonal diversity, improved cytolytic activity, and/or increased CD8⁺ cell expansion, on those already identified for young TILs.

II.I.3. Harvest and Cryopreservation

In some aspects, the expanded TILs are harvested. TILs can be harvested using any method, including by centrifugation. In some aspects, TILs are harvest using an automated system. Cell harvesters and/or cell processing systems are commercially available from a variety of sources, and any cell-based harvester can be used in the methods disclosed herein. In some aspects, the cell harvester and/or cell processing systems is a membrane-based cell harvester. In some aspects, the cell harvesting is conducted using a cell processing system, e.g., the LOVO system (Fresenius Kabi). In some aspects, the cell harvester and/or cell processing system can perform cell separation, washing, fluid-exchange, concentration, and/or other cell processing steps in a closed, sterile system.

In some aspects, the harvest is performed from a closed system bioreactor. In some aspects, a closed system is employed for the TIL expansion. In some aspects, a single bioreactor is employed. In some aspects, the closed system bioreactor is a single bioreactor. Examples of methods of expanding TILs ex vivo in open and closed systems can be found, for example, in U.S. Pat. No. 10,166,257, which is incorporated by reference herein in its entirety.

In some aspects, the expanded TILs are cryopreserved. The TILs can be cryopreserved using any methods. Various methods of cryopreserving mammalian cells, including TILs, have been described, e.g., by (i) General Protocol for the Cryopreservation of Mammalian Cells, UNC (2007), available at unclineberger.org/tissueculture/protocols/general-protocol-for-the-cryopreservation-of-mammalian-cells/; and (ii) Clarke et al., Improved post-thaw recovery of peripheral blood stem/progenitor cells using a novel intracellular-like cryopreservation solution, Cytotherapy 2009-6-6, available at sigmaaldrich.com/catalog/papers/19499402; each of which is incorporated by reference herein in its entirety.

In some aspects, the TILs are cultured according to the following:

(1) Tumor samples are isolated from a subject, and tumors are cut into fragments and/or mechanically or chemically disaggregated.
(2) The resulting tumor samples or fragments thereof are then cultured in an initial culture comprising a metabolic reprogramming media disclosed herein further supplemented with 300 ng/mL or 6000 IU/ml IL-2 and 30 ng/ml IL-21.
(3) Optionally, on day 5 following the start of the initial culture, the TILs are contacted with TRANSACT™ (1:200) and 100 ng/mL 4-1BB ligand, and the TILs are then cultured for an additional 5-9 days or until about 10×10⁶ to about 200×10⁶ cells are reached. TILs are then pooled.
(4) At least 0.5×10⁶ TILs from step 3 are then mixed with 100-200 times excess of irradiated PBMC feeder cells and cultured in media (e.g., a metabolic reprogramming media disclosed herein) supplemented with 30 ng/ml anti-CD3 antibody (e.g., OKT3), 75 ng/mL IL-2, 10 ng/mL IL-21, and 0.4 ng/mL IL-15. This secondary (REP) culture is continued until a therapeutically effective amount of TILs is obtained, as described herein.

### III. Compositions of the Disclosure

Some aspects of the present disclosure are directed to a composition comprising a population of TILs, which is enriched in CD8⁺ TILs. In some aspects, the composition comprises a population of TILs cultured according to any method disclosed herein. In some aspects, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the TILs are CD8⁺ TILs. In some aspects, at least about 20% of the TILs are CD8⁺ TILs. In some aspects, at least about 30% of TILs are CD8⁺ TILs. In some aspects, at least about 40% of the TILs are CD8⁺ TILs. In some aspects, at least about 50% of the TILs are CD8⁺ TILs. In some aspects, at least about 60% of the TILs are CD8⁺ TILs. In some aspects, at least about 70% of the TILs are CD8⁺ TILs. In some aspects, at least about 80% of the TILs are CD8⁺ TILs. In some aspects, at least about 90% of the TILs are CD8⁺ TILs. In some aspects, at least about 95% of TILs are CD8⁺ TILs.

Some aspects of the present disclosure are directed to a composition comprising a population of expanded TILs, wherein the population of expanded TILs has an increased clonal diversity, as compared to the clonal diversity of a population of TILs expanded using control methods (e.g., cultured in a medium comprising potassium ion at a concentration of less than about 5 mM). In some aspects, the population of expanded TILs has a clonal diversity that is the same as the clonal diversity of TILs in a tumor sample. In some aspects, the population of expanded TILs has a clonal diversity that is at least about 99% to about 100%, at least about 98% to about 100%, at least about 97% to about 100%, at least about 96% to about 100%, at least about 95% to about 100%, at least about 94% to about 100%, at least about 93% to about 100%, at least about 92% to about 100%, at least about 91% to about 100%, at least about 90% to about 100%, at least about 85% to about 100%, at least about 80% to about 100%, at least about 75% to about 100%, at least about 70% to about 100%, at least about 65% to about 100%, at least about 60% to about 100%, at least about 55% to about 100%, at least about 50% to about 100%, at least about 45% to about 100%, or at least about 40% to about 100% of the clonal diversity of TILs in a tumor sample. In certain aspects, the population of expanded TILs has a clonal diversity that is at least about 95% of the clonal diversity of TILs in a tumor sample. In certain aspects, the population of expanded TILs has a clonal diversity that is at least about 90% of the clonal diversity of TILs in a tumor sample. In certain aspects, the population of expanded TILs has a clonal diversity that is at least about 85% of the clonal diversity of TILs in a tumor sample. In certain aspects, the population of expanded TILs has a clonal diversity that is at least about 80% of the clonal diversity of TILs in a tumor sample. In certain aspects, the population of expanded TILs has a clonal diversity that is at least about 75% of the clonal diversity of TILs in a tumor sample. In certain aspects, the population of expanded TILs has a clonal diversity that is at least about 70% of the clonal diversity of TILs in a tumor sample. In certain aspects, the population of expanded TILs has a clonal diversity that is at least about 60% of the clonal diversity of TILs in a tumor sample. In certain aspects, the population of expanded TILs has a clonal diversity that is at least about 50% of the clonal diversity of TILs in a tumor sample. In certain aspects, the population of expanded TILs has a clonal diversity that is at least about 40% of the clonal diversity of TILs in a tumor sample.

In some aspects, the population of expanded TILs has a clonal diversity score of less than about 0.5, less than about 0.45, less than about 0.4, less than about 0.35, less than about 0.3, less than about 0.275, less than about 0.25, less than about 0.225, less than about 0.2, less than about 0.175, less than about 0.15, less than about 0.125, less than about 0.1, less than about 0.075, less than about 0.07, less than about 0.06, or less than about 0.05 as measured by Simpsons clonality. In some aspects, the population of expanded TILs has a clonal diversity score of less than about 0.5, as measured by Simpsons clonality. In some aspects, the population of expanded TILs has a clonal diversity score of less than about 0.4, as measured by Simpsons clonality. In some aspects, the population of expanded TILs has a clonal diversity score of less than about 0.3, as measured by Simpsons clonality. In some aspects, the population of expanded TILs has a clonal diversity score of less than about 0.275, as measured by Simpsons clonality. In some aspects, the population of expanded TILs has a clonal diversity score of less than about 0.25, as measured by Simpsons clonality. In some aspects, the population of expanded TILs has a clonal diversity score of less than about 0.24, as measured by Simpsons clonality. In some aspects, the population of expanded TILs has a clonal diversity score of less than about 0.23, as measured by Simpsons clonality. In some aspects, the population of expanded TILs has a clonal diversity score of less than about 0.22, as measured by Simpsons clonality. In some aspects, the population of expanded TILs has a clonal diversity score of less than about 0.21, as measured by Simpsons clonality. In some aspects, the population of expanded TILs has a clonal diversity score of less than about 0.2, as measured by Simpsons clonality. In some aspects, the population of expanded TILs has a clonal diversity score of less than about 0.19, as measured by Simpsons clonality. In some aspects, the population of expanded TILs has a clonal diversity score of less than about 0.18, as measured by Simpsons clonality. In some aspects, the population of expanded TILs has a clonal diversity score of less than about 0.17, as measured by Simpsons clonality. In some aspects, the population of expanded TILs has a clonal diversity score of less than about 0.16, as measured by Simpsons clonality. In some aspects, the population of expanded TILs has a clonal diversity score of less than about 0.15, as measured by Simpsons clonality. In some aspects, the population of expanded TILs has a clonal diversity score of less than about 0.14, as measured by Simpsons clonality. In some aspects, the population of expanded TILs has a clonal diversity score of less than about 0.13, as measured by Simpsons clonality. In some aspects, the population of expanded TILs has a clonal diversity score of less than about 0.12, as measured by Simpsons clonality. In some aspects, the population of expanded TILs has a clonal diversity score of less than about 0.11, as measured by Simpsons clonality. In some aspects, the population of expanded TILs has a clonal diversity score of less than about 0.1, as measured by Simpsons clonality. In some aspects, the population of expanded TILs has a clonal diversity score of less than about 0.09, as measured by Simpsons clonality. In some aspects, the population of expanded TILs has a clonal diversity score of less than about 0.08, as measured by Simpsons clonality. In some aspects, the population of expanded TILs has a clonal diversity score of less than about 0.07, as measured by Simpsons clonality. In some aspects, the population of expanded TILs has a clonal diversity score of less than about 0.06, as measured by Simpsons clonality. In some aspects, the population of expanded TILs has a clonal diversity score of less than about 0.05, as measured by Simpsons clonality.

In some aspects, the methods described herein selectively increase the expansion of tumor-reactive TIL clones by about 2-fold to about 500-fold, about 2-fold to about 250-fold, about 2-fold to about 200-fold, about 2-fold to about 150-fold, about 2-fold to about 100-fold, about 2-fold to about 90-fold, about 2-fold to about 80-fold, about 2-fold to about 70-fold, about 2-fold to about 60-fold, about 2-fold to about 50-fold, about 2-fold to about 40-fold, about 2-fold to about 30-fold, about 2-fold to about 20-fold, about 2-fold to about 10-fold, about 5-fold to about 200-fold, about 5-fold to about 150-fold, about 5-fold to about 100-fold, about 5-fold to about 90-fold, about 5-fold to about 80-fold, about 5-fold to about 70-fold, about 5-fold to about 60-fold, about 5-fold to about 50-fold, about 5-fold to about 40-fold, about 5-fold to about 30-fold, about 5-fold to about 20-fold, about 5-fold to about 10-fold, about 10-fold to about 150-fold, about 10-fold to about 100-fold, about 10-fold to about 90-fold, about 10-fold to about 80-fold, about 10-fold to about 70-fold, about 10-fold to about 60-fold, about 10-fold to about 50-fold, about 10-fold to about 40-fold, about 10-fold to about 30-fold, or about 10-fold to about 20-fold, as compared to selective expansion of tumor-reactive TIL clones expanded or cultured using control methods (e.g., in medium comprising less than 40 mM potassium ion, e.g., 4 mM potassium ion). In some aspects, the methods described herein selectively increase the expansion of tumor reactive TIL clones by about 2-fold. In some aspects, the methods described herein selectively increase the expansion of tumor reactive TIL clones by about 5-fold. In some aspects, the methods described herein selectively increase the expansion of tumor reactive TIL clones by about 10-fold.

In some aspects, the methods described herein selectively increase the expansion of tumor-reactive TIL clones, wherein clonal diversity is maintained. In some aspects, the methods described herein selectively increase the expansion of tumor-reactive TIL clones by about 2-fold to about 50-fold, wherein clonal diversity is maintained by about 70% to about 100%. In some aspects, the methods described herein selectively increase the expansion of tumor-reactive TIL clones by about 2-fold, wherein clonal diversity is maintained by about 70% to about 100%. In some aspects, the methods described herein selectively increase the expansion of tumor-reactive TIL clones by about 5-fold, wherein clonal diversity is maintained by about 70% to about 100%. In some aspects, the methods described herein selectively increase the expansion of tumor-reactive TIL clones by about 10-fold, wherein clonal diversity is maintained by about 70% to about 100%.

In some aspects, the TILs exhibit increased expression of one or more biomarker indicative of a less-differentiated phenotype. In some aspects, the TILs exhibit increased expression of TCF7. In some aspects, the TILs (e.g., the CD8⁺ TILs) cultured according to the methods disclosed herein exhibit an at least about 2-fold, at least about 3-fold, at least about 4-fold, at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold increase in the expression of TCF7. In some aspects, the TILs (e.g., the CD8⁺ TILs) cultured according to the methods disclosed herein exhibit an at least about 40-fold increase in the expression of TCF7. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺ TCF7⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are TCF7⁺.

In some aspects, the TILs exhibit increased expression of CD45RO. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD45RO. In some aspects, the TILs (e.g., the CD8⁺ TILs) cultured according to the methods disclosed herein exhibit an at least about 10-fold increase in the expression of CD45RO. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺ CD45RO⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD45RO⁺.

In some aspects, the TILs exhibit increased expression of CD62L. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD62L. In some aspects, the TILs (e.g., the CD8⁺ TILs) cultured according to the methods disclosed herein exhibit an at least about 10-fold increase in the expression of CD62L. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺ CD62L⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD62L⁺.

In some aspects, the TILs exhibit increased expression of CD27. In some aspects, the TILs (e.g., the CD8⁺ TILs) cultured according to the methods disclosed herein, exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD27. In some aspects, the TILs (e.g., the CD8⁺ TILs) cultured according to the methods disclosed herein exhibit an at least about 10-fold increase in the expression of CD27. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺ CD27⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD27⁺.

In some aspects, the TILs exhibit increased expression of CD62L and CD27. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD62L and CD27. In some aspects, the TILs (e.g., the CD8⁺ TILs) cultured according to the methods disclosed herein exhibit an at least about 10-fold increase in the expression of CD27. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺/CD62L⁺/CD27⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD62L⁺ CD27⁺.

In some aspects, the TILs exhibit increased expression of CD28. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD28. In some aspects, the TILs (e.g., the CD8⁺ TILs) cultured according to the methods disclosed herein exhibit an at least about 10-fold increase in the expression of CD28. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺/CD28⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD28⁺.

In some aspects, the TILs exhibit increased expression of CD27 and CD28. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD27 and CD28. In some aspects, the TILs (e.g., the CD8⁺ TILs) cultured according to the methods disclosed herein exhibit an at least about 10-fold increase in the expression of CD27 and CD28. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺ CD27⁺ CD28⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD27⁺ CD28⁺.

In some aspects, the TILs exhibit increased expression of CD27, CD28, PD1, and CD103. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD27, CD28, PD1, and CD103. In some aspects, the TILs (e.g., the CD8⁺ TILs) cultured according to the methods disclosed herein exhibit an at least about 10-fold increase in the expression of CD27, CD28, PD1, and CD103. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺ CD27⁺ CD28⁺ PD1⁺ CD103⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD27⁺ CD28⁺ PD1⁺ CD103⁺.

In some aspects, the TILs exhibit increased expression of CD27, CD28, PD1, and TCF7. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD27, CD28, PD1, and TCF7. In some aspects, the TILs (e.g., the CD8⁺ TILs) cultured according to the methods disclosed herein exhibit an at least about 10-fold increase in the expression of CD27, CD28, PD1, and TCF7. In some aspects, at least about 5%, at least about 10%, at least about 15%, at least about 20%, at least about 25%, at least about 30%, at least about 35%, at least ab out 40%, at least about 45%, at least about 50%, at least about 55%, at least about 60%, at least about 65%, at least about 70%, or at least about 75% of the TILs are CD8⁺ CD27⁺ CD28⁺ PD1⁺ TCF7⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD27⁺ CD28⁺ PD1⁺ TCF7⁺.

In some aspects, the TILs exhibit increased expression of CD27, CD28, PD1, CD103, and TCF7. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD27, CD28, PD1, CD103, and TCF7. In some aspects, the TILs (e.g., the CD8⁺ TILs) cultured according to the methods disclosed herein exhibit an at least about 10-fold increase in the expression of CD27, CD28, PD1, CD103, and TCF7. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺ CD27⁺ CD28⁺ PD1⁺ CD103⁺ TCF7⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD27⁺ CD28⁺ PD1⁺ CD103⁺ TCF7⁺.

In some aspects, the TILs exhibit increased expression of CD39. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD39. In some aspects, the TILs (e.g., the CD8⁺ TILs) cultured according to the methods disclosed herein exhibit an at least about 10-fold increase in the expression of CD39. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺ CD39⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD39⁺.

In some aspects, the TILs exhibit increased expression of CD39 and PD1. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD39 and PD1. In some aspects, the e TILs (e.g., the CD8⁺ TILs) cultured according to the methods disclosed herein exhibit an at least about 10-fold increase in the expression of CD39 and PD1. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺ CD39⁺ PD1⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are CD39⁺ PD1⁺.

In some aspects, the TILs exhibit increased expression of PD1. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of PD1. In some aspects, the TILs (e.g., the CD8⁺ TILs) cultured according to the methods disclosed herein exhibit an at least about 10-fold increase in the expression of PD1. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the immune cells are CD8⁺/PD1⁺ TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are PD1⁺.

In some aspects, the TILs exhibit increased expression of PD1 and CD27. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of PD1 and CD27. In some aspects, the TILs (e.g., the CD8⁺ TILs) cultured according to the methods disclosed herein exhibit an at least about 10-fold increase in the expression of PD1 and CD27. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺ PD1⁺ CD27⁺. TILs. In some aspects, at least about 5%, at least about 10%, at least about 15%, at least about 20%, at least about 25%, at least about 30%, at least about 35%, at least about 40%, at least about 45%, at least about 50% the CD8⁺ TILs are PD1⁺ CD27⁺.

In some aspects, the TILs exhibit increased expression of CD103. In some aspects, the TILs (e.g., the CD8⁺ TILs) cultured according to the methods disclosed herein exhibit an at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, or at least about 15-fold increase in the expression of CD103. In some aspects, the TILs (e.g., the CD8⁺ TILs) cultured according to the methods disclosed herein exhibit an at least about 10-fold increase in the expression of CD103. In some aspects, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% of the TILs are CD8⁺/CD103⁺ TILs. In some aspects, at least about 5%, at least about 10%, at least about 15%, at least about 20%, at least about 25%, at least about 30%, at least about 35%, at least about 40%, at least about 45%, at least about 50% the CD8+ TILs are CD103⁺.

In some aspects, the TILs (e.g., CD8⁺ TILs) cultured according to the methods and/or in the medium disclosed herein have an increased number of less-differentiated cells as compared to comparable immune cells cultured according to conventional methods. In some aspects, the TILs (e.g., CD8⁺ TILs) cultured according to the methods disclosed herein exhibit increased expression of one or more marker typical of a stem-like phenotype. In some aspects, TIL (e.g., CD8⁺ TIL) populations cultured according to the methods and/or in a metabolic reprogramming medium disclosed herein have an increased number of effector-like cells as compared to comparable cells cultured according to conventional methods, e.g., in media containing less than 5 mM In some aspects, TIL (e.g., CD8⁺ TIL) populations cultured according to the methods and/or in a metabolic reprogramming medium disclosed herein have both an increased number of stem-like and effector-like cells as compared to comparable cells cultured according to conventional methods, e.g., in media containing less than 5 mM In some aspects, TILs (e.g., CD8⁺ TILs) cultured according to the methods disclosed herein exhibit greater proliferative potential compared to cells cultured according to conventional methods. In some aspects, the TILs (e.g., CD8⁺ TILs) cultured according to the methods disclosed herein exhibit increased in vivo viability upon transplantation in a subject. In some aspects, the TILs (e.g., CD8⁺ TILs) cultured according to the methods disclosed herein exhibit increased cell potency. In some aspects, the TILs (e.g., CD8⁺ TILs) cultured according to the methods disclosed herein exhibit decreased cell exhaustion. In some aspects, the TILs (e.g., CD8⁺ TILs) cultured according to the methods disclosed herein exhibit increased in vivo persistence upon transplantation in a subject. In some aspects, the TILs (e.g., CD8⁺ TILs) cultured according to the methods disclosed herein exhibit increased in vivo activity upon transplantation in a subject. In some aspects, the TILs (e.g., CD8⁺ TILs) cultured according to the methods disclosed herein exhibit a more durable in vivo response upon transplantation in a subject. In some aspects, the subject is a human.

In some aspects, at least about 5% of the TILs (e.g., CD8⁺ TILs) in the composition have a stem-like phenotype. In some aspects, at least about 10% of the TILs (e.g., CD8⁺ TILs) in the composition have a stem-like phenotype. In some aspects, at least about 15% of the TILs (e.g., CD8⁺ TILs) in the composition have a stem-like phenotype. In some aspects, at least about 20% of the TILs (e.g., CD8⁺ TILs) in the composition have a stem-like phenotype. In some aspects, at least about 25% of the TILs (e.g., CD8⁺ TILs) in the composition have a stem-like phenotype. In some aspects, at least about 30% of the TILs (e.g., CD8⁺ TILs) in the composition have a stem-like phenotype. In some aspects, at least about 35% of the TILs (e.g., CD8⁺ TILs) in the composition have a stem-like phenotype. In some aspects, at least about 40% of the TILs (e.g., CD8⁺ TILs) in the composition have a stem-like phenotype. In some aspects, at least about 45% of the TILs (e.g., CD8⁺ TILs) in the composition have a stem-like phenotype. In some aspects, at least about 50% of the TILs (e.g., CD8⁺ TILs) in the composition have a stem-like phenotype. In some aspects, at least about 55% of the TILs (e.g., CD8⁺ TILs) in the composition have a stem-like phenotype. In some aspects, at least about 60% of the TILs (e.g., CD8⁺ TILs) in the composition have a stem-like phenotype. In some aspects, at least about 65% of the TILs (e.g., CD8⁺ TILs) in the composition have a stem-like phenotype. In some aspects, at least about 70% of the TILs (e.g., CD8⁺ TILs) in the composition have a stem-like phenotype.

In some aspects, following culture of TILs (e.g., CD8⁺ TILs) according to the methods disclosed herein, stem-like TILs (e.g., CD8⁺ TILs) constitute at least about 10% to at least about 70% of the total number of TILs (e.g., CD8⁺ TILs) in the culture. In some aspects, following culture of TILs (e.g., CD8⁺ TILs) according to the methods disclosed herein, stem-like TILs (e.g., CD8⁺ TILs) constitute at least about 10%, at least about 20%, at least about 30%, at least about 40%, at least about 50%, at least about 60%, or at least about 70% of the total number of TILs (e.g., CD8⁺ TILs) in the culture. In some aspects, following culture of TILs (e.g., CD8⁺ TILs) according to the methods disclosed herein, stem-like TILs (e.g., CD8⁺ TILs) constitute at least about 10%, at least about 20%, at least about 30%, at least about 40%, at least about 50%, at least about 60%, or at least about 70% of the total number of CD8⁺ TILs in the culture.

In some aspects, the number of TILs (e.g., CD8⁺ TILs) having a stem-like phenotype in the composition is increased at least about 1.5-fold as compared to the number of TILs (e.g., CD8⁺ TILs) in the composition prior to the culture. In some aspects, the number of TILs (e.g., CD8⁺ TILs) having a stem-like phenotype in the composition is increased at least about 2.0-fold as compared to the number of TILs (e.g., CD8⁺ TILs) in the composition prior to the culture. In some aspects, the number of TILs (e.g., CD8⁺ TILs) having a stem-like phenotype in the composition is increased at least about 2.5-fold as compared to the number of TILs (e.g., CD8⁺ TILs) in the composition prior to the culture. In some aspects, the number of TILs (e.g., CD8⁺ TILs) having a stem-like phenotype in the composition is increased at least about 3.0-fold as compared to the number of TILs (e.g., CD8⁺ TILs) in the composition prior to the culture. In some aspects, the number of TILs (e.g., CD8⁺ TILs) having a stem-like phenotype in the composition is increased at least about 3.5-fold as compared to the number of TILs (e.g., CD8⁺ TILs) in the composition prior to the culture. In some aspects, the number of TILs (e.g., CD8⁺ TILs) having a stem-like phenotype in the composition is increased at least about 4.0-fold as compared to the number of TILs (e.g., CD8⁺ TILs) in the composition prior to the culture. In some aspects, the number of cells having a stem-like phenotype in the composition is increased at least about 4.5-fold as compared to the number of TILs (e.g., CD8⁺ TILs) in the composition prior to the culture. In some aspects, the number of TILs (e.g., CD8⁺ TILs) having a stem-like phenotype in the composition is increased at least about 5.0-fold as compared to the number of TILs (e.g., CD8⁺ TILs) in the composition prior to the culture. In some aspects, the number of TILs (e.g., CD8⁺ TILs) having a stem-like phenotype in the composition is increased at least about 5.5-fold as compared to the number of TILs (e.g., CD8⁺ TILs) in the composition prior to the culture. In some aspects, the number of TILs (e.g., CD8⁺ TILs) having a stem-like phenotype in the composition is increased at least about 6.0-fold as compared to the number of TILs (e.g., CD8⁺ TILs) in the composition prior to the culture. In some aspects, the number of TILs (e.g., CD8⁺ TILs) having a stem-like phenotype in the composition is increased at least about 6.5-fold as compared to the number of TILs (e.g., CD8⁺ TILs) in the composition prior to the culture. In some aspects, the number of TILs (e.g., CD8⁺ TILs) having a stem-like phenotype in the composition is increased at least about 7.0-fold as compared to the number of cells in the composition prior to the culture. In some aspects, the number of TILs (e.g., CD8⁺ TILs), having a stem-like phenotype in the composition is increased at least about 7.5-fold as compared to the number of cells in the composition prior to the culture. In some aspects, the number of TILs (e.g., CD8⁺ TILs) having a stem-like phenotype in the composition is increased at least about 8.0-fold as compared to the number of TILs (e.g., CD8⁺ TILs) in the composition prior to the culture. In some aspects, the number of TILs (e.g., CD8⁺ TILs) having a stem-like phenotype in the composition is increased at least about 9.0-fold as compared to the number of TILs (e.g., CD8⁺ TILs) in the composition prior to the culture. In some aspects, the number of cells having a stem-like phenotype in the composition is increased at least about 10-fold as compared to the number of TILs (e.g., CD8⁺ TILs) in the composition prior to the culture. In some aspects, the number of cells having a stem-like phenotype in the composition is increased at least about 15-fold as compared to the number of TILs (e.g., CD8⁺ TILs) in the composition prior to the culture. In some aspects, the number of cells having a stem-like phenotype in the composition is increased at least about 20-fold as compared to the number of TILs (e.g., CD8⁺ TILs) in the composition prior to the culture. In some aspects, the number of TILs having a stem-like phenotype in the composition is increased at least about 30-fold as compared to the number of TILs in the composition prior to the culture. In some aspects, the number of TILs having a stem-like phenotype in the composition is increased at least about 40-fold as compared to the number of cells in the composition prior to the culture. In some aspects, the number of TILs having a stem-like phenotype in the composition is increased at least about 50-fold as compared to the number of TILs in the composition prior to the culture. In some aspects, the number of TILs having a stem-like phenotype in the composition is increased at least about 75-fold as compared to the number of TILs in the composition prior to the culture. In some aspects, the number of TILs having a stem-like phenotype in the composition is increased at least about 100-fold as compared to the number of TILs in the composition prior to the culture. In some aspects, the number of TILs having a stem-like phenotype in the composition is increased at least about 500-fold as compared to the number of TILs in the composition prior to the culture. In some aspects, the number of TILs having a stem-like phenotype in the composition is increased at least about 1000-fold as compared to the number of TILs in the composition prior to the culture.

In some aspects, following culture of TILs (e.g., CD8⁺ TILs) according to the methods disclosed herein, at least about 10% to at least about 70% of the total number of TILs (e.g., CD8⁺ TILs) in the culture are CD39⁻/TCF7⁺ T cells. In some aspects, following culture of TILs (e.g., CD8⁺ TILs) according to the methods disclosed herein, at least about 10%, at least about 15%, at least about 20%, at least about 25%, at least about 30%, at least about 35%, or at least about 40% of the total number of TILs (e.g., CD8⁺ TILs) in the culture are CD39⁻/TCF7⁺ TILs (e.g., CD8⁺ TILs).

In some aspects, the cell composition comprises an increased percentage of TILs which express CD95. In some aspects, the cell composition comprises an increased percentage of TILs which do not express CD45RO. In some aspects, the cell composition comprises an increased percentage of TILs which express CD45RA. In some aspects, the cell composition comprises an increased percentage of TILs which express CCR7. In some aspects, the cell composition comprises an increased percentage of TILs which express CD62L. In some aspects, the cell composition comprises an increased percentage of TILs which express TCF7. In some aspects, the cell composition comprises an increased percentage of TILs which express CD3. In some aspects, the cell composition comprises an increased percentage of TILs which express CD27. In some aspects, the cell composition comprises an increased percentage of TILs which express CD45RA. In some aspects, the cell composition comprises an increased percentage of TILs which express CD95 and CD45RA. In some aspects, the cell composition comprises an increased percentage of TILs which express CD45RA and CCR7. In some aspects, the cell composition comprises an increased percentage of TILs which express CD95, CD45RA, and CCR7. In some aspects, the cell composition comprises an increased percentage of TILs which express CD45RA, CCR7, and CD62L. In some aspects, the cell composition comprises an increased percentage of TILs which express CD95, CD45RA, CCR7, and CD62L. In some aspects, the cell composition comprises an increased percentage of TILs which express CD45RA, CCR7, CD62L, and TCF7. In some aspects, the cell composition comprises an increased percentage of TILs which express CD95, CD45RA, CCR7, CD62L, and TCF7. In some aspects, the cell composition comprises an increased percentage of TILs which express CD45RA, CCR7, CD62L, TCF7, and CD27. In some aspects, the cell composition comprises an increased percentage of TILs which express CD95, CD45RA, CCR7, CD62L, TCF7, and CD27. In some aspects, the cell composition comprises an increased percentage of TILs which express CD45RA, CCR7, CD62L, TCF7, and CD27, and which are CD45RO^{low}. In some aspects, the cell composition comprises an increased percentage of TILs which express CD95, CD45RA, CCR7, CD62L, TCF7, and CD27, and which are CD45RO^{low}. In some aspects, the cell composition comprises an increased percentage of TILs which express CD45RA, CCR7, CD62L, TCF7, and CD27, and which do not express CD45RO. In some aspects, the cell composition comprises an increased percentage of TILs which express CD95, CD45RA, CCR7, CD62L, TCF7, and CD27, and which do not express CD45RO.

In some aspects, the cell composition comprises an increase in the percent of TILs which do not express CD39 and CD69. In some aspects, the cell composition comprises an increase in the percent of TILs which express CD8, and which do not express CD39 and CD69. In some aspects, following culture of TILs according to the methods disclosed herein, at least about 10% to at least about 40% of the total number of TILs in the culture are CD39⁻/CD69⁻ TILs. In some aspects, following culture of TILs according to the methods disclosed herein, at least about 10%, at least about 15%, at least about 20%, at least about 25%, at least about 30%, at least about 35%, or at least about 40% of the total number of TILs in the culture are CD39⁻/CD69⁻ TILs.

In some aspects, the TILs (e.g., CD8⁺ TILs) cultured according to the methods and/or in the medium disclosed herein express one or more stem-like markers and one or more effector-like markers. In some aspects, the TILs (e.g., CD8⁺ TILs) cultured according to the methods and/or in the medium disclosed herein express at least two stem-like markers and one or more effector-like markers. In some aspects, the TILs (e.g., CD8⁺ TILs) cultured according to the methods and/or in the medium disclosed herein express at least three stem-like markers and one or more effector-like markers. In some aspects, the TILs (e.g., CD8⁺ TILs) cultured according to the methods and/or in the medium disclosed herein express at least four stem-like markers and one or more effector-like markers. In some aspects, the TILs (e.g., CD8⁺ TILs) cultured according to the methods and/or in the medium disclosed herein express one or more stem-like markers and at least two effector-like markers. In some aspects, at least about 40%, at least about 50%, at least about 60%, at least about 70%, at least about 80%, at least about 85%, at least about 90%, at least about 95%, at least about 99%, or about 100% of the TILs express one or more stem-like markers and one or more effector-like markers. In some aspects, at least about 40% of the TILs express one or more stem-like markers and one or more effector-like markers. In some aspects, at least about 50% of the TILs express one or more stem-like markers and one or more effector-like markers. In some aspects, at least about 60% of the TILs express one or more stem-like markers and one or more effector-like markers. In some aspects, at least about 70% of the TILs express one or more stem-like markers and one or more effector-like markers. In some aspects, at least about 75% of the TILs express one or more stem-like markers and one or more effector-like markers. In some aspects, at least about 80% of the TILs express one or more stem-like markers and one or more effector-like markers. In some aspects, at least about 85% of the TILs express one or more stem-like markers and one or more effector-like markers. In some aspects, at least about 90% of the TILs express one or more stem-like markers and one or more effector-like markers. In some aspects, at least about 95% of the TILs express one or more stem-like markers and one or more effector-like markers. In some aspects, at least about 96% of the TILs express one or more stem-like markers and one or more effector-like markers. In some aspects, at least about 97% of the TILs express one or more stem-like markers and one or more effector-like markers. In some aspects, at least about 98% of the TILs express one or more stem-like markers and one or more effector-like markers. In some aspects, at least about 99% of the TILs express one or more stem-like markers and one or more effector-like markers.

In some aspects, the stem-like markers are selected from CD45RA+, CD62L+, CCR7+, CD27+, CD28+, BACH2+, LEF1+, TCF7+, and any combination thereof. In some aspects the stem-like markers comprise CD45RA+, CD62L+, CCR7+, and TCF7+, or any combination thereof. In some aspects, the TIL expresses CD45RO^{low}. In some aspects, the stem-like markers comprise one or more genes listed herein as part of a gene-signature (see supra; see, e.g., Gattinoni, L., et al., Nat Med 17(10): 1290-1297 (2011) or Galletti et al. Nat Immunol 21, 1552-1562 (2020)). In some aspects, the effector-like markers are selected from pSTAT5+, STAT5+, pSTAT3+, STAT3+, and any combination thereof. In some aspects, the effector-like marker comprises a STAT target selected from the group consisting of AKT1, AKT2, AKT3, BCL2L1, CBL, CBLB, CBLC, CCND1, CCND2, CCND3, CISH, CLCF1, CNTF, CNTFR, CREBBP, CRLF2, CSF2, CSF2RA, CSF2RB, CSF3, CSF3R, CSH1, CTF1, EP300, EPO, EPOR, GH1, GH2, GHR, GRB2, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNLR1, IFNW1, IL10, IL 10RA, IL 10RB, IL11, IL11RA, IL12A, IL12B, IL12RB1, IL12RB2, IL13, IL13RA1, IL13RA2, IL15, IL15RA, IL19, IL2, IL20, IL20RA, IL20RB, IL21, IL21R, IL22, IL22RA1, IL22RA2, IL23A, IL23R, IL24, IL26, IL2RA, IL2RB, IL2RG, IL3, IL3RA, IL4, IL4R, IL5, IL5RA, IL6, IL6R, IL6ST, IL7, IL7R, IL9, IL9R, IRF9, JAK1, JAK2, JAK3, LEP, LEPR, LIF, LIFR, MPL, MYC, OSM, OSMR, PIAS1, PIAS2, PIAS3, PIAS4, PIK3CA, PIK3CB, PIK3CD, PIK3CG, PIK3R1, PIK3R2, PIK3R3, PIK3R5, PIM1, PRL, PRLR, PTPN11, PTPN6, SOCS1, SOCS2, SOCS3, SOCS4, SOCS5, SOCS7, SOS1, SOS2, SPRED1, SPRED2, SPRY1, SPRY2, SPRY3, SPRY4, STAM, STAM2, STAT1, STAT2, STAT3, STAT4, STAT5A, STAT5B, STATE, TPO, TSLP, TYK2, and any combination thereof. In some aspects, the TILs (e.g., CD8⁺ TILs) cultured according to the methods and/or in the medium disclosed herein are CD45RA+, STAT5+, and STAT3+. In some aspects, the TILs (e.g., CD8⁺ TILs) cultured according to the methods and/or in the medium disclosed herein are CD62L+, STAT5+, and STAT3+. In some aspects, the TILs (e.g., CD8⁺ TILs) cultured according to the methods and/or in the medium disclosed herein are TCF7+, STAT5+, and STAT3+. In some aspects, the TILs (e.g., CD8⁺ TILs) cultured according to the methods and/or in the medium disclosed herein are CD45RA+, CD62L+, CCR7+, CD27+, CD28+, BACH2+, LEF1+, TCF7+, STAT5+, and STAT3+. In some aspects the TILs (e.g., CD8⁺ TILs) cultured according to the methods and/or in the medium disclosed herein are CD45RA+, CD62L+, CCR7+, CD27+, CD28+, BACH2+, LEF1+, TCF7+, pSTAT5+, STAT5+, pSTAT3+, and STAT3+. In some aspects, the TILs (e.g., CD8⁺ TILs) cultured according to the methods and/or in the medium disclosed herein are CD45RA+, CD62L+, CCR7+, CD27+, CD28+, BACH2+, LEF1+, TCF7+, pSTAT5+, STAT5+, pSTAT3+, and STAT3+. In some aspects, the TILs (e.g., CD8⁺ TILs) cultured according to the methods and/or in the medium disclosed herein are CD45RA+, CD45RO^{low}, CD62L+, CCR7+, CD27+, CD28+, BACH2+, LEF1+, TCF7+, pSTAT5+, STAT5+, pSTAT3+, and STAT3+.

In some aspects, an TIL comprises one or more markers selected from CD45RA+, CD62L+, CCR7+, CD27+, CD28+, BACH2+, LEF1+, TCF7+, and any combination thereof and one or more markers selected from pSTAT5+, STAT5+, pSTAT3+, STAT3+, and any combination thereof. In some aspects, a TIL comprises one or more markers selected from CD45RA+, CD62L+, CCR7+, CD27+, CD28+, BACH2+, LEF1+, TCF7+, and any combination thereof and one or more effector-like markers. In some aspects, a TIL comprises one or more stem-like markers and one or more markers selected from pSTAT5+, STAT5+, pSTAT3+, STAT3+, and any combination thereof. In some aspects, the effector-like marker comprises a STAT target selected from the group consisting of AKT1, AKT2, AKT3, BCL2L1, CBL, CBLB, CBLC, CCND1, CCND2, CCND3, CISH, CLCF1, CNTF, CNTFR, CREBBP, CRLF2, CSF2, CSF2RA, CSF2RB, CSF3, CSF3R, CSH1, CTF1, EP300, EPO, EPOR, GH1, GH2, GHR, GRB2, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNLR1, IFNW1, IL10, IL10RA, IL10RB, IL11, IL11RA, IL12A, IL12B, IL12RB 1, IL12RB2, IL13, IL13RA1, IL13RA2, IL15, IL15RA, IL19, IL2, IL20, IL20RA, IL20RB, IL21, IL21R, IL22, IL22RA1, IL22RA2, IL23A, IL23R, IL24, IL26, IL2RA, IL2RB, IL2RG, IL3, IL3RA, IL4, IL4R, IL5, IL5RA, IL6, IL6R, IL6ST, IL7, IL7R, IL9, IL9R, IRF9, JAK1, JAK2, JAK3, LEP, LEPR, LIF, LIFR, MPL, MYC, OSM, OSMR, PIAS1, PIAS2, PIAS3, PIAS4, PIK3CA, PIK3CB, PIK3CD, PIK3CG, PIK3R1, PIK3R2, PIK3R3, PIK3R5, PIM1, PRL, PRLR, PTPN11, PTPN6, SOCS1, SOCS2, SOCS3, SOCS4, SOCS5, SOCS7, SOS1, SOS2, SPRED1, SPRED2, SPRY1, SPRY2, SPRY3, SPRY4, STAM, STAM2, STAT1, STAT2, STAT3, STAT4, STAT5A, STAT5B, STATE, TPO, TSLP, TYK2, and any combination thereof.

In some aspects, the TILs that expresses one or more stem-like markers and one or more effector-like marker is a T stem/effector (T_{SE}) cell. In some aspects, the T_{SE} cell retains a less differentiated state (e.g., expresses one or more stem-like markers, is capable of proliferation, is capable of differentiation, or any combination thereof) and the cell has effector function (e.g., expresses one or more effector-like markers, is capable of targeting and/or killing tumor cells, exhibits polyfunctionality, or a combination thereof). In some aspects, a T_{SE} cell disclosed herein expresses CD45RA+, CD62L+, CCR7+, CD27+, CD28+, BACH2+, LEF1+, TCF7+, pSTAT5+, STAT5+, pSTAT3+, and STAT3+. In some aspects, a T_{SE} cell disclosed herein expresses CD45RA+, CD62L+, CCR7+, TCF7+, pSTAT5+, STAT5+, pSTAT3+, and STAT3+. In some aspects, the T_{SE} cell is CD45RO^{low}.

Some aspects of the present disclosure are directed to an expanded population of TILs comprising one or more T_{SE} cell. In some aspects, at least about 40%, at least about 50%, at least about 60%, at least about 70%, at least about 80%, at least about 85%, at least about 90%, at least about 95%, at least about 99%, or about 100% of the expanded population of TILs are T_{SE} cells. In some aspects, at least about 40% of the expanded population of TILs are T_{SE} cells. In some aspects, at least about 50% of the expanded population of TILs are T_{SE} cells. In some aspects, at least about 60% of the expanded population of TILs are T_{SE} cells. In some aspects, at least about 70% of the expanded population of TILs are T_{SE} cells. In some aspects, at least about 75% of the expanded population of TILs are T_{SE} cells. In some aspects, at least about 80% of the expanded population of TILs are T_{SE} cells. In some aspects, at least about 85% of the expanded population of TILs are T_{SE} cells. In some aspects, at least about 90% of the expanded population of TILs are T_{SE} cells. In some aspects, at least about 95% of the expanded population of TILs are T_{SE} cells. In some aspects, at least about 98% of the expanded population of TILs are T_{SE} cells. In some aspects, at least about 99% of the expanded population of TILs are T_{SE} cells. In some aspects, about 100% of the expanded population of TILs in the population are T_{SE} cells.

Some aspects of the present disclosure are directed to a TIL, which expresses one or more stem-like markers and one or more effector-like marker. In some aspects, the TIL expresses at least two stem-like markers and one or more effector-like markers. In some aspects, the TIL expresses at least three stem-like markers and one or more effector-like markers. In some aspects, the TIL expresses at least four stem-like markers and one or more effector-like markers. In some aspects, the TIL expresses one or more stem-like markers and at least two effector-like markers. In some aspects, the stem-like markers are selected from CD45RA+, CD62L+, CCR7+, CD27+, CD28+, BACH2+, LEF1+, TCF7+, and any combination thereof. In some aspects, the stem-like markers are selected from CD45RA+, CD62L+, CCR7+, TCF7+, and any combination thereof. In some aspects, the stem-like markers comprise one or more genes listed herein as part of a gene-signature (see supra; see, e.g., Gattinoni, L., et al., Nat Med 17(10): 1290-1297 (2011) or Galletti et al. Nat Immunol 21, 1552-1562 (2020)). In some aspects, the effector-like markers are selected from pSTAT5+, STAT5+, pSTAT3+, STAT3+, and any combination thereof. In some aspects, the TIL expresses CD45RA+, STAT5+, and STAT3+. In some aspects, the TIL expresses CD62L+, STAT5+, and STAT3+. In some aspects, the TIL expresses TCF7+, STAT5+, and STAT3+. In some aspects, the TIL expresses CD45RA+, CD62L+, CCR7+, CD27+, CD28+, BACH2+, LEF1+, TCF7+, STAT5+, and STAT3+. In some aspects, the TIL expresses CD45RA+, CD62L+, CCR7+, CD27+, CD28+, BACH2+, LEF1+, TCF7+, pSTAT5+, STAT5+, pSTAT3+, and STAT3+. In some aspects, the TIL expresses CD45RA+, CD62L+, CCR7+, CD27+, CD28+, BACH2+, LEF1+, TCF7+, CD45RO^{low}, pSTAT5+, STAT5+, pSTAT3+, and STAT3+.

Some aspects of the present disclosure are directed to a cell composition comprising a population of TILs, wherein the population of TILs comprises (i) a first sub-population of TILs expressing one or more stem-like markers (e.g., stem-like TILs) and (ii) a second sub-population of TILs expressing one or more effector-like marker (e.g., effector-like TILs), wherein the population of TILs comprises a higher percentage (i.e., the number of stem-like TILs/the total number of TILs) of the first sub-population of TILs expressing one or more stem-like markers, as compared to a population of TILs cultured in a control media. In some aspects, the TILs cultured according to the methods disclosed herein result in these cell compositions. In some aspects, TILs cultured according to the methods disclosed herein have increased expression, e.g., a higher percentage of TILs that express, GZMB, MHC-II, LAG3, TIGIT, and/or NKG7, and decreased expression, e.g., a lower percentage of TILs that express, IL-32. Cells highest for NKG7 have been shown to be better killers (Malarkannan et al. 2020 Nat. Immuno.), whereas cells higher in IL-32 have been shown to have activation-induced cell death (Goda et al., 2006 Int. Immunol). In some aspects the TILs with higher expression of GZMB, MHC-II, LAG3, TIGIT, and/or NKG7 are CD8+ TILs expressing effector-like markers. In some aspects the TILs with lower expression of IL-32 are CD8+ TILs expressing effector-like markers.

Some aspects of the present disclosure are directed to a cell composition comprising TILs expressing one or more stem-like markers and one or more effector-like marker. Some aspects of the present disclosure are directed to a population of cells comprising the TIL, e.g., the TIL expressing one or more stem-like markers and one or more effector-like marker. In some aspects, at least about 40%, at least about 50%, at least about 60%, at least about 70%, at least about 80%, at least about 85%, at least about 90%, at least about 95%, at least about 99%, or about 100% of the cell composition comprise TILs expressing one or more stem-like markers and one or more effector-like marker.

In some aspects, the TIL that expresses one or more stem-like markers and one or more effector-like markers is a T stem/effector (T_{SE}) cell. In some aspects, the T_{SE} cell retains a less differentiated state (e.g., expresses one or more stem-like markers, is capable of proliferation, is capable of differentiation, or any combination thereof) and the cell has effector function (e.g., expresses one or more effector-like markers, is capable of targeting and/or killing tumor cells, or a combination thereof). In some aspects, a T_{SE} cell disclosed herein expresses CD45RA+, CD62L+, CCR7+, CD27+, CD28+, BACH2+, LEF1+, TCF7+, pSTAT5+, STAT5+, pSTAT3+, and STAT3+. In some aspects, a T_{SE} cell disclosed herein expresses CD45RA+, CD62L+, CCR7+, CD27+, CD28+, BACH2+, LEF1+, TCF7+, STAT5+, and STAT3+. In some aspects, a T_{SE} cell disclosed herein expresses CD45RA+, CD62L+, CCR7+, CD27+, CD28+, BACH2+, LEF1+, TCF7+, pSTAT5+, STAT5+, pSTAT3+, and STAT3+. In some aspects, a T_{SE} cell disclosed herein expresses CD45RA+, CD62L+, CCR7+, CD27+, CD28+, BACH2+, LEF1+, TCF7+, CD45RO^{low}, pSTAT5+, STAT5+, pSTAT3+, and STAT3+. Some aspects of the present disclosure are directed to a population of expanded TILs comprising one or more T_{SE} cell. In some aspects, at least about 40%, at least about 50%, at least about 60%, at least about 70%, at least about 80%, at least about 85%, at least about 90%, at least about 95%, at least about 99%, or about 100% of the expanded TILs are T_{SE} cells. In some aspects, at least about 40% of the expanded TILs are T_{SE} cells. In some aspects, at least about 50% of the expanded TILs are T_{SE} cells. In some aspects, at least about 60% of the expanded TILs are T_{SE} cells. In some aspects, at least about 70% of the expanded TILs are T_{SE} cells. In some aspects, at least about 75% of the expanded TILs are T_{SE} cells. In some aspects, at least about 80% of the expanded TILs are T_{SE} cells. In some aspects, at least about 85% of the expanded TILs are T_{SE} cells. In some aspects, at least about 90% of the expanded TILs are T_{SE} cells. In some aspects, at least about 95% of the expanded TILs are T_{SE} cells. In some aspects, at least about 98% the expanded TILs are T_{SE} cells. In some aspects, at least about 99% of the expanded TILs are T_{SE} cells. In some aspects, about 100% the expanded TILs are T_{SE} cells.

In some aspects, the TIL is an engineered TIL. As used herein, an “engineered” TIL refers to a TIL that has been manipulated in a way, e.g., according to the methods disclosed herein, that confers on the TIL one or more physical and/or functional properties that are not characteristic of a naturally occurring TIL. For example, in some aspects, an engineered TIL can be generated by modifying a TIL to express one or more proteins heterologous to the cell (e.g., chimeric antigen receptor or T cell receptor) so that the engineered TIL is not naturally occurring. In some aspects, an engineered TIL can be generated by culturing a TIL in a particular way, e.g., culturing in hyperkalemic medium, wherein the resulting engineered TIL has one or more physical and/or functional properties that are not shown in naturally occurring cells.

In some aspects, the cell composition after the initial culture comprises at least about 2×10⁶, at least about 3×10⁶, at least about 4×10⁶, at least about 5×10⁶, at least about 6×10⁶, at least about 7×10⁶, at least about 8×10⁶, at least about 9×10⁶, or at least about 10×10⁶ cells (e.g., TILs). In some aspects, the cell composition after the initial culture comprises about 2×10⁶ to about 10×10⁶, e.g., about 2×10⁶, about 3×10⁶, about 4×10⁶, about 5×10⁶, about 6×10⁶, about 7×10⁶, about 8×10⁶, about 9×10⁶, or about 10×10⁶, cells (e.g., TILs). In some aspects, the composition after the initial culture comprises about 2×10⁶ cells (e.g., TILs) to about 3×10⁶ cells (e.g., TILs). In some aspects, the composition after the initial culture comprises about 3×10⁶ cells (e.g., TILs) to about 4×10⁶ cells (e.g., TILs). In some aspects, the composition after the initial culture comprises about 4×10⁶ cells (e.g., TILs) to about 5×10⁶ cells (e.g., TILs). In some aspects, the composition after the initial culture comprises about 5×10⁶ cells (e.g., TILs) to about 6×10⁶ cells (e.g., TILs). In some aspects, the composition after the initial culture comprises about 6×10⁶ cells (e.g., TILs) to about 7×10⁶ cells (e.g., TILs). In some aspects, the composition after the initial culture comprises about 7×10⁶ cells (e.g., TILs) to about 8×10⁶ cells (e.g., TILs). In some aspects, the composition after the initial culture comprises about 8×10⁶ cells (e.g., TILs) to about 9×10⁶ cells (e.g., TILs). In some aspects, the composition after the initial culture comprises about 9×10⁶ cells (e.g., TILs) to about 10×10⁶ cells (e.g., TILs).

In some aspects, the cell composition after the second TIL expansion comprises at least about 5×10⁷, at least about 3×10⁷, at least about 4×10⁷, at least about 5×10⁷, at least about 6×10⁷, at least about 7×10⁷, at least about 8×10⁷, at least about 9×10⁷, at least about 10×10⁷, at least about 11×10⁷, at least about 12×10⁷, at least about 13×10⁷, at least about 14×10⁷, at least about 15×10⁷, at least about 16×10⁷, at least about 17×10⁷, at least about 18×10⁷, at least about 19×10⁷, or at least about 20×10⁷ cells (e.g., TILs). In some aspects, the cell composition after the second expansion comprises about 5×10⁷ to about 20×10⁷, e.g., about 5×10⁷, about 6×10⁷, about 7×10⁷, about 8×10⁷, about 9×10⁷, about 10×10⁷, about 11×10⁷, about 12×10⁷, about 13×10⁷, about 14×10⁷, about 15×10⁷, about 16×10⁷, about 17×10⁷, about 18×10⁷, about 19×10⁷, or about 20×10⁷ cells (e.g., TILs). In some aspects, the composition after the second expansion comprises about 5×10⁷ to about 6×10⁷ cells (e.g., TILs), about 6×10⁷ to about 7×10⁷ cells (e.g., TILs), about 7×10⁷ to about 8×10⁷ cells (e.g., TILs), about 8×10⁷ to about 9×10⁷ cells (e.g., TILs), about 9×10⁷ to about 10×10⁷ cells (e.g., TILs), about 10×10⁷ to about 11×10⁷ cells (e.g., TILs), about 11×10⁷ to about 12×10⁷ cells (e.g., TILs), about 12×10⁷ to about 13×10⁷ cells (e.g., TILs), about 13×10⁷ to about 14×10⁷ cells (e.g., TILs), about 14×10⁷ to about 15×10⁷ cells (e.g., TILs), about 15×10⁷ to about 16×10⁷ cells (e.g., TILs), about 16×10⁷ to about 17×10⁷ cells (e.g., TILs), about 17×10⁷ to about 18×10⁷ cells (e.g., TILs), about 18×10⁷ to about 19×10⁷ cells (e.g., TILs), or about 19×10⁷ to about 20×10⁷ cells (e.g., TILs). In some aspects, the composition after the second expansion comprises about 5×10⁷ to about 6×10⁷ cells (e.g., TILs). In some aspects, the composition after the second expansion comprises about 6×10⁷ to about 7×10⁷ cells (e.g., TILs). In some aspects, the composition after the second expansion comprises about 7×10⁷ to about 8×10⁷ cells (e.g., TILs). In some aspects, the composition after the second expansion comprises about 8×10⁷ to about 9×10⁷ cells (e.g., TILs). In some aspects, the composition after the second expansion comprises about 9×10⁷ to about 10×10⁷ cells (e.g., TILs). In some aspects, the composition after the second expansion comprises about 10×10⁷ to about 11×10⁷ cells (e.g., TILs). In some aspects, the composition after the second expansion comprises about 11×10⁷ to about 12×10⁷ cells (e.g., TILs). In some aspects, the composition after the second expansion comprises about 12×10⁷ to about 13×10⁷ cells (e.g., TILs). In some aspects, the composition after the second expansion comprises about 13×10⁷ to about 14×10⁷ cells (e.g., TILs). In some aspects, the composition after the second expansion comprises about 14×10⁷ to about 15×10⁷ cells (e.g., TILs). In some aspects, the composition after the second expansion comprises about 15×10⁷ to about 16×10⁷ cells (e.g., TILs). In some aspects, the composition after the second expansion comprises about 16×10⁷ to about 17×10⁷ cells (e.g., TILs). In some aspects, the composition after the second expansion comprises about 17×10⁷ to about 18×10⁷ cells (e.g., TILs). In some aspects, the composition after the second expansion comprises about 18×10⁷ to about 19×10⁷ cells (e.g., TILs). In some aspects, the composition after the second expansion comprises about 19×10⁷ to about 20×10⁷ cells (e.g., TILs).

In some aspects, the cell composition after the final TIL expansion comprises at least about 40×10⁹, at least about 50×10⁹, at least about 60×10⁹, at least about 70×10⁹, at least about 80×10⁹, at least about 90×10⁹, or at least about 100×10⁹ cells (e.g., TILs). In some aspects, the cell composition after the final expansion comprises about 40×10⁹ to about 100×10⁹, e.g., about 40×10⁹, about 50×10⁹, about 60×10⁹, about 70×10⁹, about 80×10⁹, about 90×10⁹, or about 100×10⁹ cells (e.g., TILs). In some aspects, the composition after the final expansion comprises about 40×10⁹ to about 50×10⁹ cells (e.g., TILs), about 50×10⁹ to about 60×10⁹ cells (e.g., TILs), about 60×10⁹ to about 70×10⁹ cells (e.g., TILs), about 70×10⁹ to about 80×10⁹ cells (e.g., TILs), about 80×10⁹ to about 90×10⁹ cells (e.g., TILs), or about 90×10⁹ to about 100×10⁹ cells (e.g., TILs). In some aspects, the composition after the final expansion comprises about 40×10⁹ to about 50×10⁹ cells (e.g., TILs). In some aspects, the composition after the final expansion comprises about 50×10⁹ to about 60×10⁹ cells (e.g., TILs). In some aspects, the composition after the final expansion comprises about 60×10⁹ to about 70×10⁹ cells (e.g., TILs). In some aspects, the composition after the final expansion comprises about 70×10⁹ to about 80×10⁹ cells (e.g., TILs). In some aspects, the composition after the final expansion comprises about 80×10⁹ to about 90×10⁹ cells (e.g., TILs). In some aspects, the composition after the final expansion comprises about 90×10⁹ to about 100×10⁹ cells (e.g., TILs).

In some aspects, the cell composition suitable for administration to a subject comprises at least about 2×10⁹, at least about 3×10⁹, at least about 4×10⁹, at least about 5×10⁹, at least about 6×10⁹, at least about 7×10⁹, at least about 8×10⁹, at least about 9×10⁹, or at least about 1×10¹⁰, or at least about 10×10¹⁰, or at least about 15×10¹⁰, or at least about 20×10¹⁰, or at least about 25×10¹⁰, or at least about 30×10¹⁰ CD8⁺ TILs. In some aspects, the cell composition suitable for administration to a subject comprises at least about 2×10⁹ CD8⁺ TILs. In some aspects, the cell composition suitable for administration to a subject comprises at least about 5×10⁹ CD8⁺ TILs. In some aspects, the cell composition suitable for administration to a subject comprises at least about 9×10⁹ CD8⁺ TILs. In some aspects, the cell composition suitable for administration to a subject comprises at least about 1×10¹⁰ CD8⁺ TILs. In some aspects, the cell composition suitable for administration to a subject comprises at least about 10×10¹⁰ CD8⁺ TILs. In some aspects, the cell composition suitable for administration to a subject comprises at least about 20×10¹⁰ CD8⁺ TILs. In some aspects, the cell composition suitable for administration to a subject comprises at least about 30×10¹⁰ CD8⁺ TILs.

In some aspects, the methods disclosed herein yield a composition comprising TILs that are at least about 80%, at least about 85%, at least about 90%, at least about 94%, at least about 95%, at least about 96%, at least about 97%, at least about 98%, or at least about 99% viable.

### IV. Methods of Treatment

Some aspects of the present disclosure are directed to a population of TILs cultured according to any of the methods disclosed herein. In some aspects, the TILs are tumor-infiltrating T cells. In some aspects, the TILs comprise both CD4⁺ T cells and CD8⁺ T cells. In some aspects, the TILs comprise CD8⁺ T cells. In some aspects, the TILs are enriched for tumor reactive (e.g., tumor specific) TILs. In some aspects, the TILs are enriched for stem-like TILs.

In some aspects, the composition comprising the population of TILs is administered to a subject in need thereof. In some aspects the TILs prepared using the methods disclose herein are administered to a subject to treat a cancer, e.g., a tumor. In some aspects, the method of treating comprises administering to the subject an effective amount of a TIL composition of the disclosure, e.g., a composition comprising a population of TILs prepared according to the methods disclosed herein, e.g., a population of TILs enriched for CD8⁺ TILs, tumor-specific TILs, and/or stem-like TILs.

The present disclosure also provides a method of stimulating a T cell-mediated immune response to a target cell population or tissue in a subject, comprising administering an effective amount of a TIL composition of the disclosure, e.g., a population of TILs prepared according to the methods disclosed herein, e.g., a population of TILs enriched for CD8⁺ TILs.

In some aspects, the population of TILs administered in the cell composition of the disclosure comprises autologous TILs.

In some aspects, the method comprises administering at least about 1×10⁴, at least about 5×10⁴, at least about 1×10⁵, at least about 5×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, at least about 5×10⁷, at least about 1×10⁸ TILs to the subject. In some aspects, the method comprises administering at least about 1×10⁴, at least about 5×10⁴, at least about 1×10⁵, at least about 5×10⁵, at least about 1×10⁶, at least about 2×10⁶, at least about 3×10⁶, at least about 4×10⁶, at least about 5×10⁶, at least about 6×10⁶, at least about 7×10⁶, at least about 8×10⁶, at least about 9×10⁶, at least about 1×10⁷, at least about 5×10⁷, at least about 1×10⁸, at least about 1×10⁹, at least about 2×10⁹, at least about 3×10⁹, at least about 4×10⁹, at least about 5×10⁹, at least about 6×10⁹, at least about 7×10⁹, at least about 8×10⁹, at least about 9×10⁹, at least about 1×10¹⁰, at least about 10×10¹⁰, at least about 15×10¹⁰, at least about 20×10¹⁰, at least about 25×10¹⁰, or at least about 30×10¹⁰ cells to the subject. In some aspects, the cells are TILs. In some aspects, the TILs are CD8⁺ TILs.

In some aspects, the method comprises administering at least about 10×10⁹, at least about 20×10⁹, at least about 30×10⁹, at least about 40×10⁹, at least about 50×10⁹, at least about 60×10⁹, at least about 70×10⁹, at least about 80×10⁹, at least about 90×10⁹, or at least about 100×10⁹ cells (e.g., TILs). In some aspects, the method comprises administering about 10×10⁹ to about 100×10⁹, e.g., about 10×10⁹, about 20×10⁹, about 30×10⁹, about 40×10⁹, about 50×10⁹, about 60×10⁹, about 70×10⁹, about 80×10⁹, about 90×10⁹, or about 100×10⁹, cells (e.g., TILs). In some aspects, the method comprises administering about 10×10⁹ to about 20×10⁹ cells (e.g., TILs), about 20×10⁹ to about 30×10⁹ cells (e.g., TILs), about 30×10⁹ to about 40×10⁹ cells (e.g., TILs), about 40×10⁹ to about 50×10⁹ cells (e.g., TILs), about 50×10⁹ to about 60×10⁹ cells (e.g., TILs), about 60×10⁹ to about 70×10⁹ cells (e.g., TILs), about 70×10⁹ to about 80×10⁹ cells (e.g., TILs), about 80×10⁹ to about 90×10⁹ cells (e.g., TILs), or about 90×10⁹ to about 100×10⁹ cells (e.g., TILs). In some aspects, the method comprises administering about 10×10⁹ to about 20×10⁹ cells (e.g., TILs). In some aspects, the method comprises administering about 20×10⁹ to about 30×10⁹ cells (e.g., TILs). In some aspects, the method comprises administering about 30×10⁹ to about 40×10⁹ cells (e.g., TILs). In some aspects, the method comprises administering about 40×10⁹ to about 50×10⁹ cells (e.g., TILs). In some aspects, the method comprises administering about 50×10⁹ to about 60×10⁹ cells (e.g., TILs). In some aspects, the method comprises administering about 60×10⁹ to about 70×10⁹ cells (e.g., TILs). In some aspects, the method comprises administering about 70×10⁹ to about 80×10⁹ cells (e.g., TILs). In some aspects, the method comprises administering about 80×10⁹ to about 90×10⁹ cells (e.g., TILs). In some aspects, the method comprises administering about 90×10⁹ to about 100×10⁹ cells (e.g., TILs). In some aspects, the TILs are CD8⁺ TILs.

In some aspects, the TILs are administered at a ratio of TILs to tumor cells of at least about 2:1, at least about 2.5:1, at least about 3:1, at least about 3.5:1, or at least about 4:1. In some aspects, the TILs are administered at a ratio of TILs to tumor cells of at least about 2:1. In some aspects, the TILs are administered at a ratio of TILs to tumor cells of at least about 2.5:1. In some aspects, the TILs are administered at a ratio of TILs to tumor cells of at least about 3:1. In some aspects, the TILs are administered at a ratio of TILs to tumor cells of at least about 3.5:1. In some aspects, the TILs are administered at a ratio of TILs to tumor cells of at least about 4:1.

In some aspects, administering the cell composition of the disclosure (e.g., comprising a population of TILs prepared according to the methods disclosed herein (e.g., enriched for CD8⁺ TILs, tumor-specific TILs, and/or stem-like TILs)) reduces a tumor volume in the subject compared to a reference tumor volume. In some aspects, the reference tumor volume is the tumor volume in the subject prior to the administration of the engineered cell. In further aspects, the reference tumor volume is the tumor volume in a corresponding subject that did not receive the administration. In some aspects, the tumor volume in the subject is reduced by at least about 5%, at least about 10%, at least about 15%, at least about 30%, at least about 40%, at least about 50%, at least about 60%, at least about 70%, at least about 80%, at least about 90%, or at least about 100% after the administration compared to the reference tumor volume.

In some aspects, treating a tumor comprises reducing a tumor weight in the subject. In some aspects, administering the cell composition of the disclosure (e.g., comprising a population of TILs prepared according to the methods disclosed herein (e.g., enriched for CD8⁺ TILs, tumor-specific TILs, and/or stem-like TILs)) can reduce the tumor weight in a subject when administered to the subject. In some aspects, the tumor weight is reduced by at least about 5%, at least about 10%, at least about 15%, at least about 30%, at least about 40%, at least about 50%, at least about 60%, at least about 70%, at least about 80%, at least about 90%, or at least about 100% after the administration compared to a reference tumor weight. In some aspects, the reference tumor weight is the tumor weight in the subject prior to the administration of the cell composition of the disclosure. In further aspects, the reference tumor weight is the tumor weight in a corresponding subject that did not receive the administration.

In some aspects, administering the cell composition of the disclosure (e.g., comprising a population of TILs prepared according to the methods disclosed herein (e.g., enriched for CD8⁺ TILs, tumor-specific TILs, and/or stem-like TILs)) to a subject, e.g., suffering from a tumor, can increase the number and/or percentage of TILs (e.g., CD8⁺ TILs) in a tumor and/or a tumor microenvironment (TME) of the subject. In some aspects, the number and/or percentage of TILs (e.g., CD8⁺ TILs) in a tumor and/or TME is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least 220%, at least about 230%, at least about 240%, at least about 250%, at least about 260%, at least about 270%, at least about 280%, at least about 290%, or at least about 300% or more compared to a reference (e.g., corresponding value in a subject that did not receive the cell composition of the present disclosure or the same subject prior to the administration of the cell composition of the present disclosure).

In some aspects, administering the cell composition of the disclosure (e.g., comprising a population of TILs prepared according to the methods disclosed herein (e.g., enriched for CD8⁺ TILs, tumor-specific TILs, and/or stem-like TILs)) to a subject, e.g., suffering from a tumor, can increase the duration of an immune response in a subject relative to the duration of an immune response in a subject administered a similar cell therapy comprising cells prepared according to conventional methods, e.g., cultured in a medium not comprising a potassium ion concentration of at least about 40 mM to at least about 90 mM, e.g., at least 50 mM. In some aspects, the duration of the immune response is increased by at least about 10%, at least about 20%, at least about 30%, at least about 40%, at least about 50%, at least about 75%, at least about 100%, at least about 150%, at least about 200%, at least about 300%, at least about 400%, at least about 500%, or at least about 1000% or more compared to a reference (e.g., a subject administered a similar cell therapy comprising cells prepared according to conventional methods, e.g., cultured in a medium not comprising a potassium ion concentration of at least 50 mM). In some aspects, the duration of the immune response is increased by at least about 2-fold, at least about 3-fold, at least about 4-fold, at least about 5-fold, at least about 6-fold, at least about 7-fold, at least about 8-fold, at least about 9-fold, or at least about 10-fold or more compared to a reference (e.g., a subject administered a similar cell therapy comprising cells prepared according to conventional methods, e.g., cultured in a medium not comprising a potassium ion concentration of at least about 40 mM to at least about 90 mM, e.g., at least 50 mM).

In addition to the above, administering the cell composition of the disclosure (e.g., comprising a population of TILs prepared according to the methods disclosed herein (e.g., enriched for CD8⁺ TILs, tumor-specific TILs, and/or stem-like TILs)) can have other effects which are conducive for the treatment of a tumor.

As described herein, the cell composition of the disclosure (e.g., comprising a population of TILs prepared according to the methods disclosed herein (e.g., enriched for CD8⁺ TILs, stem-like TILs, tumor-specific TILs, and/or naïve TILs)) can be used to treat variety of cancer types, e.g., a tumor derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof. In some aspects, the cancer comprises a solid tumor. In some aspects, the cancer comprises a solid tumor derived from a melanoma, a colon cancer, a lung cancer, a cervical cancer, a gastrointestinal cancer, a breast cancer, a prostate cancer, a liver cancer, bone cancer, a pancreatic cancer, a small cell carcinoma of the head and neck, lung squamous cell carcinoma, lung adenocarcinoma, pancreatic adenocarcinoma, head and neck squamous cell carcinoma, testicular germ cell tumors, stomach adenocarcinoma, skin cutaneous melanoma, mesothelioma, kidney renal clear cell carcinoma, cervical squamous cell carcinoma and endocervical adenocarcinoma, esophageal carcinoma, bladder urothelial carcinoma, breast invasive carcinoma, kidney renal papillary cell carcinoma, colon adenocarcinoma, or any combination thereof. In some aspects, the cancer comprises a melanoma. In some aspects, the cancer comprises colorectal cancer. In some aspects, the cancer comprises a colon cancer. In some aspects, the cancer comprises pancreatic cancer. In some aspects, the cancer comprises head and neck cancer. In some aspects, the cancer comprises cervical cancer. In some aspects, the cancer comprises ovarian cancer. In some aspects, the cancer comprises a lung cancer. In some aspects, the cancer comprises a gastrointestinal cancer. In some aspects, the cancer comprises a breast cancer. In some aspects, the cancer comprises a prostate cancer. In some aspects, the cancer comprises a liver cancer. In some aspects, the cancer comprises bone cancer. In some aspects, the cancer comprises a small cell carcinoma of the head and neck. In some aspects, the cancer comprises lung squamous cell carcinoma. In some aspects, the cancer comprises lung adenocarcinoma. In some aspects, the cancer comprises pancreatic adenocarcinoma. In some aspects, the cancer comprises head and neck squamous cell carcinoma. In some aspects, the cancer comprises a testicular germ cell tumor. In some aspects, the cancer comprises stomach adenocarcinoma. In some aspects, the cancer comprises skin cutaneous melanoma. In some aspects, the cancer comprises mesothelioma. In some aspects, the cancer comprises kidney renal clear cell carcinoma. In some aspects, the cancer comprises cervical squamous cell carcinoma. In some aspects, the cancer comprises endocervical adenocarcinoma. In some aspects, the cancer comprises esophageal carcinoma. In some aspects, the cancer comprises bladder urothelial carcinoma. In some aspects, the cancer comprises breast invasive carcinoma. In some aspects, the cancer comprises kidney renal papillary cell carcinoma. In some aspects, the cancer comprises colon adenocarcinoma. In some aspects, the cancer comprises a uterine cancer. In some aspects, the cancer comprises a brain. In some aspects, the cancer comprises a thyroid cancer. In some aspects, the cancer comprises an esophageal cancer. In some aspects, the cancer comprises an eye cancer. In some aspects, the cancer comprises a stomach (gastric) cancer. In some aspects, the cancer comprises a gastrointestinal cancer. In some aspects, the cancer comprises a sarcoma. In some aspects, the cancer comprises a leukemia. In some aspects, the cancer comprises a lymphoma. In some aspects, the cancer comprises a myeloma.

As such, some aspects of the present disclosure are directed to methods of treating a melanoma in a subject in need thereof, comprising administering to the subject a cell composition disclosed herein. Some aspects of the present disclosure are directed to methods of treating a colorectal cancer in a subject in need thereof, comprising administering to the subject a cell composition disclosed herein. Some aspects of the present disclosure are directed to methods of treating a colon cancer in a subject in need thereof, comprising administering to the subject a cell composition disclosed herein. Some aspects of the present disclosure are directed to methods of treating pancreatic cancer in a subject in need thereof, comprising administering to the subject a cell composition disclosed herein. Some aspects of the present disclosure are directed to methods of treating head and neck cancer in a subject in need thereof, comprising administering to the subject a cell composition disclosed herein. Some aspects of the present disclosure are directed to methods of treating cervical cancer in a subject in need thereof, comprising administering to the subject a cell composition disclosed herein. Some aspects of the present disclosure are directed to methods of treating ovarian cancer in a subject in need thereof, comprising administering to the subject a cell composition disclosed herein. Some aspects of the present disclosure are directed to methods of treating a lung cancer in a subject in need thereof, comprising administering to the subject a cell composition disclosed herein. Some aspects of the present disclosure are directed to methods of treating a gastrointestinal cancer in a subject in need thereof, comprising administering to the subject a cell composition disclosed herein. Some aspects of the present disclosure are directed to methods of treating a breast cancer in a subject in need thereof, comprising administering to the subject a cell composition disclosed herein. Some aspects of the present disclosure are directed to methods of treating a prostate cancer in a subject in need thereof, comprising administering to the subject a cell composition disclosed herein. Some aspects of the present disclosure are directed to methods of treating a liver cancer in a subject in need thereof, comprising administering to the subject a cell composition disclosed herein. Some aspects of the present disclosure are directed to methods of treating bone cancer in a subject in need thereof, comprising administering to the subject a cell composition disclosed herein. Some aspects of the present disclosure are directed to methods of treating a small cell carcinoma of the head and neck in a subject in need thereof, comprising administering to the subject a cell composition disclosed herein. Some aspects of the present disclosure are directed to methods of treating lung squamous cell carcinoma in a subject in need thereof, comprising administering to the subject a cell composition disclosed herein. Some aspects of the present disclosure are directed to methods of treating lung adenocarcinoma in a subject in need thereof, comprising administering to the subject a cell composition disclosed herein. Some aspects of the present disclosure are directed to methods of treating pancreatic adenocarcinoma in a subject in need thereof, comprising administering to the subject a cell composition disclosed herein. Some aspects of the present disclosure are directed to methods of treating head and neck squamous cell carcinoma in a subject in need thereof, comprising administering to the subject a cell composition disclosed herein. Some aspects of the present disclosure are directed to methods of treating a testicular germ cell tumor in a subject in need thereof, comprising administering to the subject a cell composition disclosed herein. Some aspects of the present disclosure are directed to methods of treating stomach adenocarcinoma in a subject in need thereof, comprising administering to the subject a cell composition disclosed herein. Some aspects of the present disclosure are directed to methods of treating skin cutaneous melanoma in a subject in need thereof, comprising administering to the subject a cell composition disclosed herein. Some aspects of the present disclosure are directed to methods of treating mesothelioma in a subject in need thereof, comprising administering to the subject a cell composition disclosed herein. Some aspects of the present disclosure are directed to methods of treating kidney renal clear cell carcinoma in a subject in need thereof, comprising administering to the subject a cell composition disclosed herein. Some aspects of the present disclosure are directed to methods of treating cervical squamous cell carcinoma in a subject in need thereof, comprising administering to the subject a cell composition disclosed herein. Some aspects of the present disclosure are directed to methods of treating endocervical adenocarcinoma in a subject in need thereof, comprising administering to the subject a cell composition disclosed herein. Some aspects of the present disclosure are directed to methods of treating esophageal carcinoma in a subject in need thereof, comprising administering to the subject a cell composition disclosed herein. Some aspects of the present disclosure are directed to methods of treating bladder urothelial carcinoma in a subject in need thereof, comprising administering to the subject a cell composition disclosed herein. Some aspects of the present disclosure are directed to methods of treating breast invasive carcinoma in a subject in need thereof, comprising administering to the subject a cell composition disclosed herein. Some aspects of the present disclosure are directed to methods of treating kidney renal papillary cell carcinoma in a subject in need thereof, comprising administering to the subject a cell composition disclosed herein. Some aspects of the present disclosure are directed to methods of treating colon adenocarcinoma in a subject in need thereof, comprising administering to the subject a cell composition disclosed herein. Some aspects of the present disclosure are directed to methods of treating a uterine cancer in a subject in need thereof, comprising administering to the subject a cell composition disclosed herein. Some aspects of the present disclosure are directed to methods of treating a brain tumor in a subject in need thereof, comprising administering to the subject a cell composition disclosed herein. Some aspects of the present disclosure are directed to methods of treating an esophageal cancer in a subject in need thereof, comprising administering to the subject a cell composition disclosed herein. Some aspects of the present disclosure are directed to methods of treating a thyroid cancer in a subject in need thereof, comprising administering to the subject a cell composition disclosed herein. Some aspects of the present disclosure are directed to methods of treating an eye cancer in a subject in need thereof, comprising administering to the subject a cell composition disclosed herein. Some aspects of the present disclosure are directed to methods of treating a stomach (gastric) cancer in a subject in need thereof, comprising administering to the subject a cell composition disclosed herein. Some aspects of the present disclosure are directed to methods of treating a gastrointestinal cancer in a subject in need thereof, comprising administering to the subject a cell composition disclosed herein. Some aspects of the present disclosure are directed to methods of treating a sarcoma in a subject in need thereof, comprising administering to the subject a cell composition disclosed herein. Some aspects of the present disclosure are directed to methods of treating a leukemia in a subject in need thereof, comprising administering to the subject a cell composition disclosed herein. Some aspects of the present disclosure are directed to methods of treating a lymphoma in a subject in need thereof, comprising administering to the subject a cell composition disclosed herein. Some aspects of the present disclosure are directed to methods of treating a myeloma in a subject in need thereof, comprising administering to the subject a cell composition disclosed herein.

In some aspects, the cell composition of the disclosure (e.g., comprising a population of TILs prepared according to the methods disclosed herein (e.g., enriched for CD8⁺ TILs, stem-like TILs, tumor-specific TILs, and/or naïve TILs)) can be used in combination with other therapeutic agents (e.g., anti-cancer agents and/or immunomodulating agents). Accordingly, in some aspects, a method of treating a tumor disclosed herein comprises administering the cell composition of the disclosure in combination with one or more additional therapeutic agents.

In some aspects, the cell composition of the disclosure (e.g., comprising a population of TILs prepared according to the methods disclosed herein (e.g., enriched for CD8⁺ TILs, stem-like TILs, tumor-specific TILs, and/or naïve TILs)) can be used in combination with one or more anti-cancer agents, such that multiple elements of the immune pathway can be targeted. In some aspects, an anti-cancer agent comprises an immune checkpoint inhibitor (i.e., blocks signaling through the particular immune checkpoint pathway).

Non-limiting examples of immune checkpoint inhibitors that can be used in the present methods comprise a CTLA-4 antagonist (e.g., anti-CTLA-4 antibody), PD1 antagonist (e.g., anti-PD1 antibody, anti-PD-L1 antibody), TIM-3 antagonist (e.g., anti-TIM-3 antibody), or combinations thereof. In some aspects, the checkpoint inhibitor is a PD1 antagonist. In some aspects, the checkpoint inhibitor is an anti-PD1 antibody. A comprehensive and non-limiting list of combination treatment is disclosed in detail elsewhere in this application.

In some aspects, the cell composition of the disclosure (e.g., comprising a population of TILs prepared according to the methods disclosed herein (e.g., enriched for CD8⁺ TILs, stem-like TILs, tumor-specific TILs, and/or naïve TILs)) is administered to the subject prior to or after the administration of the additional therapeutic agent. In other aspects, the cell composition of the disclosure (e.g., comprising a population of TILs prepared according to the methods disclosed herein (e.g., enriched for CD8⁺ TILs, stem-like TILs, tumor-specific TILs, and/or naïve TILs)) is administered to the subject concurrently with the additional therapeutic agent. In some aspects, the cell composition of the disclosure (e.g., comprising a population of TILs prepared according to the methods disclosed herein (e.g., enriched for CD8⁺ TILs, stem-like TILs, tumor-specific TILs, and/or naïve TILs)) and the additional therapeutic agent can be administered concurrently as a single composition in a pharmaceutically acceptable carrier. In other aspects, the cell composition of the disclosure (e.g., comprising a population of TILs prepared according to the methods disclosed herein (e.g., enriched for CD8⁺ TILs, stem-like TILs, tumor-specific TILs, and/or naïve TILs)) and the additional therapeutic agent are administered concurrently as separate compositions.

In some aspects, the subject is a nonhuman animal such as a rat or a mouse. In some aspects, the subject is a human.

In some aspects, a cell composition disclosed herein (e.g., comprising a population of TILs prepared according to the methods disclosed herein (e.g., enriched for CD8⁺ TILs, stem-like TILs, tumor-specific TILs, and/or naïve TILs)) can be used in combination with other therapeutic agents (e.g., anti-cancer agents and/or immunomodulating agents). Accordingly, in some aspects, a method of treating a tumor disclosed herein comprises administering a cell composition of the present disclosure (e.g., comprising a population of TILs prepared according to the methods disclosed herein (e.g., enriched for CD8⁺ TILs, stem-like TILs, tumor-specific TILs, and/or naïve TILs)) in combination with one or more additional therapeutic agents to a subject. Such agents can include, for example, chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof.

In some aspects, a cell composition disclosed herein (e.g., comprising a population of TILs prepared according to the methods disclosed herein (e.g., enriched for CD8⁺ TILs, stem-like TILs, tumor-specific TILs, and/or naïve TILs)) can be used in combination with a standard of care treatment (e.g., surgery, radiation, and chemotherapy). Methods described herein can also be used as a maintenance therapy, e.g., a therapy that is intended to prevent the occurrence or recurrence of tumors.

In some aspects, a cell composition of the present disclosure (e.g., comprising a population of TILs prepared according to the methods disclosed herein (e.g., enriched for CD8⁺ TILs, stem-like TILs, tumor-specific TILs, and/or naïve TILs)) can be used in combination with one or more anti-cancer agents, such that multiple elements of the immune pathway can be targeted. Non-limiting of such combinations include: a therapy that enhances tumor antigen presentation (e.g., dendritic cell vaccine, GM-C S F secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); an additional adoptive T cell or NK cell transfer including genetically engineered cells, e.g., cells engineered to express a chimeric antigen receptor (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; blocking of immuno repressive cytokines; or any combination thereof.

In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD1 antagonist (e.g., anti-PD1 antibody, anti-PD-L1 antibody), TIM-3 antagonist (e.g., anti-TIM-3 antibody), or combinations thereof. Non-limiting examples of such immune checkpoint inhibitors include the following: anti-PD1 antibody (e.g., nivolumab)(OPDIVO®, pembrolizumab (KEYTRUIDA®; MK-3475), pidilizumab (CT-011), PDR001, MEDI0680 (AMP-514), TSR-042, REGN2810, JS001, AMP-224 (GSK-2661380), PF-06801591, BGB-A317, BI 754091, SHR-1210, and combinations thereof); anti-PD-L1 antibody (e.g., atezolizumab (TECENTRTQ®; RG7446; MPDL3280A; R05541267), durvalumab (MEDI4736, IMFINZI®), BMS-936559, avelumab) BAVENCI®), LY3300054, CX-072 (Proclaim-CX-072), FAZ053, KN035, MDX-1105, and combinations thereof); and anti-CTLA-4 antibody (e.g., ipilimumab) YERVOY®), tremelimumab (ticilimumab; CP-675,206), AGEN-1884, ATOR-1015, and combinations thereof).

In some aspects, the cell composition of the present disclosure (e.g., comprising a population of TILs prepared according to the methods disclosed herein (e.g., enriched for CD8⁺ TILs, stem-like TILs, tumor-specific TILs, and/or naïve TILs)) is administered to a subject in combination with a PD1 antagonist. In some aspects, the cell composition of the present disclosure (e.g., comprising a population of TILs prepared according to the methods disclosed herein (e.g., enriched for CD8⁺ TILs, stem-like TILs, tumor-specific TILs, and/or naïve TILs)) is administered to a subject in combination with an anti-PD1 antibody. In some aspects, the cell composition of the present disclosure (e.g., comprising a population of TILs prepared according to the methods disclosed herein (e.g., enriched for CD8⁺ TILs, stem-like TILs, tumor-specific TILs, and/or naïve TILs)) is administered to a subject in combination with nivolumab. In some aspects, the cell composition of the present disclosure (e.g., comprising a population of TILs prepared according to the methods disclosed herein (e.g., enriched for CD8⁺ TILs, stem-like TILs, tumor-specific TILs, and/or naïve TILs)) is administered to a subject in combination with pembrolizumab.

In some aspects, the cell composition of the present disclosure (e.g., comprising a population of TILs prepared according to the methods disclosed herein (e.g., enriched for CD8⁺ TILs, stem-like TILs, tumor-specific TILs, and/or naïve TILs)) is administered to a subject in combination with a PD-L1 antagonist. In some aspects, the cell composition of the present disclosure (e.g., comprising a population of TILs prepared according to the methods disclosed herein (e.g., enriched for CD8⁺ TILs, stem-like TILs, tumor-specific TILs, and/or naïve TILs)) is administered to a subject in combination with an anti-PD-L1 antibody. In some aspects, the cell composition of the present disclosure (e.g., comprising a population of TILs prepared according to the methods disclosed herein (e.g., enriched for CD8⁺ TILs, tumor-specific TILs, and/or naïve TILs)) is administered to a subject in combination with atezolizumab. In some aspects, the cell composition of the present disclosure (e.g., comprising a population of TILs prepared according to the methods disclosed herein (e.g., enriched for CD8⁺ TILs, tumor-specific TILs, and/or naïve TILs)) is administered to a subject in combination with durvalumab. In some aspects, the cell composition of the present disclosure (e.g., comprising a population of TILs prepared according to the methods disclosed herein (e.g., enriched for CD8⁺ TILs, tumor-specific TILs, and/or naïve TILs)) is administered to a subject in combination with avelumab.

In some aspects, the cell composition of the present disclosure (e.g., comprising a population of TILs prepared according to the methods disclosed herein (e.g., enriched for CD8⁺ TILs, stem-like TILs, tumor-specific TILs, and/or naïve TILs)) is administered to a subject in combination with a CTLA-4 antagonist. In some aspects, the cell composition of the present disclosure (e.g., comprising a population of TILs prepared according to the methods disclosed herein (e.g., enriched for CD8⁺ TILs, stem-like TILs, tumor-specific TILs, and/or naïve TILs)) is administered to a subject in combination with an anti-CTLA-4 antibody. In some aspects, the cell composition of the present disclosure (e.g., comprising a population of TILs prepared according to the methods disclosed herein (e.g., enriched for CD8⁺ TILs, stem-like TILs, tumor-specific TILs, and/or naïve TILs)) is administered to a subject in combination with ipilimumab. In some aspects, the cell composition of the present disclosure (e.g., comprising a population of TILs prepared according to the methods disclosed herein (e.g., enriched for CD8⁺ TILs, stem-like TILs, tumor-specific TILs, and/or naïve TILs)) is administered to a subject in combination with tremelimumab.

In some aspects, an anti-cancer agent comprises an immune checkpoint activator (i.e., promotes signaling through the particular immune checkpoint pathway). In some aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), TIM3 agonist (e.g., anti-TIM3 antibody), or combinations thereof. In some aspects, the additional therapeutic agent comprises a cytokine. In some aspects, the cytokine comprises IL-2, 11-21, 11-7, 11-15, or any combination thereof.

In some aspects, a cell composition disclosed herein (e.g., comprising a population of TILs prepared according to the methods disclosed herein (e.g., enriched for CD8⁺ TILs, stem-like TILs, tumor-specific TILs, and/or naïve TILs)) is administered to the subject prior to or after the administration of the additional therapeutic agent. In other aspects, cell composition disclosed herein (e.g., comprising a population of TILs prepared according to the methods disclosed herein (e.g., enriched for CD8⁺ TILs, stem-like TILs, tumor-specific TILs, and/or naïve TILs)) is administered to the subject concurrently with the additional therapeutic agent. In some aspects, the cell composition disclosed herein (e.g., comprising a population of TILs prepared according to the methods disclosed herein (e.g., enriched for CD8⁺ TILs, stem-like TILs, tumor-specific TILs, and/or naïve TILs)) and the additional therapeutic agent can be administered concurrently as a single composition in a pharmaceutically acceptable carrier. In other aspects, the cell composition disclosed herein (e.g., comprising a population of TILs prepared according to the methods disclosed herein (e.g., enriched for CD8+ TILs, stem-like TILs, tumor-specific TILs, and/or naïve TILs)) and the additional therapeutic agent are administered concurrently as separate compositions. In some aspects, the additional therapeutic agent and the cell composition disclosed herein (e.g., comprising a population of TILs prepared according to the methods disclosed herein (e.g., enriched for CD8+ TILs, stem-like TILs, tumor-specific TILs, and/or naïve TILs)) are administered sequentially.

In some aspects, a cell composition disclosed herein (e.g., comprising a population of TILs prepared according to the methods disclosed herein (e.g., enriched for CD8⁺ TILs, stem-like TILs, tumor-specific TILs, and/or naïve TILs)) is administered to the subject in combination with a checkpoint inhibitor (e.g., an anti-PD1 antibody). In some aspects, the cell composition is administered before the checkpoint inhibitor (e.g., an anti-PD1 antibody). In some aspects, the cell composition is administered after the checkpoint inhibitor (e.g., an anti-PD1 antibody).

In some aspects, the subject is administered a lymphodepleting therapy prior to receiving the cell composition. Any lymphodepleting therapy can be used in the method disclosed herein. In some aspects, the lymphodepleting therapy comprises a chemotherapy. In some aspects, the lymphodepleting therapy comprises cyclophosphamide. In some aspects, the lymphodepleting therapy comprises fludarabine. In some aspects, the lymphodepleting therapy comprises cyclophosphamide and fludarabine. In some aspects, the lymphodepleting therapy is administered at least about 3 days, at least about 4 days, at least about 5 days, at least about 7 days, at least about 8 days, at least about 9 days, at least about 10 days, at least about 11 days, at least about 12 days, at least about 13 days, or at least about 14 days prior to the cell composition.

### Examples

### Example 1. Methods

Media preparation: T cell conditioned media (TCM) was supplemented with immune Cell Serum Replacement (Thermo Fisher), 2 mM L-glutamine (Gibco), 2 mM Glutamax (Gibco), MEM Non-Essential Amino Acids Solution (Gibco), Sodium pyruvate (Gibco), IL-2, 200 IU/mL; IL-7, 120 IU/ml; IL-15, 20 IU/ml.

For hypotonic conditioning medium, TCM media with varying concentrations of sodium, potassium, glucose and calcium were adjusted by adding NaCl, glucose, and calcium free RPMI. After adding defined NaCl free RPMI to TCM, the final concentrations were in the range of: NaCl (40-80 mM), KCl (40-80 mM), Calcium (0.5-2.8 mM), Glucose (10-24 mM) and osmolality (˜250-260 mOsmol). See Table. 1.

TABLE 1
Hypotonic conditioning medium with varying concentrations
of potassium, sodium, glucose, and calcium

| | | | Glucose | | Osmolality | Tonicity* |
|---|---|---|---|---|---|---|
| Media | K (mM) | NaCl (mM) | (mM) | Ca (mM) | (mOsmol) | (mOsmol) |
| Basal Media | 4 | 118.47 | ~24 mM | ~2.8 mM | 245 | 245 |
| Hyper K | 80 mM | 55.6 mM | 15 mM | 1.2 mM | ~262.26 | 271.2 |
| Hyper K | 75 | 59.3 | 15.4 | 1.3 | ~260 | 268.6 |
| Hyper K | 70 | 63.9 | 15.9 | 1.4 | ~259.7 | 267.8 |
| Hyper K | 65 | 67.6 | 16.3 | 1.5 | ~257.5 | 265.2 |
| Hyper K | 60 | 72.2 | 16.8 | 1.6 | ~257.2 | 264.4 |
| Hyper K | 55 | 76 | 17.2 | 1.7 | ~255.2 | 262 |
| Hyper K | 50 | 80.5 | 17.7 | 1.8 | ~254.7 | 261 |
| RPMI Gibco + | 5.34 | 103 | 11.1 | 0.4 | | 216.7 |
| ICSR | | | | | | |
| RPMI 1640 + 50 mM | 55.34 | 103 | | | | 316.7 |
| K+ | | | | | | |

*Tonicity is calculated based on the following formula: 2 × (concentration of K + concentration of NaCl)

We also tested the effect of tonicity on T cells by maintaining constant tonicity conditions (250 mOsmol—hypotonic, 280 mOsmol—isotonic, 320 mOsmol—hypertonic) with varying potassium concentrations. Final concentrations in hypotonic conditions, NaCl (35-75 mM), KCl (50-90 mM), final concentrations in isotonic conditions NaCl (50-90 mM), KCl (50-90 mM), final concentrations in hypertonic conditions NaCl (70-110 mM), KCl (50-90 mM). See Table. 2.

TABLE 2
Hypotonic, isotonic, hypertonic solutions with varying
concentrations of potassium and NaCl

| | | K | NaCl |
|---|---|---|---|
| Tonicity* | mOsmol | (mM) | (mM) |
| Hypotonic | 250 | 50 | 75 |
| | | 60 | 65 |
| | | 70 | 55 |
| | | 80 | 45 |
| | | 90 | 35 |
| Isotonic | 280 | 50 | 90 |
| | | 60 | 80 |
| | | 70 | 70 |
| | | 80 | 60 |
| | | 90 | 50 |
| Hypertonic | 320 | 50 | 110 |
| | | 60 | 100 |
| | | 70 | 90 |
| | | 80 | 80 |
| | | 90 | 70 |

*Tonicity is calculated according to the formula: Tonicity = ([K] + [NaCl]) × 2 wherein “[K]” is the potassium concentration and “[NaCl]” is the sodium chloride concentation of the media.

Cell culture and Transduction: Healthy donor cryopreserved human CD4 and CD8 cells were activated with TransAct (Miltenyi) in T cell conditioned media-TCM, basal media, or hypotonic conditioning medium. After 24 hours of activation in the TCM, basal media, or hypotonic conditioning medium, T cells were transduced with lentiviral particles to introduce chimeric antigen receptor (anti-CD19 CAR) in Grex plates (Wilson Wolf). The following day after transduction, T cells were supplemented with fresh media to dilute the TransAct and end T-cell activation. Depending on the cell growth and density, T cells were fed with warm 2× cytokine media by aspirating half of the media in the Grex plate. On day 7, cells were harvested, counted and analyzed for the expression of stemness markers by flow cytometry.

Intracellular Cytokine assays: On day 7, T cells were washed and placed in control media and subjected to a 5 hour re-stimulation with phorbol myrystate acetate (PMA) and ionomycin in the presence of brefeldin A to measure intracellular cytokines, IL-2, IFNγ, and TNFα. T cells were stained with surface antibody staining in FACS buffer containing fixable live/dead solution. Cells were stained with respective antibodies for intracellular cytokines following fixation and permeabilization. Quantification of intracellular cytokine expression was assessed using flow cytometry.

Sternness phenotype CAR expression measurement via flow cytometry: On day 7, live T cells from the respective treatments were assessed via flow cytometry. Cells were first washed with cell staining buffer and stained with anti-CCR7 for 15 minutes at 37° C. Following this, a 2× master mix of the antibodies against several other antigens (as detailed below) was added to cells and incubated for 20 minutes at 4° C. Cells were washed with cell staining buffer and permeabilized with the foxp3 staining kit (ebioscience) as per manufacturers' protocol. After fixing, the cells were stained for TCF7 for twenty minutes at 4° C. following which, cells were analyzed by flow cytometry on aurora (cytek). The following are the list of antibodies used for assessing the stemness markers: CD8 (BD-#563795), CD4 (BD-#612936), CD27 (BD-#612829), CD3 (Thermo-#612893), CD28 (Biolegend-#302936), CD62L, CAR-EGFR (Thermo-#352911), CD45RO (BD #564290), CD39 (Biolegend-#328236), TCF7 (Cell signaling-#14456), CCR7 (BD-#562381), CD127 (Bio legend-#351324), CD45RA (BD-#560673).

### Example 2. Methods of Preparing Media and Culturing TILs

Control Media: Commercially available T cell media (e.g., CTS™ OPTIMIZER™, IMMUNOCULT™ or TEXMACS™). Metabolic reprogramming media (“MRM”): The inorganic salt ion concentrations of T cell media were adjusted using NaCl free T cell media. The final concentrations of MRM were the following: NaCl (40-80 mM), KCl (40-90 mM), Calcium (0.5-2.8 mM), Glucose (10-24 mM) and osmolality (˜250-340 mOsmol).

TIL media preparation used for initial culture and secondary and final TIL expansions: Either Control media or MRM was supplemented with 2.5% serum supplement (CTS™ Immune Cell SR, Thermo Fisher), 2 mM L-glutamine (Gibco), 2 mM L-glutamax (Gibco), MEM Non-Essential Amino Acids Solution (Gibco), Pen-strep (Gibco), 20 μg/ml FUNGIN™ (InvivoGen), Sodium pyruvate (Gibco), and 1 mM of O-Acetyl-L-carnitine hydrochloride (Sigma).

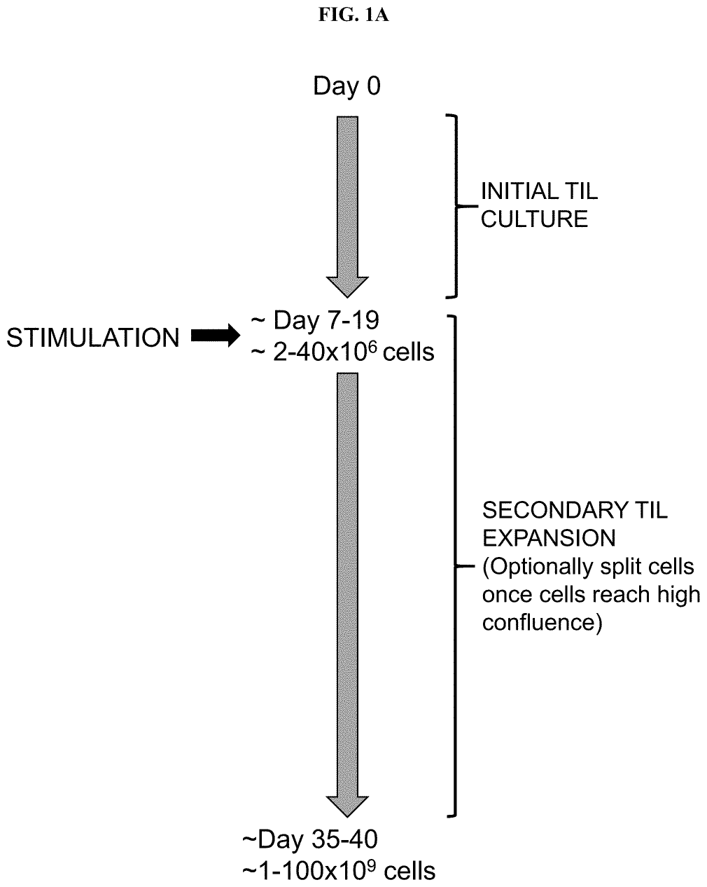

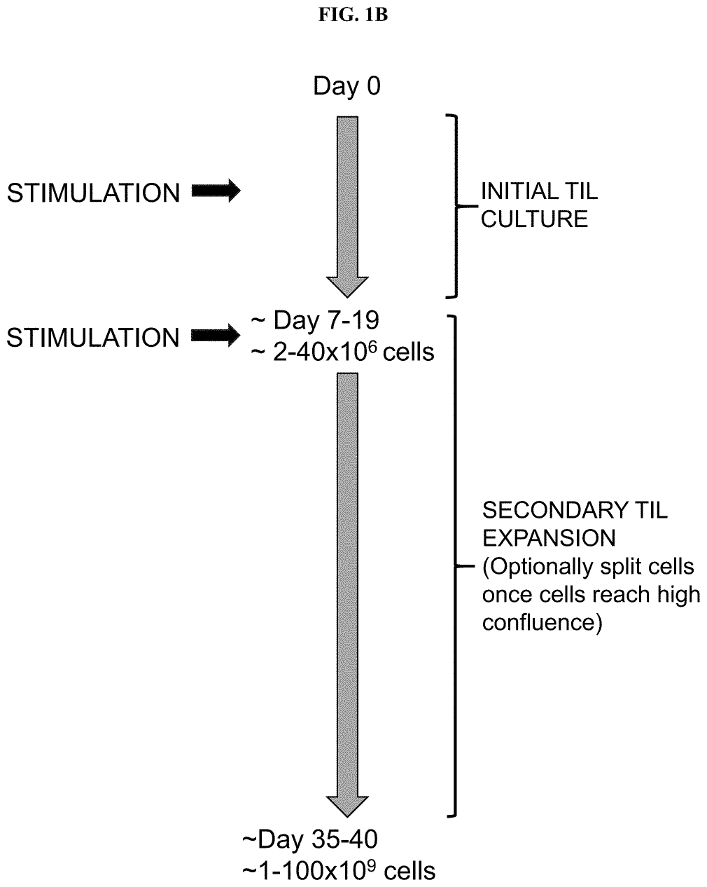

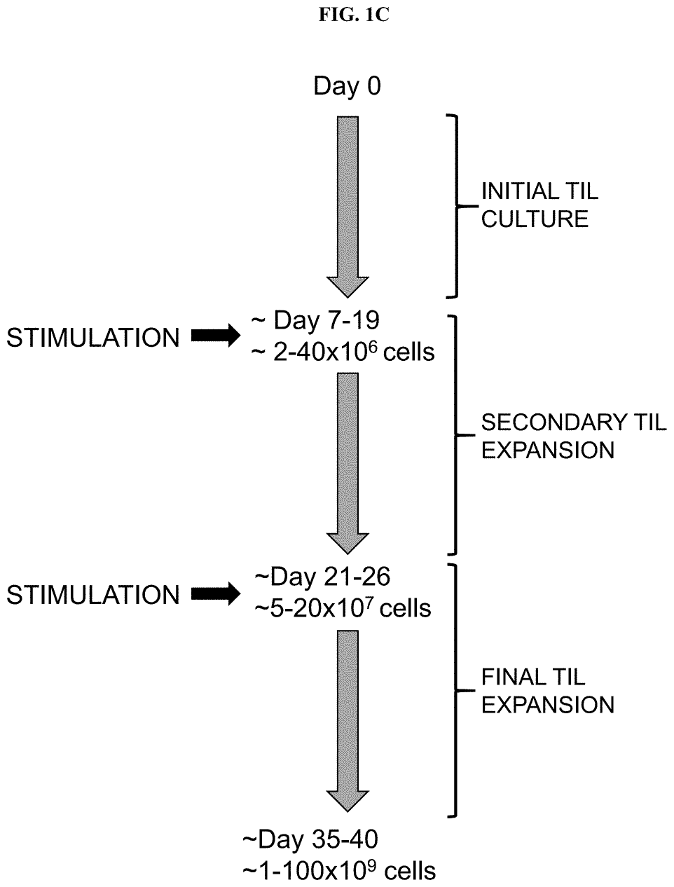

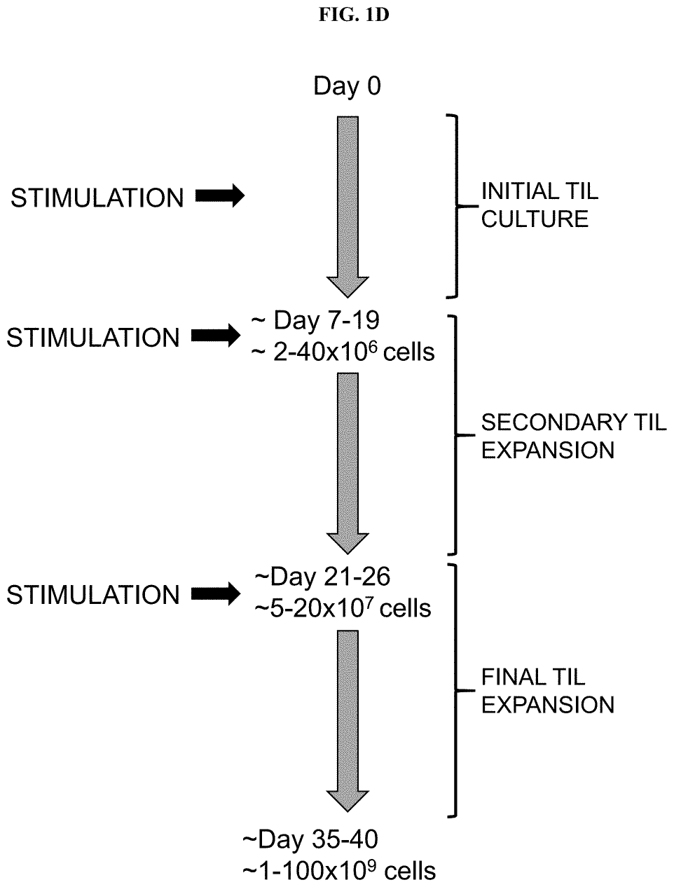

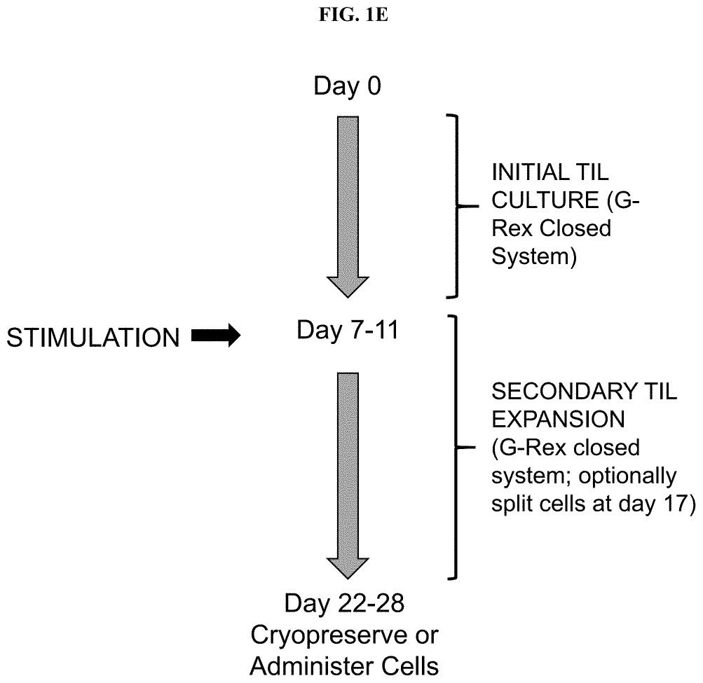

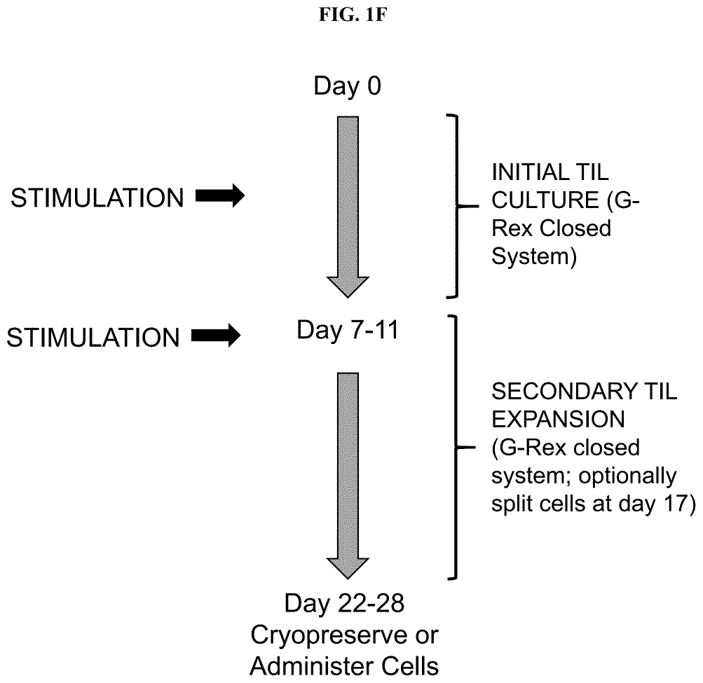

Initial TIL Culture: FIG. 1 is a schematic depicting generally certain aspects of the methods of culturing TILs described herein. Multiple tumors surgically resected from various tumor types (colon, lung, hepatocellular carcinoma, renal, pancreas, breast, melanoma, and prostate) with an average size of 1-10 mm³ were seeded in 24-well plates in 2 ml of either control media or MRM as described above, both supplemented with IL-2 (300 ng/mL) and IL-21 (30 ng/ml). Tumor fragments were cultured in a heat jacketed incubator at 37° C. incubator with 5% CO₂ until colony formation was visible. Fresh media (control or MRM) supplemented with IL-2 (300 ng/mL) and IL-21 (30 ng/ml) were replenished every 3 days depending on the growth of the cells. This method resulted in a yield of about 2×10⁶-10×10⁶ cells per fragment at the end of the initial culture. A subset of cells for analysis were passed through a 40 μm strainer and pheonotyped with multi color flow cytometry using various biomarkers including CD62L, CD27, CD28, CD45RO, CD39, TIM3, CD127, PD1, CD103, CD45RA, and TCF7.

Secondary TIL Expansion: When cell yield from the initial culture reached about 2×10⁶-10×10⁶ cells per cultured fragment (usually at about day 14 to day 19), the TILs cultured in either control media or MRM, both supplemented with IL-2 (73.6 ng/ml), IL-21 (10 ng/ml), and IL-15 (0.4 ng/ml), were stimulated by adding 1:100 T cell TRANSACT™ (Miltenyi Biotec), 5 μg/ml recombinant human CD27 ligand (R&D systems), and 1 μg/ml recombinant human 4-1BB ligand/TNFSF9 (R&D systems). Cells were maintained in culture until about 5×10⁷ to 20×10⁷ cells were obtained (about 7 to 11 days post-stimulation). At the end of the secondary expansion period, TILs were analyzed with multicolor flow cytometry using various biomarkers including, CD62L, CD27, CD28, CD45RO, CD39, TIM3, CD127, PD1, CD103, CD45RA and TCF7. Only live and CD3⁺ cells were analyzed.

Final TIL Expansion: When the cultures reached a yield of about 5×10⁷ to 20×10⁷ cells, the TILs cultured in either control media or MRM were transferred to fresh control media supplemented with IL-2 (73.6 ng/ml), IL-21 (10 ng/ml), and IL-15 (0.4 ng/ml). TILs were stimulated for a second time with 1:100 TRANSACT™ (Miltenyi Biotec), 5 μg/ml recombinant human CD27 ligand (R&D systems), and 1 μg/ml recombinant human 4-1BB ligand/TNFSF9 (R&D systems). The cells were cultured in static GREX or stirred tank until a yield of about 10×10⁹-100×10⁹ cells per fragment was achieved (about 14 days) and analyzed with multi color flow cytometry for various biomarkers, including CD62L, CD27, CD28, CD45RO, CD39, TIM3, CD127, PD1, CD103, CD45RA, and TCF7. To check for polyfunctionality of the cells, the resultant TILs were stimulated with PMA/ionomycin (1:500) for 4 hours and intracellular staining was performed using the following markers: CD4, CD8, CD27, IL2, IFNγ, TNFα and TCF7. Only live and CD3⁺ cells were analyzed.

### Example 3. MRM Results in Expansion of CD8+, Tumor-Reactive, Less Differentiated TILs

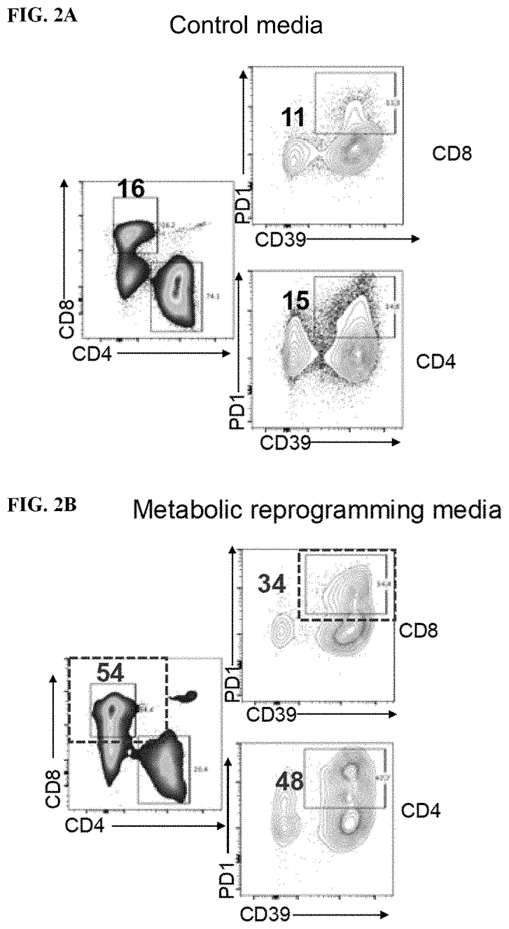

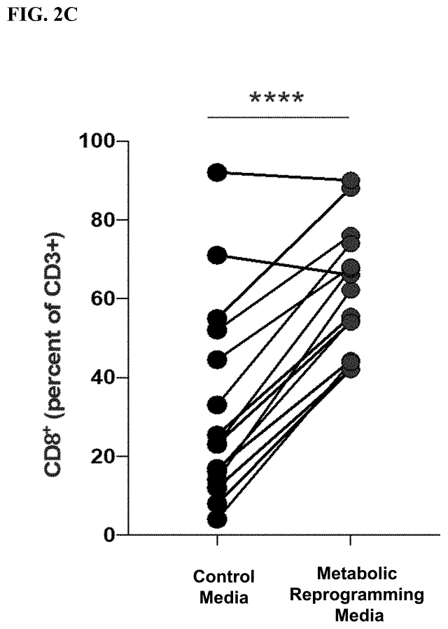

TILs were grown as described in Example 1 (FIG. 1). After the initial TIL culture (i.e., 14 days), multiparameter flow cytometry was performed to quantify the percentages of CD4⁺ and CD8⁺ TILs present in the cell culture. Cells cultured in MRM had significantly enriched CD8⁺ TILs by ˜20-80% as compared to the cells cultured in control media (FIGS. 2A-2C and data not shown). Although CD4⁺ TILs are capable of eradicating solid tumors, superior cytolytic activity towards tumors is primarily mediated by CD8⁺ TILs. Tumor cells predominantly express MHC class I associated tumor antigens, which are recognized by CD8⁺ TILs. Thus, having a greater proportion of CD8+ TILs in the TIL therapy infusion product is therapeutically beneficial. Use of MRM to culture TILs unexpectedly enriched CD8+ TILs as compared to TILs cultured in control media (FIG. 2C).

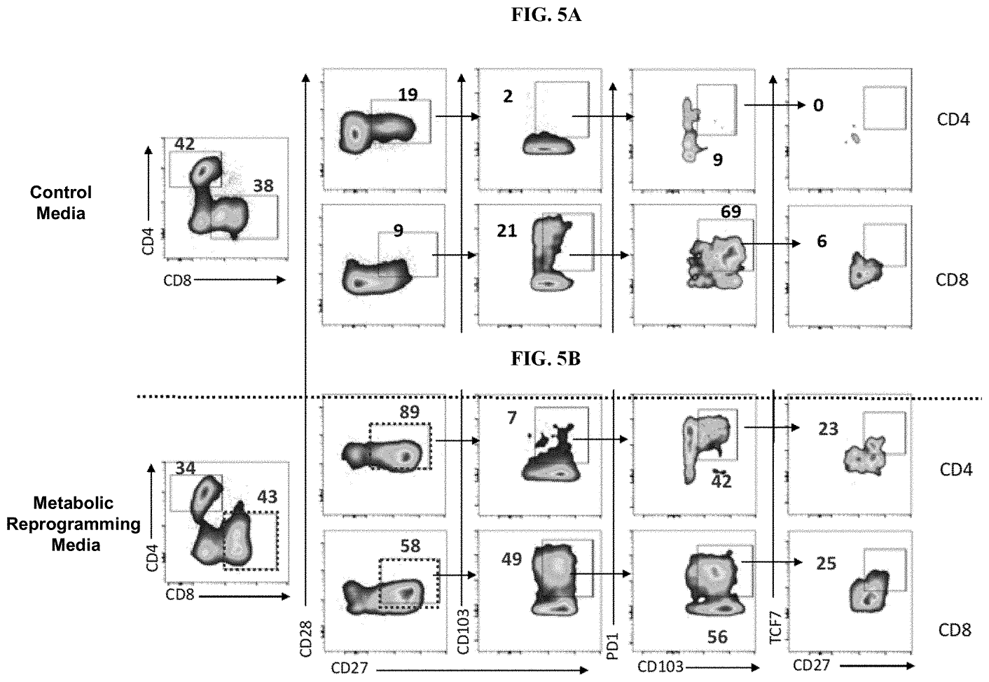

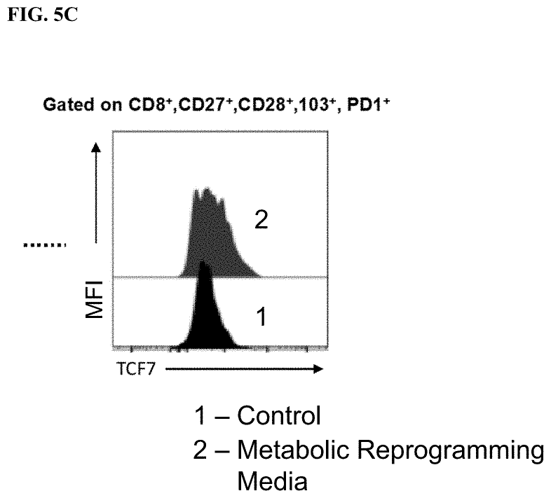

TILs obtained at the end of the initial culture in MRM (about day 14) also demonstrated consistent expression of several cell surface markers of tumor-reactive TILs (e.g., CD39, CD103, CD226, and/or PD1). Initial culturing in MRM produced TILs with enhanced expression of CD39 and PD1 (greater than 20%) as compared to TILs cultured in control media (FIGS. 2A-2B). Previously used methods show that PD1 expression is completely lost in the PD1 subsets during initial TIL culture, indicating undesirable loss of tumor-reactive TILs (see, e.g., Poschke et al, Oncoimmunology 5(12):e1240859 (2016); and Gros et al, JCI 124(5):2246-59 (2014)). We obseverved that maintenance of PD1 expression after T cell stimulation was dependent on donor (data not shown). Similar results were obtained for expression of other markers of tumor reactivity including CD39 and CD103 (see FIGS. 2A-2B and 5A-5B, and data not shown). However, clonal repertoire observed at day 14 is maintained throughout entire TIL process (data not shown).

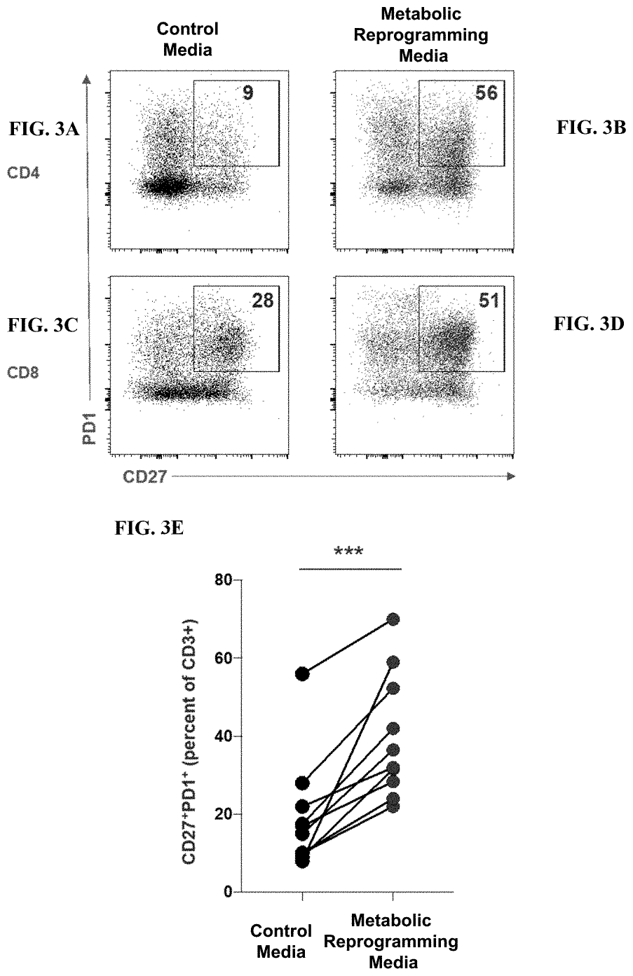

We also observed co-expression of PD1 (indicative of a tumor-reactive metabolic state) and CD27, in both CD4⁺ and CD8⁺ TILs cultured in MRM (FIGS. 3A-3E). CD27 expression, which is constitutively expressed on naïve and memory committed T-cells, is indicative of a stem-like phenotype in T cells. Previous reports indicate that CD27 expression is reduced in CD8⁺ T cells during cell expansion following T cell stimulation (see, e.g., Tran et al., J. Immunotherapy 31(8):742-51 (2008); and Rosenberg et al., Clinical Cancer Research 17(13):4550-557 (2011), Huang et al, J. Immunology 176(12):7726-35 (2006)). In contrast, TILs cultured in MRM, disclosed herein, have preserved CD27 expression throughout the culturing process, allowing for selective expansion of stem-like tumor-reactive clones. As expected, not all CD27⁺ cells at day 14 co-expressed PD1, indicating that not all stem-like cells displayed a tumor-reactive metabolic state.

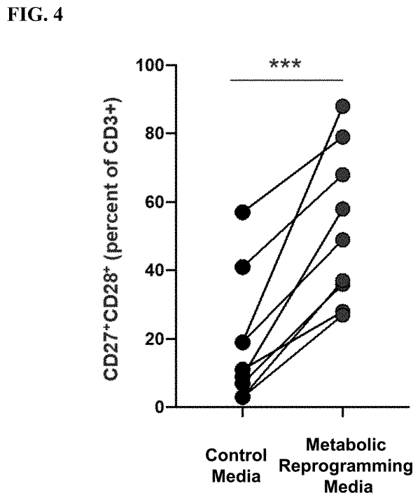

Anti-tumor function and survival of TILs are dependent on the consolidated signals received by the TCR, cytokine, and costimulatory receptors. Inadequate exposure of any of these signals will result in anergy and atrophy of the TILs. Previously used methods of culturing and expanding TILs result in loss of CD27 expression in the TILs. However, TILs that maintain CD27 expression in an infusion product, e.g., in minimally expanded TILs, have been shown to be associated with tumor regression following adoptive T cell therapy (see, e.g., Tran et al., J. Immunotherapy 31(8):742-51 (2008); and Rosenberg et al., Clinical Cancer Research 17(13):4550-557 (2011)). In addition, elevated expression of costimulatory receptors, e.g., CD27 and CD28, is associated with in vivo therapeutic efficacy (Tran et al., J. Immunotherapy 31(8):742-51 (2008), Geltink et al., Cell 171, 385-397 (2017)). Expression of these costimulatory receptors has also been associated with stemness and longer telomere lengths that correlated with young TIL cultures. In contrast to previously used methods, e.g., control media, the use of MRM described herein enriched TILs with CD27 and CD28 expression to about 20% to about 80% of the total number of TILs across several tumor types (FIG. 4 and data not shown). Enrichment of CD27 and CD28 was not unique to the CD8⁺ T cell subset but was also observed in the CD4⁺ subset (data not shown).

Expression of CD27 and CD62L is correlated with less differentiated T cells and is associated with efficient trafficking of the T cells to tumor tissues and lymph nodes. Expression of CD27, CD28, and CD62L is also linked to longer telomere length, which is indirectly linked to the age of the TIL and in vivo therapeutic efficacy (see, e.g., Tran et al., J. Immunotherapy 31(8):742-51 (2008); and Rosenberg et al., Clinical Cancer Research 17(13):4550-557 (2011)). However, TILs cultured in MRM, as disclosed herein, maintained both CD27 and CD62L expression throughout the process, similar to that of minimally cultured TILs. We observed a ˜50% increase in CD27 and CD62L expression in TILs cultured in MRM as compared to those cultured in control media (data not shown).

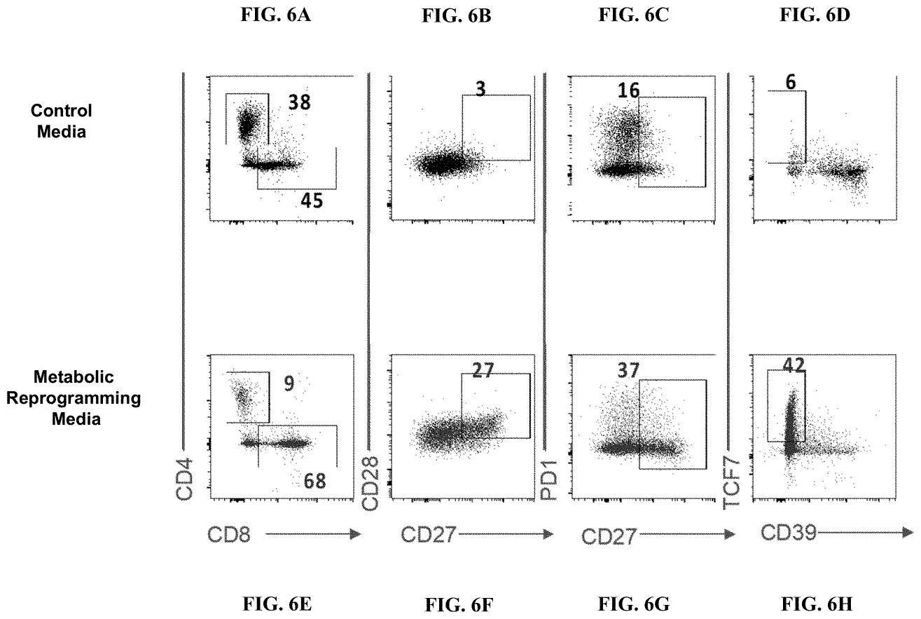

Tumor-reactive clones that mediate tumor regression post immune checkpoint blockade are believed to be derived from the CD8⁺ T cells that expressed PD1 and transcription factor TCF-7 (Im et al., Nature 537:417-21 (2016); and Feldman et al., Cell 175(4):998-1013 (2018)). TILs cultured in MRM disclosed herein displayed enrichment of tumor-reactive TIL biomarkers (PD1 and CD103) with a concurrent 4-fold to 50-fold higher level of TCF7 expression (FIGS. 5A-5C, 6D, 6H, 10). Expression of TCF7 is indicative of more stem-like cells. Despite the expression of PD1, these cells retained proliferative capacity and maintained less differentiated cells upon further stimulation (FIGS. 6A-6H).

### Example 4: MRM Preserves Tumor Reactivity of TILs

Tumors are heterogenous in nature and often contain common mutations in genes such as KRAS, P53, and BRAF (public neoantigens). The methods using MRM as disclosed herein enriched for TILs that recognize such neoantigens.

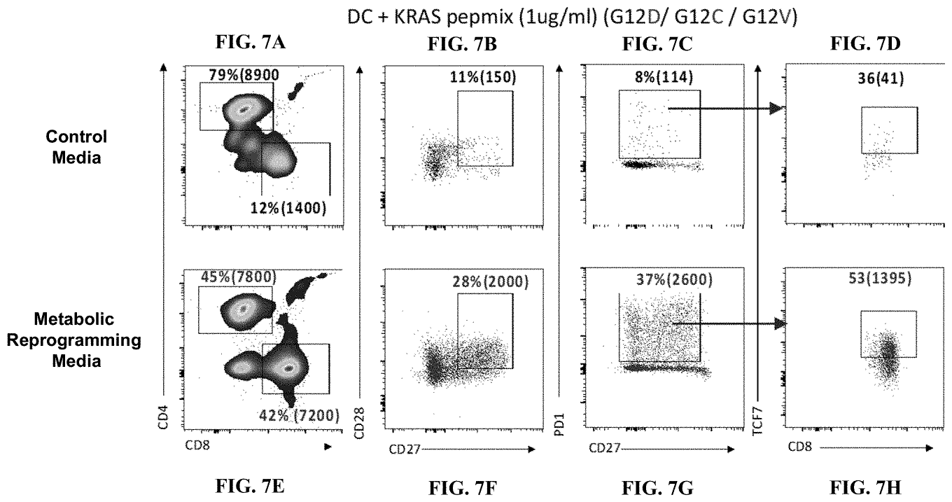

Tumor resections were obtained from a patient with pancreatic adenocarcinoma, a cancer that predominantly has tumor cells with KRAS^{G12V}, KRAS^{G12C}, and KRAS^{G12D} mutations. HPLC-purified 9mer, 10mer, and 25mer peptides of the above-mentioned KRAS hot spot mutations were purified and used to pulse immature dendritic cells (DCs) generated from patient-matched peripheral blood monocytes. These peptide pool-pulsed DCs were co-cultured with TILs obtained from the same pancreatic adenocarcinoma patient and cultured according to the methods described in the Examples above. Culture of the TILs in MRM resulted in ˜30% more CD8⁺ T cells with significant co-expression of CD27, CD28, PD1, and TCF-7 (FIGS. 7A-7H). These results indicate that MRM disclosed herein preserves tumor-reactive TCR clones that recognize public antigens.

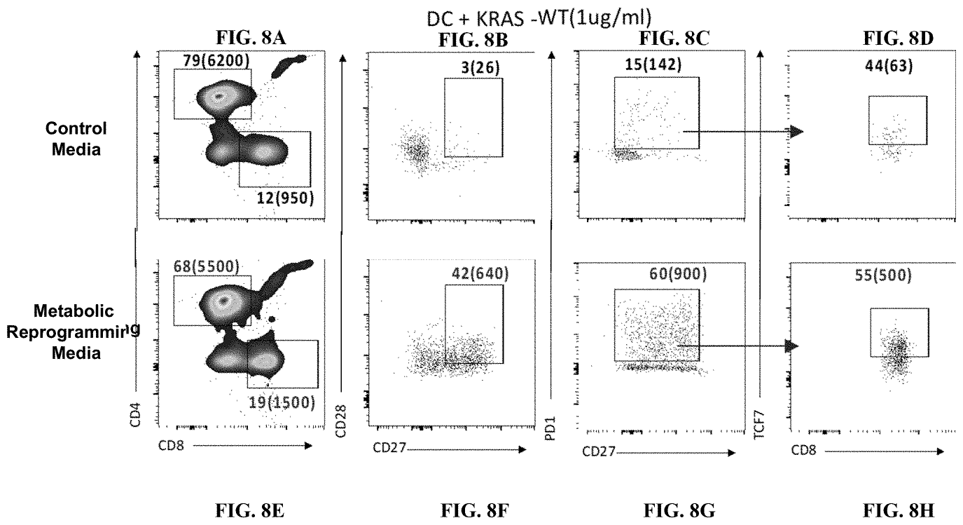

As expected, TILs pulsed with wild type KRAS peptides did not result in specific expansion of KRAS-specific TILs. However, we consistently observed increased expression of CD27, CD28, PD1, and TCF7 expression by TILs cultured in MRM. These results indicate that culturing cells in MRM preserves the growth and proliferation of TILs that recognize rare public neoantigens (FIGS. 8A-8H).

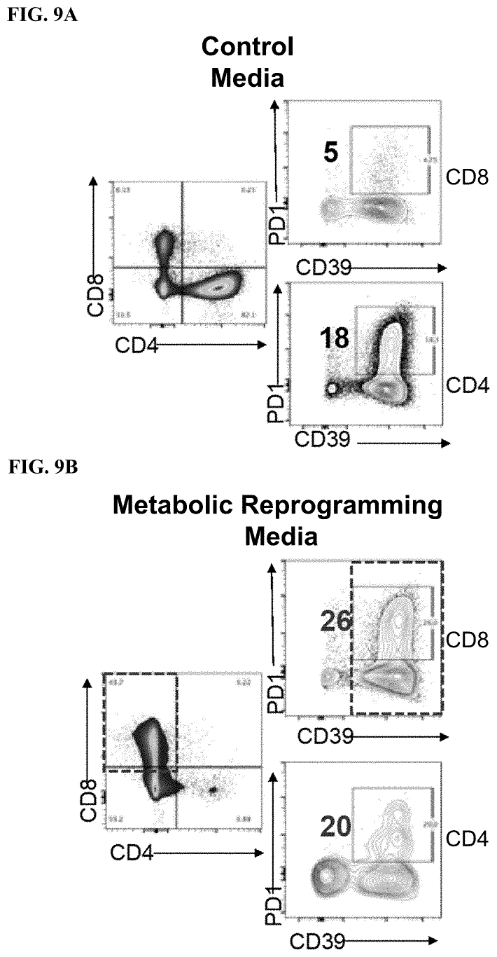

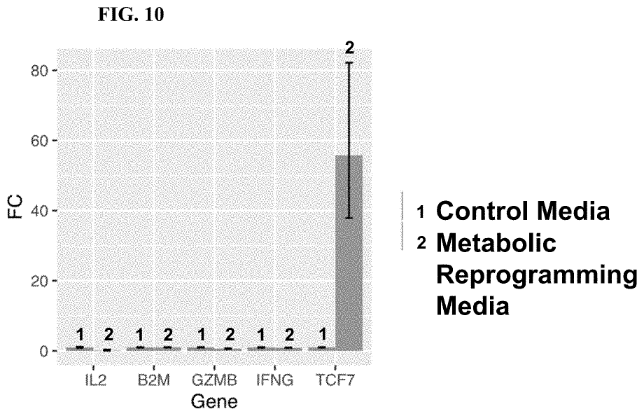

Preferential enrichment of CD8⁺ TILs and markers associated with tumor reactivity (CD39 and PD1) were maintained during this expansion process (FIGS. 9A-9B). These results show that MRM further enhances PD1 expression in tumor-reactive TILs even after initial culture. In addition, TILs cultured in MRM according to the methods disclosed herein exhibited a greater than 50-fold increase in TCF7 expression as measured by qPCR (FIG. 10). These data are consistent with the role of TCF7, which is a master transcriptional regulator required for self-renewal and proliferative burst of the TILs post re-stimulation.

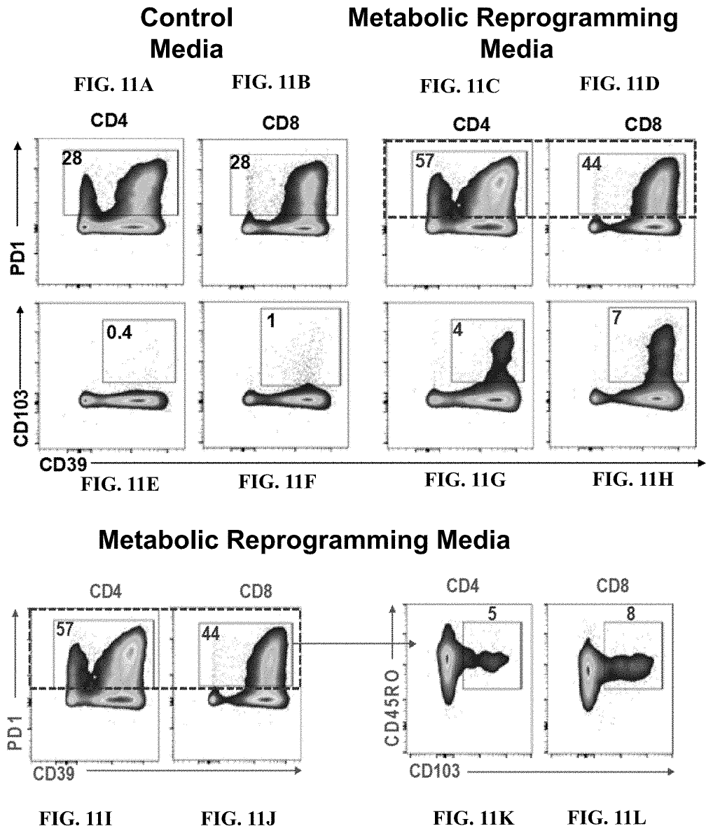

Expression of CD103 in TILs is reported to be correlated with TIL infiltration in tumors with high mutation antigen burden. In addition to CD39 and PD1 expression, our data demonstrated that expansion of TILs in MRM increased the number of PD1⁺CD39⁺CD103⁺ cells (FIGS. 11A-11L) while also increasing expression of stemness-associated genes. Therefore, culture of TILs in MRM may be sufficient to increase the number of cells with both tumor-reactivity and higher proportion of stem-like cells in the resulting expanded cell products.

The data presented herein demonstrate that expansion of TILs in MRM generates cell populations with not only increased stemness, but also increased expression of several cell surface markers associated with tumor reactivity. The methods described herein using MRM produce TILs that have characteristics and properties of an improved therapeutic product.

### Example 5. Methods of Treatment

TILs that are used for infusion are derived from a pateint's tumor excised from primary or metastatic tuomors or lymph nodes. TIL cultures are initiated by plating the small tumor fragments (˜1-10 mm³) in 24 well plates containing MRM as described above in Example 1. These fragments are grown until about 2×10⁶ to about 10×10⁶ cells/tumor fragment are obtained (typically about 2-3 weeks). The resulting cells from all the fragments are pooled and plated at a density of 2×10⁶/well for T cell stimulation, e.g., by adding TRANSACT™, and optionally CD27 agonist, 41BB agonist, and/or OX-40 agonist. Cells are maintained in culture until about 5×10⁷ to about 20×10⁷ cells are obtained. These cells are further stimulated, e.g., using TRANSACT™ and optionally CD27 agonist, 41BB agonist, and/or OX-40 agonist to achieve about 1000-2000 fold increase in the number of cells (˜1-150×10⁹ TILs) for the infusion product.

Prior to administration of the TIL infusion product, patients are administered a lymphodepletion treatment, e.g., comprising cyclophosphamide and fludarabine. In addition to TIL infusion, patients may also receive an anti-PD1 checkpoint inhibitor (e.g., pembrolizumab or nivolumab) after infusion of TILs cultured as disclosed herein.

### Example 6. Analysis of Clonal Diversity

To assess the clonal diversity of TILs cultured in MRM, according to the methods disclosed above, tumor fragments and TILs obtained from the tumor fragments were cultured in either control media or metabolic reprogramming media (MRM). Total genomic DNA was isolated from tumor and TIL samples using DNeasy Blood and Tissue Kit (QIAGEN) and sequenced using Immuno-seq for TCRβ and CDR3 regions (Adaptive Biotechnologies, Seattle, Wash.).

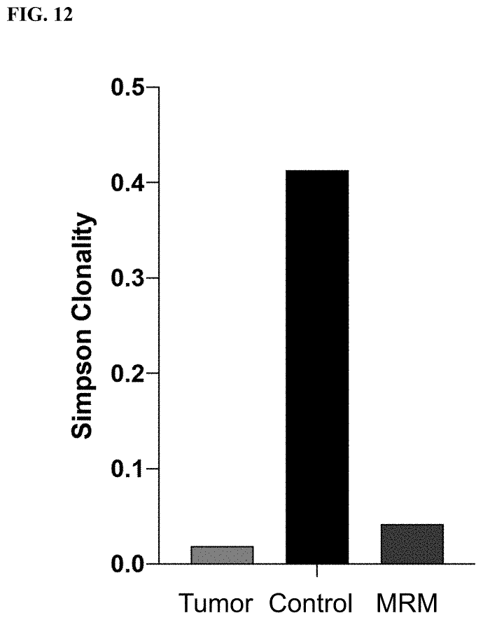

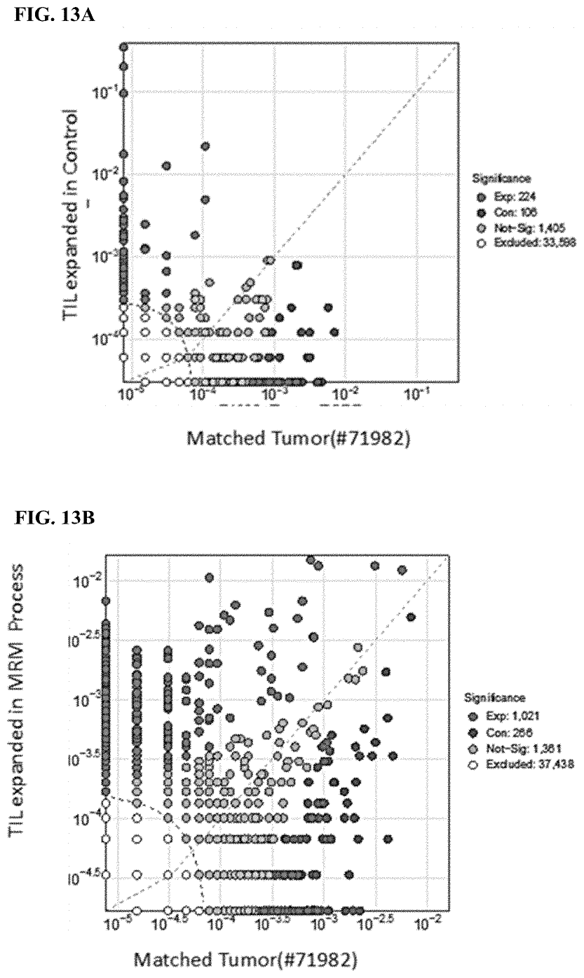

The diversity metrics of the samples were assessed by Simpsons clonality where pi is the proportional abundance of clone i in a given sample. Simpsons clonality and productive rearrangements were examined in tumor fragments (“tumor”), TILs cultured in control media (“control”), and TILs cultured in MRM (“MRM”) (FIG. 12). TILs cultured in MRM (post-expansion) displayed Simpsons clonality of approximately 0.04, which indicates high clonal diversity, similar to what is seen in the tumor fragments (FIG. 12). Maintenance of TCRβ diversity represent preservation of TIL clonotypes that infiltrated the tumors and are tumor reactive. In contrast, TILs cultured in control media significantly lose clonal diversity and displayed Simpsons clonality of approximately 0.4 (i.e., a significantly less diverse clonality, indicating that many tumor reactive clones are lost in the culturing process using control media) (FIG. 12).

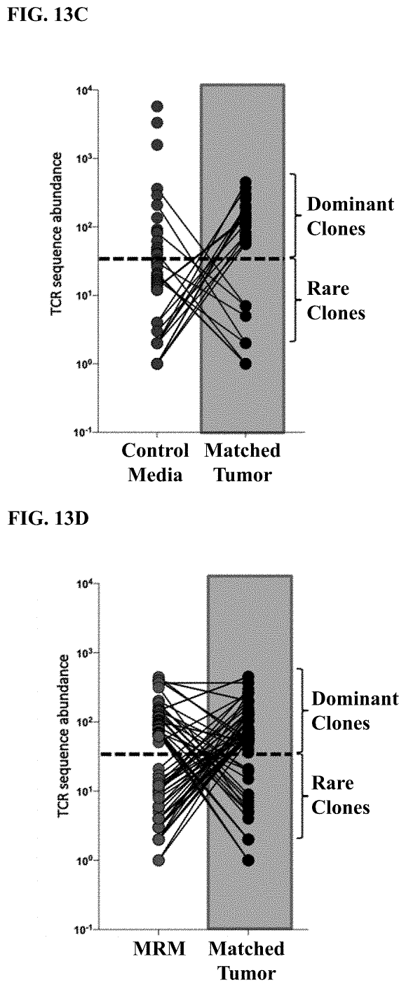

Differential abundance (DA) plots were generated using the data presented in FIG. 12 (ImmunoSeq, Adaptive Biotechnolgies). Such DA analysis calculates TCR overlap (see, e.g., Emerson et al, J. Path. (2013), which is incorporated by reference herein in its entirety), Morisita's Index, and the Jaccard Index for any pair of samples. These plots show that the differential abundance of clones are significantly different between tumor vs TILs expanded in control media (FIG. 13A) as compared to tumor vs TILs expanded in MRM (FIG. 13B).

The TIL repertoire present in tumor fragments were represented approximately 4-fold more in TILs cultured in MRM process compared to TILs cultured in control media. When expanded, TILs are typically outgrown by infrequent clonotypes with preferential proliferative potential (these clones are shown expanded along the y-axis in FIGS. 13A-13B). In contrast, TILs cultured in MRM showed significantly better preservation and expansion of a wider repertoire of clonotypes that are present in the tumor. The preservation and expansion of clonal diversity of the TIL population is critical for optimization for effective therapy.

To further analyze TIL clonal diversity, the top 50 most dominant prevalent TCRs in initial tumor digests were compared to TILs expanded in control media versus MRM. Culture in MRM preserves both dominant (i.e. prevalent) and rare TIL clonotypes. The density of lines in FIG. 13D show that the majority of T cell clones expanded in MRM recognize tumor antigens where they do not in T cell clones cultured in control media (FIG. 13C). The majority of dominant tumor clones are recognized by T cell clones expanded in MRM, as indicated by the connections above the dotted line of FIG. 13D, as compared to the T cell clones cultured in control media (FIG. 13C). Of the top 50 dominant tumor TCRs, 2% are preserved in TILs cultured in control media (FIG. 13C) as compared to 57% in TILs cultured in MRM (FIG. 13D). These data demonstrate that MRM expanded TILs retain a more faithful representation of initial tumor TCR clonotypes than TILs expanded in control media.

### Example 7. MRM Preserves Tumor Reactivity of TILs

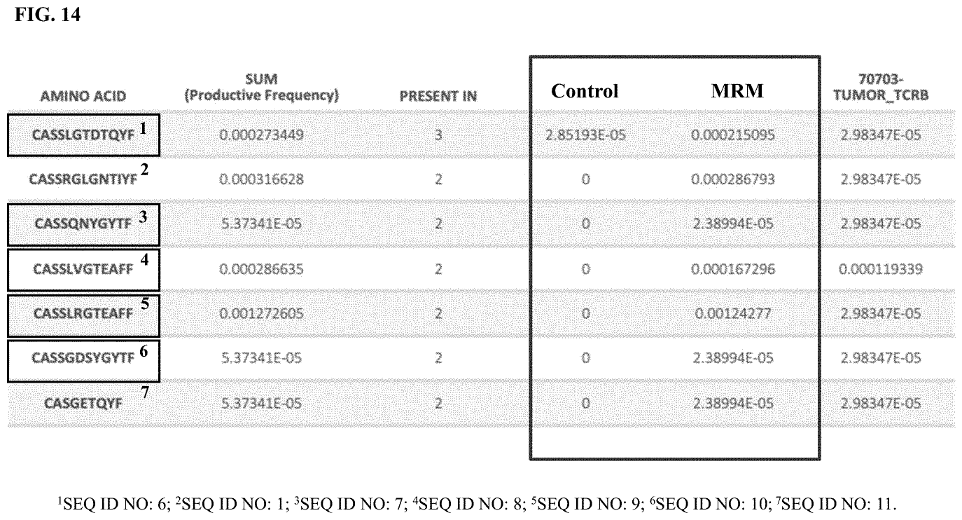

The ability of TILs generated using the methods described herein to preserve KRAS mutant reactivity was evaluated. TILs expanded in control media or MRM were mixed with autologous dendritic cells (DCs) that had been pulsed with mutant KRAS peptide. After 10 days total genomic DNA was isolated from the TILs using DNeasy Blood and Tissue Kit (Qiagen) and the TCR-Vb CDR3 motif was sequenced and analyzed using Immuno-seq for TCRβ and CDR3 regions (Adaptive Biotechnologies, Seattle, Wash.). FIG. 14 shows that TILs cultured in MRM preserve KRAS mutant reactivity, whereas TILs cultured in control media are not able to do so. Specifically, TILs cultured in MRM shared similar CDR3 amino acid motifs as seen in TILs from the tumor fragment. These were not detected in TILs cultured in control media. Thus, the data show that culture in MRM preserves TIL clonotypes that are represented in the original tumor fragment.

### Example 8. TILs Generated Using MRM Demonstrate Increased Tumor Recognition and Tumor Killing

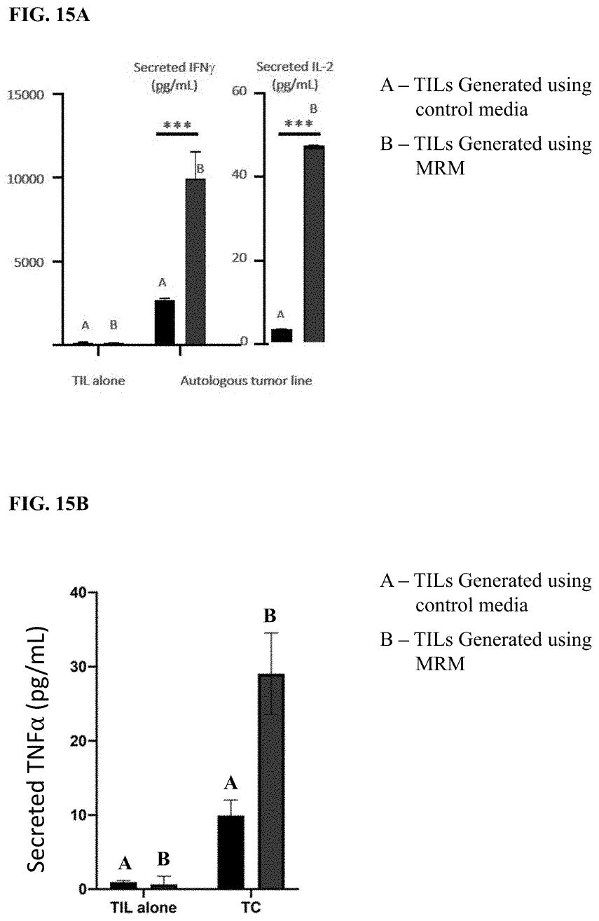

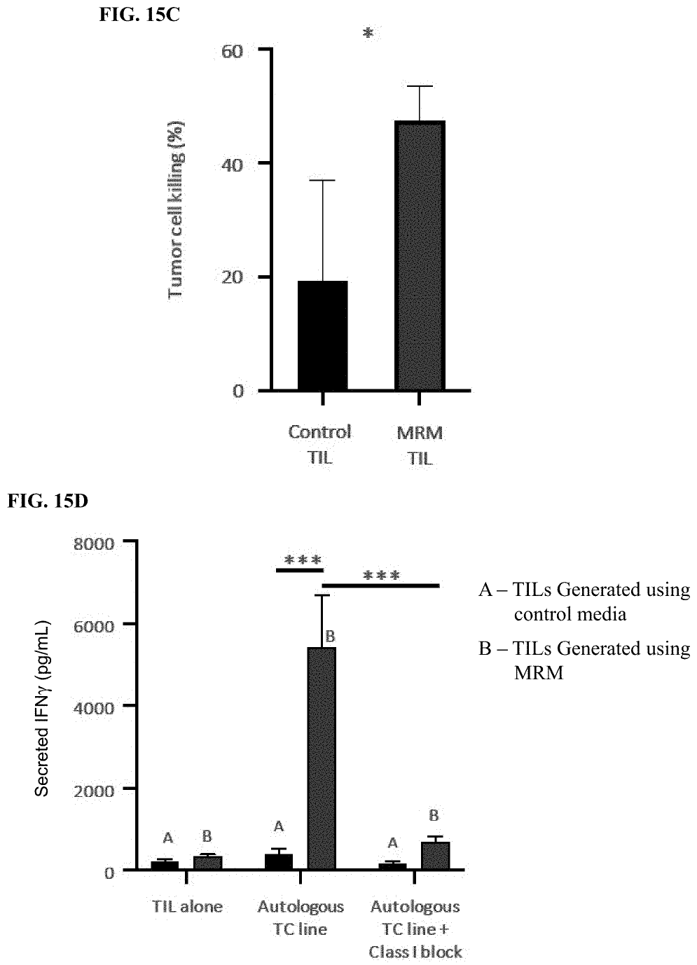

TILs obtained from a melanoma tumor were obtained and expanded with either control media or MRM as described above. An autologous tumor cell line was derived from the same melanoma sample as the TILs. Briefly, the melanoma cell line was generated by enzymatically digesting a fragment of melanoma (using collagenase and DNAse), plating the resulting single-cell suspension, followed by serial passaging of the outgrowing cells. The ability of the TILs derived from the melanoma tumor to generate inflammatory cytokines in response to the melanoma derived cell line was assessed. TILs generated using either control media or MRM were co-cultured with the autologous melanoma cell line for 24 hours. 100,000 tumor cells from the autologous melanoma cell line were plated in a 48 well plate in 300 ul of complete RPMI media (w/ 10% FBS) and allowed to incubate overnight. The next day, the supernatant was removed and TILs were added (some TILs were plated in the absence of tumor cells as a control). After 24 hrs, media supernatant was collected from the wells and secreted interferon gamma and TNF-alpha was measured using MSD. In addition, TILs generated with MRM were incubated in control media overnight, and IL-2 secretion was assessed by MSD. These data show that TILs expanded using MRM exhibit significantly increased ability to respond to autologous tumor cells in terms of inflammatory cytokine production (FIGS. 15A-15B). Moreover, the TILs generated using MRM secrete significantly more IL-2 at steady state with no stimulation indicating that these cells are more fit.

The ability of the TILs derived from the melanoma tumor to kill the melanoma derived cell line was also assessed. The number of live tumor cells remaining after co-culture was assessed by detaching the tumor cells with trypsin and counting live cells by flow cytometry. Based on this live tumor cell count, percent tumor killing by the TILs (normalized to wells of tumor cells that did not experience TIL co-culture) was calculated. TILs generated using MRM show a significantly better ability to kill the tumor cells (FIG. 15C).

The above experiment was repeated as described above, however some wells were treated with the W6/32 antibody to block HLA Class I interactions. FIG. 15D demonstrates that the recognition of autologous tumor cells by TILs generated using MRM is dependent on HLA Class I interactions since blocking Class I eliminates the increase in interferon gamma production.

### Example 9. TIL Clearance of Tumor Cells Ex Vivo

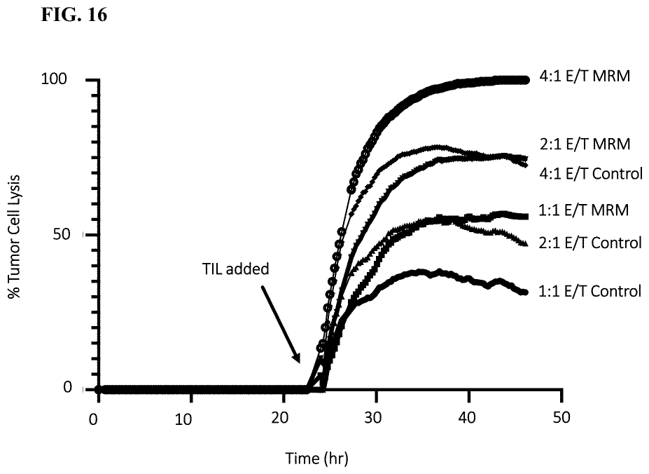

Patient autologous melanoma tumor cells were plated at 10,000 cells/well in the xCELLigence impedance assay well plate. The following day, matched TILs that were expanded in control media or MRM were added to the plate at various effector T cell (E) to tumor cell (T) ratios, including 1:1, 2:1, and 4:1. TIL clearance of tumor cells was monitored over time. FIG. 16 shows that TILs cultured in MRM displayed superior tumor cell lysis in an ex vivo assay as compared to TILs cultured in control media across a range of relative doses, with a 4:1 ratio of effector T cells cultured in MRM to tumor cells resulting in complete tumor eradication.

### Example 10. TIL Clearance of Tumor Cells Ex Vivo

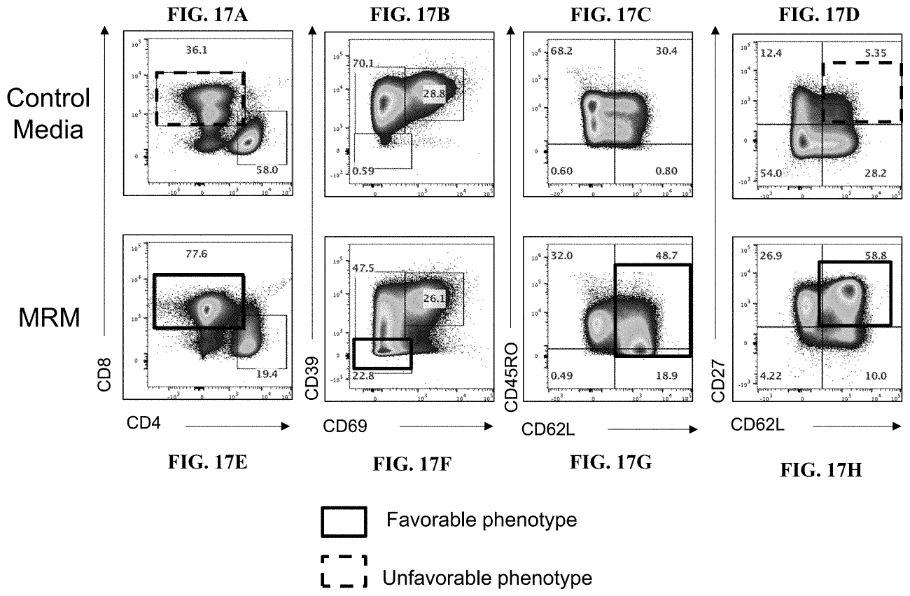

Non-small cell lung cancer (NSCLC) TILs produced following standard TIL expansion methods were compared to TIL produced in MRM, according to the methods disclosed herein. While the control process generated TILs highly enriched for CD8+ T cells, these cells were not enriched for a stemlike CD8+CD39−CD69− T cell population and expressed low levels of central memory markers and co-stimulatory receptor, CD27 (FIGS. 17A-17D). The control expansion process generated T cell products with enriched stemlike CD8+CD39−CD69− T cells and higher expression of co-stimulatory receptor CD27, but resulted in reduced expansion of CD8+ T cells. TILs cultured in MRM yield highly enriched populations of CD8+ T cells with enhanced abundance of CD8+CD39−CD69− stemlike T cells, central memory markers (CD45RO+CD62L+), and markedly higher expression of co-stimulatory receptor CD27 (FIGS. 17E-17H). Collectively these markers demonstrate attributes of stem-like cells that are correlated with clinical responses.

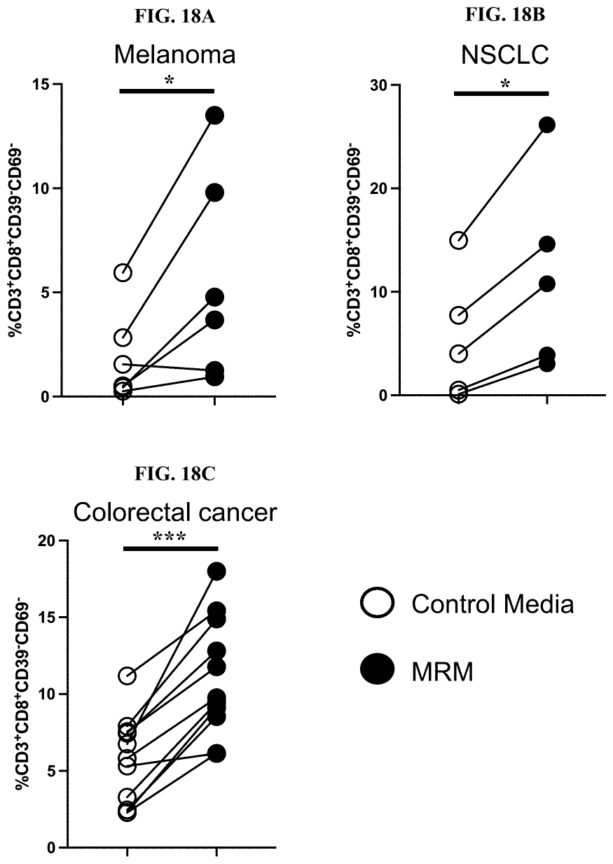

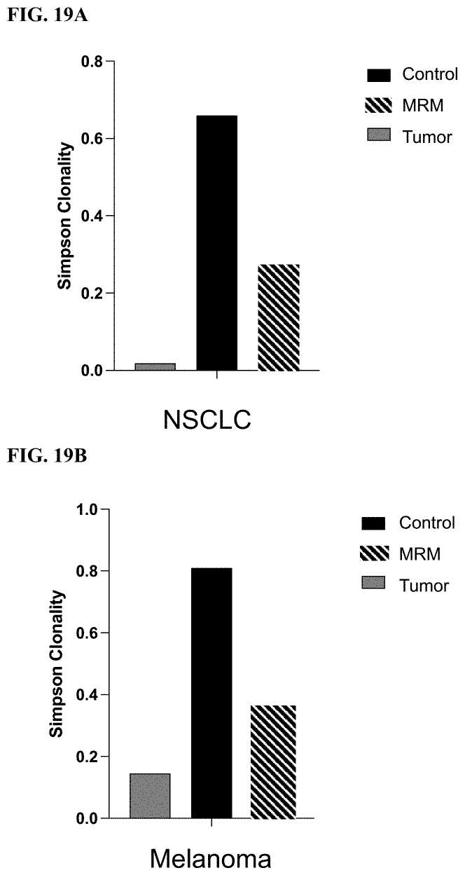

TIL products with enhanced stemlike properties, including retention of key co-stimulatory receptors CD27, elongated telomeres, memory cell phenotypes and the presence of populations of cells enriched with CD39⁻/CD69⁻ CD8⁺ T cells, are associated with improved clinical responses. Additional differentiation state-linked characteristics of T cells, including ability to secrete polyfunctional cytokines, and expression of co-stimulatory receptor CD28 have also been correlated with anti-tumor potency. TILs expanded in MRM exhibit favorable phenotypic attributes, including an increased abundance of CD8⁺CD39⁻CD69⁻ T cells when compared with control TIL expansion process in melanoma, NSCLC, and colorectal cancers (FIGS. 18A-18C).

### Example 11. TIL Culture in MRM

The data presented herein show that culturing TILs in a culture medium comprising increased potassium generates a TIL population that has increased expansion of CD8⁺ TILs and increased stemness, relative to TILs cultured in medium having lower levels of potassium (e.g., less than about 40 mM potassium ion, e.g., 5 mM potassium ion). Further, data suggest that exceedingly high levels of potassium (e.g., greater than about 80 mM, greater than about 90 mM, or greater than about 100 mM potassium ion) in the culture medium can lead to decreased TIL yield, likely due to decreased TIL growth and expansion, data not shown. To further characterize the effects of MRM on TIL culture, (1) TILs will be cultured in control medium (an isotonic RPMI formulation comprising 55 mM potassium ion) during an initial expansion stage followed by culture in MRM medium comprising between 50-70 mM (e.g., 50 mM, 55 mM, 60 mM, 65 mM, or 70 mM) potassium ion and between 50-70 mM (e.g., 50 mM, 55 mM, 60 mM, 65 mM, or 70 mM) NaCl during a final expansion stage; (2) TILs will be cultured in MRM medium comprising between 50-70 mM (e.g., 50 mM, 55 mM, 60 mM, 65 mM, or 70 mM) potassium ion and between 50-70 mM (e.g., 50 mM, 55 mM, 60 mM, 65 mM, or 70 mM) NaCl during an initial expansion stage followed by culture in control medium during a final expansion stage; and (3) TILs will be cultured in MRM medium comprising between 50-70 mM (e.g., 50 mM, 55 mM, 60 mM, 65 mM, or 70 mM) potassium ion and between 50-70 mM NaCl during an initial expansion stage followed by culture in MRM medium comprising between 50-70 mM (e.g., 50 mM, 55 mM, 60 mM, 65 mM, or 70 mM) potassium ion and between 50-70 mM (e.g., 50 mM, 55 mM, 60 mM, 65 mM, or 70 mM) NaCl during a final expansion stage. In all experiments, initial culture conditions will further comprise 6000 IU/mL IL-2, and final culture conditions will further comprise 3000 IU/mL IL-2. TILs cultured according to this method will be characterized for expression of stemness and effector markers, cytotoxicity, and yield.

### Example 12 Analysis of Clonal Diversity

To assess the clonal diversity of TILs cultured in MRM, according to the methods disclosed above, tumor fragments and TILs obtained from the tumor fragments were cultured in either control media or metabolic reprogramming media (MRM). Total genomic DNA was isolated from tumor and TIL samples using DNeasy Blood and Tissue Kit (QIAGEN) and sequenced using Immuno-seq for TCRβ and CDR3 regions (Adaptive Biotechnologies, Seattle, Wash.).

The diversity metrics of the non-small cell lung cancer (NSCLC) and melanoma samples were assessed by Simpsons clonality √Σpi2 where pi is the proportional abundance of clone i in a given sample. Simpsons clonality and productive rearrangements were examined in tumor fragments (“tumor”), TILs cultured in control media (“control”), and TILs cultured in MRM (“MRM”) for NSCLC and melanoma (FIG. 19A and FIG. 19B, respectively). TILs cultured in MRM (post-expansion) displayed Simpsons clonality of approximately 0.28 for NSCLC and approximately 0.38 for melanoma, which indicates high clonal diversity (FIG. 19A and FIG. 19B). Maintenance of TCRβ diversity represent preservation of TIL clonotypes that infilatrated the tumors and are tumor reactive. In contrast, TILs cultured in control media significantly lose clonal diversity and displayed Simpsons clonality of approximately 0.65 for NSCLC and approximately 0.8 for melanoma (i.e., a significantly less diverse clonality, indicating that many tumor reactive clones are lost in the culturing process using control media) (FIG. 19A and FIG. 19B).

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

The contents of all cited references (including literature references, U.S. or foreign patents or patent applications, and websites) that are cited throughout this application are hereby expressly incorporated by reference as if written herein in their entireties for any purpose, as are the references cited therein. Where any inconsistencies arise, material literally disclosed herein controls.

1. A method of culturing tumor infiltrating lymphocytes (TILs) ex vivo or in vitro comprising placing a heterogeneous population of TILs in a metabolic reprogramming medium (“MRM”) comprising potassium ion at a concentration of about 30 mM to about 100 mM.

2-5. (canceled)

6. The method of claim 1, wherein the heterogeneous population of TILs is obtained from one or more tumor sample obtained from a subject, and wherein the tumor sample is subjected to an initial TIL culture.

7. (canceled)

8. The method of claim 6, wherein the initial TIL culture comprises culturing the tumor sample in the MRM and IL-2.

9. (canceled)

10. The method of claim 8, wherein the MRM further comprises IL-7, IL-15, IL-21, or any combination thereof during the initial TIL culture.

11-14. (canceled)

15. The method of claim 6, wherein the TILs are stimulated following the initial TIL culture.

16-17. (canceled)

18. The method of claim 6, wherein the tumor sample is fragmented prior to culturing.

19-20. (canceled)

21. The method of claim 1, wherein the MRM further comprises sodium ion, calcium ion, glucose, a cell expansion agent, or any combination thereof.

22-25. (canceled)

26. The method of claim 1, wherein the concentration of potassium ion is at least about 60 mM.

27-31. (canceled)

32. The method of claim 21, wherein:
(a) the concentration of the sodium ion is from about 25 mM to about 100 mM,
(b) the concentration of glucose is from about 10 mM to about 25 mM,
(c) the concentration of calcium ion is from about 0.4 mM to about 2.5 mM, or
(d) any combination of (a)-(c).

33-34. (canceled)

35. The method of claim 32, wherein the concentration of the sodium ion is about 55 mM, about 60 mM, or about 65 mM.

36-45. (canceled)

46. The method of claim 1, wherein the MRM comprises about 40 mM to about 90 mM potassium ion and about 40 mM to about 80 mM sodium ion;
(ii) about 10 mM to about 24 mM glucose;
(iii) about 0.5 mM to about 2.8 mM calcium ion; or
(iv) any combination of (i)-(iii).

47. A method of expanding TILs obtained from a human subject comprising:
a. culturing the TILs in an initial TIL culture media;
b. culturing the TILs in a secondary TIL culture media;
c. culturing the TILs in a third (or final) TIL culture media,
wherein the initial TIL culture media, the secondary TIL expansion media, and/or the third TIL expansion media are MRM.

48-52. (canceled)

53. The method of claim 47, wherein the TILs are cultured in the initial TIL culture media until cell yield in the initial culture reaches at least about 1×10⁵ cells per fragment.

54. The method claim 47, wherein the TILs are stimulated with:
(i) a CD3 agonist,
(ii) a CD28 agonist,
(iii) a CD27 agonist,
(iv) a 4-1BB agonist, or
(v) any combination of (i)-(iv)
in or prior to the secondary TIL culture media in (b), in the third TIL culture media (c), or in both (b) and (c).

55-57. (canceled)

58. The method of claim 47, wherein the TILs are cultured in the secondary culture media until cell yield reaches at least about 1×10⁷ cells.

59-62. (canceled)

63. The method of claim 1, wherein:
(i) the TILs exhibit increased expression of TCF7 following culture in the MRM, relative to TCF7 expression in a population of TILs following culture in a control medium that is not hyperkalemic;
(ii) the population of TILs comprises an increased proportion of CD8⁺ CD62L⁺ TILs following culture in the MRM, relative to the proportion of CD8⁺ CD62L⁺ TILs following culture in a control medium that is not hyperkalemic;
(iii) the population of TILs comprises an increased proportion of CD8⁺ PD1⁺ TILs following culture in the MRM, relative to the proportion of CD8⁺ PD1⁺ TILs following culture in a control medium that is not hyperkalemic;
(iv) the population of TILs has an increased clonal diversity after being placed in the MRM, as compared to the clonal diversity of a population of TILs placed in a control medium;
(v) the population of TILs comprises an increased proportion of CD39−/CD69− TILs following culture in the MRM, relative to the proportion of CD39−/CD69− TILs following culture in a control medium; or
(vi) any combination of (i)-(v).

64-65. (canceled)

66. A composition of immune cells, comprising one or more CD8+ TIL cultured according to the method of claim 1.

67. (canceled)

68. A composition comprising a population of immune cells, wherein at least about 30% of the immune cells are CD8+ TILs.

69-81. (canceled)

82. A method of treating a cancer in a subject in need thereof, comprising administering a population of TILs to the subject, wherein the population of TILs are cultured according to the method of claim 1.

83-151. (canceled)

152. The method of claim 1, wherein the MRM comprises NaCl, wherein total concentration of potassium ion and NaCl is between 110 mM and 140 mM.

153-155. (canceled)


SEQUENCE LISTING (7 sequences)

SEQ ID NO: 1 - protein, 12 aa, Artificial Sequence
  KRAS Peptide Sequence
CASSLGTDTQYF

SEQ ID NO: 2 - protein, 13 aa, Artificial Sequence
  KRAS Peptide Sequence
CASSRGLGNTIYF

SEQ ID NO: 3 - protein, 11 aa, Artificial Sequence
  KRAS Peptide Sequence
CASSQNYGYTF

SEQ ID NO: 4 - protein, 12 aa, Artificial Sequence
  KRAS Peptide Sequence
CASSLVGTEAFF

SEQ ID NO: 5 - protein, 12 aa, Artificial Sequence
  KRAS Peptide Sequence
CASSLRGTEAFF

SEQ ID NO: 6 - protein, 12 aa, Artificial Sequence
  KRAS Peptide Sequence
CASSGDSYGYTF

SEQ ID NO: 7 - protein, 9 aa, Artificial Sequence
  KRAS Peptide Sequence
CASGETQYF